SEQUENCE LISTING (162 sequences)

SEQ ID NO: 1 - DNA, 379 bp, Artificial Sequence
  SG2
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagctgggttcccttcacctgctgctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 2 - DNA, 376 bp, Artificial Sequence
  SG3
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccgccagcaacgccagcgtctgggagtgctgcagcacgggcagctgggttcccttcacctgctgctgacgcccgcacaccgttccaccgatgagaggtgacagtcccatggcatccatccttgagctccaggacctggaggtcgagcgcgccagctcggccgccgactccgactggagcctgtgggagtgctgtagcacgggaagcctgttcgcctgctgctgaacagcgct

SEQ ID NO: 3 - DNA, 220 bp, Artificial Sequence
  SG4
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccgacagcaacgccagcgtctgggagtgctgcagcacgggcagctgggttcccttcacctgctgctgaacagcgct

SEQ ID NO: 4 - DNA, 214 bp, Artificial Sequence
  SG5
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgtccgactggagcctgtgggagtgctgtagcacgggaagcctgttcgcctgctgctgaacagcgct

SEQ ID NO: 5 - DNA, 376 bp, Artificial Sequence
  SG6
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgtccgactggagcctgtgggagtgctgtagcacgggaagcctgttcgcctgctgctgacgcccgcacaccgttccaccgatgagaggtgacagtcccatggcgtcgatcctggaactccagaacctggacgtcgagcacgcccgcggcgagaaccgcagcaacgccagcgtctgggagtgctgcagcacgggcagctgggttcccttcacctgctgctgaacagcgct

SEQ ID NO: 6 - DNA, 355 bp, Artificial Sequence
  SG7
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgtgcagcacgggcagctgggttcccttcacctgctgctgacgcccgcacaccgttccaccgatgagaggtgacagtcccatggcgtcgatcctggaactccagaacctggacgtcgagcacgcccgcggcgagaaccgcatgtccgactggagcctgtgggagtgctgtagcacgggaagcctgttcgcctgctgctgaacagcgct

SEQ ID NO: 7 - DNA, 373 bp, Artificial Sequence
  SG8
ccctgggcggccaccccctgagactccccttcctccatccggaggaaggggcgagcacgcgaccccgggggaggaggtgaacatccaccatggcacttctcgacctgcagacgatggaagccgacgagacgaccggtaccggcgggcccagctccctgagcgtgctgtcctgtgtgagcgcggccagcatcacgctctgcctctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 8 - DNA, 403 bp, Artificial Sequence
  SG9
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagctgggttcccttcacctgctgcagcaacgccagcgtctgggagtgctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 9 - DNA, 439 bp, Artificial Sequence
  SG10
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagctgggttcccttcacctgctgcagcaacgccagcgtctgggagtgcgccagcacgggcagctgggttcccttcacctgcgcctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 10 - DNA, 376 bp, Artificial Sequence
  SG11
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccgacagcaacgccagcgtctgggagtgctgtagcacgggaagcctgttcgcctgctgctgacgcccgcacaccgttccaccgatgagaggtgacagtcccatggcgtcgatcctggaactccagaacctggacgtcgagcacgcccgcggcgagaaccgctccgactggagcctgtgggagtgctgcagcacgggcagctgggttcccttcacctgctgctgaacagcgct

SEQ ID NO: 11 - DNA, 382 bp, Artificial Sequence
  SG12
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagctggtgctgcagcacgggcagctgggttcccttcacctgctgctgacgcccgcacaccgttccaccgatgagaggtgacagtcccatggcgtcgatcctggaactccagaacctggacgtcgagcacgcccgcggcgagaaccgcagcaacgccagcgtcgtcgcctgggagtgctgcagcacgggcagctgggttcccttcacctgctgctgaacagcgct

SEQ ID NO: 12 - DNA, 379 bp, Artificial Sequence
  SG13
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagctggacctgctgctgacgcccgcacaccgttccaccgatgagaggtgacagtcccatggcgtcgatcctggaactccagaacctggacgtcgagcacgcccgcggcgagaaccgcagcaacgccagcgtctgggagtgctgcagcacgggcagctgggttgccgtccccttcacctgctgctgaacagcgct

SEQ ID NO: 13 - DNA, 382 bp, Artificial Sequence
  SG14
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgcgccagcacgggcagctgggttcccttcacctgcgcctgacgcccgcacaccgttccaccgatgagaggtgacagtcccatggcgtcgatcctggaactccagaacctggacgtcgagcacgcccgcggcgagaaccgctgcaacgccgtcagctgggagagctgcagcacgggcagctgggttcccttcacctgctgctgaacagcgct

SEQ ID NO: 14 - DNA, 394 bp, Artificial Sequence
  SG15
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtcgtcagcgcctgggagtgctgcagcacgggcagctgggttcccttcacctgctgctgacgcccgcacaccgttccaccgatgagaggtgacagtcccatggcgtcgatcctggaactccagaacctggacgtcgagcacgcccgcggcgagaaccgcagcaacgccagcgtctgggagtgctgcagcacgggcagctgggttagccccttcacctgctgctgaacagcgct

SEQ ID NO: 15 - DNA, 373 bp, Artificial Sequence
  SG16
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcagcggcagctgggttcccttcacctgctgctgacgcccgcacaccgttccaccgatgagaggtgacagtcccatggcgtcgatcctggaactccagaacctggacgtcgagcacgcccgcggcgagaaccgcagcaacgccagctgctgcagcacgggcagctgggttcccttcacctgctgctgaacagcgct

SEQ ID NO: 16 - DNA, 370 bp, Artificial Sequence
  SG17
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagctgggttcccttcagctgctgctgacgcccgcacaccgttccaccgatgagaggtgacagtcccatggcgtcgatcctggaactccagaacctggacgtcgagcacgcccgcggcgagaaccgcagcaacgccagcgtctgggagtgctgcagcacgggcagctggtgctgctgaacagcgct

SEQ ID NO: 17 - DNA, 370 bp, Artificial Sequence
  SG18
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagctccctgagcgtgctgtcctgtgtgagcgcggccagcatcacgctctgcctctgacgcccgcacaccgttccaccgatgagaggtgacagtcccatggcgtcgatcctggaactccagaacctggacgtcgagcacgcccgcggcgagaaccgcagctccctgagcgtgctgtcctgttgcagcgcggccagcatcacgctctgctgctgaacagcgct

SEQ ID NO: 18 - DNA, 385 bp, Artificial Sequence
  SG19
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgaccggcagccgcgcgagcctcctgctctgcggcgacagcagcctgagcatcaccacctgtaactgacgcccgcacaccgttccaccgatgagaggtgacagtcccatggcgtcgatcctggaactccagaacctggacgtcgagcacgcccgcggcgagaaccgcaccggcagccgcgcgagcctcctgctctgctgcagcagcctgagcatcaccacctgttgctgaacagcgct

SEQ ID NO: 19 - DNA, 376 bp, Artificial Sequence
  SG20
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgcatgtgcagcacgggcagctgggttcccttcacctgctgctgacgcccgcacaccgttccaccgatgagaggtgacagtcccatggcgtcaatattggaactccagaacctggacgtcgagcacgcccgcggcgagaaccgctccgactggagcctgtgggagtgcatgagcacgggaagcctgttcgcctgctgaacagcgct

SEQ ID NO: 20 - DNA, 376 bp, Artificial Sequence
  LabA2_T11A
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgtccgactggagcctgtgggagtgctgtagcgccggaagcctgttcgcctgctgctgacgcccgcacaccgttccaccgatgagaggtgacagtcccatggcgtcgatcctggaactccagaacctggacgtcgagcacgcccgcggcgagaaccgcagcaacgccagcgtctgggagtgctgcagcacgggcagctgggttcccttcacctgctgctgaacagcgct

SEQ ID NO: 21 - DNA, 379 bp, Artificial Sequence
  LabA1_G12A
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacggccagctgggttcccttcacctgctgctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 22 - DNA, 379 bp, Artificial Sequence
  LabA1_W14A
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagcgccgttcccttcacctgctgctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 23 - DNA, 379 bp, Artificial Sequence
  LabA1_P16A
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagctgggttgccttcacctgctgctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 24 - DNA, 379 bp, Artificial Sequence
  LabA1_T18A
ccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccatgagcaacgccagcgtctgggagtgctgcagcacgggcagctgggttcccttcgcctgctgctgacgcccgcacaccgttccaccgatgagaggtgacagtcccatggcgtcaatattggaactccagaacctggacgtcgagcacgcccgcggcgagaaccgcatgtccgactggagcctgtgggagtgctgtagcacgggaagcctgttcgcctgctgctgaacagcgct

SEQ ID NO: 25 - DNA, 1393 bp, Artificial Sequence
  aac(3)IV with changed PasI (CCCTCGG) and Eco47III (AGCGGT) restriction sites
acgcgtcgattatctcgagaatgaccactgctgtgagcggtttgccttggcggacaggtggctcaaggagaagagccttcagaaggaaggtccagtcggtcatgcctttgctcggttgatccgctcccgcgacattgtggcgacagccctcggtcaactgggccgagatccgttgatcttcctgcatccgccagaggcgggatgcgaagaatgcgatgccgctcgccagtcgattggctgagctcatgagcggagaacgagatgacgttggaggggcaaggtcgcgctgattgctggggcaacacgtggagcggatcggggattgtctttcttcagctcgctgatgatatgctgacgctcaatgcgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggtggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccaggcacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttctaga

SEQ ID NO: 26 - DNA, 26 bp, Artificial Sequence
  LabA1_N2A_fw
ccgccatgagcgcggccagcgtctgg

SEQ ID NO: 27 - DNA, 26 bp, Artificial Sequence
  LabA1_N2A_rv
ccagacgctggccgcgctcatggcgg

SEQ ID NO: 28 - DNA, 25 bp, Artificial Sequence
  LabA1_V5A_fw
gcaacgccagcgcctgggagtgctg

SEQ ID NO: 29 - DNA, 25 bp, Artificial Sequence
  LabA1_V5A_rv
cagcactcccaggcgctggcgttgc

SEQ ID NO: 30 - DNA, 27 bp, Artificial Sequence
  LabA1_W6A_fw
caacgccagcgtcgcggagtgctgcag

SEQ ID NO: 31 - DNA, 27 bp, Artificial Sequence
  LabA1_W6A_rv
ctgcagcactccgcgacgctggcgttg

SEQ ID NO: 32 - DNA, 25 bp, Artificial Sequence
  LabA1_E7A_fw
cagcgtctgggcctgctgcagcacg

SEQ ID NO: 33 - DNA, 25 bp, Artificial Sequence
  LabA1_E7A_rv
cgtgctgcagcaggcccagacgctg

SEQ ID NO: 34 - DNA, 25 bp, Artificial Sequence
  LabA1_T11A_fw
gagtgctgcagcgcgggcagctggg

SEQ ID NO: 35 - DNA, 25 bp, Artificial Sequence
  LabA1_T11A_rv
cccagctgcccgcgctgcagcactc

SEQ ID NO: 36 - DNA, 30 bp, Artificial Sequence
  LabA1_V15A_fw
cacgggcagctgggcacccttcacctgctg

SEQ ID NO: 37 - DNA, 30 bp, Artificial Sequence
  LabA1_V15A_rv
cagcaggtgaagggtgcccagctgcccgtg

SEQ ID NO: 38 - DNA, 25 bp, Artificial Sequence
  LabA1_F17A_fw
gctgggttcccgccacctgctgctg

SEQ ID NO: 39 - DNA, 25 bp, Artificial Sequence
  LabA1_F17A_rv
cagcagcaggtggcgggaacccagc

SEQ ID NO: 40 - DNA, 25 bp, Artificial Sequence
  LabA2_D2A_fw
ccgccatgtccgcctggagcctgtg

SEQ ID NO: 41 - DNA, 25 bp, Artificial Sequence
  LabA2_D2A_rv
cacaggctccaggcggacatggcgg

SEQ ID NO: 42 - DNA, 28 bp, Artificial Sequence
  LabA2_W3A_fw
cgccatgtccgacgccagcctgtgggag

SEQ ID NO: 43 - DNA, 28 bp, Artificial Sequence
  LabA2_W3A_rv
ctcccacaggctggcgtcggacatggcg

SEQ ID NO: 44 - DNA, 25 bp, Artificial Sequence
  LabA2_L5A_fw
ccgactggagcgcgtgggagtgctg

SEQ ID NO: 45 - DNA, 25 bp, Artificial Sequence
  LabA2_L5A_rv
cagcactcccacgcgctccagtcgg

SEQ ID NO: 46 - DNA, 30 bp, Artificial Sequence
  LabA2_W6A_fw
cgactggagcctggccgagtgctgtagcac

SEQ ID NO: 47 - DNA, 30 bp, Artificial Sequence
  LabA2_W6A_rv
gtgctacagcactcggccaggctccagtcg

SEQ ID NO: 48 - DNA, 26 bp, Artificial Sequence
  LabA2_E7A_fw
ggagcctgtgggcctgctgtagcacg

SEQ ID NO: 49 - DNA, 26 bp, Artificial Sequence
  LabA2_E7A_rv
cgtgctacagcaggcccacaggctcc

SEQ ID NO: 50 - DNA, 26 bp, Artificial Sequence
  LabA2_G12A_fw
gctgtagcacggccagcctgttcgcc

SEQ ID NO: 51 - DNA, 26 bp, Artificial Sequence
  LabA2_G1A_rv
ggcgaacaggctggccgtgctacagc

SEQ ID NO: 52 - DNA, 25 bp, Artificial Sequence
  LabA2_L14A_fw
gcacgggaagcgcgttcgcctgctg

SEQ ID NO: 53 - DNA, 25 bp, Artificial Sequence
  LabA2_L14A_rv
cagcaggcgaacgcgcttcccgtgc

SEQ ID NO: 54 - DNA, 27 bp, Artificial Sequence
  LabA2_F15A_fw
cacgggaagcctggccgcctgctgctg

SEQ ID NO: 55 - DNA, 27 bp, Artificial Sequence
  LabA2_F15A_rv
cagcagcaggcggccaggcttcccgtg

SEQ ID NO: 56 - DNA, 26 bp, Artificial Sequence
  LabA1_N2insD_fw
ccgccatgagcgacaacgccagcgtc

SEQ ID NO: 57 - DNA, 26 bp, Artificial Sequence
  LabA1_N2insD_rv
gacgctggcgttgtcgctcatggcgg

SEQ ID NO: 58 - DNA, 25 bp, Artificial Sequence
  LabA1_N2insN_fw
ccatgagcaacaacgccagcgtctg

SEQ ID NO: 59 - DNA, 25 bp, Artificial Sequence
  LabA1_N2insN_rv
cagacgctggcgttgttgctcatgg

SEQ ID NO: 60 - DNA, 28 bp, Artificial Sequence
  LabA1_A3insA_fw
catgagcaacgccgccagcgtctgggag

SEQ ID NO: 61 - DNA, 28 bp, Artificial Sequence
  LabA1_A3insA_rv
ctcccagacgctggcggcgttgctcatg

SEQ ID NO: 62 - DNA, 25 bp, Artificial Sequence
  LabA1_A3del_fw
cgccatgagcaacagcgtctgggag

SEQ ID NO: 63 - DNA, 25 bp, Artificial Sequence
  LabA1_A3del_rv
ctcccagacgctgttgctcatggcg

SEQ ID NO: 64 - DNA, 29 bp, Artificial Sequence
  LabA1_G12insA_fw
gtgctgcagcacggccggcagctgggttc

SEQ ID NO: 65 - DNA, 29 bp, Artificial Sequence
  LabA1_G12insA_rv
gaacccagctgccggccgtgctgcagcac

SEQ ID NO: 66 - DNA, 25 bp, Artificial Sequence
  LabA1_G12del_fw
gtgctgcagcacgagctgggttccc

SEQ ID NO: 67 - DNA, 25 bp, Artificial Sequence
  LabA1_G12del_rv
gggaacccagctcgtgctgcagcac

SEQ ID NO: 68 - DNA, 28 bp, Artificial Sequence
  LabA1_W6insV_fw
caacgccagcgtcgtctgggagtgctgc

SEQ ID NO: 69 - DNA, 28 bp, Artificial Sequence
  LabA1_W6insV_rv
gcagcactcccagacgacgctggcgttg

SEQ ID NO: 70 - DNA, 28 bp, Artificial Sequence
  LabA1_W6insL_fw
caacgccagcgtcctgtgggagtgctgc

SEQ ID NO: 71 - DNA, 28 bp, Artificial Sequence
  LabA1_W6insL_rv
gcagcactcccacaggacgctggcgttg

SEQ ID NO: 72 - DNA, 25 bp, Artificial Sequence
  LabA1_V5del_fw
gagcaacgccagctgggagtgctgc

SEQ ID NO: 73 - DNA, 25 bp, Artificial Sequence
  LabA1_V5del_rv
gcagcactcccagctggcgttgctc

SEQ ID NO: 74 - DNA, 25 bp, Artificial Sequence
  LabA1_P16del_fw
gggcagctgggttttcacctgctgc

SEQ ID NO: 75 - DNA, 25 bp, Artificial Sequence
  LabA1_P16del_rv
gcagcaggtgaaaacccagctgccc

SEQ ID NO: 76 - DNA, 25 bp, Artificial Sequence
  LabA1_T18del_fw
ctgggttcccttctgctgctgacgc

SEQ ID NO: 77 - DNA, 25 bp, Artificial Sequence
  LabA1_T18del_rv
gcgtcagcagcagaagggaacccag

SEQ ID NO: 78 - DNA, 25 bp, Artificial Sequence
  LabA1_P16insV_fw
gcagctgggttgtccccttcacctg

SEQ ID NO: 79 - DNA, 25 bp, Artificial Sequence
  LabA1_P16insV_rv
caggtgaaggggacaacccagctgc

SEQ ID NO: 80 - DNA, 25 bp, Artificial Sequence
  LabA1_VP15del_fw
cacgggcagctggttcacctgctgc

SEQ ID NO: 81 - DNA, 25 bp, Artificial Sequence
  LabA1_VP15del_rv
gcagcaggtgaaccagctgcccgtg

SEQ ID NO: 82 - DNA, 25 bp, Artificial Sequence
  LabA1_S1T_fw
ggccgccatgacgaacgccagcgtc

SEQ ID NO: 83 - DNA, 25 bp, Artificial Sequence
  LabA1_S1T_rv
gacgctggcgttcgtcatggcggcc

SEQ ID NO: 84 - DNA, 28 bp, Artificial Sequence
  LabA1_S10T_fw
catgagcaacgccaccgtctgggagtgc

SEQ ID NO: 85 - DNA, 28 bp, Artificial Sequence
  LabA1_S10T_rv
gcactcccagacggtggcgttgctcatg

SEQ ID NO: 86 - DNA, 26 bp, Artificial Sequence
  LabA1_S13T_fw
gcagcacgggcacctgggttcccttc

SEQ ID NO: 87 - DNA, 26 bp, Artificial Sequence
  LabA1_S13T_rv
gaagggaacccaggtgcccgtgctgc

SEQ ID NO: 88 - DNA, 30 bp, Artificial Sequence
  LabA1_C9tga_fw
cgtctgggagtgctgaagcacgggcagctg

SEQ ID NO: 89 - DNA, 30 bp, Artificial Sequence
  LabA1_C9tga_rv
cagctgcccgtgcttcagcactcccagacg

SEQ ID NO: 90 - DNA, 27 bp, Artificial Sequence
  LabA1_S4A_fw
catgagcaacgccgccgtctgggagtg

SEQ ID NO: 91 - DNA, 27 bp, Artificial Sequence
  LabA1_S4A_rv
cactcccagacggcggcgttgctcatg

SEQ ID NO: 92 - DNA, 33 bp, Artificial Sequence
  LabA1_S13A_fw
gtgctgcagcacgggcgcctgggttcccttcac

SEQ ID NO: 93 - DNA, 33 bp, Artificial Sequence
  LabA1_S13A_rv
gtgaagggaacccaggcgcccgtgctgcagcac

SEQ ID NO: 94 - DNA, 27 bp, Artificial Sequence
  LabA1_V5T_fw
gagcaacgccagcacctgggagtgctg

SEQ ID NO: 95 - DNA, 27 bp, Artificial Sequence
  LabA1_V5T_rv
cagcactcccaggtgctggcgttgctc

SEQ ID NO: 96 - DNA, 28 bp, Artificial Sequence
  LabA1_V15S_fw
cacgggcagctggtcccccttcacctgc

SEQ ID NO: 97 - DNA, 28 bp, Artificial Sequence
  LabA1_V15S_rv
gcaggtgaagggggaccagctgcccgtg

SEQ ID NO: 98 - DNA, 30 bp, Artificial Sequence
  LabA1_W6Y_fw
caacgccagcgtctacgagtgctgcagcac

SEQ ID NO: 99 - DNA, 30 bp, Artificial Sequence
  LabA1_W6Y_rv
gtgctgcagcactcgtagacgctggcgttg

SEQ ID NO: 100 - DNA, 27 bp, Artificial Sequence
  LabA1_A3H_fw
gccatgagcaaccacagcgtctgggag

SEQ ID NO: 101 - DNA, 27 bp, Artificial Sequence
  LabA1_A3H_rv
ctcccagacgctgtggttgctcatggc

SEQ ID NO: 102 - DNA, 25 bp, Artificial Sequence
  LabA1_E7R_fw
ccagcgtctggcggtgctgcagcac

SEQ ID NO: 103 - DNA, 25 bp, Artificial Sequence
  LabA1_E7R_rv
gtgctgcagcaccgccagacgctgg

SEQ ID NO: 104 - DNA, 25 bp, Artificial Sequence
  LabA1_C20insA_fw
ccttcacctgcgcctgctgacgccc

SEQ ID NO: 105 - DNA, 25 bp, Artificial Sequence
  LabA1_C20insA_rv
gggcgtcagcaggcgcaggtgaagg

SEQ ID NO: 106 - DNA, 25 bp, Artificial Sequence
  LabA1_S10insA_fw
gggagtgctgcgccagcacgggcag

SEQ ID NO: 107 - DNA, 25 bp, Artificial Sequence
  LabA1_S10insA_rv
ctgcccgtgctggcgcagcactccc

SEQ ID NO: 108 - DNA, 29 bp, Artificial Sequence
  LabA1_C20del_fw
gttcccttcacctgctgacgcccgcacac

SEQ ID NO: 109 - DNA, 29 bp, Artificial Sequence
  LabA1_C20del_rv
gtgtgcgggcgtcagcaggtgaagggaac

SEQ ID NO: 110 - DNA, 25 bp, Artificial Sequence
  LabA1_S1insC_fw
cggccgccatgtgcagcaacgccag

SEQ ID NO: 111 - DNA, 25 bp, Artificial Sequence
  LabA1_S1insC_rv
ctggcgttgctgcacatggcggccg

SEQ ID NO: 112 - DNA, 26 bp, Artificial Sequence
  LabA1_C9insV_fw
gtctgggagtgcgtctgcagcacggg

SEQ ID NO: 113 - DNA, 26 bp, Artificial Sequence
  LabA1_C9insV_rv
cccgtgctgcagacgcactcccagac

SEQ ID NO: 114 - DNA, 25 bp, Artificial Sequence
  LabA1_C9insVN_fw
gggagtgcgtcaactgcagcacggg

SEQ ID NO: 115 - DNA, 25 bp, Artificial Sequence
  LabA1_C9insVN_rv
cccgtgctgcagttgacgcactccc

SEQ ID NO: 116 - DNA, 20 bp, Artificial Sequence
  attB_For
cggtctcgaagccgcggtgc

SEQ ID NO: 117 - DNA, 19 bp, Artificial Sequence
  attB_Rev
gcccgccgtgaccgtcgag

SEQ ID NO: 118 - DNA, 27 bp, Artificial Sequence
  p18mob_EcoRI_for
catctcgaattccgctcatgagctcag

SEQ ID NO: 119 - DNA, 29 bp, Artificial Sequence
  p18mob_EcoRI_rev
agttatcgagatctgcaggagctctttgg

SEQ ID NO: 120 - DNA, 20 bp, Artificial Sequence
  pSETproof_for
cgagccggaagcataaagtg

SEQ ID NO: 121 - DNA, 20 bp, Artificial Sequence
  pSETproof_rev
gcttggagcgaacgacctac

SEQ ID NO: 122 - DNA, 20 bp, Artificial Sequence
  STK_RTPCR_fw
agcagcaagtacgccgaacg

SEQ ID NO: 123 - DNA, 20 bp, Artificial Sequence
  STK_RTPCR_rv
gcgaagtggagctggttgag

SEQ ID NO: 124 - DNA, 20 bp, Artificial Sequence
  pMA_seq
tgtgctgcaaggcgattaag

SEQ ID NO: 125 - DNA, 34 bp, Artificial Sequence
  Infusion 1 (fw)
gtcgaggcggccctgggcggccaccccctgagac

SEQ ID NO: 126 - DNA, 39 bp, Artificial Sequence
  Infusion 2 (rv)
gcggcctcggtcagcgctgttcagcagcaggcgaacagg

SEQ ID NO: 127 - DNA, 36 bp, Artificial Sequence
  Infusion 3 (rv)
tcggcggcctcggtcagcgctgttcagcagcaggtg

SEQ ID NO: 128 - DNA, 35 bp, Artificial Sequence
  Infusion SG17 (rv)
cggcggcctcggtcagcgctgttcagcagcaccag

SEQ ID NO: 129 - DNA, 35 bp, Artificial Sequence
  Infusion SG18 (rv)
tcggcggcctcggtcagcgctgttcagcagcagag

SEQ ID NO: 130 - DNA, 35 bp, Artificial Sequence
  Infusion SG19 (rv)
ttcggcggcctcggtcagcgctgttcagcaacagg

SEQ ID NO: 131 - DNA, 35 bp, Artificial Sequence
  Infusion SG20 (rv)
cggcggcctcggtcagcgctgttcagcaggcgaac

SEQ ID NO: 132 - protein, 40 aa, Actinomadura namibiensis
MASILELQDLEVERASSAADSNASVWECCSTGSWVPFTCC

SEQ ID NO: 133 - protein, 20 aa, Actinomadura namibiensis
MASILELQDLEVERASSAAD

SEQ ID NO: 134 - protein, 20 aa, Actinomadura namibiensis
SNASVWECCSTGSWVPFTCC

SEQ ID NO: 135 - protein, 38 aa, Actinomadura namibiensis
MASILELQNLDVEHARGENRSDWSLWECCSTGSLFACC

SEQ ID NO: 136 - protein, 20 aa, Actinomadura namibiensis
MASILELQNLDVEHARGENR

SEQ ID NO: 137 - protein, 18 aa, Actinomadura namibiensis
SDWSLWECCSTGSLFACC

SEQ ID NO: 138 - protein, 21 aa, Actinomadura namibiensis
DSNASVWECCSTGSWVPFTCC

SEQ ID NO: 139 - DNA, 6400 bp, Actinomadura namibiensis
  misc_feature (1)..(2589): labKC gene
atggatctgcggtaccacgcctatgcgatggccgatcccgtcttttatgactcgccgtccagcgacacccgggagaccgacggctactccgacgacctccccctgccggacggctgggaacgtcggcgcgtgggggtctgggtgatgcagggccacgacgggttgaccatgcctgaccagggctggaagatccatgtgtcggccggtctggacaacgcctggcccgtcctcgaactggtcgccaaatactgcgtcgagcaggaaatgcctttcaagttcctgcgcagcagaaggacgttgctggcccgcagcagcaagtacgccgaacgcggcggcagcggaaagttcatcacgatctaccccgccgacgaaggcgctctggaaaagaccctccatgaactcggtggaatgctggaagggcagcccggcccctatatcttgagcgatctgcgctggcgttcggggccgctgttcgtgcgctacggcgctttcaaggagaaattctgccgggacgggcgcggcgagatggtgcccgcgatcgcgcgcccggacggcgtgctggtgcccgacgccagggacccggtgttccgggtgcccgcgtgggtggaggtgcccggcttcctgcgggaggccatcgacgcccgggagaacgggaccgtcgaggacttcccctaccggatcgagaaggcgctgcacttctccaacggcggcggcctctaccgcgccgtggacgagcgcaccggccgcagggtcctggtgaaggaggcgcggccgatggcgggcctggaccgcgccgaggacgacgccgtcgtccggctcgaacgcgagcacggcctgctgctccgcctcgccgacctggactgcatccccgacctggtcgagtaccggagctggtgggagcaccgcttcctcgtgcgggagtacgtcgagggcgagaccctcacccaccacatggtgcgccgcaacccgatgctgcactacggcgcgacgccgcaggaggtcgccggctacaccgagtgggcgctcggcgtcgtcgaccgggtggagagcgcgctcggccggctgcacgaacgcggcgtggtcttcggcgacctgcaccccggcaacatcatcgtccgggacgacgactccatcgtgttcgtcgacttcgagctggtcgccgaggcggaggaggcgacgcacccggcgctcggcgcgcccggctaccaggccccgcccgactacaccgggttcgccatcgaccgctacgcgctcggctgcatcaggctcgcggtcttcacctccctcaccgcgacgctgcactgggacgaccgcaaggtcgagcagttcctcgacgtgatctgcgagtccttcccgctgccgccggactacgccgaccggatccgccgcgacctcgcccgcccggcgccggcggacggcgcgccgccgatctggcgcgagccgacgcccgccacctggccggacacgcgggccgggatcgccgccgcgatcctcgacacggccacgcccgagcgcgccgaccggctcttcccgggcgacatcgagcagttcgcgaccagcgtcggtgggatcgggttcggccacggcgcggcgggcgtgctgtgggcgctggccgaggcgggcgccggccgcttccccgaccacgaggactgggtgcgggacgccgtcgccagggcgcaacggccgccgcccggcttctacgacggcgtcgcgggcgtcgcccacgtcctggaccggctcggccgcgccgacgaggcccgcgagctcatggagcacgcgcccgccgcgacgggggcgaccgacaacagcctctaccgggggctggcgggcatcggcctcaaccagctccacttcgcccgcgtcacgggcgaggcgtcgttcgccgcggcggccgaggagaccgccggccgggtggtcgcgaacctgcgccgcaagacggagggcgcctaccgggcgggcctgatgtacggctcctccggcccggccctgttcctcgtccggatgttcgaggcgacgggcgacggccactggctggacgaggccgaacgcgccctgcaccgcgagctggacgcctgcaagtggacgcagaaggacagcacgctccaggtcgacgagggctggcgggtccttccgtacgtcgccaccggcagcgtgggcatcggcatcgcgctgcacgagttcctgcggcaccgcccggcgccgcgcttcaccgaggcgcaggaggggatccggagggcggcggctcccgcgtacttcgtgcagagcggcctgctcaacggacggtccggcatcctcgcctacctgctgcacgtcggggccggccgggaggacccggtcgtccggacgcacctgcgcaacctcggctggcacgccgtcccgtaccccggccggggcgaggacgccccggcgcccggcgcgcggcggaccgcgttcatcggggaccagctgctgcgcctgtccatggacctcgccacgggctccgccggggtcctggccacggtcgaggcggccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccgacagcaacgccagcgtctgggagtgctgcagcacgggcagctgggttcccttcacctgctgctgacgcccgcacaccgttccaccgatgagaggtgacagtcccatggcgtcgatcctggaactccagaacctggacgtcgagcacgcccgcggcgagaaccgctccgactggagcctgtgggagtgctgtagcacgggaagcctgttcgcctgctgctgaacagcgctgaccgaggccgccgaagcggcacccatgtgaaacgaccgcccggggcggtggggactcccaccgccccgggcggaccaccatcagcgaggagcacgacaggaatgcacgccgaccggctgctcgtccaggcgctgcgcgccggccccgggtggacggccctcctggtcgccgcgacgctgctgaacgcggtgtgcgcgctcgccctgcccgccgcgatcggcgcggtcaccgacgcggtgctcgcccgcgacgcgggccgcggcgccggggccccgctgggctggctggcggcggcgctcggcggcgtggcgctgggcacggcgctgtcccggctcgccgaggtgtactgcggcacggccgcgaccagggagctgcggacgcgcctggtcacgcacgtgctggccctcggggtgccggggacgcgcgcgttcacgcccggcgacctggccagccgcgccgtcaccggcgcgccgcaggccggcgcggtcgcgcggtcggtggtcagcgcggtcgcggggctgctgatgtcggtcggcggcgtggtcgcgctgtggctgatcgactggcggctggtggcggcggtgggcggcgcggtcccgatcgggctgctgctcatgcgcgtcttcgtccgggacgcctccgacctggtcaccgagtaccaggaggcgcagggcgagatcgccgcgcggatgaccgacgcgctcaccggcatccgcaccatccgcgccgccgggacctggcggcgcgaggccgaccgggtcctggccccgctgccgggcctgtcggactccgggcggcggctctggcacgcctacgggcggatgcaggggcagggccgcctgatcgtcccgctggccgagatcgcggcgctggccgtcgccgggcggggcgtcctgaccggtcggctgtcggcgggggagatgctcgcggcggcgggctacgccccgatggcgctgggcctggtcggccagatccccctgctgctggccctggcgcggctgcgggcgggggcgcgccgcctggccgaggtgctgtcggtccccgcccccggcggcggcgaccgcgcgctgccgcccggccccggcgcgctggtcctgcgcggggtgaccgtgcggacgccggacgggccgctgctcgacgcggtcgacctgaccgtcccgcccggccggacggtggcggtggtcggccggtccggcgcgggcaagacgacgctcgccgccgtggccgggcgcctcctcgaccccgacgaggggcgggtgctcctggacggggtcccgctgcgcgagctcgcgcccgccgcgctgcgcgcgcaggtcgcctacgccttcgaacgccccgacctccaggggaccacgatcgccgacgccatcgcctacggctgcccgtccgcgccgccccgcgcggtcgaacgggcggccgcgctcgcgcgggcggacggcttcgtccggctgctcccggcgggcttcgacacgcccgtcgcggacacgcccctgtccggcggggaacggcagcggctcgggctggcccgggccttcgtccggaacgcccgcctgatcatcctcgacgacgcgacctccagcctcgactcggtgaccgaggcgcaggtggcggccgcgctcgcggaggccgccgcgacccgcacccggctggtggtcgcgcaccgcgccgggaccgccgcgcgcgccgacctggtcctgtggctggactccggccgggtgcgcgcgctccgcccgcaccgcgagctgtgggccgatccggactaccgggcgatgttcgagccggcgccggcggagcgcgcgtgacggggggcgcggcgcgctggttcgccgcccagctgcggaccgaacggcgcggcctggccggggtgctggcgtggtcggtcgcggcggcgctcccggcgctggtgtccggacggctgatcgcgctcgccgtcgaccagggcttcctgcgcggacgcggcacggtcggcctggcgtggctgggggcgctcgcggcggcgacggcggtcggcgcgctgggcgcgcggcagatcccgcgcgcgctcggcggggccctggagccggtgcgcgaccggctcgtccgccggatcgtggccggcgcgctgcgccgcgcgctggccggccggcccgatccgggcgtggtcgcgaagctgaccgagcagaccgagaccgtccgggacaccggcgcggggctgctgctcgggatgcagcaggtcggcatggcggtggtcgcggccgcgatcgggctgctgctgctcgcgcccgtcacggcgctgctggtcctcccgccggtgctggcggcgctgctgctgctctcccggctgctgccggagctgatggcgcggcaccgggccctgctggcggccgaggaccgcgtcgcggcgaccgtcggcggcctgaccgcggcggtgcgcgacgtggtcgcgtgcggggccgggccccgggccggggccgaggcggcggcggtcttcgccgagcaggccgcggccgaacgcgccgtcgcccgcgccgacgcgctgcgcaccgcgctcggcatcgtcggcggccagctgtcgctggtcgtcctgctggccgccgcgccctggctggtggcctccgggcggctcagccccggcgaggtgctcggcgcgctcacctacgtgacgaccggcctgcaaccggcgctgcgcgcggccgtgcagagcaccgggggttccggcgtgcggctggcggtgaccctgcaacgcctcctggacggctccgcggacgaacccgaccccgcgcccgccggagggcgcgtcccgggccgcctcgacctgtcggtggaggggctcacgttcgcctacggcgccggtgccgaaccggtcgtgcgcgacctggacctgaccgtgccgcacgggacgcacctggccgtcgtcggccccagcgggatcggcaagtcgaccctggccgacctgctcaccggggtcgccgtcccgcaggcgggccgggtccgcgtcggcggcgtgccgctcgccgagaccgatcccgcctggaggcgccggtcggtcgcgatcatcccgcaggaggcgtacgtcttcagcggcacgctcgccgagaacctcgcctacctgcgcccggacgccgccgaggccgagctcgacgccgccgtcgccgcgctgggcctggcgccgctgcgcgcccggctcggcgggtacggggccgcgctcgggccgcgttcgggcctgacgccgggggagcggcagctcgtcgcgctcgcccgcgtctggctcagccccgcgcggatcgtggtgctggacgaggcgacctgccacctcgaccccgccgccgaggcccgcgtggagcacgccttccgggaccggcccggcaccctggtcgtcatcgcgcaccggctcagctcggcgcgccgcgccgaccgcgtcctggtgatggacgggacccgcccccggctgggcacccacggggaactcctccgggactccccgctctacgccgacctggtcggggcctggaccgttcaccccgcggcgcggtag

SEQ ID NO: 140 - DNA, 123 bp, Actinomadura namibiensis
atggcatccatccttgagctccaggacctggaggtcgagcgcgccagctcggccgccgacagcaacgccagcgtctgggagtgctgcagcacgggcagctgggttcccttcacctgctgctga

SEQ ID NO: 141 - DNA, 117 bp, Actinomadura namibiensis
atggcgtcgatcctggaactccagaacctggacgtcgagcacgcccgcggcgagaaccgctccgactggagcctgtgggagtgctgtagcacgggaagcctgttcgcctgctgctga

SEQ ID NO: 142 - DNA, 13799 bp, Artificial Sequence
  Vector pUWLab
tcgcccagcgccgcgaggaagcaactgcacgccgacggcgagagcaggacgacgaccaggacgacgacgccgacgaccgccaggagcgggccgcccggcacatcgcccggctcgcaagtgggcccacttcgcactaactcgctcccccccgccgtacgtcatcccggtgacgtacggcgggggtcggtgacgtacgcggcgacggcggccggggtcgaagccgcgggagtaatcctgggattactcgcccggggtcggccccgccggcacttcgtgcaggcggtaccgggccccccctcgaggtccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatcgacggtatcgataagcttgatatcgaattcttggaggagtggatggatctgcggtaccacgcctatgcgatggccgatcccgtcttttatgactcgccgtccagcgacacccgggagaccgacggctactccgacgacctccccctgccggacggctgggaacgtcggcgcgtgggggtctgggtgatgcagggccacgacgggttgaccatgcctgaccagggctggaagatccatgtgtcggccggtctggacaacgcctggcccgtcctcgaactggtcgccaaatactgcgtcgagcaggaaatgcctttcaagttcctgcgcagcagaaggacgttgctggcccgcagcagcaagtacgccgaacgcggcggcagcggaaagttcatcacgatctaccccgccgacgaaggcgctctggaaaagaccctccatgaactcggtggaatgctggaagggcagcccggcccctatatcttgagcgatctgcgctggcgttcggggccgctgttcgtgcgctacggcgctttcaaggagaaattctgccgggacgggcgcggcgagatggtgcccgcgatcgcgcgcccggacggcgtgctggtgcccgacgccagggacccggtgttccgggtgcccgcgtgggtggaggtgcccggcttcctgcgggaggccatcgacgcccgggagaacgggaccgtcgaggacttcccctaccggatcgagaaggcgctgcacttctccaacggcggcggcctctaccgcgccgtggacgagcgcaccggccgcagggtcctggtgaaggaggcgcggccgatggcgggcctggaccgcgccgaggacgacgccgtcgtccggctcgaacgcgagcacggcctgctgctccgcctcgccgacctggactgcatccccgacctggtcgagtaccggagctggtgggagcaccgcttcctcgtgcgggagtacgtcgagggcgagaccctcacccaccacatggtgcgccgcaacccgatgctgcactacggcgcgacgccgcaggaggtcgccggctacaccgagtgggcgctcggcgtcgtcgaccgggtggagagcgcgctcggccggctgcacgaacgcggcgtggtcttcggcgacctgcaccccggcaacatcatcgtccgggacgacgactccatcgtgttcgtcgacttcgagctggtcgccgaggcggaggaggcgacgcacccggcgctcggcgcgcccggctaccaggccccgcccgactacaccgggttcgccatcgaccgctacgcgctcggctgcatcaggctcgcggtcttcacctccctcaccgcgacgctgcactgggacgaccgcaaggtcgagcagttcctcgacgtgatctgcgagtccttcccgctgccgccggactacgccgaccggatccgccgcgacctcgcccgcccggcgccggcggacggcgcgccgccgatctggcgcgagccgacgcccgccacctggccggacacgcgggccgggatcgccgccgcgatcctcgacacggccacgcccgagcgcgccgaccggctcttcccgggcgacatcgagcagttcgcgaccagcgtcggtgggatcgggttcggccacggcgcggcgggcgtgctgtgggcgctggccgaggcgggcgccggccgcttccccgaccacgaggactgggtgcgggacgccgtcgccagggcgcaacggccgccgcccggcttctacgacggcgtcgcgggcgtcgcccacgtcctggaccggctcggccgcgccgacgaggcccgcgagctcatggagcacgcgcccgccgcgacgggggcgaccgacaacagcctctaccgggggctggcgggcatcggcctcaaccagctccacttcgcccgcgtcacgggcgaggcgtcgttcgccgcggcggccgaggagaccgccggccgggtggtcgcgaacctgcgccgcaagacggagggcgcctaccgggcgggcctgatgtacggctcctccggcccggccctgttcctcgtccggatgttcgaggcgacgggcgacggccactggctggacgaggccgaacgcgccctgcaccgcgagctggacgcctgcaagtggacgcagaaggacagcacgctccaggtcgacgagggctggcgggtccttccgtacgtcgccaccggcagcgtgggcatcggcatcgcgctgcacgagttcctgcggcaccgcccggcgccgcgcttcaccgaggcgcaggaggggatccggagggcggcggctcccgcgtacttcgtgcagagcggcctgctcaacggacggtccggcatcctcgcctacctgctgcacgtcggggccggccgggaggacccggtcgtccggacgcacctgcgcaacctcggctggcacgccgtcccgtaccccggccggggcgaggacgccccggcgcccggcgcgcggcggaccgcgttcatcggggaccagctgctgcgcctgtccatggacctcgccacgggctccgccggggtcctggccacggtcgaggcggccctgggcggccaccccctgagactccccttcctccatccggaggaaggggcgagcacgcgaccccgggggaggaggtgaacatccaccatggcatccatccttgagctccaggacctggaggtcgagcgcgccagctcggccgccgacagcaacgccagcgtctgggagtgctgcagcacgggcagctgggttcccttcacctgctgctgacgcccgcacaccgttccaccgatgagaggtgacagtcccatggcgtcgatcctggaactccagaacctggacgtcgagcacgcccgcggcgagaaccgctccgactggagcctgtgggagtgctgtagcacgggaagcctgttcgcctgctgctgaacagcgctgaccgaggccgccgaagcggcacccatgtgaaacgaccgcccggggcggtggggactcccaccgccccgggcggaccaccatcagcgaggagcacgacaggaatgcacgccgaccggctgctcgtccaggcgctgcgcgccggccccgggtggacggccctcctggtcgccgcgacgctgctgaacgcggtgtgcgcgctcgccctgcccgccgcgatcggcgcggtcaccgacgcggtgctcgcccgcgacgcgggccgcggcgccggggccccgctgggctggctggcggcggcgctcggcggcgtggcgctgggcacggcgctgtcccggctcgccgaggtgtactgcggcacggccgcgaccagggagctgcggacgcgcctggtcacgcacgtgctggccctcggggtgccggggacgcgcgcgttcacgcccggcgacctggccagccgcgccgtcaccggcgcgccgcaggccggcgcggtcgcgcggtcggtggtcagcgcggtcgcggggctgctgatgtcggtcggcggcgtggtcgcgctgtggctgatcgactggcggctggtggcggcggtgggcggcgcggtcccgatcgggctgctgctcatgcgcgtcttcgtccgggacgcctccgacctggtcaccgagtaccaggaggcgcagggcgagatcgccgcgcggatgaccgacgcgctcaccggcatccgcaccatccgcgccgccgggacctggcggcgcgaggccgaccgggtcctggccccgctgccgggcctgtcggactccgggcggcggctctggcacgcctacgggcggatgcaggggcagggccgcctgatcgtcccgctggccgagatcgcggcgctggccgtcgccgggcggggcgtcctgaccggtcggctgtcggcgggggagatgctcgcggcggcgggctacgccccgatggcgctgggcctggtcggccagatccccctgctgctggccctggcgcggctgcgggcgggggcgcgccgcctggccgaggtgctgtcggtccccgcccccggcggcggcgaccgcgcgctgccgcccggccccggcgcgctggtcctgcgcggggtgaccgtgcggacgccggacgggccgctgctcgacgcggtcgacctgaccgtcccgcccggccggacggtggcggtggtcggccggtccggcgcgggcaagacgacgctcgccgccgtggccgggcgcctcctcgaccccgacgaggggcgggtgctcctggacggggtcccgctgcgcgagctcgcgcccgccgcgctgcgcgcgcaggtcgcctacgccttcgaacgccccgacctccaggggaccacgatcgccgacgccatcgcctacggctgcccgtccgcgccgccccgcgcggtcgaacgggcggccgcgctcgcgcgggcggacggcttcgtccggctgctcccggcgggcttcgacacgcccgtcgcggacacgcccctgtccggcggggaacggcagcggctcgggctggcccgggccttcgtccggaacgcccgcctgatcatcctcgacgacgcgacctccagcctcgactcggtgaccgaggcgcaggtggcggccgcgctcgcggaggccgccgcgacccgcacccggctggtggtcgcgcaccgcgccgggaccgccgcgcgcgccgacctggtcctgtggctggactccggccgggtgcgcgcgctccgcccgcaccgcgagctgtgggccgatccggactaccgggcgatgttcgagccggcgccggcggagcgcgcgtgacggggggcgcggcgcgctggttcgccgcccagctgcggaccgaacggcgcggcctggccggggtgctggcgtggtcggtcgcggcggcgctcccggcgctggtgtccggacggctgatcgcgctcgccgtcgaccagggcttcctgcgcggacgcggcacggtcggcctggcgtggctgggggcgctcgcggcggcgacggcggtcggcgcgctgggcgcgcggcagatcccgcgcgcgctcggcggggccctggagccggtgcgcgaccggctcgtccgccggatcgtggccggcgcgctgcgccgcgcgctggccggccggcccgatccgggcgtggtcgcgaagctgaccgagcagaccgagaccgtccgggacaccggcgcggggctgctgctcgggatgcagcaggtcggcatggcggtggtcgcggccgcgatcgggctgctgctgctcgcgcccgtcacggcgctgctggtcctcccgccggtgctggcggcgctgctgctgctctcccggctgctgccggagctgatggcgcggcaccgggccctgctggcggccgaggaccgcgtcgcggcgaccgtcggcggcctgaccgcggcggtgcgcgacgtggtcgcgtgcggggccgggccccgggccggggccgaggcggcggcggtcttcgccgagcaggccgcggccgaacgcgccgtcgcccgcgccgacgcgctgcgcaccgcgctcggcatcgtcggcggccagctgtcgctggtcgtcctgctggccgccgcgccctggctggtggcctccgggcggctcagccccggcgaggtgctcggcgcgctcacctacgtgacgaccggcctgcaaccggcgctgcgcgcggccgtgcagagcaccgggggttccggcgtgcggctggcggtgaccctgcaacgcctcctggacggctccgcggacgaacccgaccccgcgcccgccggagggcgcgtcccgggccgcctcgacctgtcggtggaggggctcacgttcgcctacggcgccggtgccgaaccggtcgtgcgcgacctggacctgaccgtgccgcacgggacgcacctggccgtcgtcggccccagcgggatcggcaagtcgaccctggccgacctgctcaccggggtcgccgtcccgcaggcgggccgggtccgcgtcggcggcgtgccgctcgccgagaccgatcccgcctggaggcgccggtcggtcgcgatcatcccgcaggaggcgtacgtcttcagcggcacgctcgccgagaacctcgcctacctgcgcccggacgccgccgaggccgagctcgacgccgccgtcgccgcgctgggcctggcgccgctgcgcgcccggctcggcgggtacggggccgcgctcgggccgcgttcgggcctgacgccgggggagcggcagctcgtcgcgctcgcccgcgtctggctcagccccgcgcggatcgtggtgctggacgaggcgacctgccacctcgaccccgccgccgaggcccgcgtggagcacgccttccgggaccggcccggcaccctggtcgtcatcgcgcaccggctcagctcggcgcgccgcgccgaccgcgtcctggtgatggacgggacccgcccccggctgggcacccacggggaactcctccgggactccccgctctacgccgacctggtcggggcctggaccgttcaccccgcggcgcggtagccgctcagtctagaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgtgcctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcgcattgagcgtcagcatatcatcagcgagctgaagaaagacaatccccgatccgctccacgtgttgccccagcaatcagcgcgaccttgcccctccaacgtcatctcgttctccgctcatgagctcagccaatcgactggcgagcggcatcgcattcttcgcatcccgcctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaaccaaaaggatctaggtgaagatcctttttgataatctcatgaccaaaatcccttaacgtgagttttcgttccactgagcgtcagaccaataagggcgacacggaaatgttgaatactcatactcttcctttcaatgggctgcaggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatccccattaacgcttacaatttccattcgccattcaggctgcgcaactgttgggaagggcgatcggtgcgggcctcttcgctattacgccagagcttggccggatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcgggatcatcactgacgaatcgaggtcgaggaaccgagcgtccgaggaacagaggcgcttatcggttggccgcgagattcctgtcgatcctctcgtgcagcgcgattccgagggaaacggaaacgttgagagactcggtctggctcatcatggggatggaaaccgaggcggaagacgcctcctcgaacaggtcggaaggcccacccttttcgctgccgaacagcaaggccagccgatccggattgtccccgagttccttcacggaaatgtcgccatccgccttgagcgtcatcagctgcataccgctgtcccgaatgaaggcgatggcctcctcgcgaccggagagaacgacgggaagggagaagacgtaacctcggctggccctttggagacgccggtccgcgatgctggtgatgtcactgtcgaccaggatgatccccgacgctccgagcgcgagcgacgtgcgtactatcgcgccgatgttcccgacgatcttcaccccgtcgagaacgacgacgtccccacgccggctcgcgatatcgccgaacctggccgggcgagggacgcgggcgatgccgaatgtcttggccttccgctcccccttgaacaactggttgacgatcgaggagtcgatgaggcggaccggtatgttctgccgcccgcacagatccagcaactcagatggaaaaggactgctgtcgctgccgtagacctcgatgaactccaccccggccgcgatgctgtgcatgaggggctcgacgtcctcgatcaacgttgtctttatgttggatcgcgacggcttggtgacatcgatgatccgctgcaccgcgggatcggacggatttgcgatggtgtccaactcagtcatggtcgtcctaccggctgctgtgttcagtgacgcgattcctggggtgtgacaccctacgcgacgatggcggatggctgccctgaccggcaatcaccaacgcaaggggaagtcgtcgctctctggcaaagctccccgctcttccccgtccgggacccgcgcggtcgatccccgcatatgaagtattcgccttgatcagtcccggtggacgcgccagcggcccgccggagcgacggactccccgacctcgatcgtgtcgccctgagcgtccacgtagacgttgcgtgagagcaggactgggccgccgccgaccgcaccgccctcaccaccgaccgcgaccgcgccatggccgccgccgacggcctggtcgccgccgccgcccgccggttcggcgcctgacccgaccaacccccgcggggcgccggcacttcgtgctggcgccccgcccccacccaccaggagaccgaccatgaccgacttcgacggacgcctgaccgaggggaccgtgaacctggtccaggaccccaacggcggtggctggtccgcccactgcgctgagcccggttgcgactgggccgacttcgccggaccgctcggcttccagggcctcgtggccatcgctcgccgacacacgcactgaccgcacgtcaaagccccgccggatcaccggcggggctctcttcggccctccaagtcacaccagccccaaggggcgtcgggagtggcggagggaacctctggcccgattggtgccaggattcccaccagaccaaagagcaacgggccggacttcgcacctccgacccgtccgctcccagactcgcgccccttagccgggcgagacaggaacgttgctcgtgcccagagtacggagcgatgccgaggcattgccagatcggcccgccgggccccgctgccactgcgggaccgcaattgcccacacaccgggcaaacggccgcgtatctactgctcagaccgctgccggatggcagcgaagcgggcgatcgcgcgtgtgacgcgagatgccgcccgaggcaaaagcgaacaccttgggaaagaaacaacagagtttcccgcacccctccgacctgcggtttctccggacggggtggatggggagagcccgagaggcgacagcctctcggaagtaggaagcacgtcgcggagcgacgctgcccgactgcggaaagccgcccggtacagccgccgccggacgctgtggcggatcagcggggacgccgcgtgcaagggctgcggccgcgccctgatggaccctgcctccggcgtaatcgtcgcccagacggcggccggaacgtccgtggtcctgggcctgatgcggtgcgggcggatctggctctgcccggtctgcgccgccacgatccggcacaagcgggccgaggagatcaccgccgccgtggtcgagtggatcaagcgcggggggaccgcctacctggtcaccttcacggcccgccatgggcacacggaccggctcgcggacctcatggacgccctccagggcacccggaagacgccggacagcccccggcggccgggcgcctaccagcgactgatcacgggcggcacgtgggccggacgccgggccaaggacgggcaccgggccgccgaccgcgagggcatccgagaccggatcgggtacgtcggcatgatccgcgcgaccgaagtcaccgtggggcagatcaacggctggcacccgcacatccacgcgatcgtcctggtcggcggccggaccgagggggagcggtccgcgaagcagatcgtcgccaccttcgagccgaccggcgccgcgctcgacgagtggcaggggcactggcggtccgtgtggaccgccgccctgcgcaaggtcaaccccgccttcacgcccgacgaccggcacggcgtcgacttcaagcggctggagaccgagcgcgacgccaacgacctcgccgagtacatcgccaagacccaggacgggaaggcgcccgccctcgaactcgcccgcgccgacctcaagacggcgaccggcgggaacgtcgccccgttcgaactcctcggacggatcggggacctgaccggcggcatgaccgaggacgacgccgccggggtcggctcgctggagtggaacctctcgcgctggcacgagtacgagcgggcaacccggggacgccgggccatcgaatggacccgctacctgcggcagatgctcgggctcgacggcggcgacaccgaggccgacgacctcgatctgctcctggcggccgacgccgacggcggggagctgcgggccggggtcgccgtgaccgaggacggatggcacgcggtcacccgccgcgccctcgacctcgaggcgacccgggccgccgaaggcaaggacggcaacgaggattcggcggccgtgggcgaacgggtgcgggaggtcctggcgctggccgacgcggccgacacagtggtggtgctcacggcgggggaggtggccgaggcgtacgccgacatgctcgccgccc

SEQ ID NO: 143 - DNA, 13799 bp, Artificial Sequence
  Vector pLab
ggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgacctgcagcccattgaaaggaagagtatgagtattcaacatttccgtgtcgcccttattggtctgacgctcagtggaacgaaaactcacgttaagggattttggtcatgagattatcaaaaaggatcttcacctagatccttttggttcatgtgcagctccatcagcaaaaggggatgataagtttatcaccaccgactatttgcaacagtgccgttgatcgtgctatgatcgactgatgtcatcagcggtggagtgcaatgtcgtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcggtttgccttggcggacaggtggctcaaggagaagagccttcagaaggaaggtccagtcggtcatgcctttgctcggttgatccgctcccgcgacattgtggcgacagccctcggtcaactgggccgagatccgttgatcttcctgcatccgccagaggcgggatgcgaagaatgcgatgccgctcgccagtcgattggctgagctcatgagcggagaacgagatgacgttggaggggcaaggtcgcgctgattgctggggcaacacgtggagcggatcggggattgtctttcttcagctcgctgatgatatgctgacgctcaatgcgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggtggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccaggcacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttctagactgagcggctaccgcgccgcggggtgaacggtccaggccccgaccaggtcggcgtagagcggggagtcccggaggagttccccgtgggtgcccagccgggggcgggtcccgtccatcaccaggacgcggtcggcgcggcgcgccgagctgagccggtgcgcgatgacgaccagggtgccgggccggtcccggaaggcgtgctccacgcgggcctcggcggcggggtcgaggtggcaggtcgcctcgtccagcaccacgatccgcgcggggctgagccagacgcgggcgagcgcgacgagctgccgctcccccggcgtcaggcccgaacgcggcccgagcgcggccccgtacccgccgagccgggcgcgcagcggcgccaggcccagcgcggcgacggcggcgtcgagctcggcctcggcggcgtccgggcgcaggtaggcgaggttctcggcgagcgtgccgctgaagacgtacgcctcctgcgggatgatcgcgaccgaccggcgcctccaggcgggatcggtctcggcgagcggcacgccgccgacgcggacccggcccgcctgcgggacggcgaccccggtgagcaggtcggccagggtcgacttgccgatcccgctggggccgacgacggccaggtgcgtcccgtgcggcacggtcaggtccaggtcgcgcacgaccggttcggcaccggcgccgtaggcgaacgtgagcccctccaccgacaggtcgaggcggcccgggacgcgccctccggcgggcgcggggtcgggttcgtccgcggagccgtccaggaggcgttgcagggtcaccgccagccgcacgccggaacccccggtgctctgcacggccgcgcgcagcgccggttgcaggccggtcgtcacgtaggtgagcgcgccgagcacctcgccggggctgagccgcccggaggccaccagccagggcgcggcggccagcaggacgaccagcgacagctggccgccgacgatgccgagcgcggtgcgcagcgcgtcggcgcgggcgacggcgcgttcggccgcggcctgctcggcgaagaccgccgccgcctcggccccggcccggggcccggccccgcacgcgaccacgtcgcgcaccgccgcggtcaggccgccgacggtcgccgcgacgcggtcctcggccgccagcagggcccggtgccgcgccatcagctccggcagcagccgggagagcagcagcagcgccgccagcaccggcgggaggaccagcagcgccgtgacgggcgcgagcagcagcagcccgatcgcggccgcgaccaccgccatgccgacctgctgcatcccgagcagcagccccgcgccggtgtcccggacggtctcggtctgctcggtcagcttcgcgaccacgcccggatcgggccggccggccagcgcgcggcgcagcgcgccggccacgatccggcggacgagccggtcgcgcaccggctccagggccccgccgagcgcgcgcgggatctgccgcgcgcccagcgcgccgaccgccgtcgccgccgcgagcgcccccagccacgccaggccgaccgtgccgcgtccgcgcaggaagccctggtcgacggcgagcgcgatcagccgtccggacaccagcgccgggagcgccgccgcgaccgaccacgccagcaccccggccaggccgcgccgttcggtccgcagctgggcggcgaaccagcgcgccgcgccccccgtcacgcgcgctccgccggcgccggctcgaacatcgcccggtagtccggatcggcccacagctcgcggtgcgggcggagcgcgcgcacccggccggagtccagccacaggaccaggtcggcgcgcgcggcggtcccggcgcggtgcgcgaccaccagccgggtgcgggtcgcggcggcctccgcgagcgcggccgccacctgcgcctcggtcaccgagtcgaggctggaggtcgcgtcgtcgaggatgatcaggcgggcgttccggacgaaggcccgggccagcccgagccgctgccgttccccgccggacaggggcgtgtccgcgacgggcgtgtcgaagcccgccgggagcagccggacgaagccgtccgcccgcgcgagcgcggccgcccgttcgaccgcgcggggcggcgcggacgggcagccgtaggcgatggcgtcggcgatcgtggtcccctggaggtcggggcgttcgaaggcgtaggcgacctgcgcgcgcagcgcggcgggcgcgagctcgcgcagcgggaccccgtccaggagcacccgcccctcgtcggggtcgaggaggcgcccggccacggcggcgagcgtcgtcttgcccgcgccggaccggccgaccaccgccaccgtccggccgggcgggacggtcaggtcgaccgcgtcgagcagcggcccgtccggcgtccgcacggtcaccccgcgcaggaccagcgcgccggggccgggcggcagcgcgcggtcgccgccgccgggggcggggaccgacagcacctcggccaggcggcgcgcccccgcccgcagccgcgccagggccagcagcagggggatctggccgaccaggcccagcgccatcggggcgtagcccgccgccgcgagcatctcccccgccgacagccgaccggtcaggacgccccgcccggcgacggccagcgccgcgatctcggccagcgggacgatcaggcggccctgcccctgcatccgcccgtaggcgtgccagagccgccgcccggagtccgacaggcccggcagcggggccaggacccggtcggcctcgcgccgccaggtcccggcggcgcggatggtgcggatgccggtgagcgcgtcggtcatccgcgcggcgatctcgccctgcgcctcctggtactcggtgaccaggtcggaggcgtcccggacgaagacgcgcatgagcagcagcccgatcgggaccgcgccgcccaccgccgccaccagccgccagtcgatcagccacagcgcgaccacgccgccgaccgacatcagcagccccgcgaccgcgctgaccaccgaccgcgcgaccgcgccggcctgcggcgcgccggtgacggcgcggctggccaggtcgccgggcgtgaacgcgcgcgtccccggcaccccgagggccagcacgtgcgtgaccaggcgcgtccgcagctccctggtcgcggccgtgccgcagtacacctcggcgagccgggacagcgccgtgcccagcgccacgccgccgagcgccgccgccagccagcccagcggggccccggcgccgcggcccgcgtcgcgggcgagcaccgcgtcggtgaccgcgccgatcgcggcgggcagggcgagcgcgcacaccgcgttcagcagcgtcgcggcgaccaggagggccgtccacccggggccggcgcgcagcgcctggacgagcagccggtcggcgtgcattcctgtcgtgctcctcgctgatggtggtccgcccggggcggtgggagtccccaccgccccgggcggtcgtttcacatgggtgccgcttcggcggcctcggtcagcgctgttcagcagcaggcgaacaggcttcccgtgctacagcactcccacaggctccagtcggagcggttctcgccgcgggcgtgctcgacgtccaggttctggagttccaggatcgacgccatgggactgtcacctctcatcggtggaacggtgtgcgggcgtcagcagcaggtgaagggaacccagctgcccgtgctgcagcactcccagacgctggcgttgctgtcggcggccgagctggcgcgctcgacctccaggtcctggagctcaaggatggatgccatggtggatgttcacctcctcccccggggtcgcgtgctcgccccttcctccggatggaggaaggggagtctcagggggtggccgcccagggccgcctcgaccgtggccaggaccccggcggagcccgtggcgaggtccatggacaggcgcagcagctggtccccgatgaacgcggtccgccgcgcgccgggcgccggggcgtcctcgccccggccggggtacgggacggcgtgccagccgaggttgcgcaggtgcgtccggacgaccgggtcctcccggccggccccgacgtgcagcaggtaggcgaggatgccggaccgtccgttgagcaggccgctctgcacgaagtacgcgggagccgccgccctccggatcccctcctgcgcctcggtgaagcgcggcgccgggcggtgccgcaggaactcgtgcagcgcgatgccgatgcccacgctgccggtggcgacgtacggaaggacccgccagccctcgtcgacctggagcgtgctgtccttctgcgtccacttgcaggcgtccagctcgcggtgcagggcgcgttcggcctcgtccagccagtggccgtcgcccgtcgcctcgaacatccggacgaggaacagggccgggccggaggagccgtacatcaggcccgcccggtaggcgccctccgtcttgcggcgcaggttcgcgaccacccggccggcggtctcctcggccgccgcggcgaacgacgcctcgcccgtgacgcgggcgaagtggagctggttgaggccgatgcccgccagcccccggtagaggctgttgtcggtcgcccccgtcgcggcgggcgcgtgctccatgagctcgcgggcctcgtcggcgcggccgagccggtccaggacgtgggcgacgcccgcgacgccgtcgtagaagccgggcggcggccgttgcgccctggcgacggcgtcccgcacccagtcctcgtggtcggggaagcggccggcgcccgcctcggccagcgcccacagcacgcccgccgcgccgtggccgaacccgatcccaccgacgctggtcgcgaactgctcgatgtcgcccgggaagagccggtcggcgcgctcgggcgtggccgtgtcgaggatcgcggcggcgatcccggcccgcgtgtccggccaggtggcgggcgtcggctcgcgccagatcggcggcgcgccgtccgccggcgccgggcgggcgaggtcgcggcggatccggtcggcgtagtccggcggcagcgggaaggactcgcagatcacgtcgaggaactgctcgaccttgcggtcgtcccagtgcagcgtcgcggtgagggaggtgaagaccgcgagcctgatgcagccgagcgcgtagcggtcgatggcgaacccggtgtagtcgggcggggcctggtagccgggcgcgccgagcgccgggtgcgtcgcctcctccgcctcggcgaccagctcgaagtcgacgaacacgatggagtcgtcgtcccggacgatgatgttgccggggtgcaggtcgccgaagaccacgccgcgttcgtgcagccggccgagcgcgctctccacccggtcgacgacgccgagcgcccactcggtgtagccggcgacctcctgcggcgtcgcgccgtagtgcagcatcgggttgcggcgcaccatgtggtgggtgagggtctcgccctcgacgtactcccgcacgaggaagcggtgctcccaccagctccggtactcgaccaggtcggggatgcagtccaggtcggcgaggcggagcagcaggccgtgctcgcgttcgagccggacgacggcgtcgtcctcggcgcggtccaggcccgccatcggccgcgcctccttcaccaggaccctgcggccggtgcgctcgtccacggcgcggtagaggccgccgccgttggagaagtgcagcgccttctcgatccggtaggggaagtcctcgacggtcccgttctcccgggcgtcgatggcctcccgcaggaagccgggcacctccacccacgcgggcacccggaacaccgggtccctggcgtcgggcaccagcacgccgtccgggcgcgcgatcgcgggcaccatctcgccgcgcccgtcccggcagaatttctccttgaaagcgccgtagcgcacgaacagcggccccgaacgccagcgcagatcgctcaagatataggggccgggctgcccttccagcattccaccgagttcatggagggtcttttccagagcgccttcgtcggcggggtagatcgtgatgaactttccgctgccgccgcgttcggcgtacttgctgctgcgggccagcaacgtccttctgctgcgcaggaacttgaaaggcatttcctgctcgacgcagtatttggcgaccagttcgaggacgggccaggcgttgtccagaccggccgacacatggatcttccagccctggtcaggcatggtcaacccgtcgtggccctgcatcacccagacccccacgcgccgacgttcccagccgtccggcagggggaggtcgtcggagtagccgtcggtctcccgggtgtcgctggacggcgagtcataaaagacgggatcggccatcgcataggcgtggtaccgcagatccatccactcctccaagaattcgatatcaagcttatcgataccgtcgatcctaccaaccggcacgattgtgcccacaacagcatcgcggtgccacgtgtggaccgcgtcggtcagatcctccccgcacctctcgccagccgtcaagatcgaccgcgtgcacctgcgatcgccgatcaaccgcgactagcatcgggcgcaagccgccactcgaacggacactcgcatggacgtccccttcctggacctgcaagccgcgtacctcgaactccggtccgacatcgaccaggcgtgccggcgcgtgctcgggtcgggctggacctcgagggggggcccggtaccgcctgcacgaagtgccggcggggccgaccccgggcgagtaatcccaggattactcccgcggcttcgaccccggccgccgtcgccgcgtacgtcaccgacccccgccgtacgtcaccgggatgacgtacggcgggggggagcgagttagtgcgaagtgggcccacttgcgagccgggcgatgtgccgggcggcccgctcctggcggtcgtcggcgtcgtcgtcctggtcgtcgtcctgctctcgccgtcggcgtgcagttgcttcctcgcggcgctgggcgagggcggcgagcatgtcggcgtacgcctcggccacctcccccgccgtgagcaccaccactgtgtcggccgcgtcggccagcgccaggacctcccgcacccgttcgcccacggccgccgaatcctcgttgccgtccttgccttcggcggcccgggtcgcctcgaggtcgagggcgcggcgggtgaccgcgtgccatccgtcctcggtcacggcgaccccggcccgcagctccccgccgtcggcgtcggccgccaggagcagatcgaggtcgtcggcctcggtgtcgccgccgtcgagcccgagcatctgccgcaggtagcgggtccattcgatggcccggcgtccccgggttgcccgctcgtactcgtgccagcgcgagaggttccactccagcgagccgaccccggcggcgtcgtcctcggtcatgccgccggtcaggtccccgatccgtccgaggagttcgaacggggcgacgttcccgccggtcgccgtcttgaggtcggcgcgggcgagttcgagggcgggcgccttcccgtcctgggtcttggcgatgtactcggcgaggtcgttggcgtcgcgctcggtctccagccgcttgaagtcgacgccgtgccggtcgtcgggcgtgaaggcggggttgaccttgcgcagggcggcggtccacacggaccgccagtgcccctgccactcgtcgagcgcggcgccggtcggctcgaaggtggcgacgatctgcttcgcggaccgctccccctcggtccggccgccgaccaggacgatcgcgtggatgtgcgggtgccagccgttgatctgccccacggtgacttcggtcgcgcggatcatgccgacgtacccgatccggtctcggatgccctcgcggtcggcggcccggtgcccgtccttggcccggcgtccggcccacgtgccgcccgtgatcagtcgctggtaggcgcccggccgccgggggctgtccggcgtcttccgggtgccctggagggcgtccatgaggtccgcgagccggtccgtgtgcccatggcgggccgtgaaggtgaccaggtaggcggtccccccgcgcttgatccactcgaccacggcggcggtgatctcctcggcccgcttgtgccggatcgtggcggcgcagaccgggcagagccagatccgcccgcaccgcatcaggcccaggaccacggacgttccggccgccgtctgggcgacgattacgccggaggcagggtccatcagggcgcggccgcagcccttgcacgcggcgtccccgctgatccgccacagcgtccggcggcggctgtaccgggcggctttccgcagtcgggcagcgtcgctccgcgacgtgcttcctacttccgagaggctgtcgcctctcgggctctccccatccaccccgtccggagaaaccgcaggtcggaggggtgcgggaaactctgttgtttctttcccaaggtgttcgcttttgcctcgggcggcatctcgcgtcacacgcgcgatcgcccgcttcgctgccatccggcagcggtctgagcagtagatacgcggccgtttgcccggtgtgtgggcaattgcggtcccgcagtggcagcggggcccggcgggccgatctggcaatgcctcggcatcgctccgtactctgggcacgagcaacgttcctgtctcgcccggctaaggggcgcgagtctgggagcggacgggtcggaggtgcgaagtccggcccgttgctctttggtctggtgggaatcctggcaccaatcgggccagaggttccctccgccactcccgacgccccttggggctggtgtgacttggagggccgaagagagccccgccggtgatccggcggggctttgacgtgcggtcagtgcgtgtgtcggcgagcgatggccacgaggccctggaagccgagcggtccggcgaagtcggcccagtcgcaaccgggctcagcgcagtgggcggaccagccaccgccgttggggtcctggaccaggttcacggtcccctcggtcaggcgtccgtcgaagtcggtcatggtcggtctcctggtgggtgggggcggggcgccagcacgaagtgccggcgccccgcgggggttggtcgggtcaggcgccgaaccggcgggcggcggcggcgaccaggccgtcggcggcggccatggcgcggtcgcggtcggtggtgagggcggtgcggtcggcggcggcccagtcctgctctcacgcaacgtctacgtggacgctcagggcgacacgatcgaggtcggggagtccgtcgctccggcgggccgctggcgcgtccaccgggactgatcaaggcgaatacttcatatgcggggatcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcgcgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtcgacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaacctcatacagaaaattcatttactaacgtctggaaagacgacaaaactttagatccggccaagctctggcgtaatagcgaagaggcccgcaccgatcgcccttcccaacagttgcgcagcctgaatggcgaatggaaattgtaagcgttaatggggatcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataag

SEQ ID NO: 144 - DNA, 15787 bp, Artificial Sequence
  Vector pLab Amp
tggtggtgctcacggcgggggaggtggccgaggcgtacgccgacatgctcgccgccctcgcccagcgccgcgaggaagcaactgcacgccgacggcgagagcaggacgacgaccaggacgacgacgccgacgaccgccaggagcgggccgcccggcacatcgcccggctcgcaagtgggcccacttcgcactaactcgctcccccccgccgtacgtcatcccggtgacgtacggcgggggtcggtgacgtacgcggcgacggcggccggggtcgaagccgcgggagtaatcctgggattactcgcccggggtcggccccgccggcacttcgtgcaggcggtaccgggccccccctcgaggtccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatcgacggtatcgataagcttgatatcgaattcttggaggagtggatggatctgcggtaccacgcctatgcgatggccgatcccgtcttttatgactcgccgtccagcgacacccgggagaccgacggctactccgacgacctccccctgccggacggctgggaacgtcggcgcgtgggggtctgggtgatgcagggccacgacgggttgaccatgcctgaccagggctggaagatccatgtgtcggccggtctggacaacgcctggcccgtcctcgaactggtcgccaaatactgcgtcgagcaggaaatgcctttcaagttcctgcgcagcagaaggacgttgctggcccgcagcagcaagtacgccgaacgcggcggcagcggaaagttcatcacgatctaccccgccgacgaaggcgctctggaaaagaccctccatgaactcggtggaatgctggaagggcagcccggcccctatatcttgagcgatctgcgctggcgttcggggccgctgttcgtgcgctacggcgctttcaaggagaaattctgccgggacgggcgcggcgagatggtgcccgcgatcgcgcgcccggacggcgtgctggtgcccgacgccagggacccggtgttccgggtgcccgcgtgggtggaggtgcccggcttcctgcgggaggccatcgacgcccgggagaacgggaccgtcgaggacttcccctaccggatcgagaaggcgctgcacttctccaacggcggcggcctctaccgcgccgtggacgagcgcaccggccgcagggtcctggtgaaggaggcgcggccgatggcgggcctggaccgcgccgaggacgacgccgtcgtccggctcgaacgcgagcacggcctgctgctccgcctcgccgacctggactgcatccccgacctggtcgagtaccggagctggtgggagcaccgcttcctcgtgcgggagtacgtcgagggcgagaccctcacccaccacatggtgcgccgcaacccgatgctgcactacggcgcgacgccgcaggaggtcgccggctacaccgagtgggcgctcggcgtcgtcgaccgggtggagagcgcgctcggccggctgcacgaacgcggcgtggtcttcggcgacctgcaccccggcaacatcatcgtccgggacgacgactccatcgtgttcgtcgacttcgagctggtcgccgaggcggaggaggcgacgcacccggcgctcggcgcgcccggctaccaggccccgcccgactacaccgggttcgccatcgaccgctacgcgctcggctgcatcaggctcgcggtcttcacctccctcaccgcgacgctgcactgggacgaccgcaaggtcgagcagttcctcgacgtgatctgcgagtccttcccgctgccgccggactacgccgaccggatccgccgcgacctcgcccgcccggcgccggcggacggcgcgccgccgatctggcgcgagccgacgcccgccacctggccggacacgcgggccgggatcgccgccgcgatcctcgacacggccacgcccgagcgcgccgaccggctcttcccgggcgacatcgagcagttcgcgaccagcgtcggtgggatcgggttcggccacggcgcggcgggcgtgctgtgggcgctggccgaggcgggcgccggccgcttccccgaccacgaggactgggtgcgggacgccgtcgccagggcgcaacggccgccgcccggcttctacgacggcgtcgcgggcgtcgcccacgtcctggaccggctcggccgcgccgacgaggcccgcgagctcatggagcacgcgcccgccgcgacgggggcgaccgacaacagcctctaccgggggctggcgggcatcggcctcaaccagctccacttcgcccgcgtcacgggcgaggcgtcgttcgccgcggcggccgaggagaccgccggccgggtggtcgcgaacctgcgccgcaagacggagggcgcctaccgggcgggcctgatgtacggctcctccggcccggccctgttcctcgtccggatgttcgaggcgacgggcgacggccactggctggacgaggccgaacgcgccctgcaccgcgagctggacgcctgcaagtggacgcagaaggacagcacgctccaggtcgacgagggctggcgggtccttccgtacgtcgccaccggcagcgtgggcatcggcatcgcgctgcacgagttcctgcggcaccgcccggcgccgcgcttcaccgaggcgcaggaggggatccggagggcggcggctcccgcgtacttcgtgcagagcggcctgctcaacggacggtccggcatcctcgcctacctgctgcacgtcggggccggccgggaggacccggtcgtccggacgcacctgcgcaacctcggctggcacgccgtcccgtaccccggccggggcgaggacgccccggcgcccggcgcgcggcggaccgcgttcatcggggaccagctgctgcgcctgtccatggacctcgccacgggctccgccggggtcctggccacggtcgaggcggccctgggcgcgcctaggccttgacggccttccgccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgagcggaaggcccatgaggccagttaattaagcgctgaccgaggccgccgaagcggcacccatgtgaaacgaccgcccggggcggtggggactcccaccgccccgggcggaccaccatcagcgaggagcacgacaggaatgcacgccgaccggctgctcgtccaggcgctgcgcgccggccccgggtggacggccctcctggtcgccgcgacgctgctgaacgcggtgtgcgcgctcgccctgcccgccgcgatcggcgcggtcaccgacgcggtgctcgcccgcgacgcgggccgcggcgccggggccccgctgggctggctggcggcggcgctcggcggcgtggcgctgggcacggcgctgtcccggctcgccgaggtgtactgcggcacggccgcgaccagggagctgcggacgcgcctggtcacgcacgtgctggccctcggggtgccggggacgcgcgcgttcacgcccggcgacctggccagccgcgccgtcaccggcgcgccgcaggccggcgcggtcgcgcggtcggtggtcagcgcggtcgcggggctgctgatgtcggtcggcggcgtggtcgcgctgtggctgatcgactggcggctggtggcggcggtgggcggcgcggtcccgatcgggctgctgctcatgcgcgtcttcgtccgggacgcctccgacctggtcaccgagtaccaggaggcgcagggcgagatcgccgcgcggatgaccgacgcgctcaccggcatccgcaccatccgcgccgccgggacctggcggcgcgaggccgaccgggtcctggccccgctgccgggcctgtcggactccgggcggcggctctggcacgcctacgggcggatgcaggggcagggccgcctgatcgtcccgctggccgagatcgcggcgctggccgtcgccgggcggggcgtcctgaccggtcggctgtcggcgggggagatgctcgcggcggcgggctacgccccgatggcgctgggcctggtcggccagatccccctgctgctggccctggcgcggctgcgggcgggggcgcgccgcctggccgaggtgctgtcggtccccgcccccggcggcggcgaccgcgcgctgccgcccggccccggcgcgctggtcctgcgcggggtgaccgtgcggacgccggacgggccgctgctcgacgcggtcgacctgaccgtcccgcccggccggacggtggcggtggtcggccggtccggcgcgggcaagacgacgctcgccgccgtggccgggcgcctcctcgaccccgacgaggggcgggtgctcctggacggggtcccgctgcgcgagctcgcgcccgccgcgctgcgcgcgcaggtcgcctacgccttcgaacgccccgacctccaggggaccacgatcgccgacgccatcgcctacggctgcccgtccgcgccgccccgcgcggtcgaacgggcggccgcgctcgcgcgggcggacggcttcgtccggctgctcccggcgggcttcgacacgcccgtcgcggacacgcccctgtccggcggggaacggcagcggctcgggctggcccgggccttcgtccggaacgcccgcctgatcatcctcgacgacgcgacctccagcctcgactcggtgaccgaggcgcaggtggcggccgcgctcgcggaggccgccgcgacccgcacccggctggtggtcgcgcaccgcgccgggaccgccgcgcgcgccgacctggtcctgtggctggactccggccgggtgcgcgcgctccgcccgcaccgcgagctgtgggccgatccggactaccgggcgatgttcgagccggcgccggcggagcgcgcgtgacggggggcgcggcgcgctggttcgccgcccagctgcggaccgaacggcgcggcctggccggggtgctggcgtggtcggtcgcggcggcgctcccggcgctggtgtccggacggctgatcgcgctcgccgtcgaccagggcttcctgcgcggacgcggcacggtcggcctggcgtggctgggggcgctcgcggcggcgacggcggtcggcgcgctgggcgcgcggcagatcccgcgcgcgctcggcggggccctggagccggtgcgcgaccggctcgtccgccggatcgtggccggcgcgctgcgccgcgcgctggccggccggcccgatccgggcgtggtcgcgaagctgaccgagcagaccgagaccgtccgggacaccggcgcggggctgctgctcgggatgcagcaggtcggcatggcggtggtcgcggccgcgatcgggctgctgctgctcgcgcccgtcacggcgctgctggtcctcccgccggtgctggcggcgctgctgctgctctcccggctgctgccggagctgatggcgcggcaccgggccctgctggcggccgaggaccgcgtcgcggcgaccgtcggcggcctgaccgcggcggtgcgcgacgtggtcgcgtgcggggccgggccccgggccggggccgaggcggcggcggtcttcgccgagcaggccgcggccgaacgcgccgtcgcccgcgccgacgcgctgcgcaccgcgctcggcatcgtcggcggccagctgtcgctggtcgtcctgctggccgccgcgccctggctggtggcctccgggcggctcagccccggcgaggtgctcggcgcgctcacctacgtgacgaccggcctgcaaccggcgctgcgcgcggccgtgcagagcaccgggggttccggcgtgcggctggcggtgaccctgcaacgcctcctggacggctccgcggacgaacccgaccccgcgcccgccggagggcgcgtcccgggccgcctcgacctgtcggtggaggggctcacgttcgcctacggcgccggtgccgaaccggtcgtgcgcgacctggacctgaccgtgccgcacgggacgcacctggccgtcgtcggccccagcgggatcggcaagtcgaccctggccgacctgctcaccggggtcgccgtcccgcaggcgggccgggtccgcgtcggcggcgtgccgctcgccgagaccgatcccgcctggaggcgccggtcggtcgcgatcatcccgcaggaggcgtacgtcttcagcggcacgctcgccgagaacctcgcctacctgcgcccggacgccgccgaggccgagctcgacgccgccgtcgccgcgctgggcctggcgccgctgcgcgcccggctcggcgggtacggggccgcgctcgggccgcgttcgggcctgacgccgggggagcggcagctcgtcgcgctcgcccgcgtctggctcagccccgcgcggatcgtggtgctggacgaggcgacctgccacctcgaccccgccgccgaggcccgcgtggagcacgccttccgggaccggcccggcaccctggtcgtcatcgcgcaccggctcagctcggcgcgccgcgccgaccgcgtcctggtgatggacgggacccgcccccggctgggcacccacggggaactcctccgggactccccgctctacgccgacctggtcggggcctggaccgttcaccccgcggcgcggtagccgctcagtctagaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgtgcctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcgcattgagcgtcagcatatcatcagcgagctgaagaaagacaatccccgatccgctccacgtgttgccccagcaatcagcgcgaccttgcccctccaacgtcatctcgttctccgctcatgagctcagccaatcgactggcgagcggcatcgcattcttcgcatcccgcctctggcggatgcaggaagatcaacggatctcggcccagttgaccgagggctgtcgccacaatgtcgcgggagcggatcaaccgagcaaaggcatgaccgactggaccttccttctgaaggctcttctccttgagccacctgtccgccaaggcaaaccgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaaccaaaaggatctaggtgaagatcctttttgataatctcatgaccaaaatcccttaacgtgagttttcgttccactgagcgtcagaccaataagggcgacacggaaatgttgaatactcatactcttcctttcaatgggctgcaggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatccccattaacgcttacaatttccattcgccattcaggctgcgcaactgttgggaagggcgatcggtgcgggcctcttcgctattacgccagagcttggccggatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcgggatcatcactgacgaatcgaggtcgaggaaccgagcgtccgaggaacagaggcgcttatcggttggccgcgagattcctgtcgatcctctcgtgcagcgcgattccgagggaaacggaaacgttgagagactcggtctggctcatcatggggatggaaaccgaggcggaagacgcctcctcgaacaggtcggaaggcccacccttttcgctgccgaacagcaaggccagccgatccggattgtccccgagttccttcacggaaatgtcgccatccgccttgagcgtcatcagctgcataccgctgtcccgaatgaaggcgatggcctcctcgcgaccggagagaacgacgggaagggagaagacgtaacctcggctggccctttggagacgccggtccgcgatgctggtgatgtcactgtcgaccaggatgatccccgacgctccgagcgcgagcgacgtgcgtactatcgcgccgatgttcccgacgatcttcaccccgtcgagaacgacgacgtccccacgccggctcgcgatatcgccgaacctggccgggcgagggacgcgggcgatgccgaatgtcttggccttccgctcccccttgaacaactggttgacgatcgaggagtcgatgaggcggaccggtatgttctgccgcccgcacagatccagcaactcagatggaaaaggactgctgtcgctgccgtagacctcgatgaactccaccccggccgcgatgctgtgcatgaggggctcgacgtcctcgatcaacgttgtctttatgttggatcgcgacggcttggtgacatcgatgatccgctgcaccgcgggatcggacggatttgcgatggtgtccaactcagtcatggtcgtcctaccggctgctgtgttcagtgacgcgattcctggggtgtgacaccctacgcgacgatggcggatggctgccctgaccggcaatcaccaacgcaaggggaagtcgtcgctctctggcaaagctccccgctcttccccgtccgggacccgcgcggtcgatccccgcatatgaagtattcgccttgatcagtcccggtggacgcgccagcggcccgccggagcgacggactccccgacctcgatcgtgtcgccctgagcgtccacgtagacgttgcgtgagagcaggactgggccgccgccgaccgcaccgccctcaccaccgaccgcgaccgcgccatggccgccgccgacggcctggtcgccgccgccgcccgccggttcggcgcctgacccgaccaacccccgcggggcgccggcacttcgtgctggcgccccgcccccacccaccaggagaccgaccatgaccgacttcgacggacgcctgaccgaggggaccgtgaacctggtccaggaccccaacggcggtggctggtccgcccactgcgctgagcccggttgcgactgggccgacttcgccggaccgctcggcttccagggcctcgtggccatcgctcgccgacacacgcactgaccgcacgtcaaagccccgccggatcaccggcggggctctcttcggccctccaagtcacaccagccccaaggggcgtcgggagtggcggagggaacctctggcccgattggtgccaggattcccaccagaccaaagagcaacgggccggacttcgcacctccgacccgtccgctcccagactcgcgccccttagccgggcgagacaggaacgttgctcgtgcccagagtacggagcgatgccgaggcattgccagatcggcccgccgggccccgctgccactgcgggaccgcaattgcccacacaccgggcaaacggccgcgtatctactgctcagaccgctgccggatggcagcgaagcgggcgatcgcgcgtgtgacgcgagatgccgcccgaggcaaaagcgaacaccttgggaaagaaacaacagagtttcccgcacccctccgacctgcggtttctccggacggggtggatggggagagcccgagaggcgacagcctctcggaagtaggaagcacgtcgcggagcgacgctgcccgactgcggaaagccgcccggtacagccgccgccggacgctgtggcggatcagcggggacgccgcgtgcaagggctgcggccgcgccctgatggaccctgcctccggcgtaatcgtcgcccagacggcggccggaacgtccgtggtcctgggcctgatgcggtgcgggcggatctggctctgcccggtctgcgccgccacgatccggcacaagcgggccgaggagatcaccgccgccgtggtcgagtggatcaagcgcggggggaccgcctacctggtcaccttcacggcccgccatgggcacacggaccggctcgcggacctcatggacgccctccagggcacccggaagacgccggacagcccccggcggccgggcgcctaccagcgactgatcacgggcggcacgtgggccggacgccgggccaaggacgggcaccgggccgccgaccgcgagggcatccgagaccggatcgggtacgtcggcatgatccgcgcgaccgaagtcaccgtggggcagatcaacggctggcacccgcacatccacgcgatcgtcctggtcggcggccggaccgagggggagcggtccgcgaagcagatcgtcgccaccttcgagccgaccggcgccgcgctcgacgagtggcaggggcactggcggtccgtgtggaccgccgccctgcgcaaggtcaaccccgccttcacgcccgacgaccggcacggcgtcgacttcaagcggctggagaccgagcgcgacgccaacgacctcgccgagtacatcgccaagacccaggacgggaaggcgcccgccctcgaactcgcccgcgccgacctcaagacggcgaccggcgggaacgtcgccccgttcgaactcctcggacggatcggggacctgaccggcggcatgaccgaggacgacgccgccggggtcggctcgctggagtggaacctctcgcgctggcacgagtacgagcgggcaacccggggacgccgggccatcgaatggacccgctacctgcggcagatgctcgggctcgacggcggcgacaccgaggccgacgacctcgatctgctcctggcggccgacgccgacggcggggagctgcgggccggggtcgccgtgaccgaggacggatggcacgcggtcacccgccgcgccctcgacctcgaggcgacccgggccgccgaaggcaaggacggcaacgaggattcggcggccgtgggcgaacgggtgcgggaggtcctggcgctggccgacgcggccgacacag

SEQ ID NO: 145 - DNA, 20 bp, Artificial Sequence
  Iko-Fw1
gttcgttcgacggaccaatg

SEQ ID NO: 146 - DNA, 20 bp, Artificial Sequence
  Iko Rev1
cctgctcgacgcagtatttg

SEQ ID NO: 147 - DNA, 20 bp, Artificial Sequence
  Iko Fw2
cgcaggacgaacggtttcag

SEQ ID NO: 148 - DNA, 20 bp, Artificial Sequence
  Iko Rev2
ccatgggactgtcacctctc

SEQ ID NO: 149 - DNA, 20 bp, Artificial Sequence
  Iko Fw3
catccaccatggcatccatc

SEQ ID NO: 150 - DNA, 20 bp, Artificial Sequence
  Iko Rev3′
gcgtcgtcgaggatgatcag

SEQ ID NO: 151 - DNA, 20 bp, Artificial Sequence
  Iko Fw3′
actaccgggcgatgttcgag

SEQ ID NO: 152 - DNA, 20 bp, Artificial Sequence
  Iko Rev3
agcagccgggagagcagcag

SEQ ID NO: 153 - DNA, 20 bp, Artificial Sequence
  Iko Fw4
actaccgggcgatgttcgag

SEQ ID NO: 154 - DNA, 20 bp, Artificial Sequence
  Iko Rev4′
ctgaagacgtacgcctcctg

SEQ ID NO: 155 - DNA, 20 bp, Artificial Sequence
  Iko Fw4′
caggaggcgtacgtcttcag

SEQ ID NO: 156 - DNA, 20 bp, Artificial Sequence
  Iko Rev4
agatgaagcgggcgatcgag

SEQ ID NO: 157 - DNA, 20 bp, Artificial Sequence
  LabKC_Fw2
cctgccggacggctgggaac

SEQ ID NO: 158 - DNA, 20 bp, Artificial Sequence
  LabKC_Fw3
gggagaacgggaccgtcgag

SEQ ID NO: 159 - DNA, 20 bp, Artificial Sequence
  LabKC_Fw4
cgcccgactacaccgggttc

SEQ ID NO: 160 - DNA, 20 bp, Artificial Sequence
  LabKC_Fw5
cccgcgagctcatggagcac

SEQ ID NO: 161 - DNA, 20 bp, Artificial Sequence
  LabKC_Fw6
ccggcatcctcgcctacctg

SEQ ID NO: 162 - DNA, 7403 bp, Artificial Sequence
  pUWLoriT plasmid
ctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatcgacggtatcgataagcttgatatcgaattcctgcagcccgggggatccactagttctagaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgtgcctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcgcattgagcgtcagcatatcatcagcgagctgaagaaagacaatccccgatccgctccacgtgttgccccagcaatcagcgcgaccttgcccctccaacgtcatctcgttctccgctcatgagctcagccaatcgactggcgagcggcatcgcattcttcgcatcccgcctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaaccaaaaggatctaggtgaagatcctttttgataatctcatgaccaaaatcccttaacgtgagttttcgttccactgagcgtcagaccaataagggcgacacggaaatgttgaatactcatactcttcctttcaatgggctgcaggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatccccattaacgcttacaatttccattcgccattcaggctgcgcaactgttgggaagggcgatcggtgcgggcctcttcgctattacgccagagcttggccggatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcgggatcatcactgacgaatcgaggtcgaggaaccgagcgtccgaggaacagaggcgcttatcggttggccgcgagattcctgtcgatcctctcgtgcagcgcgattccgagggaaacggaaacgttgagagactcggtctggctcatcatggggatggaaaccgaggcggaagacgcctcctcgaacaggtcggaaggcccacccttttcgctgccgaacagcaaggccagccgatccggattgtccccgagttccttcacggaaatgtcgccatccgccttgagcgtcatcagctgcataccgctgtcccgaatgaaggcgatggcctcctcgcgaccggagagaacgacgggaagggagaagacgtaacctcggctggccctttggagacgccggtccgcgatgctggtgatgtcactgtcgaccaggatgatccccgacgctccgagcgcgagcgacgtgcgtactatcgcgccgatgttcccgacgatcttcaccccgtcgagaacgacgacgtccccacgccggctcgcgatatcgccgaacctggccgggcgagggacgcgggcgatgccgaatgtcttggccttccgctcccccttgaacaactggttgacgatcgaggagtcgatgaggcggaccggtatgttctgccgcccgcacagatccagcaactcagatggaaaaggactgctgtcgctgccgtagacctcgatgaactccaccccggccgcgatgctgtgcatgaggggctcgacgtcctcgatcaacgttgtctttatgttggatcgcgacggcttggtgacatcgatgatccgctgcaccgcgggatcggacggatttgcgatggtgtccaactcagtcatggtcgtcctaccggctgctgtgttcagtgacgcgattcctggggtgtgacaccctacgcgacgatggcggatggctgccctgaccggcaatcaccaacgcaaggggaagtcgtcgctctctggcaaagctccccgctcttccccgtccgggacccgcgcggtcgatccccgcatatgaagtattcgccttgatcagtcccggtggacgcgccagcggcccgccggagcgacggactccccgacctcgatcgtgtcgccctgagcgtccacgtagacgttgcgtgagagcaggactgggccgccgccgaccgcaccgccctcaccaccgaccgcgaccgcgccatggccgccgccgacggcctggtcgccgccgccgcccgccggttcggcgcctgacccgaccaacccccgcggggcgccggcacttcgtgctggcgccccgcccccacccaccaggagaccgaccatgaccgacttcgacggacgcctgaccgaggggaccgtgaacctggtccaggaccccaacggcggtggctggtccgcccactgcgctgagcccggttgcgactgggccgacttcgccggaccgctcggcttccagggcctcgtggccatcgctcgccgacacacgcactgaccgcacgtcaaagccccgccggatcaccggcggggctctcttcggccctccaagtcacaccagccccaaggggcgtcgggagtggcggagggaacctctggcccgattggtgccaggattcccaccagaccaaagagcaacgggccggacttcgcacctccgacccgtccgctcccagactcgcgccccttagccgggcgagacaggaacgttgctcgtgcccagagtacggagcgatgccgaggcattgccagatcggcccgccgggccccgctgccactgcgggaccgcaattgcccacacaccgggcaaacggccgcgtatctactgctcagaccgctgccggatggcagcgaagcgggcgatcgcgcgtgtgacgcgagatgccgcccgaggcaaaagcgaacaccttgggaaagaaacaacagagtttcccgcacccctccgacctgcggtttctccggacggggtggatggggagagcccgagaggcgacagcctctcggaagtaggaagcacgtcgcggagcgacgctgcccgactgcggaaagccgcccggtacagccgccgccggacgctgtggcggatcagcggggacgccgcgtgcaagggctgcggccgcgccctgatggaccctgcctccggcgtaatcgtcgcccagacggcggccggaacgtccgtggtcctgggcctgatgcggtgcgggcggatctggctctgcccggtctgcgccgccacgatccggcacaagcgggccgaggagatcaccgccgccgtggtcgagtggatcaagcgcggggggaccgcctacctggtcaccttcacggcccgccatgggcacacggaccggctcgcggacctcatggacgccctccagggcacccggaagacgccggacagcccccggcggccgggcgcctaccagcgactgatcacgggcggcacgtgggccggacgccgggccaaggacgggcaccgggccgccgaccgcgagggcatccgagaccggatcgggtacgtcggcatgatccgcgcgaccgaagtcaccgtggggcagatcaacggctggcacccgcacatccacgcgatcgtcctggtcggcggccggaccgagggggagcggtccgcgaagcagatcgtcgccaccttcgagccgaccggcgccgcgctcgacgagtggcaggggcactggcggtccgtgtggaccgccgccctgcgcaaggtcaaccccgccttcacgcccgacgaccggcacggcgtcgacttcaagcggctggagaccgagcgcgacgccaacgacctcgccgagtacatcgccaagacccaggacgggaaggcgcccgccctcgaactcgcccgcgccgacctcaagacggcgaccggcgggaacgtcgccccgttcgaactcctcggacggatcggggacctgaccggcggcatgaccgaggacgacgccgccggggtcggctcgctggagtggaacctctcgcgctggcacgagtacgagcgggcaacccggggacgccgggccatcgaatggacccgctacctgcggcagatgctcgggctcgacggcggcgacaccgaggccgacgacctcgatctgctcctggcggccgacgccgacggcggggagctgcgggccggggtcgccgtgaccgaggacggatggcacgcggtcacccgccgcgccctcgacctcgaggcgacccgggccgccgaaggcaaggacggcaacgaggattcggcggccgtgggcgaacgggtgcgggaggtcctggcgctggccgacgcggccgacacagtggtggtgctcacggcgggggaggtggccgaggcgtacgccgacatgctcgccgccctcgcccagcgccgcgaggaagcaactgcacgccgacggcgagagcaggacgacgaccaggacgacgacgccgacgaccgccaggagcgggccgcccggcacatcgcccggctcgcaagtgggcccacttcgcactaactcgctcccccccgccgtacgtcatcccggtgacgtacggcgggggtcggtgacgtacgcggcgacggcggccggggtcgaagccgcgggagtaatcctgggattactcgcccggggtcggccccgccggcacttcgtgcaggcggtaccgggccccccctcgaggtccagcccgacccgagcacgcgccggcacgc

(19) United States
(12) Patent Application Publication
(10) Pub. No.: US 2015/0274787 A1
(43) Pub. Date: Oct. 1, 2015

(54) METHOD FOR RECOMBINANT PRODUCTION OF LABYRINTHOPEPTINS AND FUNCTIONAL DERIVATIVES THEREOF

(71) Applicant: SANOFI, Paris (FR)

(72) Inventors: Joachim Wink, Rodermark (DE); Mark Broenstrup, Frankfurt (DE); Stefan Bartoschek, Frankfurt (DE); Luigi Toti, Hocheim (DE); Roderich Suessmuth, Berlin (DE); Joanna Krawczyk, Berlin (DE)

(21) Appl. No.: 14/367,470

(22) PCT Filed: Dec. 19, 2012
(86) PCT No.: PCT/EP2012/076099

§ 371 Date: Jun. 20, 2014

(30) Foreign Application Priority Data
Dec. 21, 2011 (EP) 11306712.8

(51) Int. Cl. C07K 14/36; C07K 7/54

(52) U.S. Cl. CPC C07K 14/36; C07K 7/54

(57) ABSTRACT

The present invention relates to a method for recombinant production of Labyrinthopeptins and functional derivatives thereof. Moreover, the present invention relates to novel functional derivatives of Labyrinthopeptins.

The present invention relates to a method for recombinant production of Labyrinthopeptins and functional derivatives thereof. Moreover, the present invention relates to novel functional derivatives of Labyrinthopeptins.

Lantibiotics are peptides that are ribosomally synthesized from bacteria such as staphylococci, lactobacilli, and actinomycetes. The common structural characteristic of lantibiotics is the noncanonical amino acid lanthionine (Lan), which confers conformational stability to the peptide. Labyrinthopeptins are class III lantibiotics isolated from a desert bacterium Actinomadura namibiensis. They are first representatives of a new type of lantibiotics with a unique carbacyclic post-translationally modified amino acid named labionin. The chemical structre of Labionin is as follows:

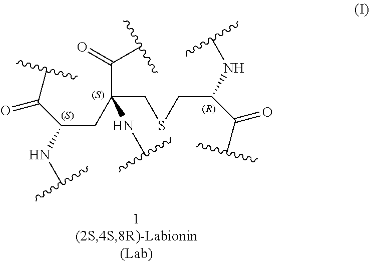

The biological characterization showed that Labyrinthopeptin A2 (LabA2) revealed an efficacy in an in vivo neuropathic pain model. Since these compounds can be considered as new leads for drug discovery there is a strong need to perform the structure-function relationship studies to identify motifs essential for their bioactivity. However, due to the presence of complex post-translational modifications, a chemical synthesis of lantibiotic analogues is complicated and not efficient, or like it is for Labyrinthopeptins not known at all.

There is therefore a need for methods allowing recombinant production of Labyrinthopeptins, as well as for the generation of functional derivatives of Labyrinthopeptins.

The present invention relates to a nucleic acid comprising at least one Labyrinthopeptin (lab) gene cluster, in particular a gene cluster having a sequence according to SEQ ID No. 139, of an actinomycete, in particular of Actinomaduras namibiensis, wherein:
(a) the nucleic acid further comprises a constitutive promoter for expression in Streptomyces cells, in particular an ermE promotor, and wherein the gene cluster is under control of said promoter, and/or
(b) the nucleic acid encoding LabA1 and/or LabA2 or a functional derivative thereof is mutated, such that the C-terminal amino acid of the leader peptide of LabA1 and/or LabA2 is substituted with a methionine, and/or
(c) the nucleic acid further comprises at least one hybrid gene sequence, wherein the hybrid gene sequence:
   (i) encodes a pre-pro-peptide comprising a LabA2 leader sequence and labA1 pre-peptide or functional derivative thereof and/or
   (ii) encodes a pre-pro-peptide comprising a LabA1 leader sequence and labA2 pre-peptide or functional derivative thereof

In a preferred embodiment, the Streptomyces cells are selected from Streptomyces lividans and Streptomyces albus cells, in particular the cells are Streptomyces lividans cells.

It was found that LabA1 and/or LabA2 and functional derivatives thereof can be successfully produced by recombinant production, as shown in Examples 2 to 4. In particular, the expression was successfully performed using a constitutive promotor ermE for expression in Streptomyces.

The lab gene cluster comprises the genes labKC, labA1, labA2, labT1 and labT2. Structural genes labA1 and labA2 code for labyrinthopeptin A1/A3 and labyrinthopeptin A2 prepropeptides, respectively. labT1 and labT2 encode proteins with homology to transporter proteins. The gene labKC codes for a trifunctional protein with an N-terminal lyase domain, Ser/Thr kinase domain and a C-terminal putative lanthionine cyclase domain necessary for posttranslational modification of Labyrinthopeptins, and therefore for generation of mature Labyrinthopeptin.

The native labA1 and/or labA2 genes have a sequence according to SEQ ID No. 140 and SEQ ID No. 141, respectively. The expression of these genes resulting in mature LabA1 and LabA2 could be performed successfully by recombinant expression in Streptomyces cells according to the Examples. Moreover, functional derivatives could be generated.

Thus, in a further embodiment, the nucleic acid encoding LabA1 and/or LabA2 has a sequence according to SEQ ID No. 140 or SEQ ID No. 141, or has a sequence exhibiting at least 85%, preferably at least 90%, even more preferably at least 95% sequence identity to SEQ ID No. 140 and/or SEQ ID No. 141.

Moreover, it could be shown in the examples, that mutating the last, C-terminal amino acid of the leader peptide to methionine suprisingly results in the production of mature LabA1 and LabA2, without remaining amino acids of the leader peptide.

Also, it could suprisingly be shown that it is possible to transfer the nucleic acid encoding the pro-peptide sequence LabA2 to the nucleic acid encoding the leader peptide of LabA1, and to transfer the nucleic acid encoding the pro-peptide sequence LabA1 to the nucleic acid encoding the leader peptide of LabA2, thereby generating hybrid pre-pro-peptides.

In a further embodiment, the invention relates to a vector, in particular a plasmid, comprising a nucleic acid construct as described above. In the examples, the vectors pUWLab and pLab were successfully used for expression of Labyrinthopeptins and derivatives thereof in Streptomyces species. The vectors were constructed starting from the known vector pUWLoriT, as described in the Examples, by inserting a lab gene cluster, such that the gene cluster is under control of the constitutive promotor ermE.

Thus, in a preferred embodiment, the vector of the invention is:
(a) pLab, having a sequence according to SEQ ID No. 143, or pUWLab, having a sequence according to SEQ ID No. 143, or pLabAmp, having a sequence according to SEQ ID No. 144, and/or
(b) obtainable from pUWLoriT by inserting at least one lab gene cluster, such that the gene cluster is under control of the constitutive promotor.

In particular, the vector is a plasmid.

After several unsuccessful attempts, as described in Example 1, expression of Labyrinthopeptins and functional derivatives thereof could be achieved by transformation of Streptomyces lividans and Streptomyces albus cells (Examples 2 to 4), using the above vectors. Notably, the vectors are bifunctional and can therefore also be used for replication and propagation in E. coli.

Therefore, the present invention further relates to a bacterial cell, in particular a Streptomyces or E. coli cell, more preferably a Streptomyces lividans or Streptomyces albus cell, transformed with a vector according to the invention.

Methods for transforming Streptomyces or E. coli cells are known to a skilled person. In particular, the transformation method as explained in the Materials and Methods section of the Examples may be used.

In another embodiment, the present invention relates to the use of at least one Streptomyces lividans and/or Streptomyces albus cell for the recombinant expression of at least one Labyrinthopeptin or a functional derivative thereof.

In a further embodiment, the invention relates to a method for producing at least one Labyrinthopeptin or functional derivative thereof, comprising the steps:
(a) culturing a Streptomyces cell, preferably a Streptomyces lividans or Streptomyces albus cell, which cell is transformed with a vector according to the invention, in culture medium,
(b) harvesting the culture medium comprising at least one Labyrinthopeptin or functional derivative thereof, and
(c) optionally purifying at least one Labyrinthopeptin or functional derivative thereof from the culture medium.

Methods for culturing bacterial cells are known to a skilled person. In particular, the culturing methods and media as explained in the Materials and Methods section of the Examples may be used. A. namibiensis and Streptomyces cells may be cultured in suitable media known to a skilled person. In particular, agar media, preferably selected from MS, R2YE, R5 or KM4, as disclosed in the Examples, may be used. In another preferred embodiment, liquid media, in particular selected from YEME, CRM, KM4 or M5294 may be used, as for example disclosed the Examples. The A. namibiensis and Streptomyces cells may be cultured at about 10° C. to about 40° C., preferably at about 20° C. to about 35° C., more preferably at about 25° C. to 30° C., most preferably at about 28° C. Methods for culturing E. coli are known in the art. In particular, LB medium, as described in the Examples may be used. The cell cultures may be supplemented with antibiotics, where appropriate for selection.

Cells may in particular be cultured for about 30 minutes to 6 about months, in particular for 1 about day to about 3 months, for preferably for about 3 days to about 6 weeks depending on the used species or type of cells. E. coli cells for example are usually grown for 1 to 2 days, and Streptomyces spec. cells are usually grown for 3 days to 3 weeks.

In another preferred embodiment, the vector of the invention comprises a nucleic acid encoding LabA1 and/or LabA2 or functional derivative thereof, wherein the nucleic acid is mutated such that the C-terminal amino acid the leader peptide of LabA1 and/or LabA2 is substituted with a methionine. Such constructs can allow for expression of mature peptides without amino acids from the leader peptide.

In another preferred embodiment, the vector of the invention comprises at least one hybrid nucleic acid selected from:
(i) a hybrid nucleic acid encoding a pre-pro-peptide comprising a LabA2 leader sequence and LabA1 pre-peptide or functional derivative thereof and/or
(ii) a hybrid nucleic acid encoding a pre-pro-peptide comprising a LabA1 leader sequence and LabA2 pre-peptide or functional derivative thereof, preferably (ii).

Such hybrid molecules are in particular suitable for expressing higher amounts of LabA2 or functional derivatives thereof, as shown in Example 4.

Methods for introducing mutations in a nucleic acid are well-known in the art. For example, site-directed mutagenesis as described in the Materials and Methods section of the Examples may be performed.

Also, methods for detecting Labyrinthopeptin or functional derivatives thereof are known in the art. For example, immunological methods, like Western Blotting, and/or LC-MS and/or MS/MS and/or GC-MS, as shown in the Materials and Methods section of the Examples and the Examples 2 to 4 may be used.

For cloning and DNA amplification experiments, various methods are described in the prior art. In particular, PCR may be used for amplification and cloning as described in the Materials and Methods section of the Examples. Also, methods for cloning including the use of restriction endonucleases and/or ligases are known in the art. Preferred methods are described in the Materials and Methods section of the Examples.

In some embodiments, a bifunctional vector is used, which can be propagated both in E. coli and in Streptomyces cells.

Methods for transforming bacteria are well known in the art. For example, E. coli cells may be transformed by electroporation or chemical transformation, as disclosed in the Examples. Streptomyces strains may be transformed in particular using protoplasts. In particular, protoplasts may be prepared, followed by PEG-assisted transformation. Also, conjugation may be used. Such protocols are disclosed in the Examples.

The invention further relates to a mixture of more than one Labyrinthopeptin or functional derivatives thereof, obtainable by the method of the invention.

A number of novel Labyrinthopeptin derivatives could be generated according to the examples.

Therefore, in one embodiment, the present invention relates to a compound selected from:
(a) a Labyrinthopeptin pre-pro-peptide Labyrinthopeptin or a functional derivative thereof, wherein the C-terminal amino acid of the leader sequence is a methionine, or
(b) a nucleic acid encoding a Labyrinthopeptin pre-pro-peptide or a functional derivative thereof, wherein the C-terminal amino acid of the leader sequence of the pre-pro-peptide is a methionine.

In a further embodiment, the present invention relates to a nucleic acid comprising a sequence according to any of SEQ ID No. 1 to 24, preferably, it is consisting of a sequence according to any of SEQ ID No. 1 to 24.

In a further embodiment, the present invention relates to a functional derivative of a Labyrinthopeptin pre-pro-peptide encoded by a nucleic acid comprising a sequence according to any of SEQ ID No. 1 to 24, and which pre-pro-peptide has a sequence different from LabA1 pre-pro-peptide according to SEQ ID No. 132 or LabA2 pre-pro-peptide according to SEQ ID No. 135.

As shown in Examples 3 and 4, it was surprisingly possible to express a number of mutated forms of Labyrinthopeptins in Streptomyces cells using the nucleic acid molecules according to SEQ ID No. 1 to 24. These cassettes could be inserted into an appropriate vector, for transformation and expression in Streptomyces cells.

Particularly preferred is the use of a nucleic acid comprising a sequence according to SEQ ID No. 1 (SG2), and SEQ ID No. 5 (SG6). These cassettes were in particular useful for performing site-directed mutagenesis of labA1 and/or labA2 genes.

In another preferred embodiment, nucleic acids nucleic acid comprising a sequence according to SEQ ID No. 3 and/or SEQ ID No. 6 may be used. Such constructs have been useful for expressing LabA2 and functional derivatives thereof.

In another embodiment, the present invention relates to a functional derivative of a Labyrinthopeptin pre-peptide encoded by a nucleic acid comprising a sequence according to any of SEQ ID No. 1 to 24, and which has pre-peptide has a sequence different from LabA1 pre-peptide according to SEQ ID No. 134 or LabA2 pre-peptide according to SEQ ID No. 137, or LabA3 pre-peptide according to SEQ ID No. 138, and which pre-peptide lacks the leader sequence or at least the N-terminal 80%, in particular 90%, more preferably 95% of the leader sequence, in particular wherein the leader sequence has a sequence according to SEQ ID No. 133 or SEQ ID No. 136.

In another embodiment, the invention relates to a derivative, in particular functional derivative, of a Labyrinthopeptin peptide, in particular selected from: LabA1_N2A, LabA1_V5A, LabA1_E7A, LabA1_T11A, LabA1_V15A, LabA1_P16A, LabA1_F17A, LabA2_D2A, LabA2_W3A, LabA2_L5A, LabA2_W6A, LabA2_E7A, LabA2_T11A, LabA2_G12A, LabA2_L14A, LabA2_F15A, LabA1_V5del, LabA1_W6insV, LabA1_P16del, LabA1_P16insV, LabA1_S4T, M-LabA1_S4T, AM-LabA1_S4T, NR-LabA2_M (SG20), R-LabA2_M (SG20), LabA1_V15S, M-LabA1_V15S, LabA1_T11S (Ser), LabA1_W6Y, LabA1_A3H, LabA1_C20insA, LabA1_S10insA, LabA1_C20del, LabA1_S1insC, D-LabA1/A2 (SG11), AD-LabA1/A2 (SG11), R-LabA2/A1 (SG11), NR-LabA2/A1 (SG11), and ENR-LabA2/A1 (SG11).

In another embodiment, the present invention relates to a S. lividans cell deposited with Accession number DSM24184. In a further embodiment, the present invention relates to the use of a S. lividans cell deposited with Accession number DSM24184 for producing LabA1, LabA2 and/or LabA3 and/or derivatives thereof, in particular functional derivatives thereof.

In another embodiment, the present invention relates to a S. lividans cell deposited with Accession number DSM24580. In a further embodiment, the present invention relates to a LabA2 derivative produced by the cell, in particular to a LabA2_L14A molecule produced by the cell.

In another embodiment, the present invention relates to a S. lividans cell deposited with Accession number DSM24581. In a further embodiment, the present invention relates to a LabA2 derivative produced by the cell, in particular to a LabA2_F15A produced by the cell.

According to the invention, “LabA#_§X&” is understood as meaning a LabA# variant, wherein amino acid No. X of the pre-peptide of LabA# is mutated from § to &. For example, in LabA1_N2A, amino acid No. 2 of LabA1 pro-peptide is mutated from N to A.

The number of amino acids is understood as to be calculated starting from the N-terminus.

According to the invention, “LabA#_§Xdel” is understood as meaning a LabA# variant, wherein amino acid No. X of the pre-peptide of LabA1, which is a §, is deleted. For example, “LabA1_V5del” is understood as meaning a LabA1 variant, wherein amino acid No. 5 of the pre-peptide of LabA1, which is a Valine, is deleted.

According to the invention, “LabA#_§Xins&” is understood as meaning a LabA# variant, wherein amino acid & is inserted before amino acid No. X of the pre-peptide of LabA#, pushing original amino acid No. X (&) to position X+1. For example, “LabA1_P16insV” is understood as meaning a LabA1 variant, wherein amino acid Valine is inserted before amino acid No. 16 of the pre-peptide of LabA1, pushing original amino acid No. 16 (Proline) to position 17.

According to the invention, “§-LabA#” is understood as LabA#, having an additional amino acid § at the N-terminus Such additional amino acid may origin from the leader sequence which is not completely processed.

According to the invention, “LabA2/A1” is understood as hybrid consisting of a LabA2 leader sequence and a LabA1 pre-peptide sequence. Accordingly, LabA1/A2 is understood as hybrid consisting of a LabA1 leader sequence and a LabA2 pre-peptide sequence.

Labyrinthopeptins LabA1 and LabA2 were shown to have antimicrobial, antiviral and anti-pain effects. Moreover, LabA2 (A) and LabA2_L14A are shown to exhibit anti-pain activity in the present application (FIG. 27). Therefore, the present invention also relates to a mixture or a compound of the invention, for use in the treatment and/or prevention of bacterial infections, HIV infections or pain, in particular neuropathic pain. Further, the present invention also relates to the use of a mixture or a compound of the invention, for the preparation of a medicament for the treatment and/or prevention of bacterial infections, HIV infections or pain, in particular neuropathic pain. Also, the present invention relates to a method for treating and/or preventing bacterial infections, HIV infections or pain, in particular neuropathic pain in a mammal, in particular in a human, comprising administering to said mammal an effective amount of a mixture or a compound of the invention.

The peptides may be formulated according to methods skilled in the art. Preferably, the compounds and mixtures are present as a pharmaceutical composition, together with a pharmaceutically acceptable carrier, like e.g. water or saline.

The Labyrinthopeptins LabA1 and LabA2 are disclosed in W02008/040469A1. Moreover, the chemical structure and activity of the Labyrinthopeptins is summarized in Meindl et al. (2010).

The term “Labyrinthopeptin” is understood as encompassing LabA1, LabA2, and LabA3 peptides.

The term “mature Labyrinthopeptin” is understood as Labyrinthopeptin functional derivative thereof, which lacks the leader sequence, or a part of it, and which has undergone posttranslational modification. In particular, a mature Labyrinthopeptin contains one or more labionine amino acids.

The structure of mature LabA1 is as follows:

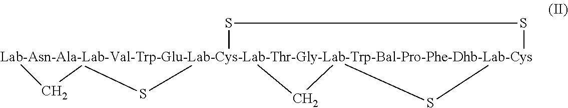

wherein Dhb is didehydrobutyrine, and Lab is Labionin.

The structure of mature LabA2 is as follows:

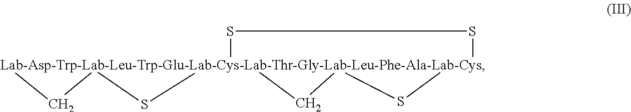

wherein Lab is Labionin.

Labionin rings A and A′ are formed by a methylene group between αC atoms of the amino acid No. 1, starting from the N-terminus (Lab 1) and the amino acid No. 4 (Lab 4) for ring A, and by a methylene group between αC atoms of the amino acid No. 10, starting from the N-terminus (Lab 10) and the amino acid No. 13 (Lab 13) for ring A′. The rings B and B′ rings are formed by a thioether bridge.

The structure of Dhb is as follows:

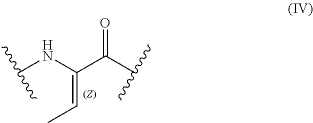 [SMILES: *N/C(=C\C)C(*)=O]

Also LabA3 has been described (Meindl et al., supra). LabA3 carries an additional Asp residue at the N-terminus compared to mature LabA1.

LabA1: C₉₂H₁₁₉N₂₃O₂₅S₄ (2073.7624 amu)
LabA2: C₈₅H₁₁₀N₂₀O₂₄S₄ (1922.6930 amu)

### Labionin: C₉H₁₆N₃O₆S

“Functional derivatives” of Labyrinthopeptins are understood peptides exhibiting at least 80%, more preferably at least 90%, more preferably at least 95% sequence identity compared to pre-peptide sequence of LabA1 according to SEQ ID No. 134, of LabA2 according to SEQ ID No. 137, or LabA3 according to SEQ ID No. 138, and/or to a pre-pro-peptide sequence of LabA1 according to SEQ ID No. 132, or LabA2 according to SEQ ID No. 135. Preferably, the functional derivatives exhibit at least about 10%, more preferably at least about 50%, even more preferably at least about 90% of the antibacterial, antiviral and/or anti-pain activity of LabA1, LabA2 and/or LabA3, as determined according to Examples 15, 16 and 17 of W02008/040469A1. In particular, functional derivatives encompass Labyrinthopeptins which contain 1 or more, in particular 1, 2, 3, or 4 amino acids of the leader sequence, but not the complete leader sequence. Functional derivatives are also orthologs of labA1 or labA2 from other actinomycetes.

A “pre-pro-peptide” according to the present invention is understood as peptide having the length of the complete translated coding region and which can be obtained by translation by the ribosomal machinery. A pre-pro-peptide includes leader sequences and does not contain post-translational modifications.

A “pro-peptide” according to the present invention is understood as peptide which lacks the leader sequence and which does not contain post-translational modifications.

A “leader sequence” according to the present invention is understood as peptide sequence released during peptide processing in A. namibiensis cells. The leader sequence is therefore not present in mature LabA1 or LabA2. The native leader sequences of LabA1 and LabA2 are depicted in SEQ ID No. 133 and SEQ ID No. 136, respectively.

“About” according to the present invention is understood as meaning the experimental error range, in particular ±5% or ±10%.

DSM24184 relates to a Streptomyces lividans strain deposited with the DSMZ Deutsche Sammlung von Mikroorganismen und Zellkulturen GmbH with Accession number DSM24184. The strain comprises the lab gene cluster.

DSM24580 relates to a Streptomyces lividans strain deposited with the DSMZ Deutsche Sammlung von Mikroorganismen und Zellkulturen GmbH with Accession number DSM24580. The strain expresses the derivative LabA2_L14A.

DSM24581: relates to a Streptomyces lividans strain deposited with the DSMZ Deutsche Sammlung von Mikroorganismen und Zellkulturen GmbH with Accession number DSM24581. The strain expresses the derivative LabA2_F15A.
SEQ ID No. 132 represents the sequence of LabA1 pre-pro-peptide.
SEQ ID No. 133 represents the sequence of LabA1 leader peptide.
SEQ ID No. 134 represents the sequence of LabA1 pre-peptide, lacking the leader sequence.
SEQ ID No. 135 represents the sequence of LabA2 pre-pro-peptide.
SEQ ID No. 136 represents the sequence of LabA2 leader peptide.
SEQ ID No. 137 represents the sequence of LabA2 pre-peptide, lacking the leader sequence.
SEQ ID No. 138 represents the sequence of LabA3 pre-peptide.
SEQ ID No. 139 represents the sequence of the lab gene cluster.
SEQ ID No. 140 represents the sequence of the labA1 gene of A. namibiensis.
SEQ ID No. 141 represents the sequence of the labA2 gene of A. namibiensis.
SEQ ID No. 142 represents the sequence of pUWLab.
SEQ ID No. 143 represents the sequence of pLab.
SEQ ID No. 144 represents the sequence of pLabAmp.
SEQ ID No. 162 represents the sequence of pUWLoriT.

The remaining sequences are described in the Examples below.

### DESCRIPTION OF FIGURES

FIG. 1: gene clusters hypothetically coding for Labyrinthopeptin-like peptides.

FIG. 2: A) A picture represents which part of the lab gene cluster was amplified by the use of primer pair I-VI. B) DNA fragments obtained after PCR with primers I-VI for S. coelicolor integration mutant. C) Southern blotting, 1-cosmid KH, 2-S. coelicolor wt, 3-cl. 3A*, 4) Clone 3B*, 5-Clone 3C*, 6-Clone 3D*, 7-Clone 3E*, 8-Clone 4A*, 9-Clone 4B*, 10-DigVII marker, * S. coelicolor/cosmid KH.

FIG. 3: Primers used for amplification of the lab gene cluster for cloning to a vector pUWLoriT.

FIG. 4: Cloning strategy to generate a vector pUWLab carrying the whole lab gene cluster.

FIG. 5: Verification of a correctness of a vector pUWL by restriction digestion.

FIG. 6: Detection of LabA2 by a Western-blot. Lanes: 1 and 4) Molecular size marker (10-170 kDa), 2) S. lividans (extract of bacterial cells from the MS agar plate), 3) S. lividans/pUWLab (extract of bacterial cells from the MS agar plate), 5) A. namibiensis (supernatant obtained from the liquid culture medium M5294).

FIG. 7: Detection of LabA1 and LabA2 in a liquid culture of A. namibiensis (9 d, medium KM4). A) Total ion chromatogram (TIC), B) MS spectrum of LabA1 and LabA2 (LabA1: [M+2H]²⁺=1038.3; LabA2: [M+2H]²⁺=962.9), C) Extracted Ion Chromatogram over the range m/z 1037.5-1038.5 corresponding to the doubly-charged LabA1, D) Extracted Ion Chromatogram over the range m/z 962.0-963.0 corresponding to the doubly-charged LabA2.

FIG. 8: Detection of LabA1 and LabA2 derivatives in a liquid culture of S. lividans/pUWLab (9 d, medium YEME). A) Total ion chromatogram (TIC), B) Extracted Ion Chromatogram over the range m/z 1095.5-1096.5 corresponding to the doubly-charged D-LabA1, C) Extracted Ion Chromatogram over the m/z range 1097.5-1098.5 corresponding to doubly-charged NR-LabA2, D) MS spectrum of D-LabA1 and AD-LabA2 (D-LabA1: [M+2H]²⁺=1096.1; AD-LabA1: [M+2H]²⁺=1131.7), E) MS spectrum of NR-LabA2 (NR-LabA1: [M+2H]²⁺=1098.2).

FIG. 9: Strategy to obtain construct pLab. pLab is suitable for preparation of different Labyrinthopeptin variants.

FIG. 10: Cloning strategy to obtain pLabAmp.

FIG. 11: Cloning strategy used to obtain pLab_SG2/SG3/SG4/SG5/SG6 vectors. A. Restriction digestion of pLabAmp with PasI and Eco47III; isolation of 13.4 kbp fragments from an agarose gel B. Restriction digestion of pMK/pMA carrying synthetic gene (SG) with PasI and Eco47III; isolation of 374 by (for pMK_SG2)/371 bp (pMA_SG3)/212 bp (pMA_SG4) fragment from a gel C. Ligation of synthetic gene to pLabAmp D. Verification of clones correctness by PCR and sequencing.

FIG. 12: Detection of LabA1 and its derivatives in a liquid culture (medium R2YE) of S. lividans carrying the vector pLab_SG2 after 5 d and 3 weeks of growth. A) Total ion chromatogram (5 d old culture), B) Extracted Ion Chromatogram over the range m/z 1037.5-1038.5 corresponding to the doubly-charged LabA1 (5 d old culture), C) MS spectrum of LabA1 and LabA1 derivatives (LabA1: [M+2H]²⁺=1038.4; M-LabA1: [M+2H]²⁺=1104.1; AM-LabA1: [M+2H]²⁺=1139.5) (5 d old culture), D) Total ion chromatogram (3 weeks old culture), E) Extracted Ion Chromatogram over the range m/z 1037.5-1038.5 corresponding to the doubly-charged LabA1 (3 weeks old culture), F) MS spectrum of LabA1 (LabA1: [M+2H]²⁺=1038.2) (3 weeks old culture).

FIG. 13: Comparison of amino acid sequences of labyrinthopeptin prepropeptides present in the wild type (pLab), and in synthetic genes SG2 and SG3.

FIG. 14: Detection of LabA1 and LabA2 derivatives in a liquid culture of S. lividans carrying the vector pLab_SG3 (3 weeks, medium R2YE). A) Total ion chromatogram (TIC), B) Extracted Ion Chromatogram over the range m/z 1037.5-1038.5 corresponding to the doubly-charged LabA1, C) MS spectrum of LabA1 and LabA2 derivatives (LabA1: [M+2H]²⁺=1038.1; A-LabA1: [M+2H]²⁺=1073.7; AA-LabA1: [M+2H]²⁺=1109.5; AD-LabA2: [M+2H]²⁺=1055.6).

FIG. 15: Graphic presentation of synthetic genes SG3, SG3(M) and SG6.

FIG. 16: Detection of LabA2 and LabA1 derivatives in a liquid culture of S. lividans/SG6 (NZ Amine medium, 3 weeks old culture). A) Total ion chromatogram (TIC), B) Extracted Ion Chromatogram over the range m/z 962.0-963.0 corresponding to the doubly-charged LabA2, C) MS spectrum of LabA2 (LabA2: [M+2H]²⁺=962.13; LabA2: [M+H]⁺=1922.30). D) Extracted Ion Chromatogram over the range m/z 1115.5-1116.5 corresponding to the doubly-charged R-LabA1, E) MS spectrum of LabA1 derivatives (R-LabA1: [M+2H]²⁺=1115.43; NR-LabA1: [M+2H]²⁺=1172.35; ENR-LabA1: [M+2H]²⁺=1236.74).

FIG. 17: Comparison of amino acid sequences of labyrinthopeptin prepropeptides present in the wild type (pLab), and in synthetic genes SG4 and SG5.

FIG. 18: Strategy of preparing different Labyrinthopeptin derivatives. A) Introduction of desired mutation to synthetic oligonucleotide by means of site-directed mutagenesis B, C) Amplification of a fragment between PasI and Eco47III restriction sites (Infusion primers) D) Restriction digestion of plasmid pLab (with enzymes PasI and Eco47III) containing the whole lab gene cluster D) Ligation independent cloning (LIC) to obtain a construct for expression of new Labyrinthopeptin derivatives; SG—synthetic genes, *—mutation.

FIG. 19: LabA1 (A) and LabA2 (B) alanine-scanning mutants prepared for further SAR studies.

FIG. 20: Comparison of production yields for LabA1 (A, B) and LabA2 (C, D) alanine mutants after 8 (A, C) and 21 (B, D) days of growth in NZ Amine medium. A,B) pLab carrying: 1. SG2, 2. LabA1_N2A, 3. LabA1_W6A, 4. LabA1_E7A, 5. LabA1_T11A, 6. LabA1_V15A, 7. LabA1_P16A, 8. LabA1_F17A; C, D) pLab carrying: 1. SG6, 2. LabA2_D2A, 3. LabA2_E7A, 4. LabA2_T11A, 5. LabA2_L14A, 6. LabA2_F15A.

FIG. 21: LabA1 mutants prepared to verify A- and A′-ring flexibility. None of labyrinthopeptin A1 derivatives with changes in the A- or A′-ring size (peptide: LabA1_A2del, LabA1_N2insN, LabA1_N2insD, LabA1_A3insA, LabA1_G12del) could be detected by HPLC-MS analysis.

FIG. 22: LabA1 mutants prepared to verify flexibility of B- and B′-rings. Production of all labyrinthopeptin derivatives with changes in the B- or B′-ring size (peptide: LabA1_V5del, LabA1_W6insV, LabA1/A2, LabA1_P16del, LabA1_P16insV) could be detected by HPLC-MS analysis.

FIG. 23: Additional substitutions. The HPLC-ESI-MS analysis revealed the production of peptides: LabA1_T11S, LabA1_V15S, LabA1_A3H, LabA1_W6Y. No production of peptide: LabA1_E7R, LabA1_S4A, LabA1_S13A was observed.

FIG. 24: Mutants prepared to assess the flexibility of the C-ring. The HPLC-ESI-MS analysis revealed the production of peptides: LabA1_C20del, LabA1_S1insC, LabA1_S10insA and LabA1_C20insA.

FIG. 25: Graphical representation of LabA1 and LabA2 hybrids. The HPLC-ESI-MS analysis revealed the presence of labyrinthopeptin hybrids: LabA/A2 and LabA2/A1.

FIG. 26: Graphical presentation of LabA1_ABA′B′AB and LabA1_ABA′B′ABA′B′. Neither production of LabA1_ABA′B′AB, nor of LabA1_ABA′B′ABA′B′ could be detected by HPLC-ESI-MS analysis.

FIG. 27: Evaluation of anti-pain activity of labyrinthopeptin A2 and its structural analogs in in vitro assay: IC50 determination for LabA2 (A), LabA2_L14A (B) and LabA2 F15A (C). IC50 is a concentration of a substance that blocks 50% of channels.

FIG. 28: Structure of LabA2_L14A.

FIG. 29: ¹H NMR spectrum of LabA2_L14A.

FIG. 30: Comparison of the HSQC spectra of LabA2 and its mutant LabA2_L14A: alpha protons region and methyl groups region. Experimental conditions: Tris buffer pD 8.0 at 285 K. A, B) HSQC spectra—alphas area (A: LabA2, B: LabA2_L14A); C, D) HSQC spectra—methyls area (C: LabA2, D: LabA2_L14A).

FIG. 31: Construction of vector pCLL4. pCLL4 is comprising two functional origins of replication: from plasmid pAkij1 (Actinomadura kijanita) and from plasmid pBR322. Figure from (90).

FIG. 32: Map of pK18mobXylE. pK18mobXylE is a vector for gene inactivation experiments. It was constructed by cloning the gene xylE into pK18mob2.

FIG. 33: Map of pSET152ermE. pSET152ermE can integrate site-specifically at the bacteriophage ΦC31-attachment site (91)(92).

FIG. 34: Map of pUWLab, carrying the lab gene cluster under a control of the ermE* promoter.

FIG. 35: A) Map of pMK_SG2, a vector pMK carrying a synthetic gene SG2, that was used for site-directed mutagenesis of LabA1. B) Map of pMK_SG6, a vector pMK carrying a synthetic gene SG6, that was used for site-directed mutagenesis of LabA2.

FIG. 36: A) LabA1 isolated from A. namibiensis, MS/MS of m/z 1038.9 [M+2H]2+, B) D-LabA1 produced by S. lividans/pUWLab (supernatant from a 9 d culture in YEME medium), MS/MS of m/z 1096.4 [M+2H]2+, C) calculated and found fragment masses for LabA1, D) calculated and found fragment masses for D-LabA1, produced by S. lividans/pUWLab (supernatant from a 9 d culture in YEME medium), MS/MS of m/z 1096.4 [M+2H]2+.

### EXAMPLES

### Materials

### Bacterial Strains

| Strain | Genotype/Phenotype | Reference/Source |
|---|---|---|
| E. coli DH5α | supE44 ΔlacU169 | (1) |
| | (Φ80lacZΔM15) hsdR17 | |
| | recA1 endA1 gyrA | |
| | thi-1 relA1 | |
| E. coli BW25113 | lacIq rrnB_{T14} ΔlacZ_{WJ16} | (2) |
| | hsdR514 ΔaraBA-D_{AH33} | |
| | ΔrhaBAD_{LD78} | |
| E coli ET12567 | F⁻ dam-13::Tn9 dmc-6 | (3) |
| | hsdM hsdR lacY1 | |
| S. coelicolor M145 | | John Innes Centre, |
| | | Norwich, UK |
| S. albus CB89 | | Combinature Biopharm AG, |
| | | Berlin, Germany |
| S. avermitilis DSM 46492 | | DSMZ, Braunschweig, |
| | | Germany |
| S. lividans ZX7 | | Combinature Biopharm AG, |
| | | Berlin, Germany |
| S. griseus DSM 40236 | | DSMZ, Braunschweig, |
| | | Germany |
| A. namibiensis | Labyrinthopeptins producer | Sanofi-Aventis, Frankfurt |
| | | am Main, Germany |

### Antibodies

Primary antibody: Polyclonal antibodies produced in rabbits against labyrinthopeptin A2 and labyrinthopeptin A2 leader peptide were generated by BioGenes (Berlin, Germany) according to standard methods.

Secondary antibody: Commercially available Anti-Rabbit IgG Alkaline Phosphatase antibody produced in goat (Sigma-Aldrich Chemie GmbH, München).

### Material:

Plasmid isolation from Streptomyces

| | |
|---|---|
| TESLR | 25 mM Tris-HCl pH 8 |
| | 25 mM EDTA pH 8 |
| | 0.3M sucrose |
| | 0.02% bromocresol green |
| | 2 mg/ml lysozyme |
| | 5 μg/ml pre-boiled RNase A |
| NaOH/SDS | 0.3N NaOH |
| | 2% SDS |
| Acid phenol/chloroform | 5 g phenol |
| | 5 ml chloroform |
| | 1 ml H₂O |
| | 5 mg 8-hydroxyquinoline |

Genomic DNA isolation from Streptomyces and A. namibiensis

| | |
|---|---|
| SET buffer | 75 mM NaCl |
| | 25 mM EDTA pH 8.0 |
| | 20 mM Tris-HCl pH 7.5 |

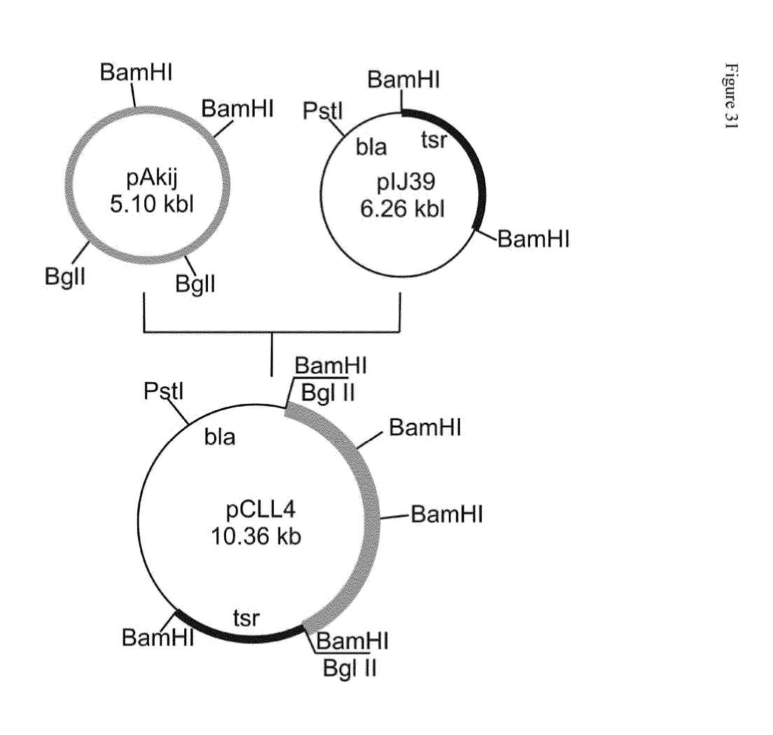

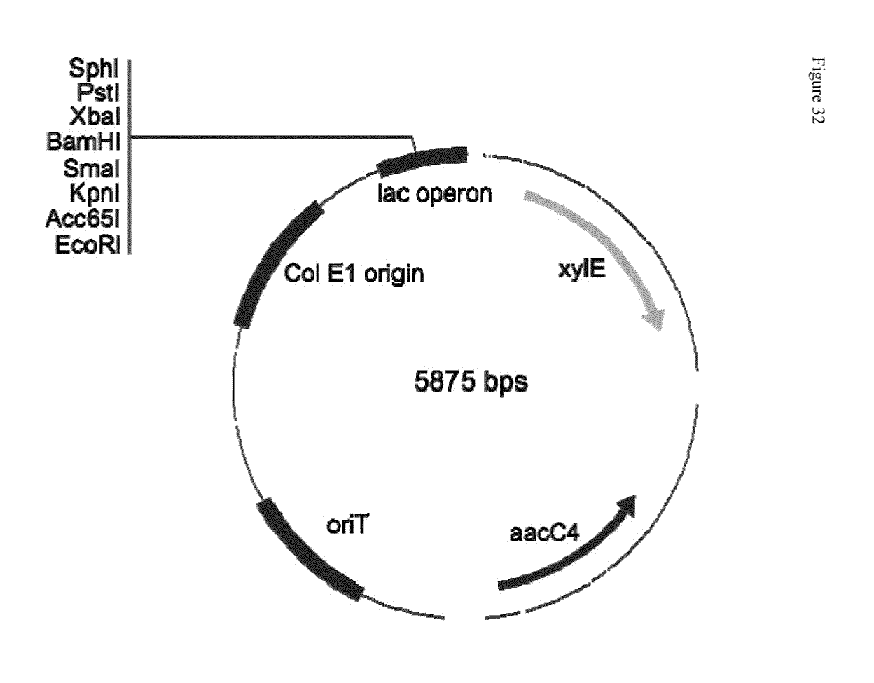

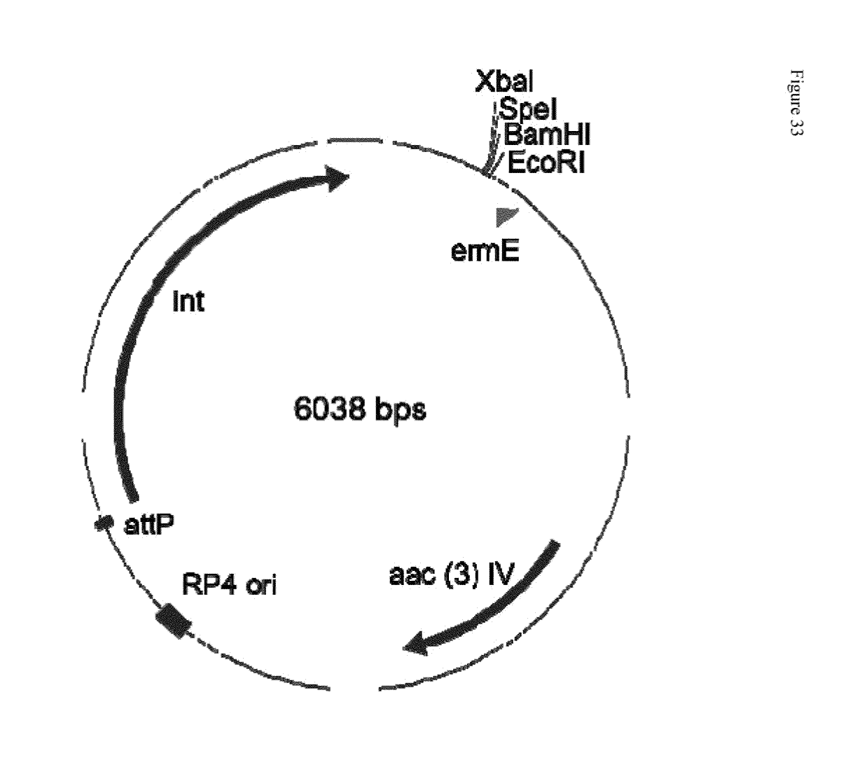

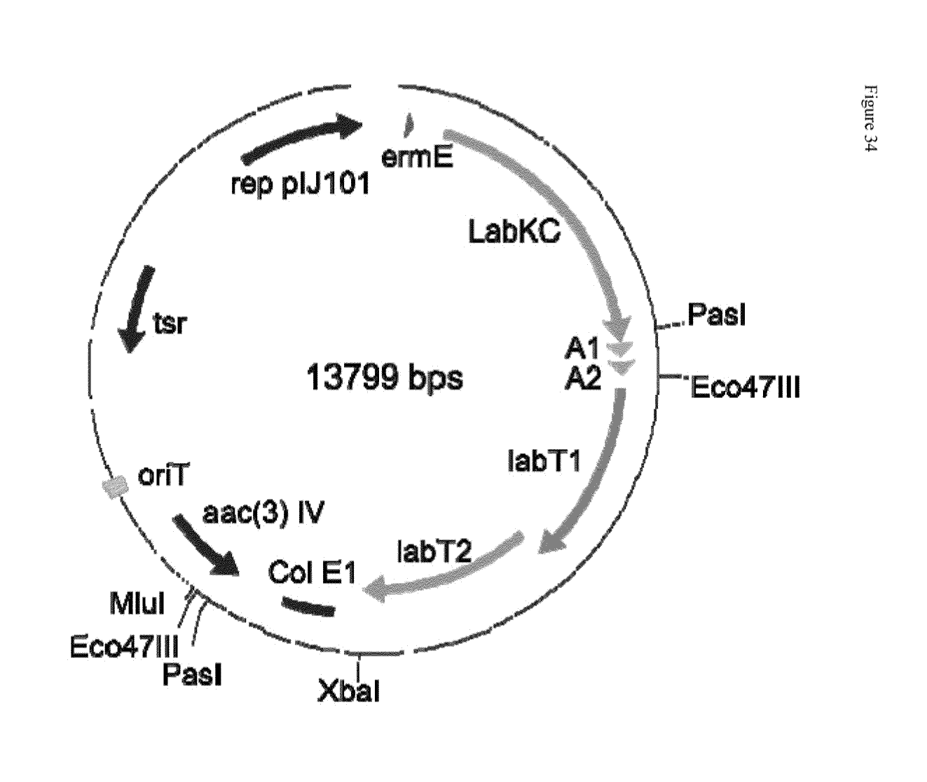

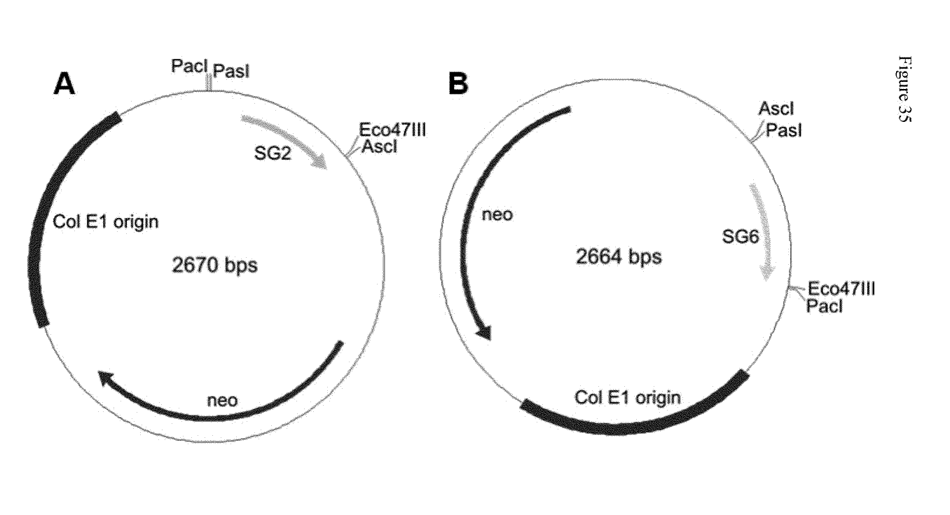

### Plasmids

| Plasmid | Characteristic | Reference/Source |
|---|---|---|
| pK18mob2 | vector for genes inactivation; | (4)/Combinature |
| | replicates in E. coli but not in | Biopharm AG, Berlin, |
| | Streptomyces; aac(3)IV | Germany |
| pK18mobXylE | vector for genes inactivation; | Combinature Biopharm |
| | replicates in E. coli but not in | AG, Berlin, Germany |
| | Streptomyces; xylE; aac(3)IV | |
| | see FIG. 31 | |
| pUZ8002 | non-transmissible oriT mobilising | (6)/John Innes |
| | plasmid; tra, neo, RP4 | Centre, Norwich, UK |
| pSET152ermE | contains oriT RK2 for conjugation | (7)/Combinature |
| | from E. coli to Streptomyces; | Biopharm AG, Berlin, |
| | ermE* promoter; attP, int, aac(3)IV | Germany |
| | See FIG. 33 | |
| pUWLoriT | bifunctional plasmid that replicate | (8)/Combinature |
| | in E. coli and Streptomyces; ermE* | Biopharm AG, Berlin, |
| | promoter; aac(3)IV, tsr | Germany |
| pMK | vector used for cloning of synthetic | Geneart AG, |
| | genes and for site-directed | Regensburg, |
| | mutagenesis; neo | Germany |
| pMA | vector used for cloning of synthetic | Geneart AG, |
| | genes and for site-directed | Regensburg, |
| | mutagenesis; bla | Germany |
| pCLL4 | vector comprising two functional | (9)/ATTC |
| | origins of replication: from plasmid | |
| | pAkij1 (A. kijanita) and from | |
| | plasmid pBR322 | |
| | see FIG. 31 | |
| pK18mob2_labKC | vector pK18mob2 carrying a 1.5 kb | This studies |
| | fragment of labKC gene; replicates | |
| | in E. coli but no in Streptomyces; | |
| | aac(3)IV | |
| pSETermEΔHindIII_oriCLL4 | vector pSET152 with an additional | This studies |
| | 0.9 kb from a vector pCLL4 | |
| | carrying an origin of replication | |
| | from A. kijanita; aac(3)IV | |
| pK18mobXylE_oripCLL4 | vector pK18mobXylE with an | This studies |
| | additional 0.9 kb from a vector | |
| | pCLL4 carrying an origin of | |
| | replication from | |
| | A. kijanita; aac(3)IV | |
| pCLL4_ori_apra | vector pCLL4 with an additional | This studies |
| | fragment containing oriT and | |
| | aac(3)IV gene (from a vector | |
| | pK18mob2); aac(3)IV, tsr | |
| pUWLab | plasmid carrying the lab gene | This study |
| | cluster under control of ermE* | |
| | promoter; vector replicates in E. | |
| | coli and Streptomyces; aac(3)IV, tsr | |
| | see FIG. 34 | |

Selective markers: Am, apramycin; Ap, ampicillin; Cm, chloramphenicol; Km, kanamycin; Th, thiostrepton.

### Oligonuceotides and Synthetic Genes

All the primers were synthesized by biomers.net GmbH (Ulm, Germany). Lyophilized primers were dissolved in sterile H₂Odd to reach a concentration 100 pmol/μl.

| | | |
|---|---|---|
| Mutant | Primers pair | Sequence (5′ → 3′) |

Alanine scanning mutagenesis of LabA1:

| | | |
|---|---|---|
| LabA1_N2A | LabA1_N2A_fw | CCGCCATGAGCGCGGCCAGCGTCTGG |
| | | (SEQ ID No. 26); |
| | LabA1_N2A_rv | CCAGACGCTGGCCGCGCTCATGGCGG |
| | | (SEQ ID No. 27) |
| LabA1_V5A | LabA1_V5A_fw | GCAACGCCAGCGCCTGGGAGTGCTG |
| | | (SEQ ID No. 28); |
| | LabA1_V5A_rv | CAGCACTCCCAGGCGCTGGCGTTGC |
| | | (SEQ ID No. 29) |
| LabA1_W6A | LabA1_W6A_fw | CAACGCCAGCGTCGCGGAGTGCTGCAG |
| | | (SEQ ID No. 30); |
| | LabA1_W6A_rv | CTGCAGCACTCCGCGACGCTGGCGTTG |
| | | (SEQ ID No. 31) |
| LabA1_E7A | LabA1_E7A_fw | CAGCGTCTGGGCCTGCTGCAGCACG |
| | | (SEQ ID No. 32); |
| | LabA1_E7A_rv | CGTGCTGCAGCAGGCCCAGACGCTG |
| | | (SEQ ID No. 33) |
| LabA1_T11A | LabA1_T11A_fw | GAGTGCTGCAGCGCGGGCAGCTGGG |
| | | (SEQ ID No. 34); |
| | LabA1_T11A_rv | CCCAGCTGCCCGCGCTGCAGCACTC |
| | | (SEQ ID No. 35) |
| LabA1_V15A | LabA1_V15A_fw | CACGGGCAGCTGGGCACCCTTCACCTGCTG |
| | | (SEQ ID No. 36); |
| | LabA1_V15A_rv | CAGCAGGTGAAGGGTGCCCAGCTGCCCGTG |
| | | (SEQ ID No. 37) |
| LabA1_F17A | LabA1_F17A_fw | GCTGGGTTCCCGCCACCTGCTGCTG |
| | | (SEQ ID No. 38); |
| | LabA1_F17A_rv | CAGCAGCAGGTGGCGGGAACCCAGC |
| | | (SEQ ID No. 39) |

Alanine scanning mutagenesis of LabA2:

| | | |
|---|---|---|
| LabA2_D2A | LabA2_D2A_fw | CCGCCATGTCCGCCTGGAGCCTGTG |
| | | (SEQ ID No. 40); |
| | LabA2_D2A_rv | CACAGGCTCCAGGCGGACATGGCGG |
| | | (SEQ ID No. 41) |
| LabA2_W3A | LabA2_W3A_fw | CGCCATGTCCGACGCCAGCCTGTGGGAG |
| | | (SEQ ID No. 42); |
| | LabA2_W3A_rv | CTCCCACAGGCTGGCGTCGGACATGGCG |
| | | (SEQ ID No. 43) |
| LabA2_L5A | LabA2_L5A_fw | CCGACTGGAGCGCGTGGGAGTGCTG |
| | | (SEQ ID No. 44); |
| | LabA2_L5A_rv | CAGCACTCCCACGCGCTCCAGTCGG |
| | | (SEQ ID No. 45) |
| LabA2_W6A | LabA2_W6A_fw | CGACTGGAGCCTGGCCGAGTGCTGTAGCAC |
| | | (SEQ ID No. 46); |
| | LabA2_W6A_rv | GTGCTACAGCACTCGGCCAGGCTCCAGTCG |
| | | (SEQ ID No. 47) |
| LabA2_E7A | LabA2_E7A_fw | GGAGCCTGTGGGCCTGCTGTAGCACG |
| | | (SEQ ID No. 48); |
| | LabA2_E7A_rv | CGTGCTACAGCAGGCCCACAGGCTCC |
| | | (SEQ ID No. 49) |
| LabA2_G12A | LabA2_G12A_fw | GCTGTAGCACGGCCAGCCTGTTCGCC |
| | | (SEQ ID No. 50); |
| | LabA2_G1A_rv | GGCGAACAGGCTGGCCGTGCTACAGC |
| | | (SEQ ID No. 51) |
| LabA2_L14A | LabA2_L14A_fw | GCACGGGAAGCGCGTTCGCCTGCTG |
| | | (SEQ ID No. 52); |
| | LabA2_L14A_rv | CAGCAGGCGAACGCGCTTCCCGTGC |
| | | (SEQ ID No. 53) |
| LabA2_F15A | LabA2_F15A_fw | CACGGGAAGCCTGGCCGCCTGCTGCTG |
| | | (SEQ ID No. 54); |
| | LabA2_F15A_rv | CAGCAGCAGGCGGCCAGGCTTCCCGTG |
| | | (SEQ ID No. 55) |

Ser/Ser/Cys motif (Ring A and A′):

| | | |
|---|---|---|
| LabA1_N2insD | LabA1_N2insD_fw | CCGCCATGAGCGACAACGCCAGCGTC |
| | | (SEQ ID No. 56); |
| | LabA1_N2insD_rv | GACGCTGGCGTTGTCGCTCATGGCGG |
| | | (SEQ ID No. 57) |
| LabA1_N2insN | LabA1_N2insN_fw | CCATGAGCAACAACGCCAGCGTCTG |
| | | (SEQ ID No. 58); |
| | LabA1_N2insN_rv | CAGACGCTGGCGTTGTTGCTCATGG |
| | | (SEQ ID No. 59) |
| LabA1_A3insA | LabA1_A3insA_fw | CATGAGCAACGCCGCCAGCGTCTGGGAG |
| | | (SEQ ID No. 60); |
| | LabA1_A3insA_rv | CTCCCAGACGCTGGCGGCGTTGCTCATG |
| | | (SEQ ID No. 61) |
| LabA1_A3del | LabA1_A3del_fw | CGCCATGAGCAACAGCGTCTGGGAG |
| | | (SEQ ID No. 62); |
| | LabA1_A3del_rv | CTCCCAGACGCTGTTGCTCATGGCG |
| | | (SEQ ID No. 63) |
| LabA1_G12insA | LabA1_G12insA_fw | GTGCTGCAGCACGGCCGGCAGCTGGGTTC |
| | | (SEQ ID No. 64) |
| | LabA1_G12insA_rv | GAACCCAGCTGCCGGCCGTGCTGCAGCAC |
| | | (SEQ ID No. 65) |
| LabA1_G12del | LabA1_G12del_fw | GTGCTGCAGCACGAGCTGGGTTCCC |
| | | (SEQ ID No. 66; |
| | LabA1_G12del_rv | GGGAACCCAGCTCGTGCTGCAGCAC |
| | | (SEQ ID No. 67) |

Ser/Ser/Cys motif (Ring B and B′):

| | | |
|---|---|---|
| LabA1_W6insV | LabA1_W6insV_fw | CAACGCCAGCGTCGTCTGGGAGTGCTGC |
| | | (SEQ ID No. 68); |
| | LabA1_W6insV_rv | GCAGCACTCCCAGACGACGCTGGCGTTG |
| | | (SEQ ID No. 69) |
| LabA1_W6insL | LabA1_W6insL_fw | CAACGCCAGCGTCCTGTGGGAGTGCTGC |
| | | (SEQ ID No. 70); |
| | LabA1_W6insL_rv | GCAGCACTCCCACAGGACGCTGGCGTTG |
| | | (SEQ ID No. 71) |
| LabA1_V5del | LabA1_V5del_fw | GAGCAACGCCAGCTGGGAGTGCTGC |
| | | (SEQID No. 72); |
| | LabA1_V5del_rv | GCAGCACTCCCAGCTGGCGTTGCTC |
| | | (SEQ ID No. 73) |
| LabA1_P16del | LabA1_P16del_fw | GGGCAGCTGGGTTTTCACCTGCTGC |
| | | (SEQ ID No. 74); |
| | LabA1_P16del_rv | GCAGCAGGTGAAAACCCAGCTGCCC |
| | | (SEQ ID No. 75) |
| LabA1_T18del | LabA1_T18del_fw | CTGGGTTCCCTTCTGCTGCTGACGC |
| | | (SEQ ID No. 76); |
| | LabA1_T18del_rv | GCGTCAGCAGCAGAAGGGAACCCAG |
| | | (SEQ ID No. 77) |
| LabA1_P16insV | LabA1_P16insV_fw | GCAGCTGGGTTGTCCCCTTCACCTG |
| | | (SEQ ID No. 78); |
| | LabA1_P16insV_rv | CAGGTGAAGGGGACAACCCAGCTGC |
| | | (SEQ ID No. 79) |
| LabA1_VP15del | LabA1_VP15del_fw | CACGGGCAGCTGGTTCACCTGCTGC |
| | | (SEQ ID No. 80); |
| | LabA1_VP15del_rv | GCAGCAGGTGAACCAGCTGCCCGTG |
| | | (SEQ ID No. 81) |

Substitute Ser with Thr:

| | | |
|---|---|---|
| LabA1_S1T | LabA1_S1T_fw | GGCCGCCATGACGAACGCCAGCGTC |
| | | (SEQ ID No. 82); |
| | LabA1_S1T_rv | GACGCTGGCGTTCGTCATGGCGGCC |
| | | (SEQ ID No. 83) |
| LabA1_S4T | LabA1_S10T_fw | CATGAGCAACGCCACCGTCTGGGAGTGC |
| | | (SEQ ID No. 84); |
| | LabA1_S10T_rv | GCACTCCCAGACGGTGGCGTTGCTCATG |
| | | (SEQ ID No. 85) |
| LabA1_S13T | LabA1_S13T_fw | GCAGCACGGGCACCTGGGTTCCCTTC |
| | | (SEQ ID No. 86); |
| | LabA1_S13T_rv | GAAGGGAACCCAGGTGCCCGTGCTGC |
| | | (SEQ ID No. 87) |

Express only east part of LabA1:

| | | |
|---|---|---|
| LabA1_C9tga | LabA1_C9tga_fw | CGTCTGGGAGTGCTGAAGCACGGGCAGCTG |
| | | (SEQ ID No. 88); |
| | LabA1_C9tga_rv | CAGCTGCCCGTGCTTCAGCACTCCCAGACG |
| | | (SEQ ID No. 89) |

Additional substitutions:

| | | |
|---|---|---|
| LabA1_S4A | LabA1_S4A_fw | CATGAGCAACGCCGCCGTCTGGGAGTG |
| | | (SEQ ID No. 90); |
| | LabA1_S4A_rv | CACTCCCAGACGGCGGCGTTGCTCATG |
| | | (SEQ ID No. 91) |
| LabA1_S13A | LabA1_S13A_fw | GTGCTGCAGCACGGGCGCCTGGGTTCCCT |
| | | (SEQ ID No. 92) |
| | LabA1_S13A_rv | TCAC;GTGAAGGGAACCCAGGCGCCCGTG |
| | | CTGCAGCAC (SEQ ID No. 93) |
| LabA1_V5T | LabA1_V5T_fw | GAGCAACGCCAGCACCTGGGAGTGCTG |
| | | (SEQ ID No. 94); |
| | LabA1_V5T_rv | CAGCACTCCCAGGTGCTGGCGTTGCTC |
| | | (SEQ ID No. 95) |
| LabA1_V15S | LabA1_V15S_fw | CACGGGCAGCTGGTCCCCCTTCACCTGC |
| | | (SEQ ID No. 96); |
| | LabA1_V15S_rv | GCAGGTGAAGGGGGACCAGCTGCCCGTG |
| | | (SEQ ID No. 97) |
| LabA1_W6Y | LabA1_W6Y_fw | CAACGCCAGCGTCTACGAGTGCTGCAGCAC |
| | | (SEQ ID No. 98); |
| | LabA1_W6Y_rv | GTGCTGCAGCACTCGTAGACGCTGGCGTTG |
| | | (SEQ ID No. 99) |
| LabA1_A3H | LabA1_A3H_fw | GCCATGAGCAACCACAGCGTCTGGGAG |
| | | (SEQ ID No. 100); |
| | LabA1_A3H_rv | CTCCCAGACGCTGTGGTTGCTCATGGC |
| | | (SEQ ID No. 101) |
| LabA1_E7R | LabA1_E7R_fw | CCAGCGTCTGGCGGTGCTGCAGCAC |
| | | (SEQ ID No. 102); |
| | LabA1_E7R_rv | GTGCTGCAGCACCGCCAGACGCTGG |
| | | (SEQ ID No. 103) |

Ring C:

| | | |
|---|---|---|
| LabA1_C20insA | LabA1_C20insA_fw | CCTTCACCTGCGCCTGCTGACGCCC |
| | | (SEQ ID No. 104); |
| | LabA1_C20insA_rv | GGGCGTCAGCAGGCGCAGGTGAAGG |
| | | (SEQ ID No. 105) |
| LabA1_S10insA | LabA1_S10insA_fw | GGGAGTGCTGCGCCAGCACGGGCAG |
| | | (SEQ ID No. 106); |
| | LabA1_S10insA_rv | CTGCCCGTGCTGGCGCAGCACTCCC |
| | | (SEQ ID No. 107) |
| LabA1_C20del | LabA1_C20del_fw | GTTCCCTTCACCTGCTGACGCCCGCACAC |
| | | (SEQ ID No. 108); |
| | LabA1_C20del_rv | GTGTGCGGGCGTCAGCAGGTGAAGGGAAC |
| | | (SEQ ID No. 109) |
| LabA1_S1insC | LabA1_S1insC_fw | CGGCCGCCATGTGCAGCAACGCCAG |
| | | (SEQ ID No. 110); |
| | LabA1_S1insC_rv | CTGGCGTTGCTGCACATGGCGGCCG |
| | | (SEQ ID No. 111) |

Spacer:

| LabA1_C9insV | LabA1_C9insV_fw | GTCTGGGAGTGCGTCTGCAGCACGGG |
|---|---|---|
| | | (SEQ ID No. 112); |
| | LabA1_C9insV_rv | CCCGTGCTGCAGACGCACTCCCAGAC |
| | | (SEQ ID No. 113) |
| LabA1_C9insVN | LabA1_C9insVN_fw | GGGAGTGCGTCAACTGCAGCACGGG |
| | | (SEQ ID No. 114); |
| | LabA1_C9insVN_rv | CCCGTGCTGCAGTTGACGCACTCCC |
| | | (SEQ ID No. 115) |
| Primer | Sequence (5′ → 3′) | Description |
| attB_For | CGGTCTCGAAGCCGCGGTGC | verification of |
| | (SEQ ID No. 116) | a presence of |
| attB_Rev | GCCCGCCGTGACCGTCGAG | phi att sites in |
| | (SEQ ID No. 117) | A. namibiensis |
| p18mob_EcoRI_for | CATCTCGAATTCCGCTCATG | amplify a fragment |
| | AGCTCAG | containing oriT and |
| | (SEQ ID No. 118) | aac(3)IV; used to |
| p18mob_EcoRI_rev | GTTATCGAGATCTGCAGGAG | generate a vector |
| | CTCTTTGG | pCLL4_ori_apra |
| | (SEQ ID No. 119) | |
| pSETproof_for | CGAGCCGGAAGCATAAAGTG | verification of a |
| | (SEQ ID No. 120) | correctness of an |
| pSETproof_rev | GCTTGGAGCGAACGACCTAC | exconjugant |
| | (SEQ ID No. 121) | A. namibiensis/ |
| | | pSET152 |
| STK_RTPCR_fw | agcagcaagtacgccgaacg | amplify a fragment |
| | (SEQ ID No. 122) | of labKC gene; used |
| STK_RTPCR_rv | gcgaagtggagctggttgag | to generate a vector |
| | (SEQ ID No. 123) | pDrive_labKC |
| pMA_seq | tgtgctgcaaggcgattaag | sequencing primer; |
| | (SEQ ID No. 124) | confirmation of |
| | | correctness of pMK_SGm |
| | | after site-directed |
| | | mutagenesis |
| Infusion 1 (fw) | gtcgaggcggccctggg | Primers used |
| | cggccaccccctgagac | for amplification |
| | (SEQ ID No. 125) | of an insert |
| Infusion 2 (rv) | gcggcctcggtcagcgctgt | SG(M) for ligation |
| | tcagcagcaggcgaacagg | independent cloning |
| | (SEQ ID No. 126) | |
| Infusion 3 (rv) | tcggcggcctcggtcagc | |
| | gctgttcagcagcaggtg | |
| | (SEQ ID No. 127) | |
| Infusion SG17 (rv) | CGGCGGCCTCGGTCAGCG | |
| | CTGTTCAGCAGCACCAG | |
| | (SEQ ID No. 128) | |
| Infusion SG18 (rv) | TCGGCGGCCTCGGTCAGC | |
| | GCTGTTCAGCAGCAGAG | |
| | (SEQ ID No. 129) | |
| Infusion SG19 (rv) | TTCGGCGGCCTCGGTCAG | |
| | CGCTGTTCAGCAACAGG | |
| | (SEQ ID No. 131) | |
| Infusion SG20 (rv) | CGGCGGCCTCGGTCAGCG | |
| | CTGTTCAGCAGGCGAAC | |
| | (SEQ ID No. 131) | |

All synthetic genes were synthesized by Geneart AG (Regensburg, Germany). Sequences of synthetic genes are summarized below.

aac(3)IV
(SEQ ID No. 25)
ACGCGTCGATTATCTCGAGAATGACCACTGCTGTGAGCGGTTTGCC
TTGGCGGACAGGTGGCTCAAGGAGAAGAGCCTTCAGAAGGAAGGTC
CAGTCGGTCATGCCTTTGCTCGGTTGATCCGCTCCCGCGACATTGT
GGCGACAGCCCTCGGTCAACTGGGCCGAGATCCGTTGATCTTCCTG
CATCCGCCAGAGGCGGGATGCGAAGAATGCGATGCCGCTCGCCAGT
CGATTGGCTGAGCTCATGAGCGGAGAACGAGATGACGTTGGAGGGG
CAAGGTCGCGCTGATTGCTGGGGCAACACGTGGAGCGGATCGGGGA
TTGTCTTTCTTCAGCTCGCTGATGATATGCTGACGCTCAATGCGCC
TCACTGATTAAGCATTGGTAACTGTCAGACCAAGTTTACTCATATA
TACTTTAGATTGATTTAAAACTTCATTTTTAATTTAAAAGGATCTA
GGTGAAGATCCTTTTTGATAATCTCATGACCAAAATCCCTTAACGT
GAGTTTTCGTTCCACTGAGCGTCAGACCCCGTAGAAAAGATCAAAG
GATCTTCTTGAGATCCTTTTTTTCTGCGCGTAATCTGCTGCTTGCA
AACAAAAAAACCACCGCTACCAGCGGTGGTTTGTTTGCCGGATCAA
GAGCTACCAACTCTTTTTCCGAAGGTAACTGGCTTCAGCAGAGCGC
AGATACCAAATACTGTTCTTCTAGTGTAGCCGTAGTTAGGCCACCA
CTTCAAGAACTCTGTAGCACCGCCTACATACCTCGCTCTGCTAATC
CTGTTACCAGTGGCTGCTGCCAGTGGCGATAAGTCGTGTCTTACCG
GGTGGACTCAAGACGATAGTTACCGGATAAGGCGCAGCGGTCGGGC
TGAACGGGGGGTTCGTGCACACAGCCCAGCTTGGAGCGAACGACCT
ACACCGAACTGAGATACCTACAGCGTGAGCTATGAGAAAGCGCCAC
GCTTCCCGAAGGGAGAAAGGCGGACAGGTATCCGGTAAGCGGCAGG
GTCGGAACAGGAGAGCGCACGAGGGAGCTTCCAGGGGGAAACGCCT
GGTATCTTTATAGTCCTGTCGGGTTTCGCCACCTCTGACTTGAGCG
TCGATTTTTGTGATGCTCGTCAGGGGGGCGGAGCCTATGGAAAAAC
GCCAGGCACGCGGCCTTTTTACGGTTCCTGGCCTTTTGCTGGCCTT
TTGCTCACATGTTCTTTCCTGCGTTATCCCCTGATTCTGTGGATAA
CCGTATTACCGCCTTTGAGTGAGCTGATACCGCTCGCCGCAGCCGA
ACGACCGAGCGCAGCGAGTCAGTGAGCGAGGAAGCGGAAGAGCGCC
CAATACGCAAACCGCCTCTCCCCGCGCGTTGGCCGATTCATTAATG
CAGAGCTTCTAGA

XbaI (TCTAGA) and MluI (ACGCGT) are underlined. Changed Past (CCCTCGG) and Eco47III (AGCGGT) restriction sites are underlined.

In the following sequences, the respective pre-peptide sequence is shown in bold. Past (CCCTGGG) and Eco47III (AGCGCT) restriction sites are underlined. labA1 gene is shown in black letters, labA2 gene is marked with white letters with black background. Propeptide sequences are shown in bold.

SG2
(SEQ ID No. 1)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
CAACGCCAGCGTCTGGGAGTGCTGCAGCACGGGCAGCTGGGTTCCCTTCA
CCTGCTGCTGACGCCCGCACACCGTTCCACCGATGAGAGGTGACAGTCCC

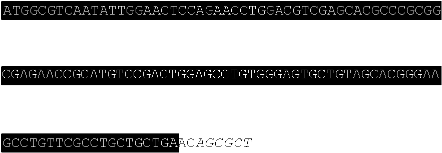

SG3
(SEQ ID No. 2)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCGCCA
GCAACGCCAGCGTCTGGGAGTGCTGCAGCACGGGCAGCTGGGTTCCCTTC
ACCTGCTGCTGACGCCCGCACACCGTTCCACCGATGAGAGGTGACAGTCC
CATGGCATCCATCCTTGAGCTCCAGGACCTGGAGGTCGAGCGCGCCAGCT

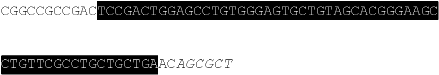

SG4
(SEQ ID No. 3)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCGACA
GCAACGCCAGCGTCTGGGAGTGCTGCAGCACGGGCAGCTGGGTTCCCTTC

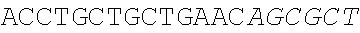

SG5
(SEQ ID No. 4)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT

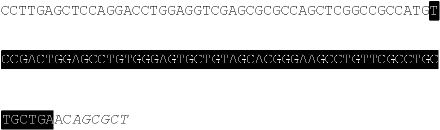

SG6
(SEQ ID No. 5)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT

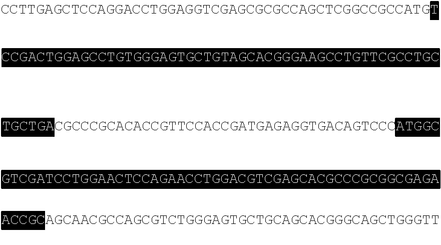

CCCTTCACCTGCTGCTGAACAGCGCT
SG7
(SEQ ID No. 6)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGT
GCAGCACGGGCAGCTGGGTTCCCTTCACCTGCTGCTGACGCCCGCACACC

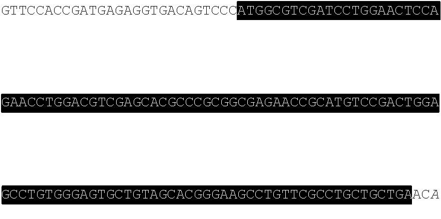

GCGCT
SG8
(SEQ ID No. 7)
CCCTGGGCGGCCACCCCCTGAGACTCCCCTTCCTCCATCCGGAGGAAGGG
GCGAGCACGCGACCCCGGGGGAGGAGGTGAACATCCACCatggcacttct
cgacctgcagacgatggaagccgacgagacgaccggtaccggcgggccca
gctccctgagcgtgctgtcctgtgtgagcgcggccagcatcacgctctgc

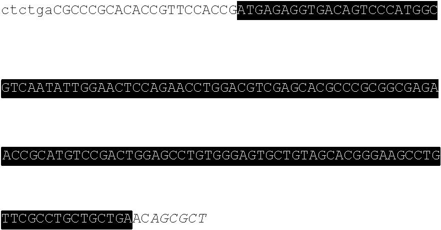

RamS2 prepropeptide sequence from S. scabiei is
marked in small letters.
SG9
(SEQ ID No. 8)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGGCAGCTGGGTTCCCTTC
ACCTGCTGCAGCAACGCCAGCGTCTGGGAGTGCTGACGCCCGCACACCGT

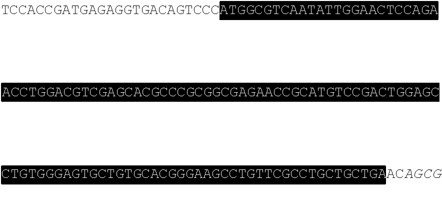

CT
SG10
(SEQ ID No. 9)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGGCAGCTGGGTTCCCTTC
ACCTGCTGCAGCAACGCCAGCGTCTGGGAGTGCGCCAGCACGGGCAGCTG
GGTTCCCTTCACCTGCGCCTGACGCCCGCACACCGTTCCACCGATGAGAG

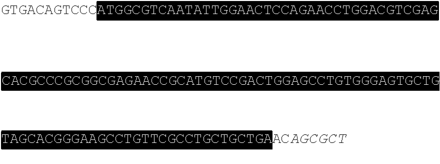

SG11
(SEQ ID No. 10)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCGACA

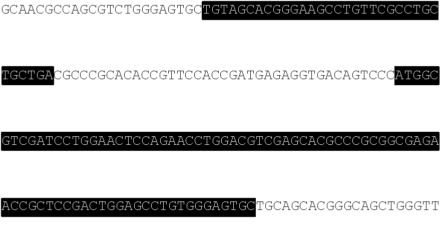

CCCTTCACCTGCTGCTGAACAGCGCT
SG12
(SEQ ID No. 11)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCTGGTGCTGCAGCACGGGCAGCTGGGTTCCCTTCACCTGC

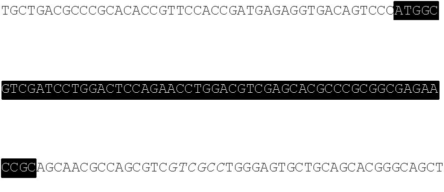

GGGTTCCCTTCACCTGCTGCTGAACAGCGCT
SG13
(SEQ ID No. 12)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGGCAGCTGGACCTGCTGC

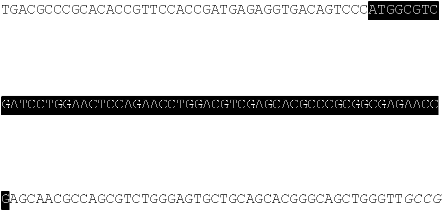

TCCCCTTCACCTGCTGCTGAACAGCGCT
SG14
(SEQ ID No. 13)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCGCCAGCACGGGCAGCTGGGTTCCCTTC
ACCTGCGCCTGACGCCCGCACACCGTTCCACCGATGAGAGGTGACAGTCC

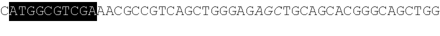

GTTCCCTTCACCTGCTGCTGAACAGCGCT
SG15
(SEQ ID No. 14)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCGTCAGCGCCTGGGAGTGCTGCAGCACGGGCAGCTGG
GTTCCCTTCACCTGCTGCTGACGCCCGCACACCGTTCCACCGATGAGAGG

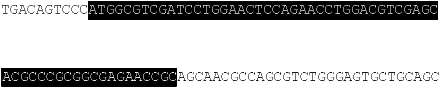

ACGGGCAGCTGGGTTAGCCCCTTCACCTGCTGCTGAACAGCGCT
SG16
(SEQ ID No. 15)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCAGCGGCAGCTGGGTTCCCTTC
ACCTGCTGCTGACGCCCGCACACCGTTCCACCGATGAGAGGTGACAGTCC

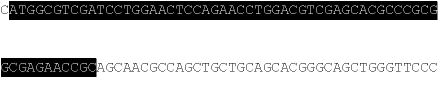

TTCACCTGCTGCTGAACAGCGCT
SG17
(SEQ ID No. 16)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGGCAGCTGGGTTCCCTTC
AGCTGCTGCTGACGCCCGCACACCGTTCCACCGATGAGAGGTGACAGTCC

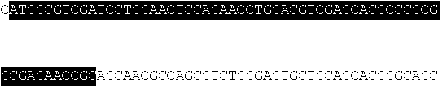

TGGTGCTGCTGAACAGCGCT
SG18
(SEQ ID No. 17)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGa
gctccctgagcgtgctgtcctgtgtgagcgcggccagcatcacgctctgc

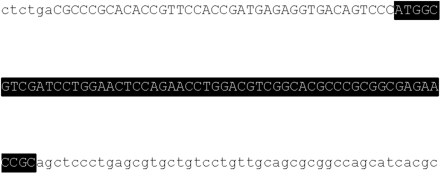

tctgctgctgaACAGCGCT
RamS2 prepeptide sequence from S. scabiei is marked
in small letters.
SG19
(SEQ ID No. 18)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGa
ccggcagccgcgcgagcctcctgctctgcggcgacagcagcctgagcatc
accacctgtaactgaCGCCCGCACACCGTTCCACCGATGAGAGGTGACAG

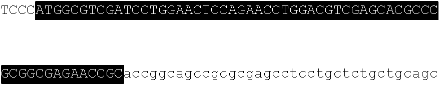

agcctgagcatcaccacctgttgctgaACAGCGCT
SapB prepeptide sequence from S. coelicolor is
marked in small letters.
SG20
(SEQ ID No. 19)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCATGTGCAGCACGGGCAGCTGGGTTCCC
TTCACCTGCTGCTGACGCCCGCACACCGTTCCACCGATGAGAGGTGACAG

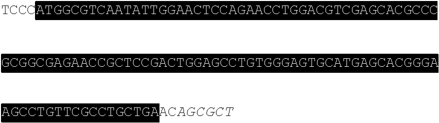

LabA2_T11A
(SEQ ID No. 20)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT

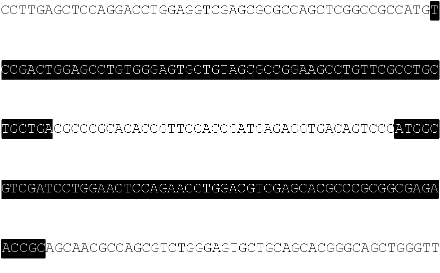

CCCTTCACCTGCTGCTGAACAGCGCT
LabA1_G12A
(SEQ ID No. 21)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGCCAGCTGGGTTCCCTTC
ACCTGCTGCTGACGCCCGCACACCGTTCCACCGATGAGAGGTGACAGTCC

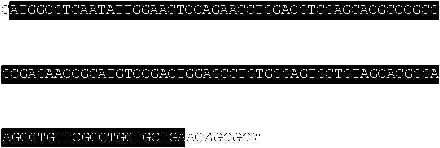

LabA1_W14A
(SEQ ID No. 22)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGGCAGCGCCGTTCCCTTC
ACCTGCTGCTGACGCCCGCACACCGTTCCACCGATGAGAGGTGACAGTCC

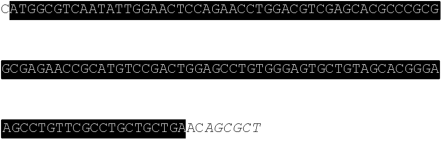

LabA1_P16A
(SEQ ID No. 23)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGGCAGCTGGGTTGCCTTC
ACCTGCTGCTGACGCCCGCACACCGTTCCACCGATGAGAGGTGACAGTCC

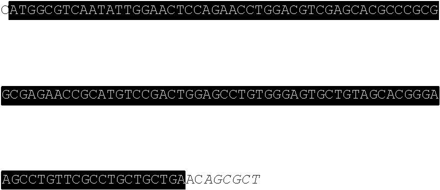

LabA1_T18A
(SEQ ID No. 24)
CCCTGGGCGGCCACCCCCTGAGACTCCCCTTCCTCCATCCGGAGGAAGGG
GCGAGCACGCGACCCCGGGGGAGGAGGTGAACATCCACCATGGCATCCAT
CCTTGAGCTCCAGGACCTGGAGGTCGAGCGCGCCAGCTCGGCCGCCATGA
GCAACGCCAGCGTCTGGGAGTGCTGCAGCACGGGCAGCTGGGTTCCCTTC
GCCTGCTGCTGACGCCCGCACACCGTTCCACCGATGAGAGGTGACAGTCC

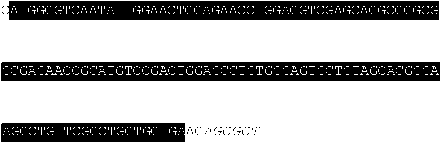

### Methods

Standard molecular biology methods were used according to Sambrook and colleagues (1989) and are not described here in detail. When temperature conditions are not specified then it means that an experiment was performed at room temperature.

### Microbiological Methods:

Growth Conditions and Preservation of E. coli

E. coli strains were grown in LB medium supplemented with appropriate antibiotics. The cultures were incubated at 37° C. with shaking at 160 rpm. E. coli were stored at −80° C. in 10% glycerol.

Growth Conditions and Preservation of Streptomyces and A. namibiensis

Streptomyces and Actinomadura namibiensis were grown on different agar media (like MS, R2YE, R5, KM4) at 28° C. and in liquid media (like YEME, CRM, KM4, M5294) at 28° C. shaking at 160 rpm. 50 ml bacterial culture was cultivated in 250 ml or 300 ml flask with a coil for a better aeration. Streptomyces containing recombinant plasmids were grown in presence of apramycin (25 μg/ml or 50 μg/ml). For first cultures prepared after conjugation Streptomyces were grown also with nalidixic acid (25 μg/ml). For cultivation of A. namibiensis no antibiotics were used. Streptomyces as well as A. namibiensis were stored at −80° C. in 10% glycerol with addition of 5% sucrose.

### Molecular Biological Methods:

Preparation of Plasmid DNA from E. coli

E. coli plasmid DNA was purified using the GeneJET™ Plasmid Miniprep Kit (Fermentas) according to the producer's protocol.

Preparation of Plasmid DNA from Streptomyces

Plasmid DNA from Streptomyces was isolated by alkaline lysis and phenol precipitation according to the protocol described by Kieser et al., 2000 (10). First step is resuspension of mycelium from 5 ml culture in a total volume of 500 μl TESLR and incubation for 30 min at 37° C. Then 250 μl of NaOH.3/SDS is added and the mixture is immediately vortex. After 15 min incubation of open tubes at 70° C. they need to be cooled to 37° C. The next step is addition of 80 μl acid phenol/chloroform, emulsification by vortexing and centrifugation for 10 min. Now supernatant (700 μl) is transferred to a new centrifuge tube containing 50 μl sodium acetate and 450 μl isopropanol. Such mixture is vortex and centrifuge for 10 min. The obtained precipitate is dissolved now in 70 μl TE. This is followed by addition of 3M potassium acetate (14 μl), acid phenol/chloroform (14 μl), emulsification and vortexing. Then upper aqueous phase is transferred to a new tube containing 56 μl of 3M potassium acetate and 630 μl of TE. Afterwards 460 μl isopropanol is added and the mixture is centrifuged for 5 min to get a pellet which should be washed with 1.2 ml 0° C. ethanol and dissolved in 150 μl TE. 150 μl 5M ammonium acetate is added to DNA solution which is then mixed and centrifuged. After that supernatant (pellet should be discarded) is transferred to a new tube. This is followed by addition of 660 μl ethanol and centrifugation for 10 min. At the very end obtained pellet is washed with 1 ml 70% ethanol, dried and dissolved in 50 μl TE.

Preparation of Genomic DNA from Streptomyces

For preparation of genomic DNA from Streptomyces salting out procedure described by Kieser et al., 2000 (10) was applied. 30 ml of bacterial culture is grown in standard Streptomyces medium (like TSB, CRM, YEME) with glycine (end conc. 0.5%) in 100 ml flasks. At the beginning mycelium from 7 ml of culture needs to be resuspended in 1.25 ml SET buffer. Then 25 μl of lysozyme solution is added and such mixture is incubated for 60 min at 37° C. (final lysozyme concentration is 2 μg/ml). The next step is addition of 35 μl proteinase K solution (final proteinase conc. is 0.5 μg/ml), mixing, addition of 150 μl 10% SDS (final SDS conc. is 1%), mixing by inversion and incubation for 2 h at 55° C. It is important to invert occasionally the sample during the whole incubation time. Afterwards 500 μl of 5 M NaCl is added, the sample is mixed thoroughly by inversion and cooled to 37° C. Next 1.25 ml of chloroform is added. Such prepared mixture needs to be mixed by inversion for 30 min at 20° C. Then after 15 min centrifugation (4500×g at 20° C.) the supernatant is transferred to a fresh tube. This is followed by addition of 0.6 vol isopropanol. After 3 min of mixing by inversion DNA can be spooled onto a sealed Pasteur pipette, rinsed in 1.25 ml 70% ethanol and air dried. At the end DNA is dissolved in 100 μl water at 55° C.

### Measuring DNA Concentration

DNA concentration was measured by means of UV spectrophotometry at 2=260 nm (Ultrospec 2100 pro Classic; Amersham Pharmacia Biotech, Freiburg, Germany). Samples were 16 times diluted (5 μl sample in 75 μl water). Pure DNA yields an A260/A280 ratio of 1.8-1.9.

### Agarose Gel Electrophoresis

Agarose gel electrophoresis was used for separation of mixed population of DNA fragments by length and for estimation of their size. Nucleic acid molecules are separated by applying an electric field to move the negatively charged molecules through an agarose matrix. Shorter molecules move faster and migrate farther than longer ones because shorter molecules migrate more easily through the pores of the gel. Samples were prepared by addition of 10% of a loading buffer. The loading buffer gives color and density to the sample to make it easy to load into the wells. Different agarose concentrations were employed: 2% agarose gels for DNA fragments smaller that 1 kp, 0.8% agarose gels for DNA fragments obtained after restriction digestion of cosmids. For all the other purposes 1% agarose gels were prepared. Gels were run in a horizontal tank (Mini-Sub Cell GT; Bio-Rad Laboratories GmbH, München, Germany) in TAE buffer (Tris/Acetate/EDTA). Separation of DNA fragments was achieved by using a voltage between 50 V and 90 V. After electrophoresis the gel was incubated in an ethidium bromide bath. DNA bands were visualized by the use of UV light (λ=315 nm). As a molecular weight marker 2-log DNA marker (New England Biolabs, Frankfurt am Main, Germany) was used.

DNA Extraction from Agarose Gels

DNA fragments were isolated from an agarose gel by the use of GeneJet Gel Extraction kit from Fermentas.

### Restriction Enzyme Digestion of DNA

Restriction digestion enzymes from Fermentas and New England BioLabs were used. Restriction digests were performed according to manufacturer's recommendations.

Introduction of DNA into E. coli
Electroporation of E. coli ET12567/pUZ8002

E. coli is grown overnight at 37° C. in 10 ml LB medium containing kanamycin (50 μg/ml) and chloramphenicol (34 μg/ml). The next day 10 ml LB medium containing kanamycin (50 μg/ml), chloramphenicol (34 μg/ml) and 20 mM MgSO₄ is inoculated with 100 μl E. coli from the overnight culture. Bacteria are grown at 37° C. shaking at 160 rpm to an OD₆₀₀ of ˜0.4. Afterwards cells are recovered by centrifugation at 4,000 rpm for 5 min at 4° C. and the pellet is resuspended by gentle mixing in 10 ml ice-cold 10% glycerol. Then the cell suspension is again centrifuged and the pellet is resuspended in 5 ml ice-cold 10% glycerol. After another centrifugation step the pellet is resuspended in the remaining ˜100 μl 110% glycerol. For electroporation 50 μl of such obtained cell suspension is mixed with 100-300 ng of plasmid DNA. Experiment is carried out in a 0.2 cm ice-cold electroporation cuvette using a power supply unit (BioRad) set to: 200 Ω, 25 μF and 2.5 kV. Just after 1 ml ice cold LB is added to shocked cells. Bacteria are incubated shaking for 1 h at 37° C. and then they are spread onto LB agar containing apramycin (100 μg/ml), kanamycin (50 μg/ml) and chloramphenicol (34 μg/ml). Plates are incubated overnight at 37° C.

### Chemical Transformation

E. coli stocks should be thawed on ice. Then to an aliquot bacterial suspension (50 μl or 100 μl per tube) 1 to 5 μl of DNA mix is added (e.g., isolated plasmid, ligation mix) and bacteria are incubated 30 min on ice. Afterwards a heat shock takes place: samples are incubated at 42° C. for 2 min. Then samples are immediately transferred on ice. After 10 min 1 ml of LB medium is added. Samples are incubated at 37° C. for 1 h with shaking at 160 rpm. The last step is platting of bacterial suspension on an agar plate with appropriate antibiotic. Plates are incubated overnight at 37° C.

Introduction of DNA into Streptomyces and A. namibiensis

### Preparation and Transformation of Protoplasts

### Preparation of Protoplasts

25 ml of medium (YEME and TSB for Streptomyces; KM4 and CRM for A. namibiensis; with 0.5% of glycine) is inoculated with bacteria from a cryostock or agar plate culture, and grown for few days. When the culture reaches the appropriate growth phase then 20 ml of a culture should be centrifuged (2,000 rpm, 10 min) Then pellet is resuspended in 15 ml 10.3% sucrose and spun in a centrifuge with the same conditions as described above. This step is repeated twice. After the centrifugation step mycelium is resuspended in 4 ml of P buffer or L buffer containing for Streptomyces 1-2 mg/ml of lysozyme and for A. namibiensis a mixture of cellosyl (1 μg/ml) and lysozym (0.5 μg/ml) solubilized in P-buffer (containing 20% of sacharose instead of a commonly used 10% solution). Such suspension is incubated either at 28° C., 30° C. or 37° C. until protoplasts are observed under the microscope. Later cells are draw in and out three times and incubated for a further 15 min. This is followed by addition of P buffer and again cells are draw in and out and incubated for a further 15 min. Next protoplasts are filtered through cotton wool (using a filter tube), transferred to a plastic tube and gently centrifuged (1,000 g, 7 min) Now supernatants is discarded and protoplasts are suspended in 1 ml P buffer. Such prepared protoplasts can be frozen or directly transformed.

### PEG-Assisted Transformation of Protoplasts

For one transformation 50 μl of protoplast sample should be used. It is recommended to spin protoplasts down immediately before a transformation experiment. First, 5 μl of DNA solution should be added to protoplasts and mixed immediately by tapping the tube. Second, 200 μl T buffer (*) is added and mixed by pipetting up and down. Then protoplasts are spread on two dried R2YE plates and incubated at 28° C. The next day (after 14-20 h) plates are overlayed for selection. (*) For preparation of Buffer T two different PEGs were used: PEG 1000 (Roth) and PEG 3350 (Sigma Aldrich).

Conjugation from E. coli

First, competent cells of E. coli ET12567/pUZ8002 need to be prepared in the presence of kanamycin (50 μg/ml) and chloramphenicol (34 μg/ml). These cells are transformed with the oriT-containing vector by electroporation. Obtained colonies should be inoculated into 10 ml LB medium containing kanamycin (50 μg/ml), chloramphenicol (34 μg/ml) and apramycin (100 μg/ml) each and grown overnight. The next day 10 ml LB medium (for one conjugation) containing kanamycin (50 μg/ml), chloramphenicol (34 μg/ml), apramycin (100 μg/ml) is inoculated with the overnight culture (the overnight culture is diluted 1:100). Bacteria are grown at 37° C. to OD₆₀₀ of 0.4-0.6. Then E. coli cells are washed twice with 10 ml of LB medium. After centrifugation step the supernatant is discarded and the pellet is resuspended in 500 μl LB medium. While washing the E. coli cells, for each conjugation approximately 10⁸ Streptomyces spores are added to 500 μl LB medium and such suspension is heat shocked at 50° C. for 10 min. (If mycelial fragments are used for conjugation, then bacteria is harvested from a 3-4 d old culture growing on MS agar using 3 ml 20% glycerol. Approximately 0.5 ml of the mycelial fragments is used for each conjugation. In a case of mycelial fragments the heat shock is omitted.) Afterwards 500 μl of E. coli cells are added to 500 μl heat-shocked spores or mycelial fragments. After mixing most of the supernatant is poured off and the pellet is resuspended in the residual medium. Now the bacteria suspension is plated out on

MS agar+10 mM MgCl₂. The plate is incubated at 28° C. for 16-20 h. The next day the plate is overlayed with 1 ml water or 4 ml of a soft agar containing 0.5 mg nalidixic acid and 1 mg apramycin. Incubation is continued at 28° C. for the next few days until potential exconjugants are observed on the plate.
Direct Transformation Attempts with A. namibiensis

Experiments were performed according to the protocol described by Madoń and Hütter, 1991 (11).

Direct transformation of A. namibiensis mycelium

100 ml of a Actinomadura namibiensis preculture in KM4 media is inoculated with bacteria growing on a KM4 agar plate and incubated shaking (160 rpm) at 28° C. for one day. The next day 100 ml of KM4 medium is inoculated with preculture and incubated in an orbital shaker at 160 rpm for three days at 28° C. Aliquots (10 ml) of the culture are centrifuged in 15 ml Falcon tubes at 5,000 rpm for 15 min. Afterwards the mycelium (from one Falcon tube) is resuspended in 4 ml of 20% aqueous glycerol solution and stored at −28° C. or it is directly used for a transformation experiment. Directly before transformation, a mycelial suspension is thawed, washed three times in 25 ml of TE buffer. Then a pellet is resuspended in 0.6 ml TE buffer. Mycelium and transformation mixtures should be prepared at room temperature. To 100 μl of a TE mycelium suspension are added: 10 μl of 0.2 M MgCl₂, 60 μl of 4.17 M CsCl, 4 μl of calf thymus DNA, up to 10 μl of plasmid DNA (pSET152: 96 ng, 320 ng; pUWLoriT: 117 ng, 390 ng), and TE buffer to final volume of 100 μl. This is followed by the addition of 200 μl of 70% (w/v) PEG-3350 in TE buffer. After each constituent is added, the mixture is mixed thoroughly. The transformation mixture is incubated at 28° C. for 90 min and then at 42° C. for 5 min and finally cooled to room temperature. The whole content of the transformation mixture is added directly to 3 ml of R2L-overlay medium and plated on S27M agar plates. Plates are dried by incubation in a laminar flow cabinet for 15 min. After incubation at 28° C. for 18 h each plate is overlayed with 1.5 ml water containing 1 mg of apramycin. It is important to use fresh S27M agar plates. Preferably prepare them on the day of transformation and dry in a laminar flow cabinet for 3 h.

Direct Transformation of A. namibiensis Protoplasts

Protoplasts prepared in the way described in section 4.2.3.1 are used. To 100 μl of refrozen protoplasts in P buffer is added: 10 μl of 0.2 M MgCl₂, 60 μl of 4.17 M CsCl, 4 μl of calf thymus DNA, up to 10 μl of a plasmid DNA (96 ng, 160 ng of pSET152 and 117 ng, 195 ng of pUWLoriT), and TE buffer to final volume of 100 μl. This is followed by the addition of 200 μl of 70% (w/v) PEG in TE buffer. After each constituent is added, the mixture is mixed thoroughly. The transformation mixture is incubated at 28° C. for 40 min and then at 42° C. for 5 min and finally cooled to room temperature. The whole content of the transformation mixture is added directly to 3 ml of R2L-overlay medium and plated on S27M agar plates. Plates are dried by incubation in a laminar flow cabinet for 15 min. After incubation at 28° C. for 18 h each plate is overlayed with 1.5 ml water containing 1 mg of apramycin.

### Electroporation of Mycelium

A slightly modified protocol has been used as previously published for Streptomyces ramous (10).

A preculture of A. namibiensis is inoculated with bacteria from a KM4 agar plate and is incubated in KM4 medium shaking (160 rpm) at 28° C. for one day. The next day three different cultures are inoculated with 5 ml of preculture: 50 ml of CRM medium, 50 ml of CRM medium with glycine (end conc. 0.5%), 100 ml of KM4 medium. These cultures are incubated in an orbital shaker at 160 rpm for four days at 28° C. Then mycelium is harvest by centrifugation (4° C., 10,000 rpm) and cells are resuspended in 50 ml ice-cold 10.32% sucrose and recentrifuged. This step is repeated twice. Afterwards the mycelium is resuspended in 4 ml 15% glycerol containing 15 mg of cellosyl. After incubation at 37° C. for 3 h the mycelium is washed twice with 7 ml ice-cold 15% glycerol. Later the pellet is resuspended in 2 ml of 30% PEG 1000, 10% glycerol, 5% sucrose. For electroporation,

50 μl of such suspension with plasmid DNA is mixed by pipetting (96 ng and 192 ng of pSET152; 117 ng and 234 ng of pUWLoriT). Sample must be placed on ice.

Electroporation is carried out in a 0.2 cm ice-cold electroporation cuvette using 2 kV electric pulse from a Gene Pulser (Bio-Rad), connected to a Pulse Controller (parallel resistance 400 Ω, 25 μF capacitor). Pulsed mycelium is immediately diluted with 1 ml ice-cold KM4 medium and it is incubated with shaking for 3 h at 28° C. Bacteria dilutions are plated on KM4 medium. The next day each plate is overlayed with 1 ml water containing 1 mg of apramycin.

### Polymerase Chain Reaction (PCR)

During these studies two kinds of DNA polymerases were used: Taq (Qiagen) and Herculase II (Stratagene). Because Taq DNA polymerase has no 3′ to 5′ exonuclease activity which could cause misincorporations it was used only for colony PCR, verification of positive transformants by PCR and preparation of DIG-labeled DNA probes. For all the other applications like site-directed mutagenesis, amplification of fragments used for cloning and sequencing Herculase II DNA polymerase was used. Standard reaction composition and Thermal Cycler conditions are summarized below:

| | | | | Herculase II |
|---|---|---|---|---|
| Taq polymerase | | | | polymerase |
| | Quantity per | | | Quantity per |
| Component | reaction | | Component | reaction |
| dH₂O | To final | | dH₂O | To final volume of |
| | volume of | | | 50.0 μl |
| | 50.0 μl | | | |
| 10x CoralLoad PCR | 5 | | 5x Herculase II | 10 μl |
| buffer | | | reaction buffer | |
| dNTP mix | 1 | | dNTP mix | 0.5 μl |
| (10 mM each) | | | (25 mM each) | |
| DNA template | 1 | | DNA template | 1 μl |
| (100 ng/μl) | | | (100 ng/μl) | |
| Primer fw | 1 | | Primer fw | 1.25 μl |
| Primer rv | 1 | | Primer rv | 1.25 μl |
| Taq polymerase | 0.25 | | Herculase II | 1 μl |
| | | | polymerase | |
| 25 mM MgCl₂ | variable | | DMSO | 2 μl |
| Total reaction | 50 | μl | Total reaction | 50 μl |
| volume | | | volume | |

| | Taq polymerase | | | | Herculase II polymerase | | | |
|---|---|---|---|---|---|---|---|---|
| Initial | 3 | min | 94° | C. | 2 | min | 95° | C. |
| denaturation | | | | | | | | |
| 3-step | | | | | | | | |
| cycling | | | | | | | | |
| Denaturation | 0.5-1 | min | 94° | C. | 30 | s | 95° | C. |
| Annealing | 0.5-1 | min | 50-68° | C. | 30 | s | 50-70° | C. |
| Extension | 1 | min/kb | 72° | C. | 40 | s/kb | 72° | C. |
| Number of | 30 | | | | 30 | | | |
| cycles | | | | | | | | |
| Final | 10 | min | 72° | C. | 3 | min | 72° | C. |
| extension | | | | | | | | |

### Site-Directed Mutagenesis

Labyrinthopeptins mutants were prepared by means of site-directed mutagenesis. For all experiments a procedure described previously (12), which is a slightly modified QuickChange Site-Directed Mutagenesis System (QCM) developed by Stratagene (La Jolla, Calif., USA) was used. This two-stage procedure allows the efficient introduction of point mutation, deletions and insertions to a sequence of interest. For design of primers the program PrimerX was used. The procedure consists of two stages. In stage one, two extension reactions were performed in separate tubes; one containing the forward primer and the other containing the reverse primer. Subsequently, the two reactions were mixed, one microliter of polymerase was added and another PCR was carried out (2^{nd} PCR). Following the PCR, 1 μl of DpnI was added and incubated at 37° C. for one hour. Five microliter of the final PCR products were transformed into 50 μl of DHSa cells, and appropriate volumes were spread on LB agar plates containing either ampicillin or kanamycin.

| | 1^{ST} PCR | | | 2^{ND} PCR | | |
|---|---|---|---|---|---|---|
| Initial | 3 min | 95° | C. | 3 min | 95° | C. |
| denaturation | | | | | | |
| 3-step | | | | | | |
| cycling | | | | | | |
| Denaturation | 20 s | 95° | C. | 20 s | 95° | C. |
| Annealing | 30 s | 55-65° | C. | 30 s | 55-65° | C. |
| Extension | 3 min 30 s | 72° | C. | 3 min 30 s | 72° | C. |
| Number of | 5 | | | 20 | | |
| cycles | | | | | | |
| Final extension | 10 min | 72° | C. | 10 min | 72° | C. |

### Cloning Experiments

### Ligation of DNA Fragments

For ligation of DNA fragments T4 DNA ligase (from Fermentas or New Englan BioLabs) was used. In most of the performed reactions the molecular ration of insert to vector was 3:1 or 5:1. Reaction mixtures were incubated at 16° C. for 2 to 16 hours.

### LIC (Ligation-Independent Cloning)

All LIC experiments were performed according to a protocol established for In Fusion® HD Cloning System (Clontech laboratories, Inc. a Takara Bio Company, Madison, Wis., USA). LIC is a form of molecular cloning (13-14), which enables directional cloning of PCR fragment or multiple fragments into a linearized vector. Importantly, no additional treatment of the PCR fragment is required (such as restriction digestion, phosphorylation). The main idea which stays behind this method is the use of the 3′-->5′-activity of T4 DNA polymerase to create very specific 10-15 base single overhangs in the expression vector, and also to create complementary overhangs in PCR product. Those 15 by overlaps in PCR products can be engineered by designing primers for amplification of the desired sequences. The annealing of the insert (PCR product) and the vector is performed in the absence of ligase by simple mixing of the DNA fragments.

### Thermal Cycler Conditions

| | | |
|---|---|---|
| Initial denaturation | 3 min | 95° C. |
| 3-step cycling | | |
| Denaturation | 20 sec | 95° C. |
| Annealing | 20 sec | 70° C. |
| Extension | 10 sec | 72° C. |
| Number of cycles | 30 | |
| Final extension | 10 min | 72° C. |

| Quantity per reaction | Component |
|---|---|
| To final volume of | dH₂O |
| 50.0 μl | |
| 10 μl | 5x Herculase II |
| | reaction buffer |
| 0.5 μl | dNTP mix |
| | (25 mM each) |
| 1 μl | DNA template |
| | (100 ng/μl) |
| 1.3 μl | Primer fw |
| 1.3 μl | Primer rv |
| 0.5 μl | Herculase II |
| | polymerase |
| 2 μl | DMSO |

### Southern Blotting

A Southern-blot is a method for detection of a specific DNA sequence in DNA samples. Southern-blotting combines the transfer of electrophoresis-separated DNA fragments to a filter membrane and subsequent fragment detection by probe hybridization. In these studies Southern-blotting was used to prove the integration of a cosmid M1104 to a genomic DNA of Streptomyces. The very first step was restriction digestion of genomic DNA of Streptomyces wild type strains (negative control) and Streptomyces carrying the cosmid M1104. As a positive control the cosmid M1104 isolated from E. coli DH5a/cosmid M1104 was used. Cosmid M1104 and genomic DNA of S. lividans and S. coelicolor were fragmented with BamHI. For digestion of genomic DNA of S. albus the enzyme SacI was used. Obtained DNA fragments were separated by electrophoresis on an agarose gel (60 V, 90 min) To estimate the size of DNA fragments DIG-labeled DNA molecular weight marker VII (Roche) was used. The gel was dyed by ethidium bromide which after tacking a picture was washed away with water. Afterwards the gel was incubated in different solutions shaking at 4° C.: first for 20 min in Southern I, then for 30 min in Southern II, and finally for 30 min in Southern III. Such prepared DNA was transferred overnight on a nylon membrane (Hybond-N, Amersham Pharmacia Biotech). To do this a sheet of nylon membrane was placed on top of the gel. Pressure was applied evenly to the gel by placing a stack of paper towels and a weight (500 g) on top of the membrane and gel, to ensure good and even contact between gel and membrane. 20×SSC buffer was used to ensure a seal and prevent drying of the gel. Buffer transferred by capillary action from a region of high water potential to a region of low water potential (paper tissues) was then used to move the DNA from the gel onto the membrane; ion exchange interactions bound the DNA to the membrane due to the negative charge of the DNA and positive charge of the membrane. The next day the membrane was exposed to UV radiation (2 min) to permanently attach the transferred DNA to the membrane. A membrane was incubated with 100 ml Church Buffer (preheated to 65° C.) for 4 h at 65° C. Then Church Buffer was removed and a membrane was exposed to a hybridization probe. As a probe a DIG-labeled cosmid M1104 was used (see below). The use of digoxigenin gave a possibility for detection of probe-target hybrids by a color reaction with an alkaline-phosphatase-conjugated antibody. As a substrate for alkaline phosphatase the NBT/BCIP mix was used. First a cosmid M1104 (8 μg) was digested with a restriction enzyme MlyI to obtain fragments with a size of 800-1000 bp. After restriction digestion 4 μl water was added and DNA fragments were denatured by incubation at 100° C. for 10 min, later cooled on ice. Then DIG-labeled nucleotides were incorporated to a cosmid M1104 sequence by mixing with 4 μl DIG High Prime Solution (Roche) which contained Klenow enzyme, randomized oligonucleotides and DIG-11-dUTP. Such mix was incubated for 16 h at 37° C. Later the concentration of labeled DNA was calculated by blot membrane and comparison with a standard. Just before the use the probe was denatured (100° C., 5 min) and added to a 50 ml DIG Easy Hyb buffer (Roche) to reach an end concentration of 25 ng/ml. After an overnight incubation at 65° C. the probe solution was removed and the membrane was incubated shaking first with 2×SSC+0.1% SDS for 15 min (twice), second with 0.5×SSC+0.1% SDS for 15 min (twice). Later the membrane was washed for 5 min with 50 ml Washing Buffer which was followed by incubation in 20 ml anti-DIG-antibody solution for 30 min. Then the membrane was washed twice with 100 ml Washing Buffer (each time 15 min) Afterwards the membrane was incubated with 100 ml Detection Buffer for 5 min and then an additional portion of Detection Buffer (10 ml) containing 200 μl NBT/BCIP was added. The membrane was incubated with a substrate until a violet color appeared. The reaction was stopped by washing with water.

### Tris-Glycine SDS-PAGE and Western Blotting

SDS-PAGE (sodium dodecyl sulfate polyacrylamide gel electrophoresis) is a technique widely used to separate proteins according to their electrophoretic mobility (a function of length of polypeptide chain or molecular weight). SDS gel electrophoresis of samples has identical charge per unit mass due to binding of SDS results in fractionation by size. This method was used for detection of LabA2 in heterologous strains. Because LabA2 is a small peptide (2.1 kDa) it was not possible to reach a proper separation by the use of the popular and commonly used glycine-SDS-PAGE system. In this situation a tris-glycine SDS-PAGE (under denaturing conditions) was performed according to the protocol described by Schagger and von Jagow, 1987 (15). Samples were prepared by incubation of either agar plate extracts or supernatants and pellets from liquid cell cultures in sample loading buffer (in 1:1 ratio). Then samples were heated at 95° C. for 5 min and cooled down to RT. A stacking and resolving gel was run under a constant electric current 15 mA and 20 mA per gel, respectively. To estimate protein size the PageRuler Prestained Protein Ladder (Fermentas) was used.

| | Resolving | | Stacking | |
|---|---|---|---|---|
| Reagent | gel (16.5%) | | gel (5%) | |
| Rotiphorese Gel 30 | 3.33 | ml | 330 | μl |
| (acrylamide:bisacrylamide = 37.5:1) | | | | |
| Buffer | 1.125 | ml | 200 | μl |
| H₂O_{dd} | 1.41 | ml | 1.44 | ml |
| 10% SDS | 120 | μl | 20 | μl |
| TEMED | 15 | μl | 4 | μl |
| APS (100 mg/ml) | 30 | μl | 10 | μl |

After separation of protein by gel electrophoresis a semi-dry Western-blot was performed. During Western-blotting experiment proteins were transferred to a membrane, where they were probed using antibodies specific to the target protein. First proteins and peptides were moved from the gel onto a membrane made of nitrocellulose (Whatman). The membrane was placed on top of the gel, and a stack of filter papers placed on top of that. The entire stack was placed in a buffer solution which moved up the paper by capillary action, bringing the proteins with it. As a result of this “blotting” process, the proteins were exposed on a thin surface layer for detection. There was a need to optimize a transfer step (25 min, 50 mA) since LabA2 is a small peptide and can go through the membrane when a standard transfer conditions are used. The blocking of non-specific binding was achieved by placing the membrane in a blocking buffer for one hour. Afterwards membrane was incubated overnight at 4° C. in 30 ml TNT buffer containing 1% BSA and a primary antibody. Polyclonal antibodies against mature LabA2 or LabA2 leader peptide were used. Different concentrations of antibodies were tested but the best results for both of them were obtained when antibodies were diluted 1:2500. The next day membrane was washed three times with 30 ml TNT buffer with 1% BSA (each time for 10 min) After washing step a membrane was incubated in 30 ml TNT buffer with 1% BSA and 6 μl of a secondary antibody for two hours. As a secondary antibody commercially available Anti-Rabbit IgG Alkaline Phosphatase antibody was used. Then membrane was washed three times with 30 ml TNT buffer (each time for 10 min) and incubated with a detection solution until the colored precipitate was observed. Development of the blot was stopped by washing away the soluble dye with water.

### Analytical Methods:

### Sample Preparation for MS Analysis

Sample Preparation from Solid Agar Cultures

For bacteria which were grown on solid media a small piece of an agar were cut (around 1 cm²) and was extracted with 500 μl methanol or methanol:acetone (1:1) by sonication for 30 min. Later a sample was centrifuged and a soluble fraction was used for MS analysis. When a content of whole plate was extracted then 15 ml of methanol or methanol:acetone (1:1) was used.

Sample Preparation from Liquid Cultures

First step was separation of bacterial cells from a supernatant. The supernatant was either directly used for MS measurements or it was diluted before in methanol (1:1). The mycelium was extracted with methanol or with methanol:acetone (1:1), sonicated for 30 min and centrifuged. The soluble fraction was used for MS analysis. The amount of the solvent used for mycelium extraction was dependent on the weight of the pellet.

### SPE (Solid Phase Extraction)

For some of the samples SPE was performed. Each time Chromabond C18 cartridge (1 ml/100 mg) from Macherey-Nagel were used. SPE procedure conditions are summarized as follows:

| | | |
|---|---|---|
| Column conditioning | 3 ml | Methanol |
| | 3 ml | H₂O |
| Sample aspiration | 1 ml | Crude extract |
| Washing | 1 ml | Dichloromethane |
| | 1 ml | Acetone |
| | 1 ml | EtOAc |
| | 1 ml | Acetonitrile |
| Elution | 1 ml | 10% Methanol |
| | 1 ml | 20% Methanol |
| | 1 ml | 50% Methanol |
| | 1 ml | 80% Methanol |
| | 1 ml | 100% Methanol |
| | 1 ml | 0.1% HCOOH in H₂O |

### Mass Spectrometry Analysis of Labyrinthopeptin Variants

### HPLC-ESI

Labyrinthopeptins were detected by the use of HPLC-ESI-MS. LC-MS system was composed with 1100 Series HPLC System (Agilent) coupled either to QTRAP 2000 (Applied Biosystems) or to Q-Tof II (The Micromass). Gradients timetables are presented below:

| Time [min] | % Solvent A | % Solvent B | Flow [μl/min] |
|---|---|---|---|
| 0.00 | 60.0 | 40.0 | 60 |
| 10.00 | 20.0 | 80.0 | 60 |
| 10.10 | 0.0 | 100.0 | 60 |
| 13.00 | 0.0 | 100.0 | 60 |
| 13.10 | 95.0 | 5.0 | 60 |
| 15.00 | 95.0 | 5.0 | 60 |

The gradient timetable for LC-ESI-MS (QTRAP 2000). Solvent A: H₂O + 0.1% HCOOH, Solvent B: ACN + 0.1% HCOOH. Injection volume 5 μl.

| Time [min] | % Solvent A | % Solvent B | Flow [ml/min] |
|---|---|---|---|
| 0.00 | 95.0 | 5.0 | 0.300 |
| 20.00 | 0.0 | 100.0 | 0.300 |
| 25.00 | 0.0 | 100.0 | 0.300 |
| 26.00 | 95.0 | 5.0 | 0.300 |
| 30.00 | 95.0 | 5.0 | 0.300 |

The gradient timetable for LC-ESI-MS (Q-Tof II). Solvent A: H₂O + 0.1% HCOOH, Solvent B: ACN + 0.1% HCOOH. Injection volume 20 μl.

### LTQ-Orbitrap

The Orbitrap is a electrostatic ion trap using fast fourier transformation (FFT) to obtain mass spectra. It provides high mass accuracy (2-5 ppm) and mass resolution (150,000) which significantly reduces false positive peptide identifications. Masses were obtained after measurements with LTQ Orbitrap XL (Thermo Scientific). The gradient timetable is shown below:

LC-MS parameters

| | |
|---|---|
| Column: | XDB C-18, 5 μL, 4.6 * 150 mm (Agilent) |
| Injection volume: | 10 μL |
| Gradient: | 0 min 5:95% (MeOH + 0.1% HCOOH):(H₂O + |
| | 0.1% HCOOH), |
| | 25 min 100:0% |
| | 38 min 100:0% |
| | 40 min 5:95% |

LC-MS parameters for the high-resolution Orbitrap-ESI-MS. Injection volume: 10 μl.

### MS/MS

In these studies MS/MS experiment was used to compare Labyrinthopeptins obtained from heterologous strain S. lividans/pUWLab with Labyrinthopeptins isolated from a natural producer A. namibiensis. To obtain better fragmentation a disulfide bridge in Ring C of LabA1 was disrupted. Sample was incubated with NaHCO₃ (20 mM) and DTT (0.5 M) solution. Such mixture was incubated for 1 h at 50° C. After centrifugation a soluble fraction was ready to use for MS/MS experiment. Experiments were recorded using a LTQ-Orbitrap XL (Thermo Scientific, Bremen, Germany) coupled to an Agilent 1260 HPLC system (Agilent Technologies, Waldbronn, Germany). For chromatographic separation, a Vydac 218M C18 5 u, 150 mm×2.1 mm column (Grace, Deerfield, USA) with a linear solvent gradient from 5% to 100% ACN+0.1% HCOOH (solvent B) in 20 min were used. Solvent A was water (0.1% HCOOH). The MS/MS spectra were recorded in FTMS-mode with fragmentation in the HCD cell (a normalized collision energy of 23% was used).

### Example 1

### Introduction of DNA in A. namibiensis

It was aimed at establishing a protocol for generation of Labyrinthopeptins variants. Since the total synthesis is not a viable approach for generating preparative amounts of Labyrinthopeptins, biological approach was chosen. Initially mutations of the A. namibiensis genome were planned to enable generation of Labyrinthopeptin diversity. An important argument to choose this strategy was based on the fact that chemical synthesis of lantibiotics is very challenging and remain industrially impractical. For instance even for the best studied lantibiotic nisin a commercial production relies on biosynthesis using its natural producer (Lactococcus lactis) although its chemical synthesis has also been described previously (16).

Actinomadura belong to the genus which is the most dominant among rare Actinomycetes (17). This is the reason why this genus is one of the most important targets in screening programs for pharmacologically active compounds. However, in contrast to other actinomycete strains, there is little information available on the basic aspects of gene expression mainly due to the lack of versatile gene manipulation systems. In this respect the lack of suitable cloning vectors and methods of transformation negatively impacts genetic work with Actinomadura. For the molecular analysis of biosynthetic genes in certain actinomycetes, different transformation systems have been reported (e.g. polyethylene glycol induced protoplast transformation, transformation by electroporation and direct transformation). In presented study methods elaborated for Streptomyces were used to establish the method for DNA transfer to A. namibiensis. This choice was based on the fact that both Actinomadura and Streptomyces belong to Actinomycetes and methods for genetic manipulations in Streptomyces are very well described.

| | | | Growth after |
|---|---|---|---|
| Antibiotic | Conc. [μg/ml] | Growth after 4 days | 7 days |
| Apramycin | 25 | − | + |
| Apramycin | 50 | − | − |
| Streptomycin | 30 | + | ++ |
| Phosphomycin | 400 | +++ | +++ |
| Thiostrepton | 25 | − | + |
| Kanamycin | 50 | +++ | +++ |
| Viomycin | 30 | + | + |
| Hydromycin | 50 | ++ | +++ |
| Spectinomycin | 100 | +++ | +++ |

Testing selection markers suitable for genetic manipulations of A. namibiensis. Resistance markers.
Legend:
− no growth,
+ slight growth,
++ medium growth,
+++ strong growth.

### Plasmids

An apramycin resistance gene was used as selectable marker for vectors used in this study because from all eight tested antibiotics A. namibiensis was sensitive only to apramycin (50 μg/ml). Initially for establishing a way in which DNA can be introduced into A. namibiensis the typical cloning vectors for Streptomyces strains were used. Plasmid pUWLoriT which can replicate autonomously in Streptomyces and plasmid pSET152 which can integrate site-specifically at the bacteriophage ΦC31 attachment site (18) (7) were used. In order to prove the presence of ΦC31 attachment sites in A. namibiensis PCR-experiment was performed (primers: attB_For (5′-CGGTCTCGAAGCCGCGGTGC-3′) and attB_Rev (5′-GCCCGCCGTGACCGTCGAG-3′), expected PCR product: 260 bp). Although Actinomaduras and Streptomyces are related it is still possible that origin of replication (ori) for Streptomyces is not recognized by A. namibiensis (9). Description of similar difficulties can be found in the literature (17). This led to the use of vectors harboring replicons of Actinomadura strains: pSETermEΔHindIII_oriCLL4, pK18mobXylE_oripCLL4 and pCLL4_ori_apra. Also a non-replicating plasmid pK18mob2 with a fragment coding for the LabKC modifying enzyme was used for transformation of A. namibiensis.

To generate a vector pK18mob2_labKC a fragment of labKC gene was amplified with primers STK_RTPCR_fw and STK_RTPCR_rv to obtain 1547 bps long PCR fragment. PCR product was cloned into the vector pDrive to obtain pDrive_labKC. pDrive_labKC was cut with EcoRI and the fragment containing labKC was cloned to a vector pK18mob2.

The vector pCLL4 contains two functional origins of replication (9). One is delivered from plasmid pAkij1 (isolated from Actinomadura kijanita ATCC 31588) and the other from plasmid pBR322 (purified from E. coli DH10B). pCLL4 was capable of replication in Escherichia coli (providing resistance to ampicillin) and expression of resistance to ampicillin, and of replication in Streptomyces lividans and expression of resistance to thiostrepton. The lack of information about pCCL4 sequence makes the use of this vector difficult. Moreover as mentioned above A. namibiensis is not sensitive to thiostrepton. Three constructs were prepared to allow usage of origin of replication from the genus Actinomadura together with the apramycin resistance gene:

pSETermEΔHindIII_oriCLL4, pK18mobXylE_oripCLL4 and pCLL4_ori_apra.

To construct pSETermEΔHindIII_oriCLL4 and pK18mobXylE_oripCLL4 plasmids pUC18 and pCLL4 were digested with KpnI. Then a 0.9 kbp fragment from pCLL4 was cloned into pUC18 to get pUC18_oripCLL4. pUC18_oripCLL4 was subsequently digested with EcoRI and XbaI and cloned to vectors pSETermEΔHindIII and pK18mobXylE previously digested with the same restriction enzymes, to obtain pSETermEΔHindIII_oriCLL4 and pK18mobXylE_oripCLL4, respectively.

The vector pCLL4_ori_apra was prepared by cloning of a fragment containing oriT and an apramycin resistance gene to EcoRI and PstI restriction sites. This fragment was amplified by means of PCR with primers pK18mob_EcoRI_for (5′-CATCTCGAATTCCGCTCATGAGCTCAG-3′) and pK18mob_EcoRI_rev (5′-AGTTATCGAGATCTGCAGGAGCTCTTTGG-3′). pCLL4_ori_apra was then transformed into ET12567/pUZ8002 and used for conjugation with S. lividans. This experiment showed that pCLL4_ori_apra is successfully transferred by conjugation and its presence in bacteria can be verified by selection with apramycin.

PEG-assisted transformation of A. namibiensis protoplasts with plasmid DNA: Many authors have investigated the parameters influencing regeneration and transformation frequency and attempted to optimize the conditions for individual Streptomyces species (19-20). Of special importance are the growth phase of the mycelium at the time of protoplasting, the temperature at which the mycelium and the regenerating protoplasts are incubated, the number of protoplasts used per transformation, the dryness of the regeneration plates, and composition of the medium. For generation, transformation and regeneration of protoplast standard protocol for Streptomyces (see above) with some modifications was used. A. namibiensis was grown each time at 28° C. rotating 160 rpm in two different kinds of media CRM and YEME. Different modifications of media composition were tested. Mycelia were grown in the presence of 0.5%, 1% and 1.5% of glycine. Protoplasting was the most effective when glycine (1%) was present in the CRM growth medium. For efficient preparation of protoplasts the age and physiological state of the mycelium is important. It was described that the best transformation frequencies in S. lividans are obtained with protoplasts prepared from mycelium grown to “late exponential” phase, whereas for transfection of S. parvulus protoplast needed to come from much younger mycelium. To estimate when A. namibiensis is in exponential phase a growing curve was prepared. Because A. namibiensis cells form clusters it was not possible to simply measure the OD, instead every 6 hours 1 ml of culture was centrifuged and pellet was weighted. Another important step is a use of lytic enzymes. For Streptomyces the use of lysozyme give satisfying results. Surprisingly, no A. namibiensis protoplasts were observed after incubation with lysozyme. Neither prolonged incubated time (1, 2, 3 hours) nor increased incubation temperature (37° C.) and lysozyme concentration (2, 10, 50 and 100 μg/ml) helped. Although A. namibiensis is extraordinarily resistant to lysozyme it was possible to develop the protocol for protoplast formation. Protoplasts were formed when A. namibiensis was treated with cellosyl or cellosyl/lysozyme mixture. Cellosyl is a bacterial lysozyme from Streptomyces coelicolor. Cellosyl is of considerable interest because it is able to degrade cell walls of Staphylococcus aureus and other bacteria which are not hydrolyzed by chicken-, goose- or phage-type lysozyme (21). The best conditions for formation of protoplasts were obtained when A. namibiensis was grown in CRM medium with 0.5% or 1% final concentration of glycine, and cells were incubated for 2 h at 28° C. with a mixture of cellosyl (1 μg/ml) and lysozyme (0.5 μg/ml) solubilized in P-buffer (containing 20% of saccharose instead of standard 10%). To determine the optimal conditions for regeneration of viable cells from protoplasts R2YE and PWP overlay medium were tested. Only growth on PWP plates with an overlay of soft agar resulted in a lawn, which is necessary for successful regeneration. Work on the transfection of protoplasts led to the discovery that the presence of polyethylene glycol (PEG) in the transformation mixture is absolutely necessary for high frequency plasmid DNA transformation (22). Most often PEG 3350 and PEG 1000 are used. In order to examine the transformation ability of the A. namibiensis protoplasts, PEGs with differences in polymerisation degree (3350, 1000, 6000) and also PEGs from different suppliers (Sigma and Roth) were tested since it was reported before, that batches of PEG from different sources are not necessarily equivalent. As a plasmid DNA the following vectors were used: pSET152, pUWLoriT and pK18mob2_labKC. Since unsuccessful transformation may be the result of an active restriction systems in the host, plasmids were isolated from a dam-donor such as E. coli ET12567. Transformation experiments were performed with the use of frozen and also freshly prepared protoplasts. Although different transformation conditions and vectors were used no transformants were obtained.

Intergeneric Conjugation Between E. coli and A. namibiensis

One of the reasons for low transformation frequencies may be the result of active restriction systems in the host. This barrier can be overcome by the use of intergeneric conjugation (23) to introduce DNA into the host in a single-stranded form. Another advantage of conjugation is that it is simple and doesn't rely on the development of procedures for protoplasts formation and regeneration. The initial conjugative transfer of a shuttle plasmid between Escherichia coli and Gram-positive bacteria was reported by Trieu-Cuot et al. (24). The intergeneric transfer of plasmids from E. coli to Streptomyces was first described by Mazodier et al. (1989) (25) (26). Later, this method has been successfully applied to a number of different Streptomyces strains and other Actinomycetes as Amycolatopsis (27), Actinoplanes (28), Nonomuraea (29), Saccharopolyspora, Actinomadura, Micromonospora, Nocardia and Rhodococcus (30). Conjugation experiments were performed according to the protocol described above. As shuttle plasmid pSET, pUWLoriT, pCLL4_ori_apra from non-methylating E. coli donor ET12567 were used. First experiments were performed with the use of spores. Although A. namibiensis was described as a sporulating strain (31) there was a need to test many different media to obtain reasonable amount of spores. The following media were used ISP1, ISP2, ISP3, ISP4, ISP5, ISP7, MS, SM and sporulation medium. Sporulation was observed only on ISP4 and on sporulation medium, but only growth on sporulation medium gave satisfactory results. Spores as well as mycelial fragments were used for conjugation since mycelial fragments although less convenient for preparation may give higher numbers of recombinants. Mycelial fragments were prepared from bacteria which were grown on agar plates (AS1 and MS agar plates) or in a liquid culture (KM4 and TSB medium).

An exconjugant was obtained only after conjugation from E. coli ET12567/pUZ8002/pSET152. The correctness of a clone was verified by PCR reaction. Primer pSETproof_for (5′-CGAGCCGGAAGCATAAAGTG-3′) and pSETproof rev (5′-GCTTGGAGCGAACGACCTAC-3′) were used to amplify the 591 bps fragment of pSET152. This result demonstrates that vectors characteristic for Streptomyces are able to replicate in A. namibiensis and can be used for further genetic manipulations in this strain. Although an exconjugant was obtained there is still a strong need to establish the protocol which allows conjugation with much higher yield.

Direct Transformation of A. namibiensis

Another method used for finding a way of A. namibiensis transformation was the direct transformation described before for Amycolatopsis japonicum (27). It is an efficient method for various Amycolatopsis strains (11, 32), which were not prone to transformation with standard techniques. This method is based on the observation that bacteria can be transformed by incubation of protoplasts together with plasmid DNA in the presence of polyethylene glycol, calcium ions and calf thymus DNA, which is working as a carrier. It has been shown that the most critical parameter of this procedure is the age of the culture. For instance Amycolatopsis japonicum (27) showed the highest transfection efficiency if early stationary phase mycelia were used, but the optimal culture age of Nocardia lactamdurans was when the culture was in the exponential phase (33). Experiments were performed when A. namibiensis was in the late exponential phase according to the protocol described above with plasmid pSET and pUWLoriT. No transformants were obtained.

### Electroporation of Mycelium

An application of a brief, high voltage pulse to a suspension of cells and DNA, which results in the formation of transient membrane pores and uptake of DNA is called electroporation. It may enable to omit the need to develop conditions for protoplasts formation and regeneration, but on the other hand appropriate conditions for electroporation can be strain-specific. Electrotransformation was developed for Actinomyces spp. (34) for example for transformation of mycelium of several Streptomyces species (35-36), Streptomyces lividans protoplasts (37) and even Streptomyces germinating spores (38). For electroporation of A. namibiensis the slightly modified protocol for Streptomyces rimosus (10) was applied. Electroporation (with a plasmid pSET152ermE and pUWLoriT) was performed on mycelial fragments as well as on protoplasts. No transformants were obtained.

### Summary

The work with Actinomadura namibiensis was impeded due to the unavailability of genetic transfer method to this strain. Although in this study several transformation protocols were tested none of them gave a satisfying solution to this problem. However, it was possible to optimize partially a protocol for conjugation and transformation of protoplasts. A method for formation of Actinomadura namibiensis protoplasts was established by the use of cellosyl or cellosyl and lysozyme mixture. The lack of transformants after protoplasts transformation can be due to an insufficient regeneration of protoplasts. A conjugation from mycelium resulted in one exconjugant. Such low efficiency shows that this method is not reproducible and reliable, but on the other hand it is a proof that it is possible to introduce foreign DNA into Actinomadura namibiensis. Another important information from this experiment is that vectors characteristic for Streptomyces (e.g. pSET152) can be used for genetic manipulations in A. namibiensis. It is important since it is not always the case for the Actinomadura genera (9). These studies show that although different transformation methods were established for many actinomycetes they are still far from universal since optimal conditions for different strains may vary significantly.

### Example 2

### Heterologous Expression of Labyrinthopeptins in Streptomyces

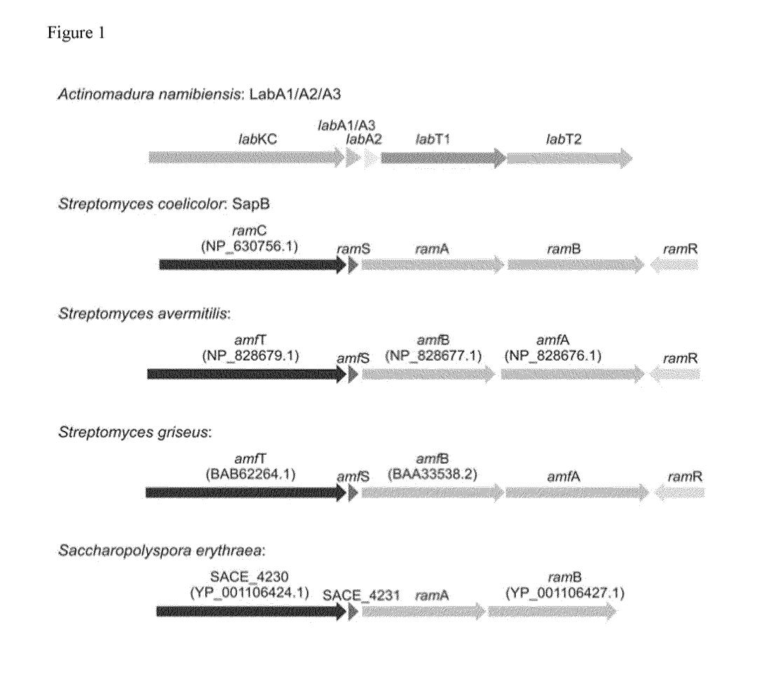

The development of efficient protocols for genetic manipulation of A. namibiensis gave unsatisfactory results which basically impeded a possibility of genetic engineering of Labyrinthopeptins in their natural producer. When the organism of interest is slow-growing or genetically intractable, the common strategy is to establish expression of the entire biosynthetic gene clusters in a more suitable host (39-40). The same strategy was followed. As heterologous hosts for Labyrinthopeptins expression Streptomyces were chosen. Strains used for these studies are easy to handle and genetic manipulation techniques are well-described (10). Several characteristics make Streptomyces a good candidate as a heterologous hosts. Importantly Streptomyces spp. and Actinomadura spp. belong to actinomycetes, they are both GC reach strains with similar codon usage. In addition the genus Streptomyces exhibits the ability to produce a wide variety of secondary metabolites (41). Although the information concerning genetic manipulations in Actinomaduras is very limited, the complementation of the biosynthetic gene clusters from A. madurae in two Streptomyces strains (S. carzinostaticus, S. globisporus) was reported (42). Another important issue is due to the fact that to obtain mature Labyrinthopeptins the leader peptide needs to be removed. No candidate gene has been identified in the lab gene cluster which may code for the protease responsible for the cleavage of the leader peptide. It is possible that the Labyrinthopeptin leader peptide is cut off by an extracellular non-specific protease like it has been already described for subtilisin from Bacillus subtilis (43). In silico analysis revealed that gene clusters homologous to the lab gene cluster are present also in other actinomycetes (FIG. 1) like Streptomyces coelicolor, Streptomyces avermitilis, Streptomyces griseus and Saccharopolyspora erythraea. This finding is of special interest because it seems that proteases from Streptomyces strains chosen as heterologous hosts could be able to complement the protease from A. namibiensis. All these features, together with a non-pathogenic nature and established fermentation technology, have made Streptomyces an obvious choice for producing Labyrinthopeptins.

### Heterologous Expression of a Cosmid M1104 Bearing the Lab Gene Cluster:

Integration of Cosmid M1104 into a Streptomyces Chromosome

The construct allowing heterologous expression (cosmid M1104) of Labyrinthopeptins was prepared already during PhD work of Dr. Timo Schmiederer (44). By means of λ-Red-mediated recombination (2, 5), the integrase gene (int) and the attachment site (attP) of phage ΦC31 (45) (46) were introduced into cosmid 1104 containing the complete lab gene cluster. The cosmid M1104 was site-specifically integrated into the chromosome of the heterologous hosts: S. lividans, S. albus and S. coelicolor. Transformation of a cosmid M1104 was performed by means of PEG-assisted transformation of protoplasts described by Kieser et al., 2000 (10). From obtained clones resistant to apramycin seven clones for S. coelicolor, three clones for S. albus and two clones for S. lividans were chosen for further experiments.

### Verification of Obtained Integration Mutants by PCR and Southern-Blot

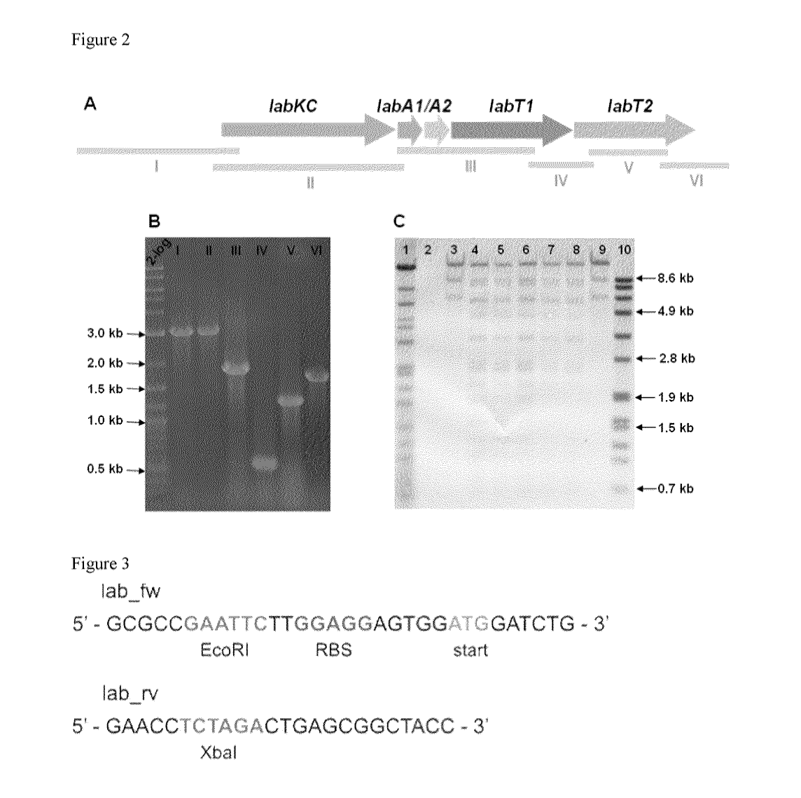

Integration mutants were verified by PCR and Southern-blot. All clones were treated in the same manner. Genomic DNA was isolated by slightly modified salting out procedure described by (10). Cosmid M1104 and genomic DNA of the Streptomyces host strain were fragmented either with BamHI (S. lividans, S. coelicolor) or SacI (S. albus). The DNA blot was probed with the cosmid M1104 digested with MlyI and DIG-labeled. The profiles of wild type strains and heterologous hosts carrying the cosmid M1104 were compared. Southern hybridization experiments with total DNA from S. coelicolor with cosmid M1104 did not reveal hybridization signals, indicating that S. coelicolor did not have DNA homologous to the introduced cosmid. This finding eliminates the possibility of either activation or complementation of cryptic genes in the host. Similar results were obtained for two other strains S. lividans and S. albus used as heterologous hosts. To verify integration events into mutants by means of PCR six primer pairs were design. Sequences of primers and the length of expected PCR products are described below. This experiment showed that all the expected DNA fragments could be observed after PCR with primer pairs I-VI for integration mutants but not for wild types. An example is shown in the FIG. 2B. PCR and Southern blot analysis proved that no significant deletions and rearrangements had taken place within integration mutants.

### Primers Used for PCR Verification of Integration Mutants:

| | | Expected |
|---|---|---|
| | | PCR |
| | | product |
| Primer | Sequence | [bps] |
| I: | Iko Fw1: | 3004 |
| Iko Fw1 + | 5′-GTTCGTTCGACGGACCAATG-3′ | |
| Iko Rev1 | (SEQ ID No. 145) | |
| | Iko Rev1: | |
| | 5′-CCTGCTCGACGCAGTATTTG-3′ | |
| | (SEQ ID No. 146) | |
| II: | Iko Fw2: | 3032 |
| Iko Fw2 + | 5′-CGCAGGACGAACGGTTTCAG-3′ | |
| Iko Rev2 | (SEQ ID No. 147) | |
| | Iko Rev2: | |
| | 5′-CCATGGGACTGTCACCTCTC-3′ | |
| | (SEQ ID No. 148) | |
| III: | Iko Fw3: | 1866 |
| Iko Fw3 + | 5′-CATCCACCATGGCATCCATC-3′ | |
| Iko Rev3′ | (SEQ ID No. 149) | |
| | Iko Rev3′: | |
| | 5′-GCGTCGTCGAGGATGATCAG-3′ | |
| | (SEQ ID No. 150) | |
| IV: | Iko Fw3′: | 567 |
| Iko Fw3′ + | 5′-ACTACCGGGCGATGTTCGAG-3′ | |
| Iko Rev3 | (SEQ ID No. 151) | |
| | Iko Rev3: | |
| | 5′-AGCAGCCGGGAGAGCAGCAG-3′ | |
| | (SEQ ID No. 152) | |
| V: | Iko Fw4: | 1302 |
| Iko Fw4 + | 5′-ACTACCGGGCGATGTTCGAG-3′ | |
| Iko Rev4′ | (SEQ ID No. 153) | |
| | Iko Rev4′: | |
| | 5′-CTGAAGACGTACGCCTCCTG-3′ | |
| | (SEQ ID No. 154) | |
| VI: | Iko Fw4′: | 1751 |
| Iko Fw4′ + | 5′-CAGGAGGCGTACGTCTTCAG-3′ | |
| Iko Rev4 | (SEQ ID No. 155) | |
| | Iko Rev4: | |
| | 5′-AGATGAAGCGGGCGATCGAG-3′ | |
| | (SEQ ID No. 156) | |

Cultivation of heterologous strains and detection of Labyrinthopeptins

Integration mutants and parental host strains were cultured in few different production media summarized below. Because the production levels can vary between clones, more that one independent integrant were isolated and tested for Labyrinthopeptins production. Production was tested by LC-ESI-MS and Western-blotting analysis. For Western blotting antibody against LabA2 leader peptide and also mature LabA2 were used. This allowed detection of not only fully modified LabA2 but also its prepropeptide and fully modified LabA2 with an attached leader peptide. The possibility to detect LabA2 with an attached leader peptide was very important because of the lack of the protease responsible for the leader peptide cleavage in the lab gene cluster (also no protease fulfilling this function could be identified in the boundaries of a cosmid M1104). The Western blot protocol, LC-MS method and the way of samples preparation are described above. The analysis of metabolites showed that, the integration mutants did not produce any Labyrinthopeptins or Labyrinthopeptins derivatives.

### Strains and Growth Conditions Used for Heterologous Expression of a Cosmid M1104:

Culture conditions

| | Number of | Liquid | |
|---|---|---|---|
| Heterologous host | tested clones | cultures | Solid media |
| | | TSB | R2YE |
| S. coelicolor | 7 | CRM | KM4 |
| S. lividans | 2 | KM4 | MS |
| S. albus | 3 | Glu-Nutrient | SMMS |
| | | Broth | R5 |

Heterologous Expression of Labyrinthopeptin by Using the Vector pUWLab:
Construction and Verification of a Vector pUWLab

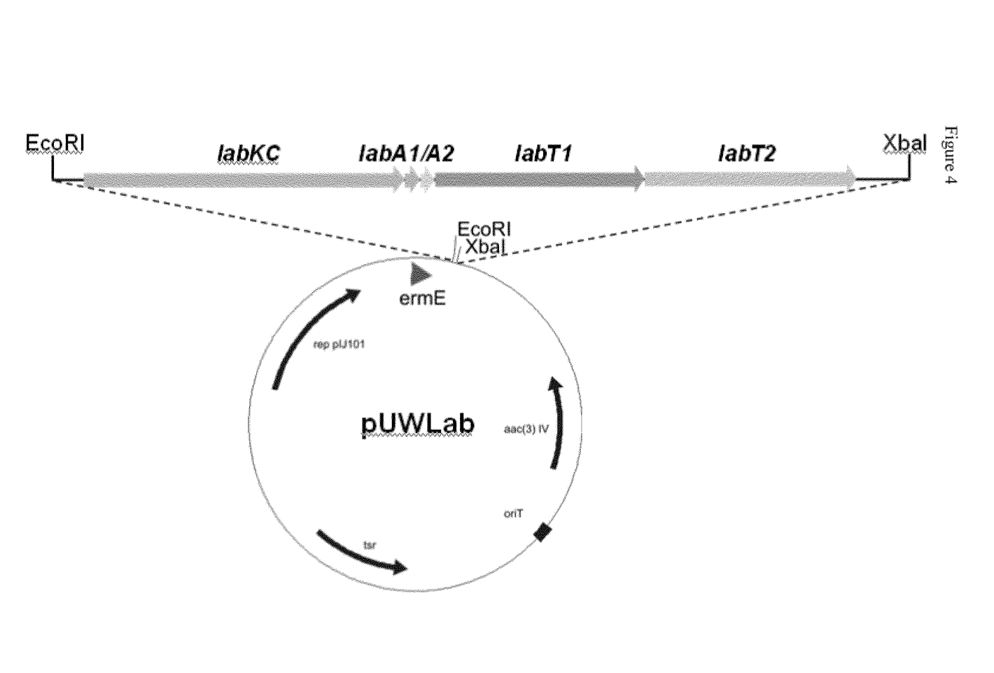

The whole lab gene cluster was amplified from a cosmid 1104 by means of PCR with the use of primers lab-fw/rev (FIG. 3) and Herculase-II-Fusion-DNA polymerase. Importantly primer lab-fw allowed an addition of a RBS characteristic for Streptomyces (GGAGG sequence five by upstream the start codon of labKC gene). Thus obtained PCR product (6.4 kb) was digested with enzyme EcoRI and XbaI and directly cloned under the control of a constitutive ermE promoter (47) into an Escherichia coli-Streptomyces shuttle vector pUWLoriT, yielding a new vector pUWLab (13.8 kb) (FIG. 4).

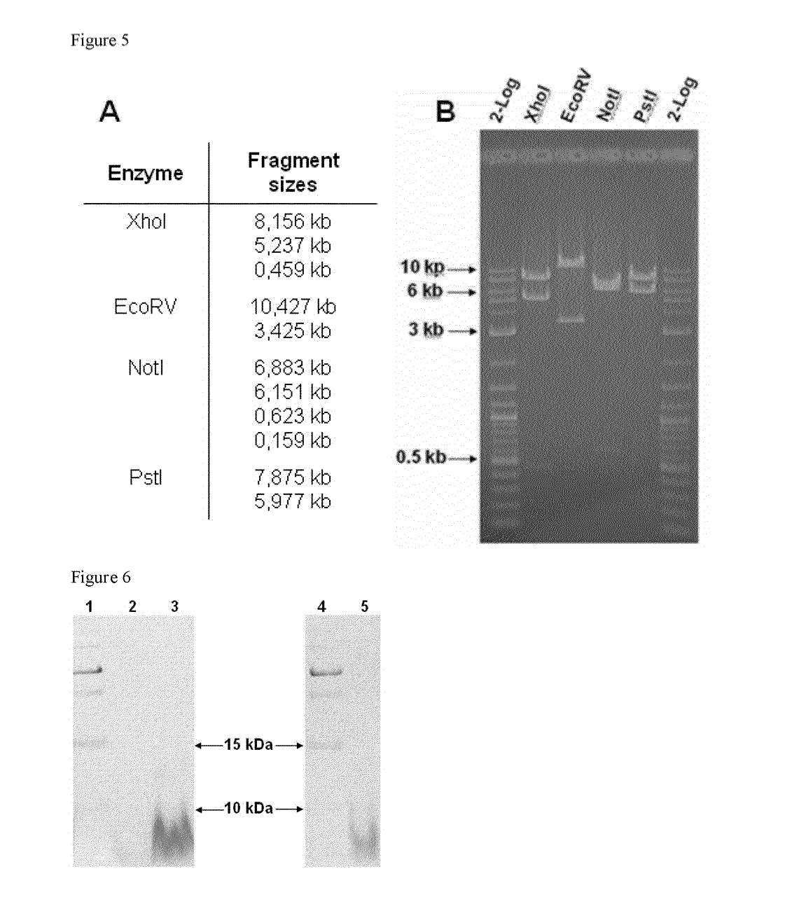

Correctness of a construct was confirmed by restriction digestion and sequencing reactions (primers: IkoRev1, LabKC_Fw2, LabKC_Fw3, LabKC_Fw4, LabKC_Fw5, LabKC_Fw6, IkoRev3′, IkoRev4′, IkoFw4′). pUWLab was digested with four different restriction enzymes (XhoI, EcoRV, NotI, PstI). For all of them expected DNA fragments could be observed like it is shown in FIG. 5. Also sequencing reaction did not reveal any mistakes (primers used for sequencing are summarized below). Plasmid pUWLab was then transformed to the methylation-defective E. coli strain ET12567/pUZ8002. Finally, the desired construct was successfully transformed to few Streptomyces strains (S. coelicolor M145, S. lividans ZX7, S. albus CB 89, S. avermitilis DSM 46492, S. griseus DSM 40236) by conjugation.

Primers Used for Amplification of a Lab Gene Cluster and Sequencing of pUWLab:

| Primer | |
|---|---|
| name | Primer sequence |
| IkoRev1 | CCTGCTCGACGCAGTATTTG (SEQ ID No. 146) |
| LabKC_Fw2 | CCTGCCGGACGGCTGGGAAC (SEQ ID No. 157) |
| LabKC_Fw3 | GGGAGAACGGGACCGTCGAG (SEQ ID No. 158) |
| LabKC_Fw4 | CGCCCGACTACACCGGGTTC (SEQ ID No. 159) |
| LabKC_Fw5 | CCCGCGAGCTCATGGAGCAC (SEQ ID No. 160) |
| LabKC_Fw6 | CCGGCATCCTCGCCTACCTG (SEQ ID No. 161) |
| III-IkoRev3′ | GCGTCGTCGAGGATGATCAG (SEQ ID No. 150) |
| IkoRev4′ | CTGAAGACGTACGCCTCCTG (SEQ ID No. 154) |
| IkoFw4′ | CAGGAGGCGTACGTCTTCAG (SEQ ID No. 155) |

Cultivation of heterologous strains and detection of Labyrinthopeptins Mutants transformed with a plasmid pUWLab and parental host strains were cultured in different production media. For each strain two types of liquid cultures (YEME, M5294) and solid agar plates (R2YE, KM4) were prepared. Expression of Labyrinthopeptins was tested by Western blotting experiments and mass spectrometry (HPLC-ESI-MS, HPLC-ESI-MS/MS, LTQ-Orbitrap-MS). All performed experiments are summarized below:

| Expression | | Culture | Detection of |
|---|---|---|---|
| vector | Heterologous host | conditions | Labyrinthopeptins |
| pUWLab | S. coelicolor | Liquid cultures | Western blotting |
| | S. lividans | (M5294, YEME) | HPLC-ESI-MS |
| | S. albus | | LTQ-Orbitrap-MS |
| | S. avermitilis | Plates | MS/MS |
| | S. griseus | (R2YE, KM4) | |

Although at least three clones were tested and many different growth conditions were applied, no expression of Labyrinthopeptins was observed for S. coelicolor, S. avermitilis and S. griseus carrying the vector pUWLab. Fortunately, the analysis of secondary metabolites showed that, in contrast to the untransformed host strains, S. lividans and S. albus containing pUWLab accumulated Labyrinthopeptins, but only expression in S. lividans gave sufficient amounts of desired products. In the following, results from expression in S. lividans are shown.

Western blotting was performed with antibodies which were raised either against mature LabA2 or LabA2 leader peptide. LabA2 is a peptide with a mass of only 2.1 kDa. The small molecular mass may induce difficulties with a good detection by the use of Western blotting technique. As a positive control pure LabA2 or supernatant from a liquid culture of A. namibiensis was used. As a negative control served a wild type culture extract. Signals were observed only after incubation with antibody against fully modified LabA2 for S. lividans/pUWLab cultures (liquid and agar media) but not for a wild type (FIG. 6).

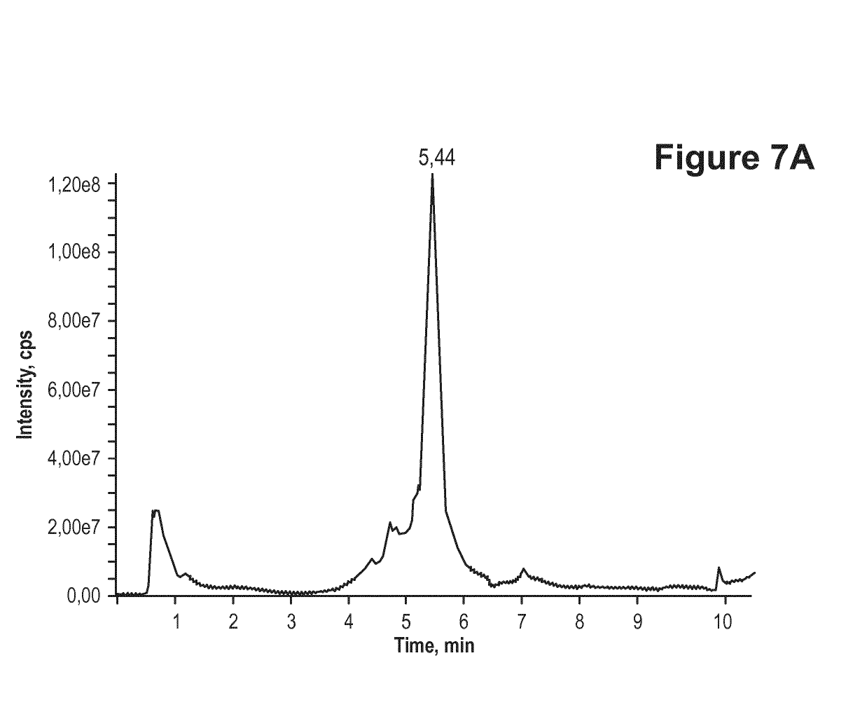

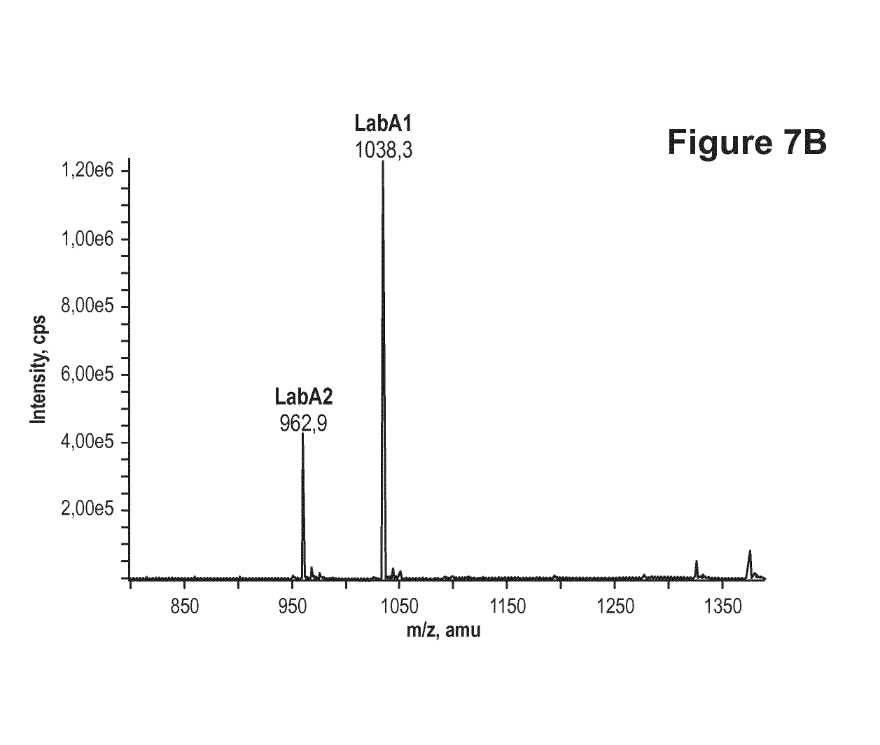

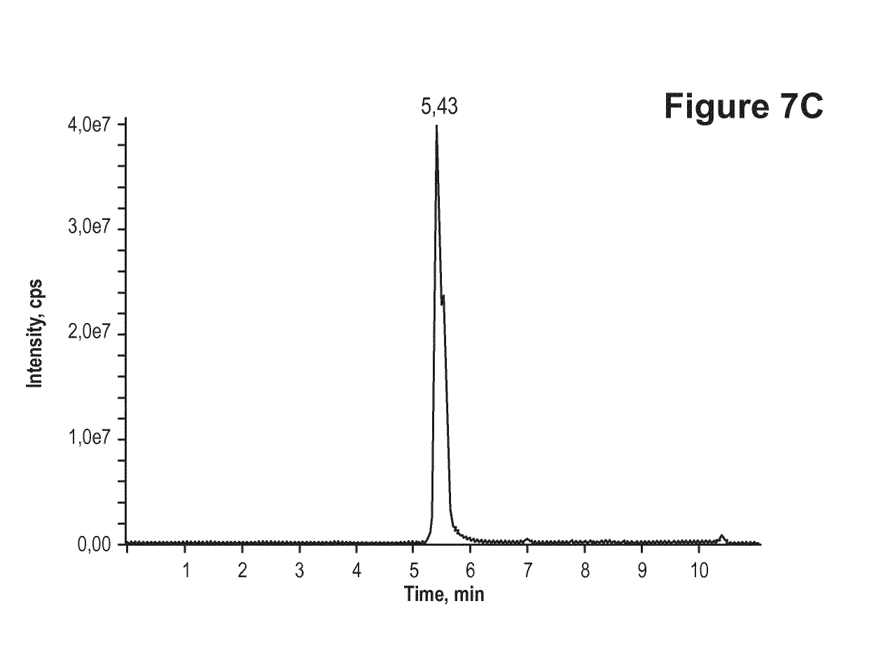

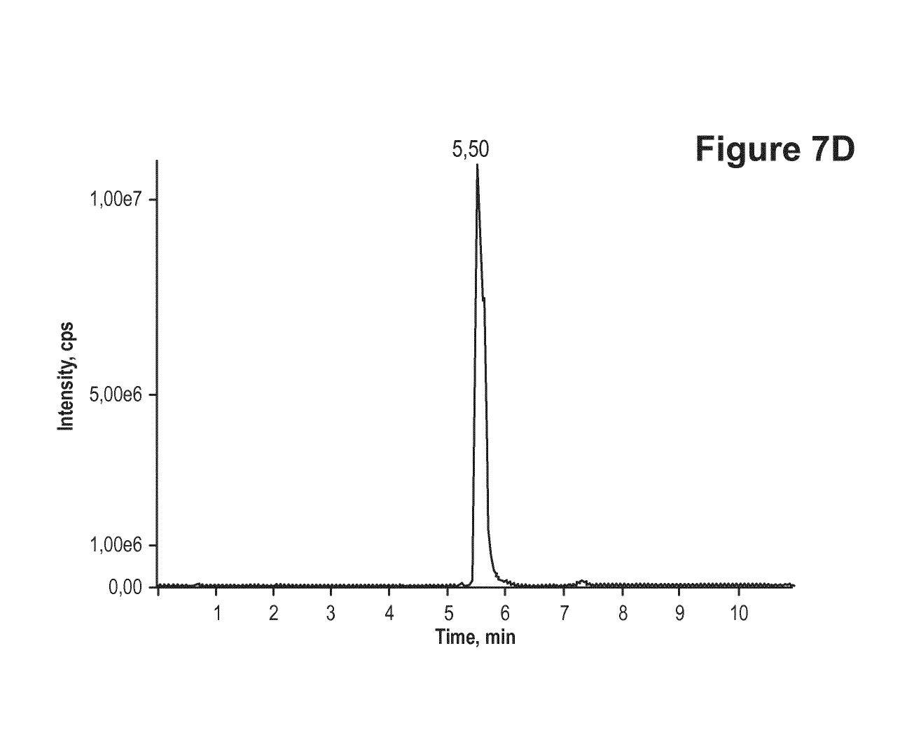

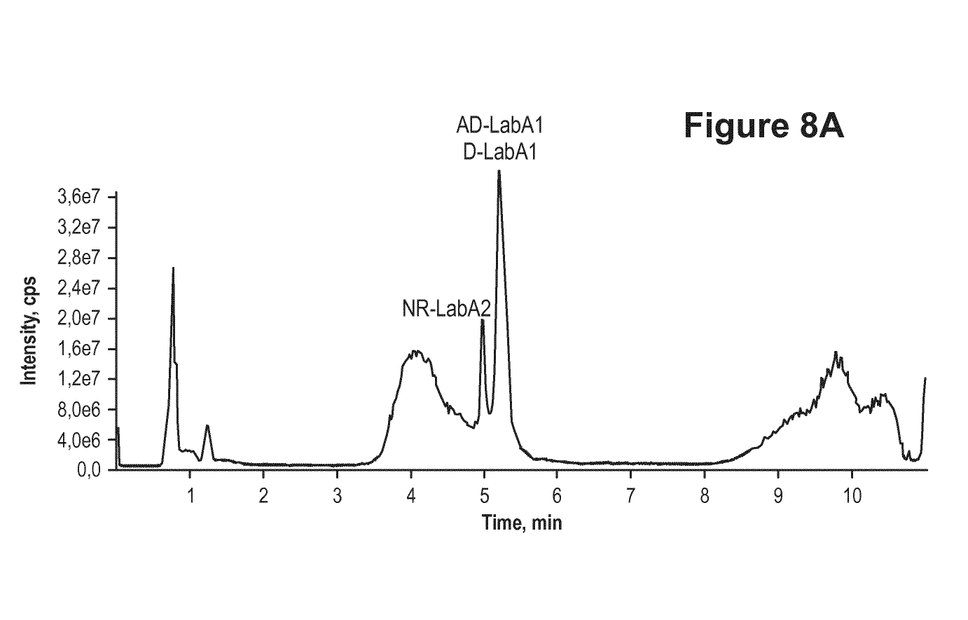

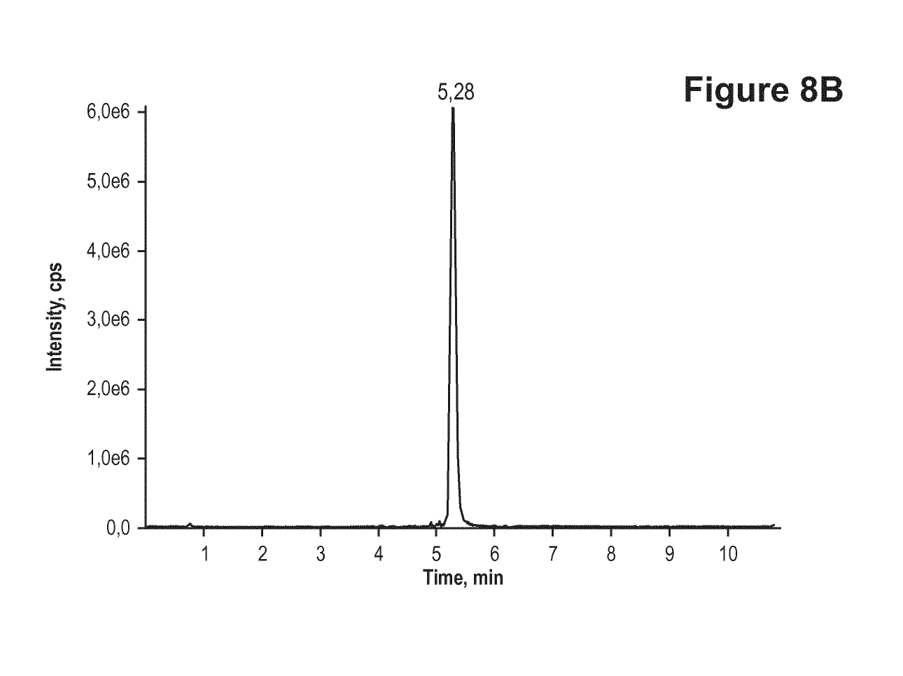

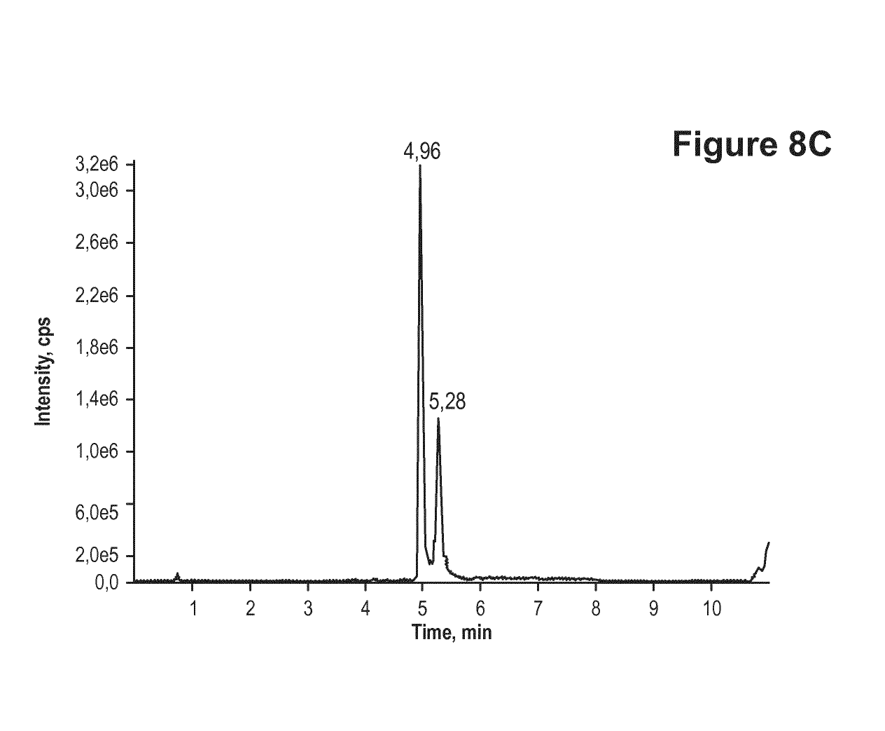

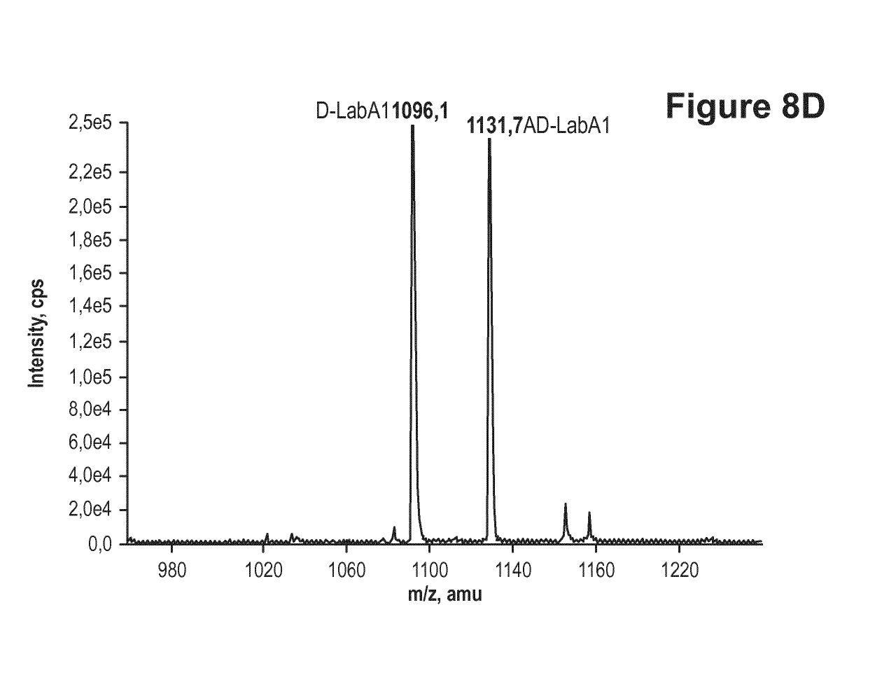

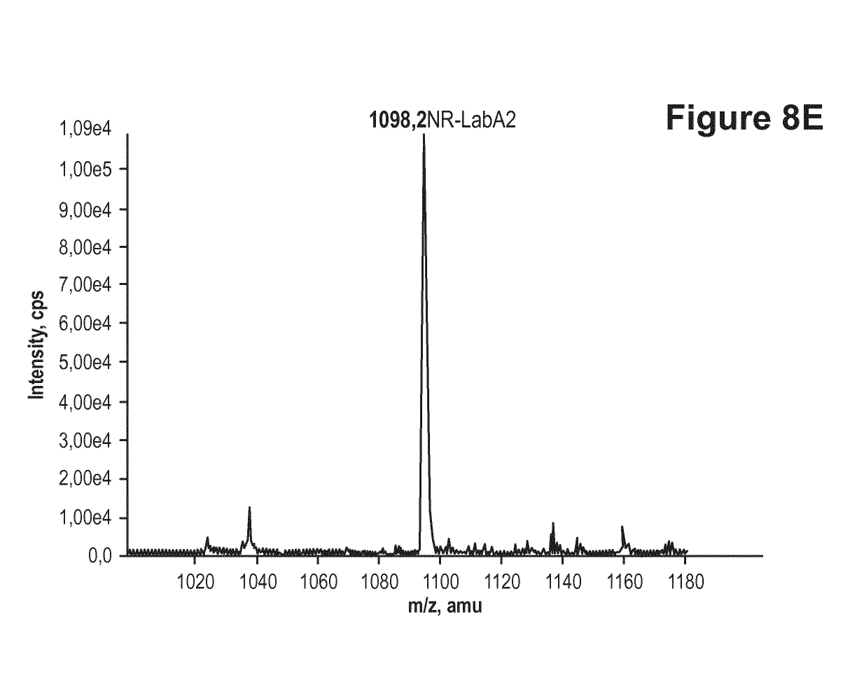

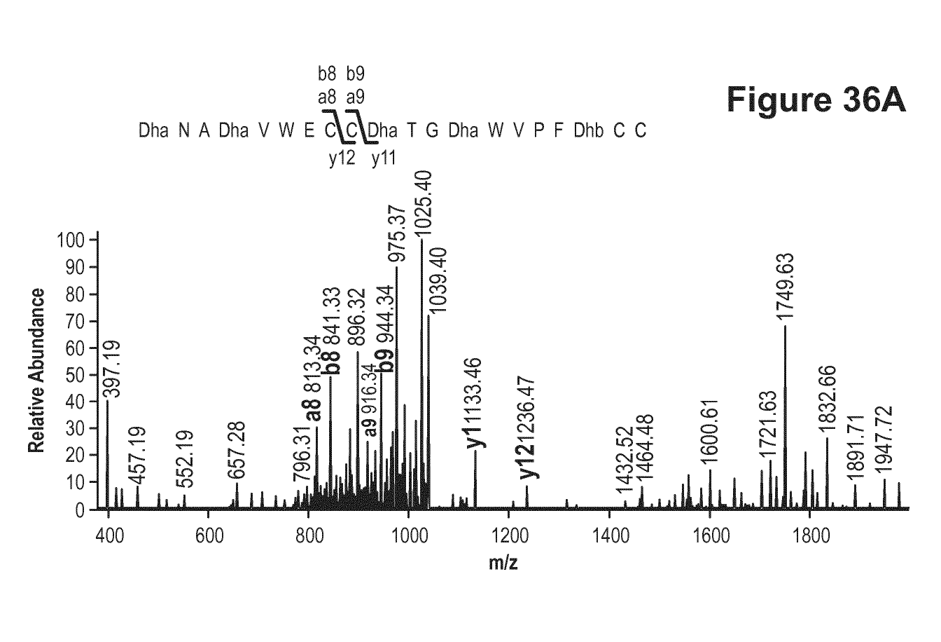

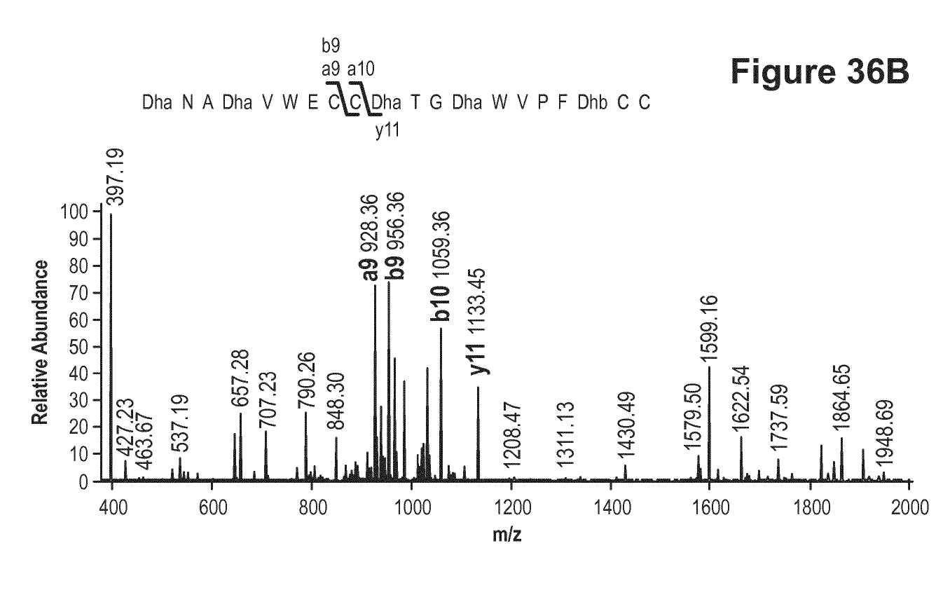

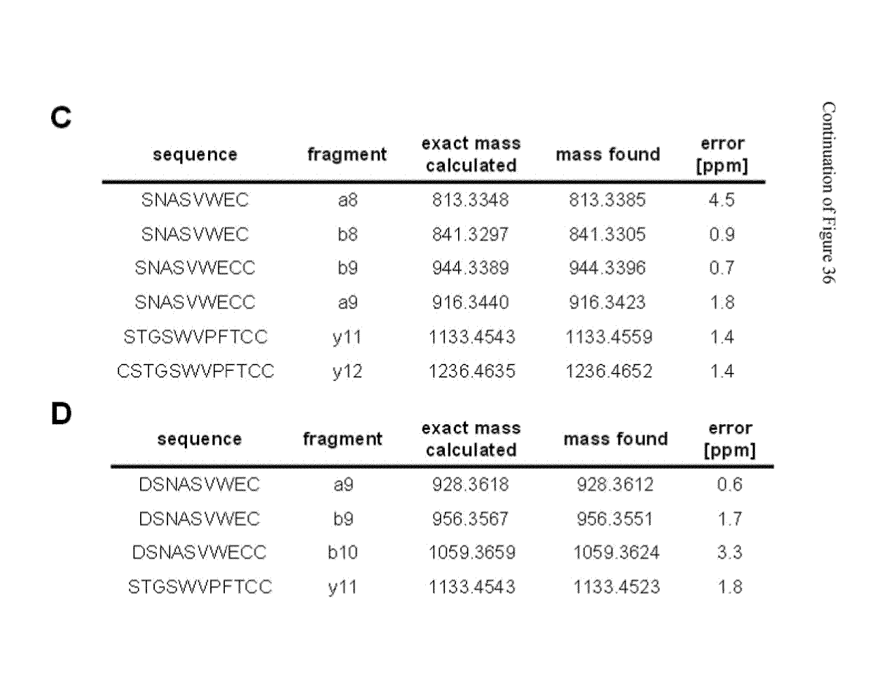

Comparison of LC-MS spectra of S. lividans wild type and S. lividans/pUWLab revealed unknown compounds in the culture filtrates of the latter. It was obvious from the evaluation of chromatograms that clones containing the lab gene cluster were producing substances which were not present in a wild type. New compounds were detected under all tested growth conditions: in liquid cultures (in the supernatant and in the pellet) and also on agar plates. These metabolites have similar retention time like Labyrinthopeptins (see FIGS. 7, 8). Surprisingly their masses did not correspond to the masses characteristic for LabA1 and LabA2. Further analysis revealed that those mysterious products were Labyrinthopeptins derivatives with additional amino acids derived from the leader peptide. S. lividans was able to express mature LabA1 with an N-terminal Ala or Ala-Asp overhang (FIG. 8). The ratio between observed LabA1 derivatives depends on the culture stage. At the beginning of growth LabA1 with two additional amino acids is the main product. In two weeks old cultures of S. lividans/pUWLab LabA1 with only one additional N-terminal amino acid is the major product. Also expression of fully modified LabA2 was successful. LabA2 derivatives with N-terminal Asn-Arg overhang were detected (FIG. 8). These results suggest that the leader sequence may be processed by a non specific protease contributed from the heterologous strain S. lividans, although the sequence of the cleavage site appears different from the natural producer. Additionally, LC-ESI-MS/MS and LC-ESI-MS Orbitrap experiments were performed in order to support the observed results. To confirm the correctness of our assumption additional experiments were performed like MS/MS and LTQ/Trap measurements. Tandem mass spectrometry (MS/MS) analysis was used to further confirm the identity of LabA1 observed in cultures of S. lividans/pUWLab. Two samples were measured and compared. The control sample was LabA1 isolated from culture broths of A. namibiensis, which was referred to a supernatant from a 9 d liquid culture (medium YEME) of S. lividans/pUWLab. Molecular masses observed in the LC-MS spectra allowed the prediction that the heterologous host strain can produce D-LabA1 and AD-LabA1. Because the level of production of D-LabA1 in older cultures was higher MS/MS experiments focused on this derivative. Before performing MS/MS measurements disulphide bridges in Labyrinthopeptins were reduced. Fragmentation pattern is in agreement with proposed ring topology and resemble the MSMS fragmentation of the wild type LabA1. High-resolution LTQ Orbitrap mass spectrometry was used to investigate supernatant from a 9 d liquid culture (medium M5294) of S. lividans/pUWLab. It was possible to identify and to calculate the molecular formula of singly charged ions which belong to D-LabA1, AD-LabA1 and NR-LabA2. Obtained relative errors have fully acceptable values. These results are in agreement with an outcome from LC-MS (FIG. 8) and MS/MS experiments, as shown in FIG. 36.

Molecular formula proposals by means of accurate molecular mass measurement compared to experimental data obtained from LC-ESI-OrbiTrap-MS.

| | | Exact mass | Found mass | Error |
|---|---|---|---|---|
| Mutant | Formula | calc. | (Charge state) | [ppm] |
| D-LabA1 | C₉₆H₁₂₄O₂₈N₂₄S₄ | 2188.7901 | 2189.9103 (+1) | 3.532 |
| AD-LabA1 | C₉₉H₁₂₉O₂₉N₂₅S₄ | 2259.8271 | 2260.8334 (+1) | 2.515 |
| NR-LabA2 | C₉₅H₁₂₈O₂₇N₂₆S₄ | 2192.8325 | 2193.8210 (+1) | 2.544 |

### Summary

The heterologous expression of Labyrinthopeptins by the use of the cosmid M1104 failed. One explanation is that promoters from A. namibiensis were not recognized by Streptomyces (9). However, it is possible that the use of other strains could result in finding of an efficient method for the heterologous expression of the lab gene cluster. Even through many production media were tested, the lack of Labyrinthopeptins expression can be caused by the use of not optimal growth conditions. Interestingly, the lab gene cluster does not contain regulatory genes which seem to play an important role for lantibiotics showing homology to Labyrinthopeptins. For example the ram gene cluster responsible for an aerial mycelium formation in S. coelicolor contains two-component response regulator (RamR) disruption of which results in strains that were 12 to 24 hours delayed in the onset of differentiation compared to the wild-type strains (48).

Heterologous expression of a vector pUWLab in S. albus and S. lividans resulted in a production of LabA1 and LabA2 derivatives. The lack of Labyrinthopeptins in cultures of S. coelicolor, S. griseus and S. avermitilis shows that heterologous expression is an unpredictable method. Labyrinthopeptins expressed in S. albus and S. lividans contain additional amino acids from a leader peptide, which is not surprising since no candidate gene coding for the protease has been identified in the lab gene cluster. Such phenomena has been already described for other lantibiotics e.g. actagardine (49) which also doesn't posses a protease in the boundaries of the gene cluster. Expression of the actagardine gene cluster in Streptomyces lividans resulted in the production of ala(0)-actagardine (actagardine with additional N-terminal alanine). The presence of additional amino acids in Labyrinthopeptins is the main drawback of the pUWLab construct in a heterologous host. This could negatively influence their biological properties and in any case cause problems for separation by chromatographic methods. Fortunately, this problem was solved in the following example.

### Example 3

### Establishing Methods for Efficient Generation of Novel Labyrinthopeptins

Lantibiotics can be used as templates for the generation of novel compounds with altered amino acid side chains. This is due to the fact that they are ribosomally synthesized and therefore can be modified by means of site directed mutagenesis. The ribosomal origin of lantibiotics—in contrast to multienzyme complexes—makes them excellent candidates for bioengineering according to current state-of-the-art technologies. Two main strategies have been developed for the production of engineered lantibiotics. They have been already applied for generation of for example nisin (50-51), mutacin II (52), epidermin (53) and Pep5 (54) variants.

The first method involves a plasmid-borne, complementary copy of a structural gene in the host strain, and the second involves replacement of the wild-type gene with a mutated copy by using gene replacement procedures. To employ the complementation strategy it is desirable to first inactivate the structural gene encoding the wild type prepropeptide. In all these systems the structural gene is plasmid-encoded and its expression occurs independently of the other enzymes involved in lantibiotic biosynthesis. The advantage of this system is that the sequences involved are more readily manipulated by a variety of molecular biology techniques. Problems may arise when the structural gene is expressed on a multicopy plasmid, because overproduction of the mature lantibiotic could result in higher processing error rates, or in higher antimicrobial activity than the hosts immunity system can cope with. Even if the natural producer is used for expression of novel lantibiotic often the yield of mutated peptide is much lower than the yield observed for the wild type peptide.

The second strategy is based on the replacement of the wild type structural gene by a new prepropeptide. In this case the new structural gene is expressed at its natural genomic location. The advantages of a gene replacement approach are that unchanged gene-dosage and regulatory responses are maintained. It also has the advantage of retaining the balance between structural, biosynthetic and immunity genes. However it is a very time consuming procedure.

Generally, better results are obtained by gene replacement than by complementation. This phenomenon was observed also in case of nisin (55). The possible reason for this is that the uncoupling of the transcription of the structural nisA gene from downstream genes might have a negative effect on biosynthetic genes expression and in consequence the production level. Because neither complementation nor replacement strategy give a possibility of easy and fast generation of novel lantibiotics without significant dropps in production yield, scientists are still looking for new solutions. Lin et al., 2011 (56) described semi-in vitro biosynthesis (SIVB) of bovicin HJ50 totally based on E. coli expression system. Although expression in E. coli facilitates an easy genetic manipulation this method is still not perfect. First it is not possible to obtain high amount of novel lantibiotics, second it increases costs since for each reaction enzymes and substrate need to be purified.

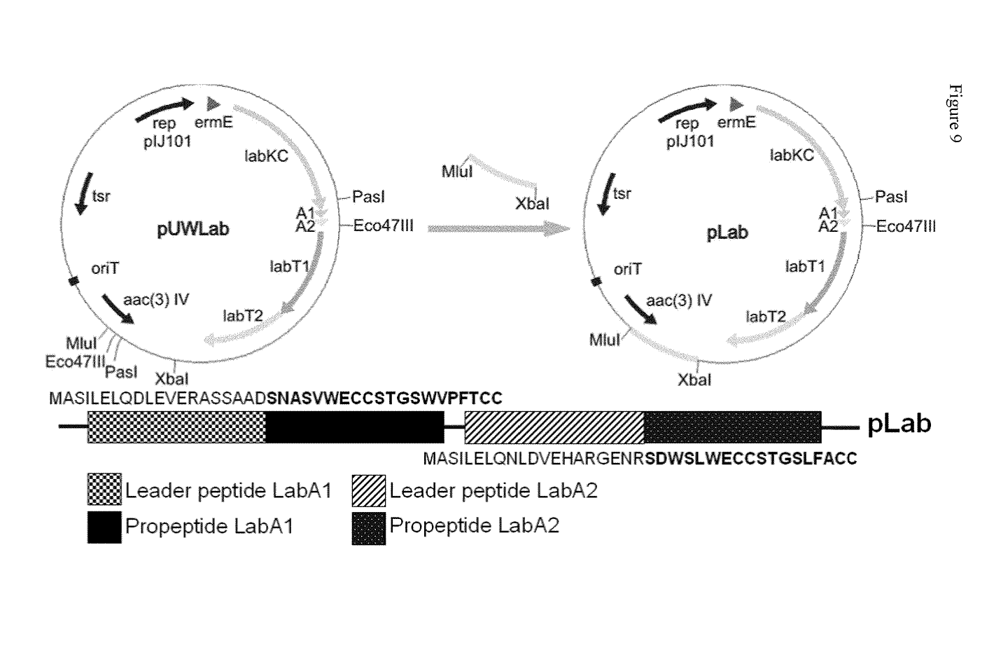

Construction of the vector pLab for heterologous expression in Streptomyces The lack of a method for DNA transfer to A. namibiensis impedet the use of this strain for generation of Labyrinthopeptins derivatives. However, the vector pUWLab enables an expression of satisfying amounts of Labyrinthopeptins in S. lividans which opened surprisingly a way for expression of novel peptides in a heterologous strain. Because of the ribosomal origin of the peptides, variant generation can be accomplished by mutagenesis of structural genes only. Furthermore, if only a part of pUWLab coding for prepropeptides needs to be modified then there are at least two possible ways of generation of novel peptides. The first strategy is the knock-out of the prepropeptide sequence in pUWLab and then complementation with a small plasmid carrying only genes coding for Labyrinthopeptins pre-pro-peptides. In this situation mutants could be easily generated by means of site-directed mutagenesis. However, this method was rejected because it is very likely that the expression of Labyrinthopeptins would be much lower than when pUWLab is used. First the knock-out could have a polar effect on downstream genes. Second, an inverted repeat located in the short noncoding segment between labA2 and labT1 was detected. The presence of a stem-loop structure between the structural gene and the downstream biosynthetic genes is a feature of a number of other lantibiotics (57) (58). It has been established that the stem-loop structure can stabilize mRNA (59) by protecting them from degradation by ribonucleases influencing yields of lantibiotics production. In order to maintain high expression levels another method for generation of mutants was considered. This would involve site directed mutagenesis using expression vector pUWLab as a template. However, one serious complication arose since pUWLab is a comparatively big plasmid with a size of 13.8 kbp. In this situation site directed mutagenesis experiments cannot be performed in one step which complicates the whole procedure and significantly prolongs the time of an experiment. In this situation it was important to determine restriction sites in the pUWLab sequence which could be used to specifically excise labA1 and labA2 genes. Then these genes could be subcloned into a smaller plasmid which allows a convenient site-directed mutagenesis experiment. After introduction of a mutation a sequence coding for a novel labyrinthopeptin prepropeptide could be cloned back to pUWLab. Fortunately, it was possible to find suitable restriction enzymes. PasI and Eco47III could be used to precisely excise the prepropeptide encoding region. Restriction sites for these enzymes appear twice in the pUWLab sequence. To remove PasI and Eco47III restriction sites from an undesired region a synthetic gene aac(3)IV was used. It contains a part of acc3(IV) gene with two silent point mutations (CGCCGG (Arg), CTGCTC (Leu)). PasI recognition sequence CCCTGGG was changed to CCCTCGG which cannot be recognized anymore by the enzyme. Also the Eco47III recognition sequence AGCGCT was disrupted by a single point mutation, which generated AGCGGT sequence. A synthetic gene aac(3)IV was cloned to a pUWLab via MluI and XbaI restriction sites to obtain pLab (cloning strategy is presented in FIG. 9).

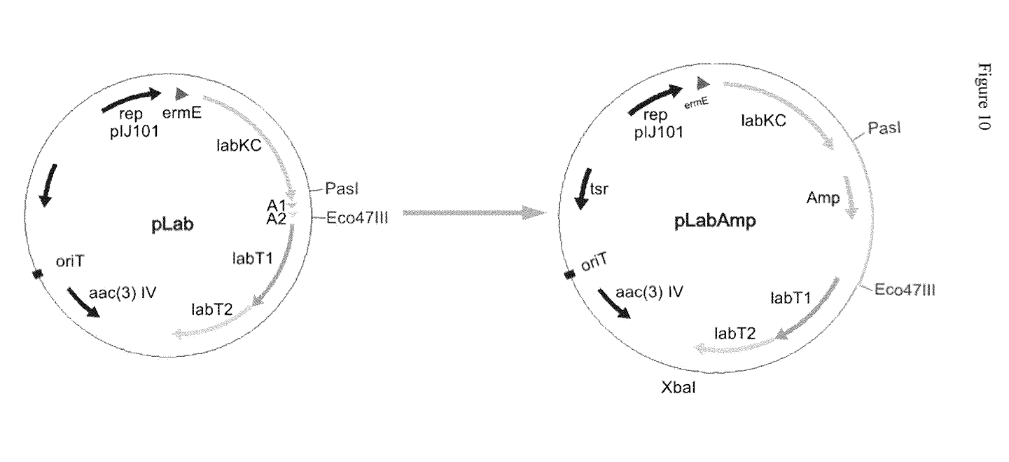

Although pLab allows cloning of new prepropeptide sequences by the use of PasI and Eco47III sites it is still not an optimal construct. After digestion of pLab with these two restriction enzymes two DNA fragments are obtained: 13431 by and 368 bp. To clone new prepeopeptide sequences to pLab first the digested vector needs to be isolated. However, it is difficult to distinguish on an preparative agarose electrophoresis gel between digested and not digested pLab. In this situation one could overdigest the pLab and use excess of prepared insert for ligation. Unfortunately this was not possible since PasI displays star activity (capability of a restriction enzyme to cleave sequences which are not identical to their defined recognition sequence). It means that after each cloning experiment obtained plasmids need to be sequenced to eliminate these which are carrying wild type sequences. To overcome this problem labA1 and labA2 were cut out with Past and Eco47III, and then these enzymes were used to clone DNA fragment coding for ampicillin resistance from a construct pMA_SG3 (FIG. 10). Such obtained pLabAmp vector has several advantages. First, because Amp has a size of 2359 bp it is possible to distinguish on the gel between digested and non digested vector (13428 bp). Second, it gives a possibility to eliminate false positives coming from a non digested vector by preparation of ampicillin replica plate. Bacteria carrying mutated sequences are resistance only to apramycin while bacteria with pLabAmp are resistance to apramycin and also ampicillin.

### Optimization of a Template for Engineering of LabA1

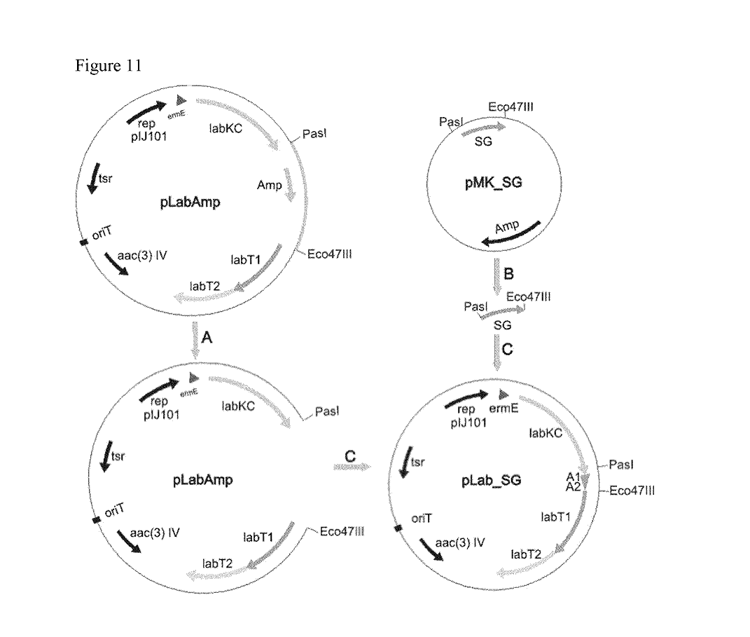

As it was described in Example 2 the drawback of pLab construct is that expressed Labyrinthopeptins contain additionally one or two amino acids at their N-termini. Presence of these amino acids can alter biological activity and imposes significant problems in the purification of such peptides. To overcome this limitation two main strategies were investigated. The first idea was to modify the leader sequence so it can be properly processed by host proteases. This would probably constitute the best option since then novel labyrinthopeptins can be directly isolated from bacterial cultures and do not need any further modifications. The second strategy is based on chemical degradation of incorrectly processed peptides. Although this method requires an additional step it is more predictable than the first one. The cloning strategy used to obtain vectors is presented in FIG. 11. Unknown proteases from a heterologous strain S. lividans are able to cut Labyrinthopeptin A1 leader sequence first between Ala-Ala and then between Ala-Asp. But they are not able to perform a cleavage between Asp-Ser, like it has been observed for the natural producer A. namibiensis. Based on this observation it was hypothesized that S. lividans would be able to cleave Ala-Ala bond. To investigate this hypothesis a synthetic gene SG3 was design where the last amino acid from a leader peptide (Asp) was substituted with Ala. SG3 was cloned to pLabAmp to create a vector pLab_SG3 (according to the cloning strategy shown above in FIG. 11). Expression of pLab_SG3 in S. lividans shown that the use of this construct allow expression of AA-LabA1 and A-LabA1. After 2-3 weeks of growth the ratio between AA-LabA1 and A-LabA1 shifts to give A-LabA1 as a main product. Interestingly in older cultures also the molecular mass corresponding to LabA1 could be found. Although the use of pLab_SG3 allows expression of LabA1 without any additional amino acids the amount of LabA1 was so low that this construct could not be used for further studies. Because no other data allowing a prediction of possible cleavage site was available, a strategy of obtaining Labyrinthopeptins completely processed in S. lividans was rejected at this stage of the project.

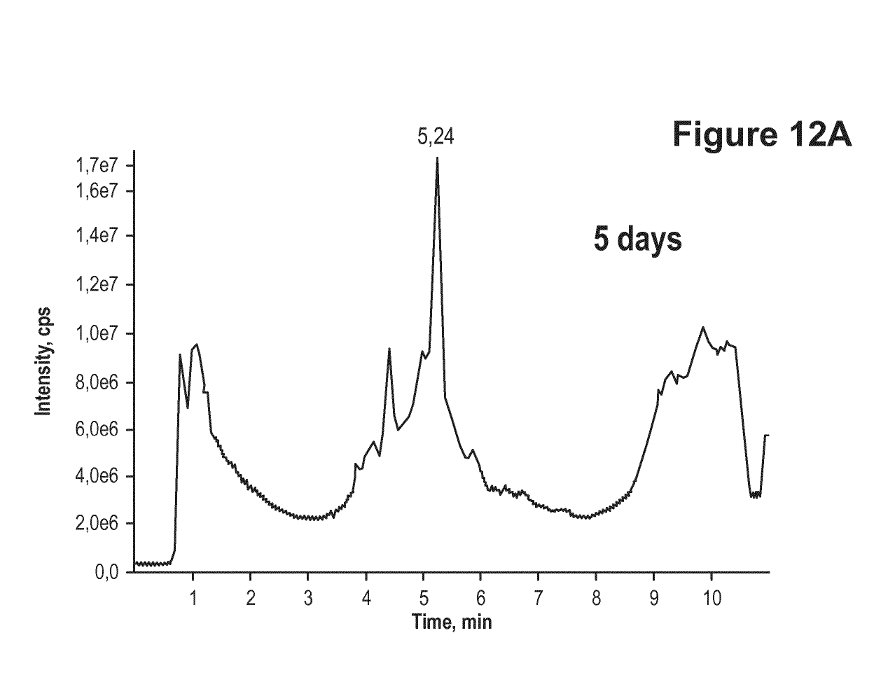

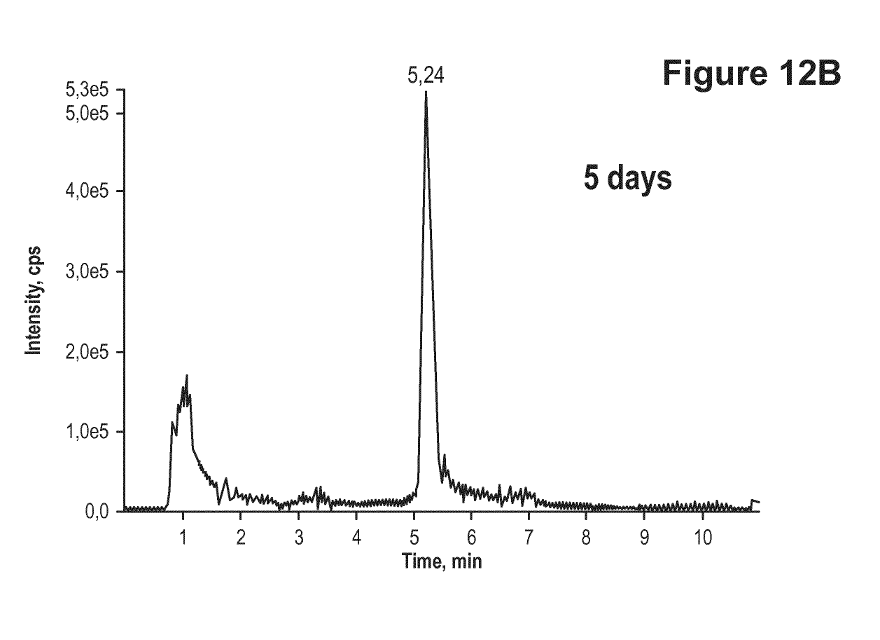

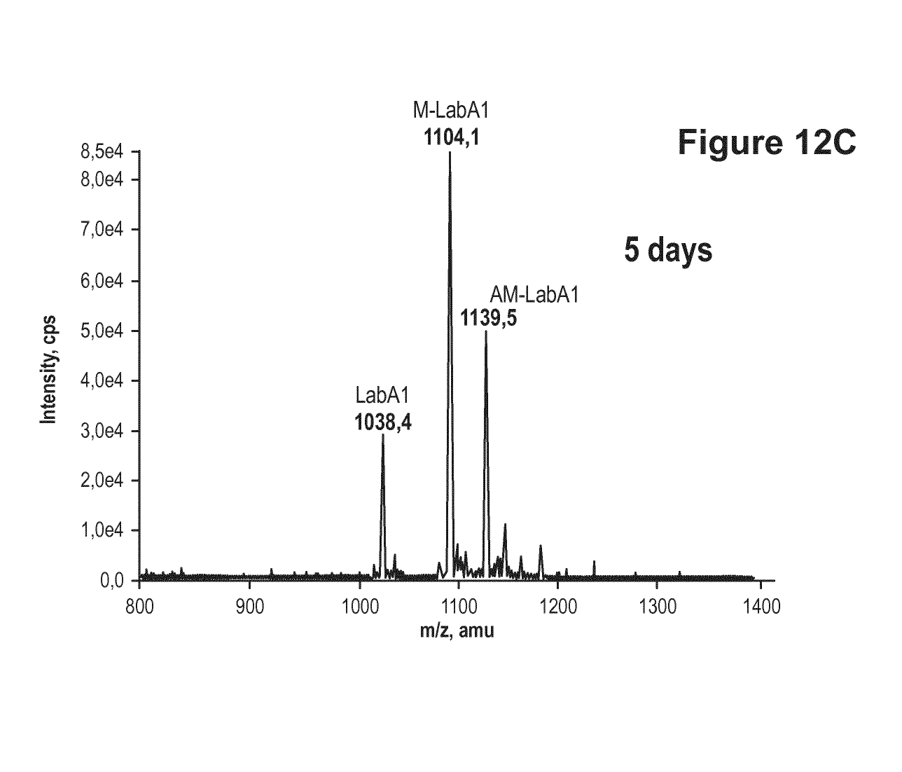

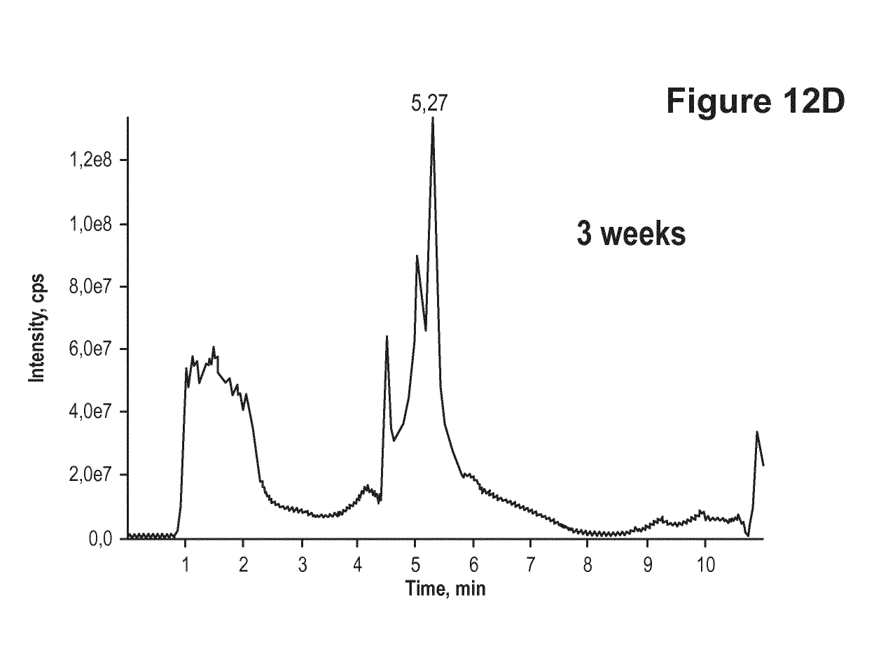

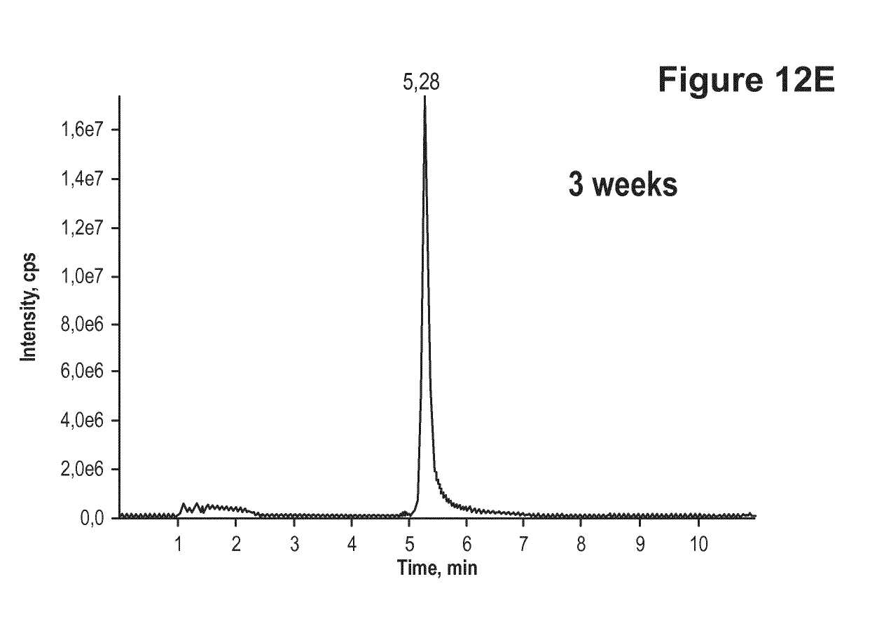

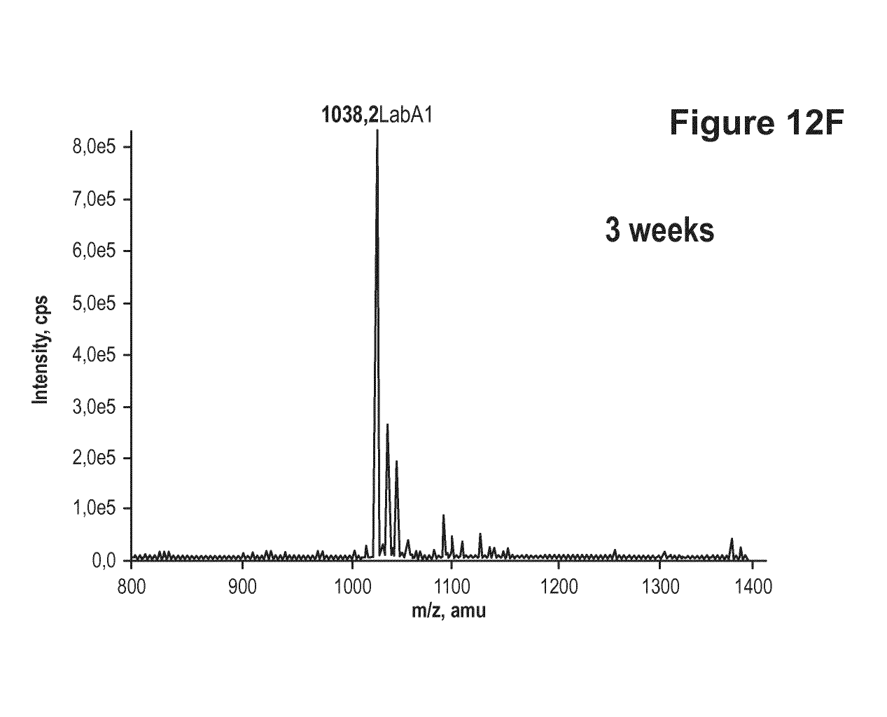

Another strategy was to isolate Labyrinthopeptins with additional amino acids allowing later reaction with CNBr, which removes N terminal methionine (60). To follow this method first Labyrinthopeptins with Met as the last, C-terminal amino acid in the leader peptide need to be expressed. A synthetic gene SG2 was ordered where in labA1 gene Asp codon was substituted with Met, and in labA2 gene Met codon was inserted between the last, C-terminal amino acid from a leader peptide and the first amino acid from a prepeptide (FIG. 13). Peptides observed in S. lividans/SG2 cultures where surprising (see FIG. 12): In one week old cultures AM-LabA1, M-LabA1 and also LabA1 could be detected. In older cultures (2-3 weeks old) almost all AM-LabA1 and M-LabA1 were converted to LabA1. Interestingly, no expression of LabA2 was detected. It seems that addition of Met completely abolish an expression of LabA2. Similar results were obtained in in vitro assays.

The use of pLab_SG2 allows an expression of LabA1 in S. lividans in the same way like in A. namibiensis. Another advantage of that construct is that LabA2 is not present in a S. lividans/pLab_SG2 culture, which makes isolation of LabA1 much easier. SG2 was used as a template for the generation of almost all LabA1 derivatives by means of site-directed mutagenesis below.

Peptides observed in a supernatant of S. lividans/pLab_SG2 and S. lividans/pLab_SG3 (ND—not detected):

| | | Peptide masses (Da) | | |
|---|---|---|---|---|
| Construct/ | | Expected | | |
| Labyrinthopeptin | | molecular | Expected | Observed |
| derivate | Formula | mass | [M + 2H]²⁺ | [M + 2H]²⁺ |
| pLab_SG2/LabA1 | C₉₂H₁₁₉N₂₃O₂₅S₄ | 2073.76 | 1037.88 | 1037.99 |
| pLab_SG2/M- | C₉₇H₁₂₈N₂₄O₂₆S₅ | 2204.96 | 1103.48 | 1103.38 |
| LabA1 | | | | |
| pLab_SG2/AM- | C₁₀₀H₁₃₃N₂₅O₂₇S₅ | 2276.04 | 1139.02 | 1138.87 |
| LabA1 | | | | |
| pLab_SG3/LabA1 | C₉₂H₁₁₉N₂₃O₂₅S₄ | 2073.76 | 1037.88 | ND |
| pLab_SG3/A- | C₉₅H₁₂₄N₂₄O₂₆S₄ | 2144.84 | 1073.42 | 1073.9 |
| LabA1 | | | | |
| pLab_SG3/ | C₉₈H₁₂₉N₂₅O₂₇S₄ | 2215.92 | 1108.96 | 1109.7 |
| AA_LabA1 | | | | |

### Optimization of a Vector Template for Engineering of LabA2

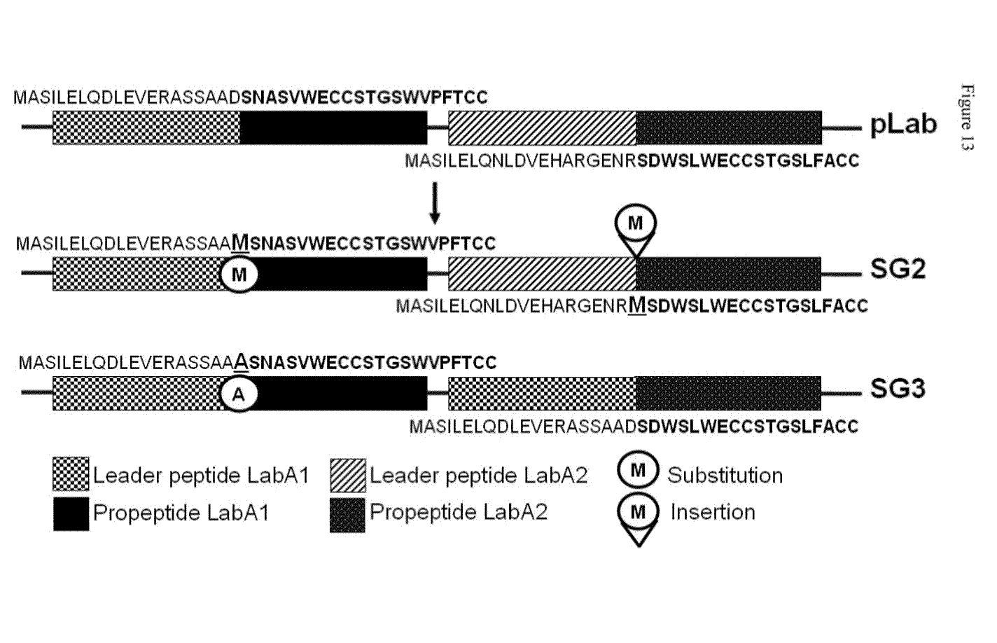

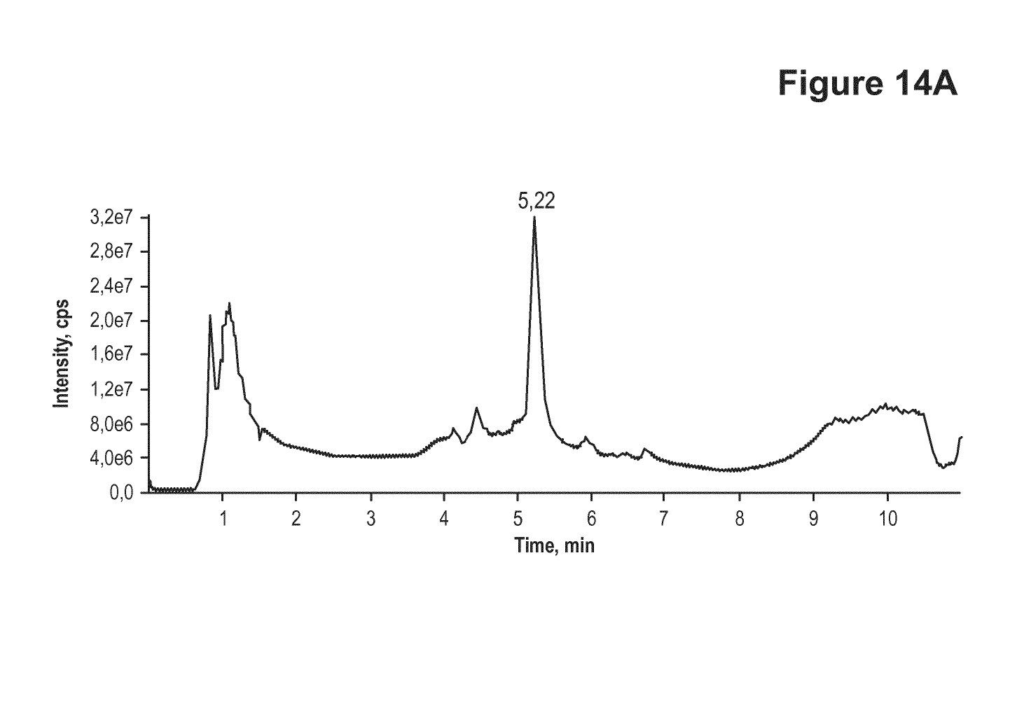

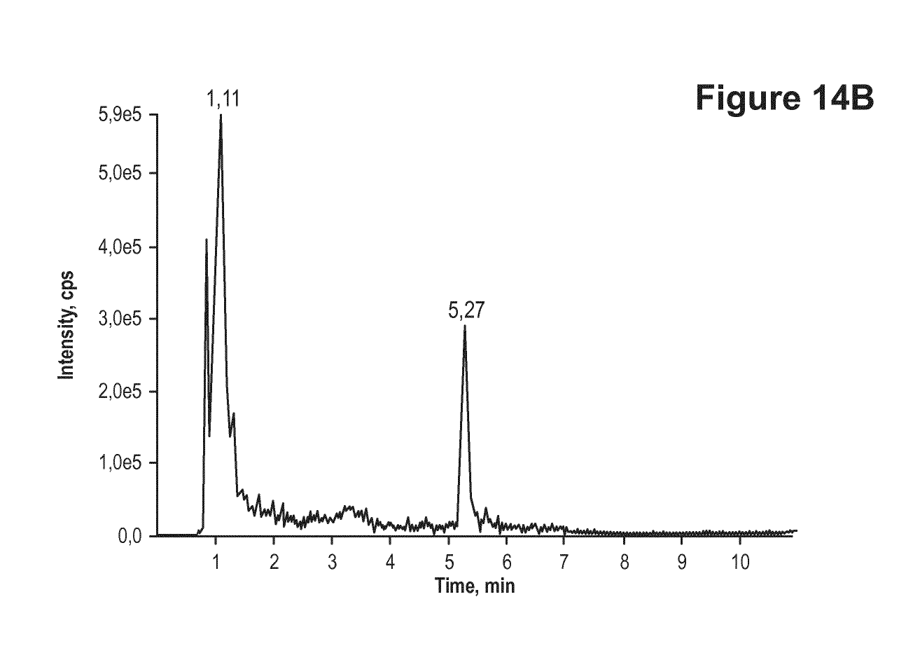

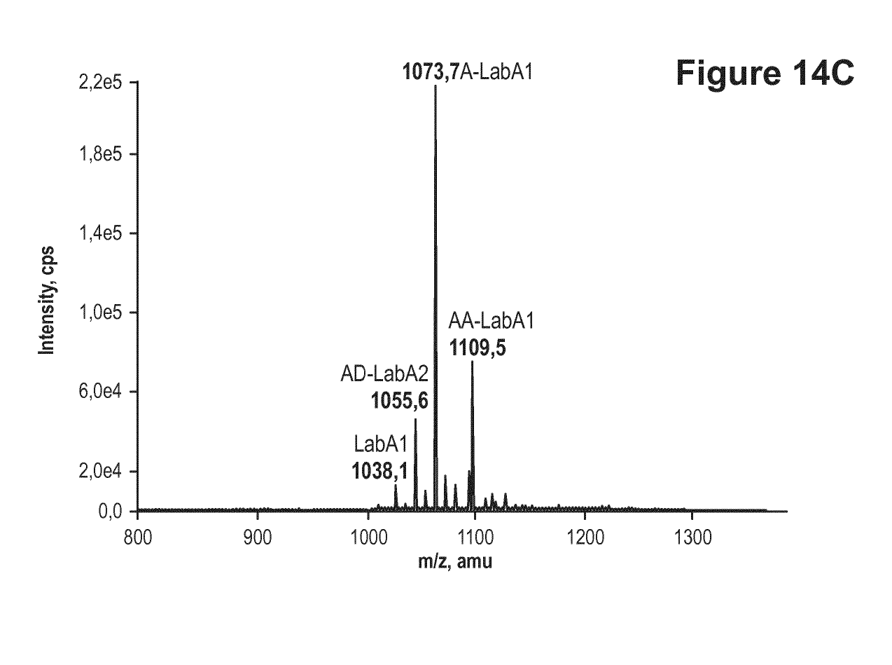

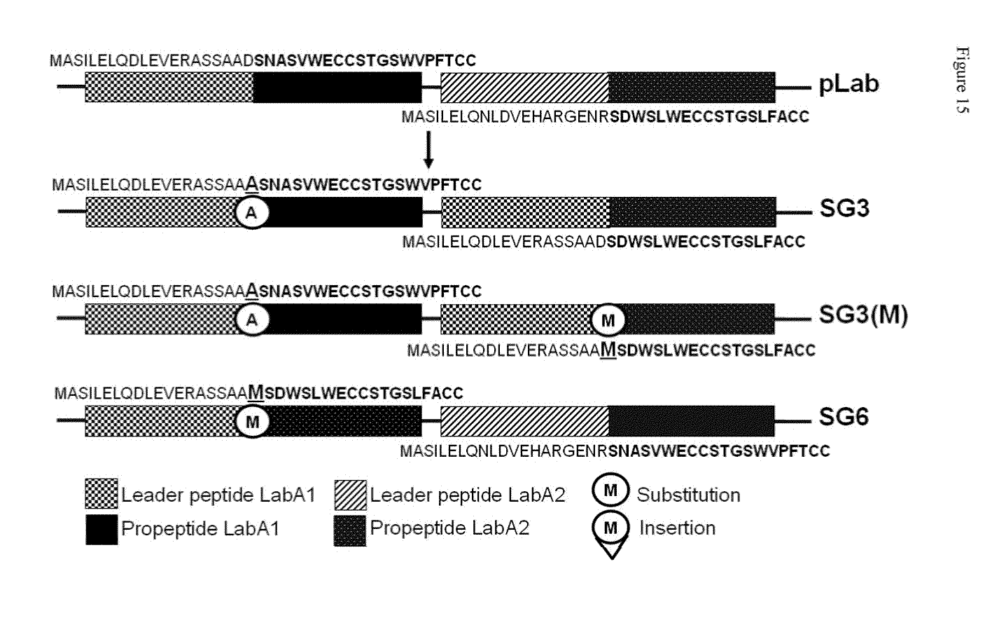

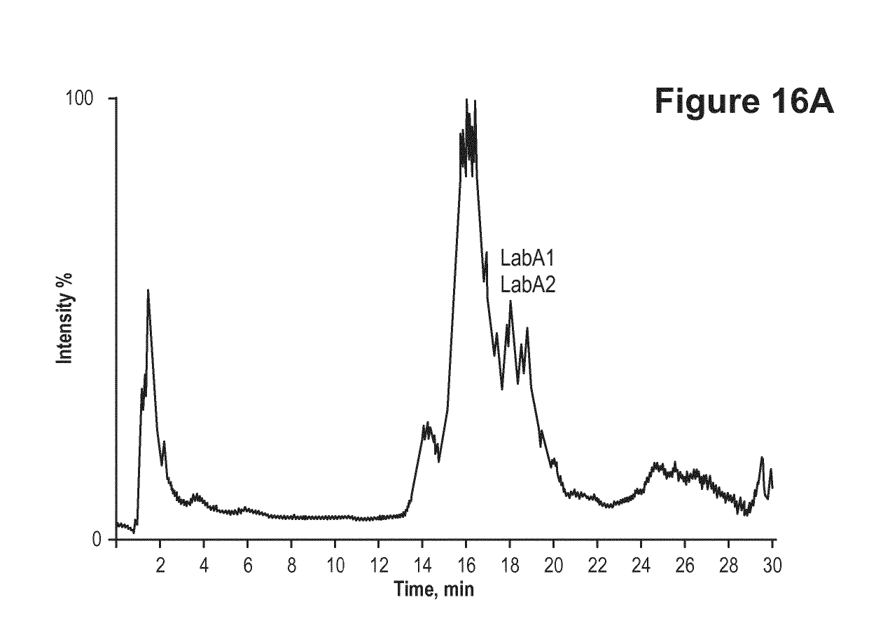

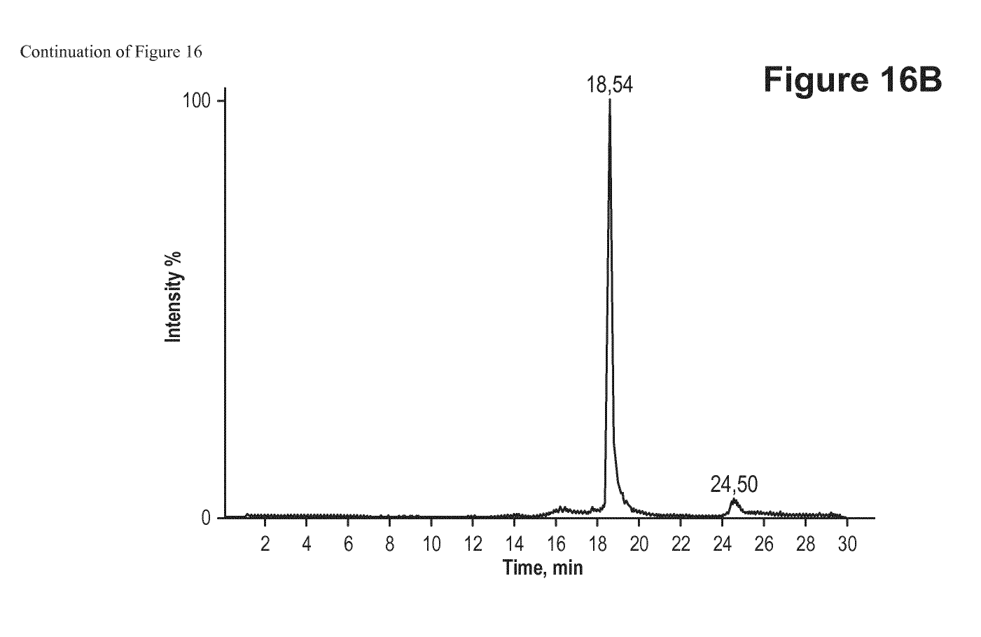

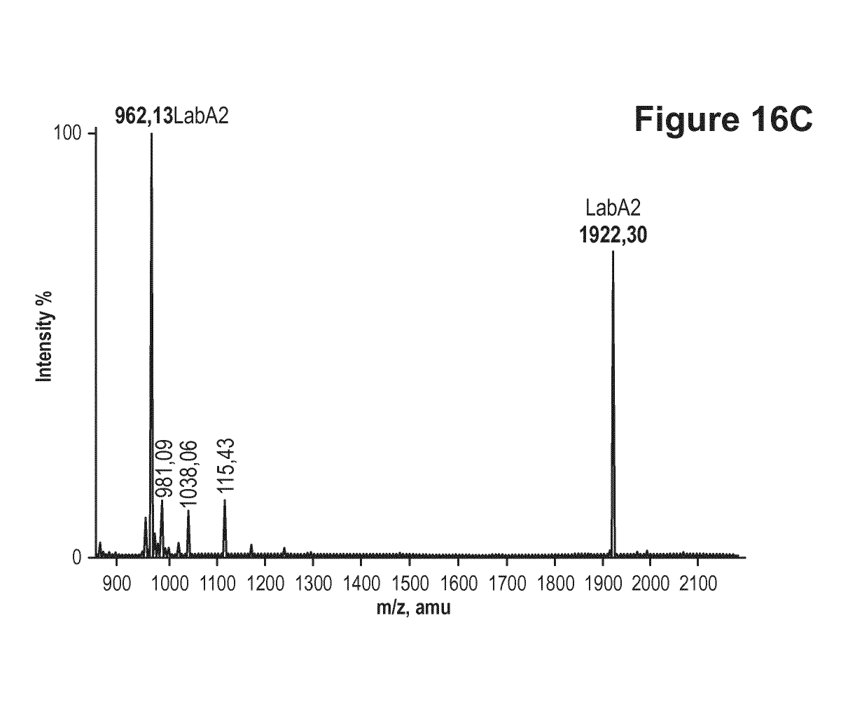

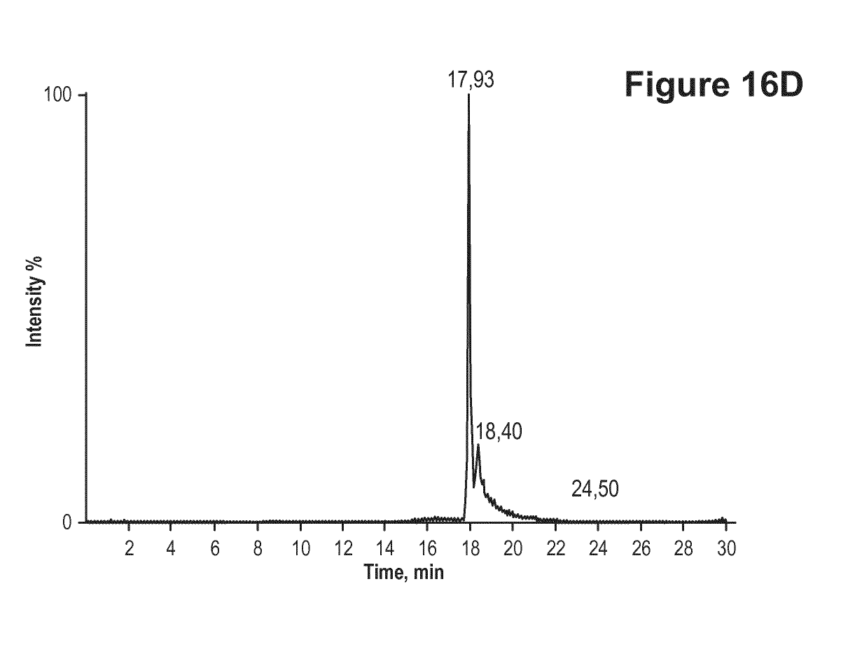

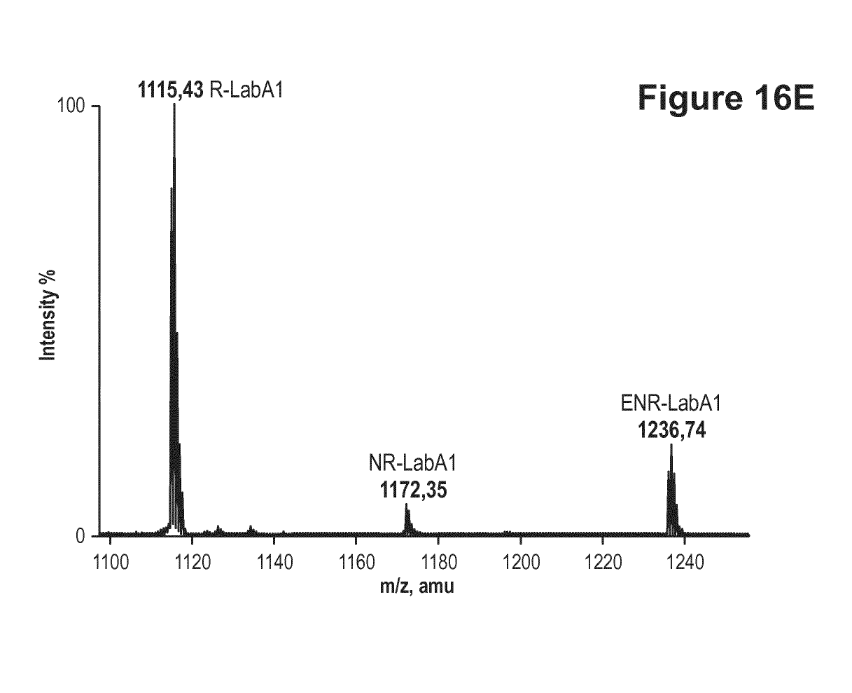

It was important to verify if the method established for proteolytic processing of LabA1 leader peptide could be applied to LabA2. The use of a gene SG3 (FIGS. 13, 14 and 15) showed that the expression of the peptide AD-LabA2 is possible when LabA2 prepeptide is attached to the LabA1 leader peptide. Another step was to generate SG3(M) by substitution of Asp with Met (like in SG2 for LabA1). This was accomplished by site-directed mutagenesis with primers D20M_fw (5′-CAGCTCGGCCGCCATGTCCGACTGGAGC-3′) and D20M_rv (5′-GCTCCAGTCGGACATGGCGGCCGAGCTG-3′) and SG3 as a template for PCR. S. lividans/pLab_SG3 was able to express LabA2 without additional amino acids from a leader sequence although expression levels were not high. It was observed for a wild type and also for heterologous strain that expression of LabA1 is higher than LabA2. It is possible that higher expression levels are due to the arrangement of genes in the lan cluster. In order to overcome a problem with low LabA2 expression, a gene SG6 was design where the order of labA1 and labA2 were swapped. Indeed LabA2 expression with the use of pLab_SG6 was higher than with pLab_SG3(M). S. lividans/pLab_SG6 expressed not only LabA2 but also LabA1 derivatives, as shown in FIG. 16. SG6 was used for generation of almost all LabA2 derivatives like it is described in an example below.

Peptides Observed in a Supernatant of S. lividans/pLab_SG3, S. lividans/pLab_SG6 and S. lividans/pLab_SG3(M):

| | | Peptide masses (Da) | | |
|---|---|---|---|---|
| Construct/ | | Expected | | |
| Labyrinthopeptin | | molecular | Expected | Observed |
| derivate | Formula | mass | [M + 2H]²⁺ | [M + 2H]²⁺ |
| pLab_SG3/AD- | C₉₂H₁₂₀N₂₂O₂₈S₄ | 2108.86 | 1055.43 | 1055.7 |
| LabA2 | | | | |
| pLab_SG3(M)/AM- | C₉₃H₁₂₄N₂₂O₂₆S₅ | 2124.97 | 1063.48 | 1063.7 |
| LabA2 | | | | |
| pLab_SG3(M)/M- | C₉₀H₁₁₉N₂₁O₂₅S₅ | 2053.89 | 1027.94 | 1028.3 |
| LabA2 | | | | |
| pLab_SG3(M)/ | C₈₅H₁₁₀N₂₀O₂₄S₄ | 1922.69 | 962.34 | 963.0 |
| LabA2 | | | | |
| pLab_SG6/M- | C₉₀H₁₁₉N₂₁O₂₅S₅ | 2053.89 | 1027.94 | 1027.99 |
| LabA2 | | | | |
| pLab_SG6/LabA2 | C₈₅H₁₁₀N₂₀O₂₄S₄ | 1922.69 | 962.34 | 962.52 |
| pLab_SG6/ENR- | C₁₀₇H₁₄₄N₃₀O₃₁S₄ | 2473.16 | 1237.58 | 1237.24 |
| LabA1 | | | | |
| pLab_SG6/NR- | C₁₀₂H₁₃₇N₂₉O₂₈S₄ | 2344.05 | 1173.02 | 1172.83 |
| LabA1 | | | | |
| pLab_SG6/R-LabA1 | C₉₈H₁₃₁N₂₇O₂₆S₄ | 2229.95 | 1115.97 | 1115.89 |

Expression of a single Labyrinthopeptin

Since LabA1 and LabA2 are similar molecules it would be helpful for isolation and downstream processing purposes to have a system to express not a mix of LabA1 and LabA2 but only a single labyrinthopeptin derivative at a time. Such a possibility was tested on two different ways. First by the use of pLab/SG4 where labA1 but not labA2 was present. The second construct was pLab/SG5, which contains only a sequence coding for LabA2 prepeptide attached to LabA1 leader peptide with Met as a C-terminal amino acid of the leader peptide (see FIG. 17). No expression of LabA1 derivatives were detected in cultures of S. lividans/pLab_SG4 as well as none LabA2 derivatives were observed in S. lividans/pLab_SG5 cultures. These results show that both genes coding for prepropeptides are needed Labyrinthopeptin expression. This phenomenon is still not explained but probably it is due to the stabilization of mRNA. It seems that the best method for expressing of only one Labyrinthopeptin is to insert Met between sequence of leader and prepeptide like it was done for LabA2 in SG2 gene. This data demonstrates that the complementation strategy, which requires structural gene knock-out, would probably fail.

Detection of Labyrinthopeptin derivatives in cultures of S. lividans/pLab_SG4 and S. lividans/pLab_SG5 (ND—not detected):

| | | Peptide masses (Da) | | |
|---|---|---|---|---|
| Construct/ | | Expected | | |
| Labyrinthopeptin | | molecular | Expected | Observed |
| derivate | Formula | mass | [M + 2H]²⁺ | [M + 2H]²⁺ |
| pLab_SG5/AM-LabA2 | C₉₃H₁₂₄N₂₂O₂₆S₅ | 2124.97 | 1063.48 | ND |
| pLab_SG5/M-LabA2 | C₉₀H₁₁₉N₂₁O₂₅S₅ | 2053.89 | 1027.94 | ND |
| pLab_SG5/LabA2 | C₈₅H₁₁₀N₂₀O₂₄S₄ | 1922.69 | 962.34 | ND |
| pLab_SG4/AD-LabA1 | C₉₉H₁₂₉O₂₉N₂₅S₄ | 2259.83 | 1130.91 | ND |
| pLab_SG4/D-LabA1 | C₉₆H₁₂₄O₂₈N₂₄S₄ | 2188.79 | 1095.39 | ND |
| pLab_SG4/LabA1 | C₉₂H₁₁₉N₂₃O₂₅S₄ | 2073.76 | 1037.88 | ND |

In summary, it was possible to establish a method for engineering of Labyrinthopeptins which overcomes three main problems very often found in related studies. First of all, because the designed method introduces as few changes as possible in the lab gene cluster, the high level of expression could be maintained. This is an extremely important aspect in terms of subsequent peptide isolations and biological activity tests. Additionally a cloning strategy was designed to be very efficient. The use of a vector pLabAmp allows fast and easy generation of labyrinthopeptin variants only by cloning of fragments containing mutated structural genes. labA1 and labA2 genes on a small plasmid (pMK or pMA) are easily manipulated by site-directed mutagenesis. Capabilities for creation of mutants by the use of a system proposed in this work are demonstrated by the number of generated novel labyrinthopeptins described herein.

The third and probably most challenging problem was to express labyrinthopeptins without any additional N-terminal overhangs. During this study a new processing site allowing correct leader peptide removal in the heterologous host was discovered. Most importantly the introduced modifications had no detectable impact on the processing by the LabKC enzyme. This strategy enabled to fully control wild type LabA1 and LabA2 productions by introducing a Met residue between leader and structural peptides and by exchanging leader peptides. It was also possible to engineer the construct to enforce production of only one labyrinthopeptin.

Presented results demonstrate further that establishing of a heterologous expression system for lantibiotic production, and in vivo engineering of these compounds is not an easy and straightforward task. What seems to be true also in similar studies is that the best strategy is to combine biochemical intuition with a trial and error approach.

### Example 4

### Engineering of New Labyrithopeptins

It is always desirable to generate new variants of natural products displaying biological activities. Systematically introduced changes can be helpful in understanding the mode of action and even in the building of a pharmacophore model for a drug candidate. Moreover, rationally designed modifications can tailor some interesting properties like activity, stability or ADME profiles. Lantibiotics seem to be good targets for engineering with the possibility of accommodating numerous changes, because of their ribosomal origin and promiscuous substrate specificity (61-62) of enzymes involved in their biosynthesis. Modifications could be introduced by using different methods e.g. chemical synthesis, genetic modification of producing strain, chemical modifications on naturally produced compounds and semisynthesis. For the presented study, a biological approach was chosen, mainly as a consequence of difficulties associated with the synthetic attempts. However, there are reports of successful synthesis of lantibiotics e.g. total synthesis of nisin A (63) and lactocin S (64) were successfully conducted. Nevertheless, these methods are still expensive, multistep and not efficient enough for drug development purposes.

The lantibiotic engineering faces many obstacles mainly because of the fact that expression systems need to be developed not only for structural genes but also for genes encoding biosynthetic enzymes, immunity and regulatory proteins. Even the well-developed expression systems do not guarantee a successful expression of the final product. It has been shown that the number of mutations which can be applied is limited (65). Many designed mutants could not be produced, probably due to disturbed interactions of the peptides with biosynthetic enzymes or secretion machinery (65). In spite of all these limitations a large variety of different lantibiotics variants has been reported and characterized, the most prominent examples being: nisin (50-51), Pep5 (54), epidermin (53), mutacin II (52), actagardine (49), lacticin 3147 (66), mersacidin (67). Unfortunately, in most cases obtained derivatives did not exhibit improved properties. One of the most notable mutants was prepared for subtilin by substitution of glutamate (Glu4) with isoleucine. This derivative possesses approximately 3-4 fold higher activity than the natural peptide and displays a 57-fold increase in chemical and biological stability (68). Few spectacular mutants were prepared also for nisin. Nisin derivative which was prepared by conversion of Dha5 to Dhb has a greater resistance to acid catalyzed chemical degradation than wild-type nisin Z (68). Similarly the solubility of nisin at neutral pH has been enhanced through mutagenesis (68). Studies on this lantibiotic demonstrated that the use of genetic engineering enables modulating the antimicrobial activity of a compound, with respect to particular target organisms. A nisin mutant G18T/M17Q was approximately 4 fold less active than nisin against B. cereus and S. thermophilus, but it was twice more active than the wild type nisin against M. flavus (68).

In order to overcome problems connected with the generation of mutants in vivo (e.g. immunity and regulatory proteins must be functional, mutated peptides may be degraded by proteases) in vitro assays were developed. Another advantage of the in vitro assay is that lantibiotic biosynthetic enzymes used in it show even greater flexibility than in the in vivo method. Moreover, biosynthesis intermediates generated by an in vitro approach commonly can be more specifically characterized by bioanalytical methods, which allows elucidation of biosynthesis mechanisms on a molecular level (69). The successes in this area include the reconstitution of activity in in vitro assays for lacticin 481 (61), nukacin ISK-1 (70), mutacin II (70), ruminococcin A (70), nisin (71), haloduracin (72), Labyrinthopeptin (73), venezuelin (74) and lantipeptides from Prochlorococcus MIT9313 (75). In some cases it was not possible to reconstitute in vitro activity despite of multiple attempts (reported examples: RamC, NisA). Worth mentioning is also the fact that the benefits are often restricted by the limited quantities of the final product.

Another reason which makes lantibiotics engineering very interesting is the presence of lanthionine, a characteristic structural motif providing chemical, proteolytic and metabolic stability. Several studies have shown that cyclic lanthionines increase both stability and activity of non-lantibiotic peptides (76-77). This suggests that this motif may be used as the structural core for the design of new therapeutic peptides. Moreover, the recently reported labionin amino acid, which was identified in Labyrinthopeptin structures, provides even higher conformational constraint and seems to be responsible for novel biological activity against neuropathic pain and viral diseases. As described in the Example above, we succeeded in the construction of a vector pLab_SG2 and pLab_SG6 which can easily be used as a template for bioengineering of LabA1 and LabA2, respectively. This enables expressing Labyrinthopeptin-like peptides for SAR studies purposes and for testing substrate specificity of the modifying enzyme. To the best of my knowledge, this is the first report of engineering of a lantibiotic demonstrating a biological function different than antimicrobial activity. These are also the first in vivo studies on generation of mutants containing labionin.

### Strategy for the Generation of Mutants

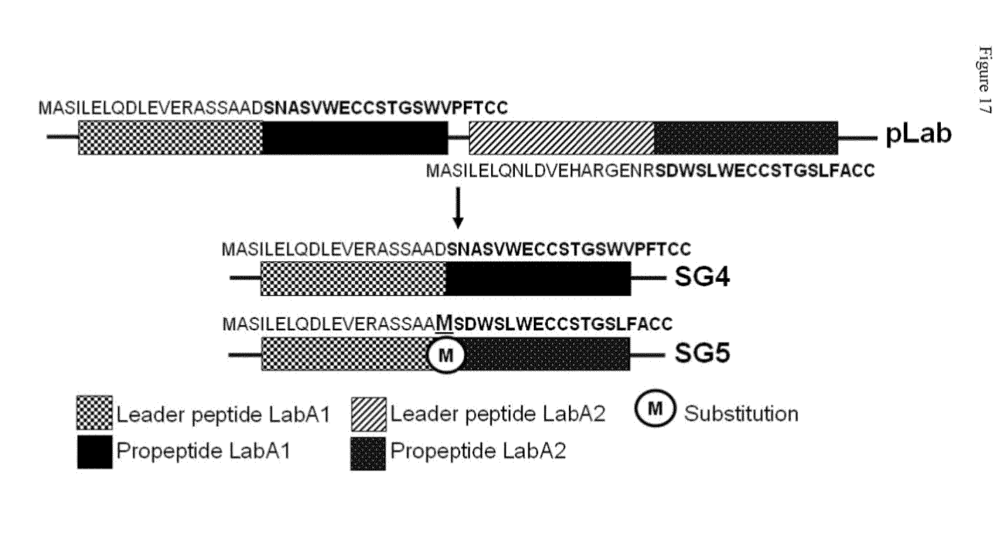

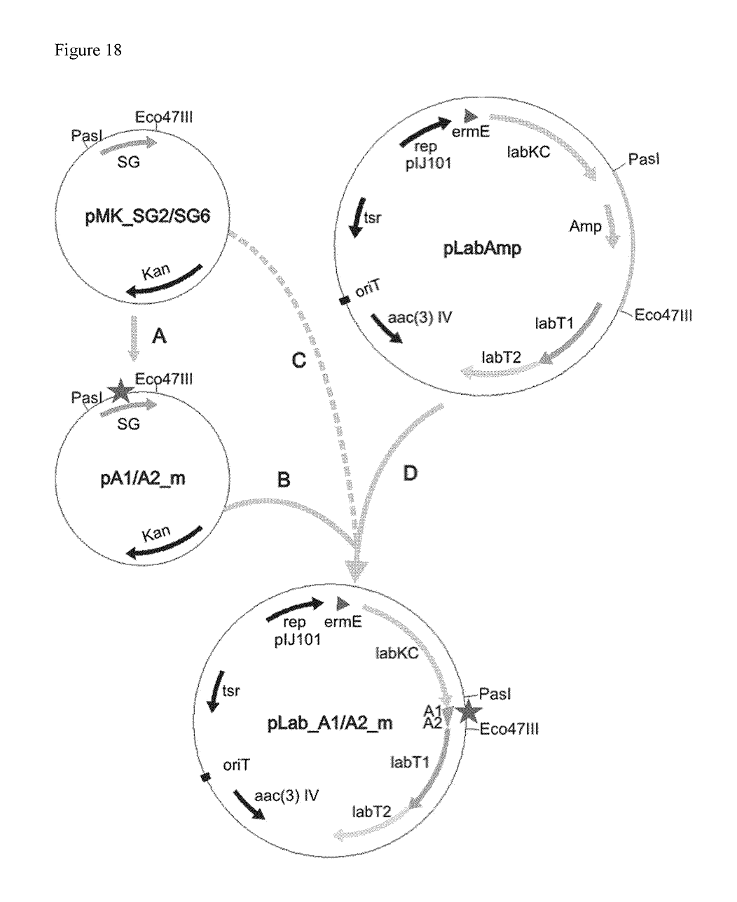

All Labyrinthopeptin derivatives were generated in the same way as shown in FIG. 18. The first step was the preparation of a linearized vector pLabAmp by restriction digestion with PasI and Eco47III. Then, fragment of the vector containing the lab gene cluster (13.4 kpb) without structural genes was extracted from agarose gel. Since yields from DNA recovery after agarose gel isolation is normally very low, most of the cloning experiments were performed using the whole restriction mixture purified with the GeneJET™ PCR Purification Kit (Fermentas). The desired insert was provided by PCR reaction from an appropriate template plasmid using Infusion1 and Infusion_rv primers. Reaction conditions were optimized, to obtain only a single product which was directly used to prepare a final construct (pLab_A1_m or pLab_A2_m, where m indicates modifications in LabA1 or LabA2 respectively) with ligation independent cloning approach (LIC, (13-14)). All constructs were verified by sequencing and/or restriction digestion. Because of high GC content and the presence of secondary structures in many cases sequencing reaction with pLab_A1/2_m failed. To overcome this problem a region coding for Labyrinthopeptins prepropeptides was amplified by PCR with Infusion1 and Infusion_rev primers, isolated and sequenced with primer Infusion1.

Two main types of template plasmids were used to generate mutants. In order to perform site-directed mutagenesis of labA1 and labA2 structural genes were provided on above described plasmids: pMK_SG2 and pMK_SG6. Small size of these vectors (pMK_SG2: 2670 bp, see FIG. 35A; pMK_SG6: 2664 bp, see FIG. 35B) allows an easy exchange of amino acids. While designing primers the codon usage of Streptomyces (see blow) was considered and most frequent codons were used. All constructs were confirmed by sequencing with pMA primer. Correct inserts were amplified by PCR and used for preparation of final constructs as described above. The second type of plasmid templates used to prepare more complicated variants were provided on plasmids pMK_SGx (“x” refers to variant number). In this case structural genes between Past and Eco47III sites were prepared by a combination of enzymatic and chemical synthesis of DNA (78) and purchased from GENEART AG (Regensburg Germany) already cloned into pMK plasmid.

The codon usage (CU) of the genus Streptomyces (88). CU—codon usage in Streptomyces (%).

| Amino | Co- | | Amino | Co- | | Amino | Co- | |
|---|---|---|---|---|---|---|---|---|
| acid | don | CU | acid | don | CU | acid | don | CU |
| Phe | TTT | 1.6 | Ser | TCT | 1.1 | Arg | CGT | 7.3 |
| | TTC | 98.4 | | TCC | 39.1 | | CGC | 45.0 |
| Glu | GAA | 17.7 | | TCA | 2.4 | | CGA | 3.7 |
| | GAG | 82.3 | | TCG | 27.4 | | CGG | 37.7 |
| Leu | TTA | 0.4 | Cys | TGT | 12.0 | Gln | CAA | 6.1 |
| | TTG | 2.6 | | TGC | 88.0 | | CAG | 93.9 |
| Tyr | TAT | 5.0 | Stop | TGA | 81.0 | Ala | GCT | 2.3 |
| | TAC | 95.0 | Trp | TGG | 100.0 | | GCC | 58.7 |
| Stop | TAA | 4.0 | Pro | CCT | 2.7 | | GCA | 4.3 |
| | TAG | 15.0 | | CCC | 42.4 | | GCG | 34.7 |
| Leu | CTT | 2.0 | | CCA | 1.6 | Ile | ATT | 4.4 |
| | CTC | 38.5 | | CCG | 53.3 | | ATC | 92.0 |
| | CTA | 0.3 | His | CAT | 6.5 | | ATA | 3.6 |
| | CTG | 56.1 | | CAC | 93.5 | Asn | AAT | 4.6 |
| Thr | ACT | 2.2 | Ser | AGT | 2.9 | | AAC | 95.4 |
| | ACC | 65.2 | | AGC | 27.1 | Val | GTC | 56.0 |
| | ACA | 2.4 | Gly | GGT | 8.7 | | GTA | 2.5 |
| | ACG | 30.2 | | GGC | 64.2 | | GTG | 38.8 |
| Lys | AAA | 5.8 | | GGA | 8.9 | Met | ATG | 100.0 |
| | AAG | 94.2 | | GGG | 18.2 | | | |

Explanation of the Meaning of Terms Used to Designate Mutation Codes in this Study:

| Mutation type | Mutation code | Description |
|---|---|---|
| Substitution | E15P | Replaces a glutamic acid (E) at position |
| | | 15 with a proline (P). |
| Deletion | E15del | Deletes a glutamic acid (E) at position |
| | | 15. |
| | EF15del | Deletes a glutamic acid and a |
| | | phenylalanine (EF) at positions 15-16. |
| Insertion | E15insP | Inserts a proline (P) at position 15, |
| | | pushing the glutamic acid (E) to |
| | | position 16. |
| | EF15insPQ | Inserts a proline and a glutamine (PQ) |
| | | at positions 15-16, pushing the |
| | | glutamic acid and the phenylalanine |
| | | (EF) to positions 17-18. |

### Alanine Scanning Mutagenesis of LabA1 and LabA2

Structure-activity relationship (SAR) studies evaluate importance and contributions of different parts of a target molecule to its bioactivity. This allows later rational modifications which may improve its properties. SAR studies may also be useful for building a pharmacophore model of lead compounds during drug development processes. The most important requirement for performing SAR studies is the availability of structural analogs. In the case of ribosomally synthesized peptides genetic manipulations leading to the generation of new peptides are very often more efficient than synthetic approaches. However, the most important issue is the recognition and proper processing by the post-translational modification machinery. One of the most widely used strategies for SAR studies of peptides and proteins is alanine scanning, when relevant residues are exchanged one by one with Ala. In most of the cases Ala is a reasonable choice since it neither disturb secondary structures nor introduce extreme steric or electrostatic changes (79).

Above presented strategy to generate mutants of Labyrinthopeptins was applied to alanine scanning procedure. Since investigated peptides display interesting antiviral activities and a remarkable activity against neuropathic pain (mouse model) this experiment is a starting point for subsequent SAR studies. Moreover, alanine scanning in this case will additionally provide information about importance of each residue for biosynthesis which is a key issue for isolation and further activity studies. Former studies showed that the production levels of lantibiotics mutants were significantly reduced or totally suppressed when residues involved in lanthionine formation were modified (66). This indicates that the presence of the lanthionine rings is important for recognition by the secretion systems (53, 80) and it might provide proteolysis stability necessary suppress degradation. Based on those findings, in this study only amino acids which are not involved in formation of rings (lanthionine, labionin and disulphide according to the X-ray crystal structure) were substituted with Ala.

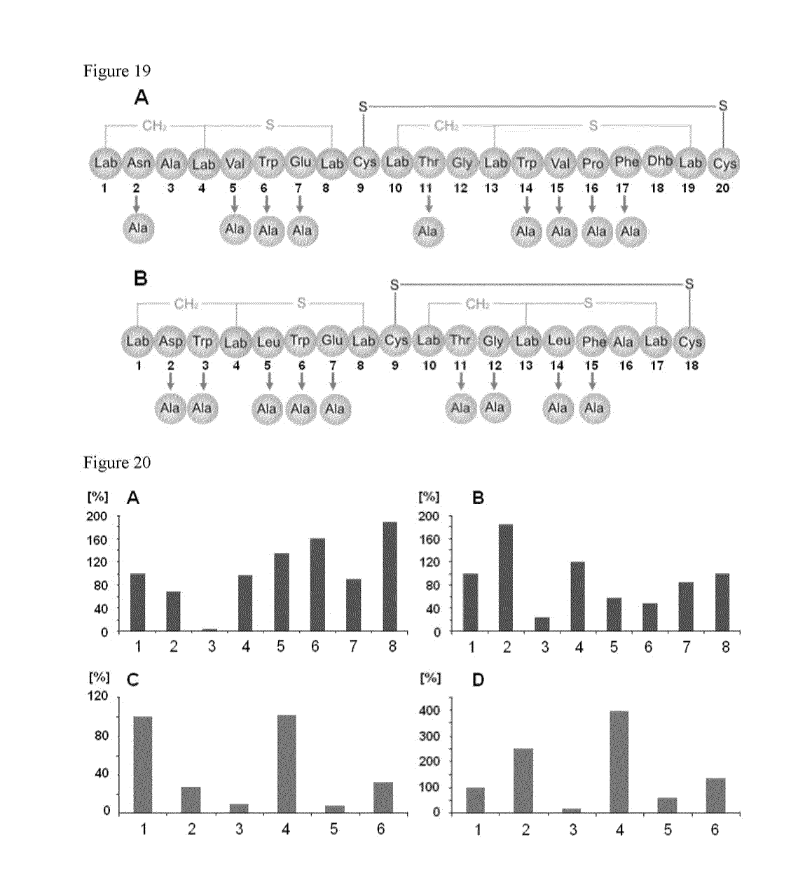

Generation of nine mutants for LabA1 and also nine mutants for LabA2 was accomplished. For two LabA1 mutants (LabA1_G12A and LabA1_T18A) it was impossible to introduce necessary mutation even though site-directed mutagenesis experiments were repeated several times. All performed mutations are summarized in FIG. 19. LC-MS was used in order to detect production of mutated peptides. In each case presence of peptides in liquid cultures and in extracts from MS plates was verified. Interestingly, LabA1 and LabA2 mutants displayed considerable differences in productions observed in liquid cultures. Molecular masses corresponding to LabA1 mutants were found in all three tested media (YEME, R2YE, NZ Amine) LabA2 mutants were produced in sufficient amounts only in NZ Amine medium (exceptions were mutants where Trp was substituted with Ala). In all cases observed molecular masses were in agreement with theoretically calculated ones. Production yields of LabA1 and LabA2 mutants varied significantly which was in agreement with observations made for other engineered peptides (81). FIG. 20 shows changes in production levels of LabA1 and LabA2 alanine mutants after 8 and 21 days of growth in NZ Amine medium. To eliminate additional factors, which could have an influence on Labyrinthopeptins production, all cultures were started in the same day and bacteria were grown in the same type of flask. Presented quantification was performed by means of LC-MS with assumption that introduced mutations do not change ionization efficiency in LC-ESI-MS investigation. Since measurements were performed in positive mode and none of mutated residues was basic this was quite a reasonable assumption. For LabA1 as well as for LabA2 all mutants where tryptophan was substituted with alanine were produced only in trace amounts. This result may indicate that these peptides are not efficiently recognized by the modification and/or secretion system. An alternative explanation might be that these residues are crucial for proper peptide conformation which is necessary for the ring formation. Another interesting finding concerns Trp3 of LabA2. As presented on FIG. 19, substitution of this residue with alanine makes A ring of LabA2 very similar to that of LabA1 (dipeptides Asn-Ala for LabA1 and Asp-Ala for LabA2). However, even in this case production is significantly suppressed after Trp substitution which is even more surprising since both peptides are modified by the same enzyme.

Structural characterization of a mutant peptide LabA2_L14A derived from LabA2 by NMR spectroscopy

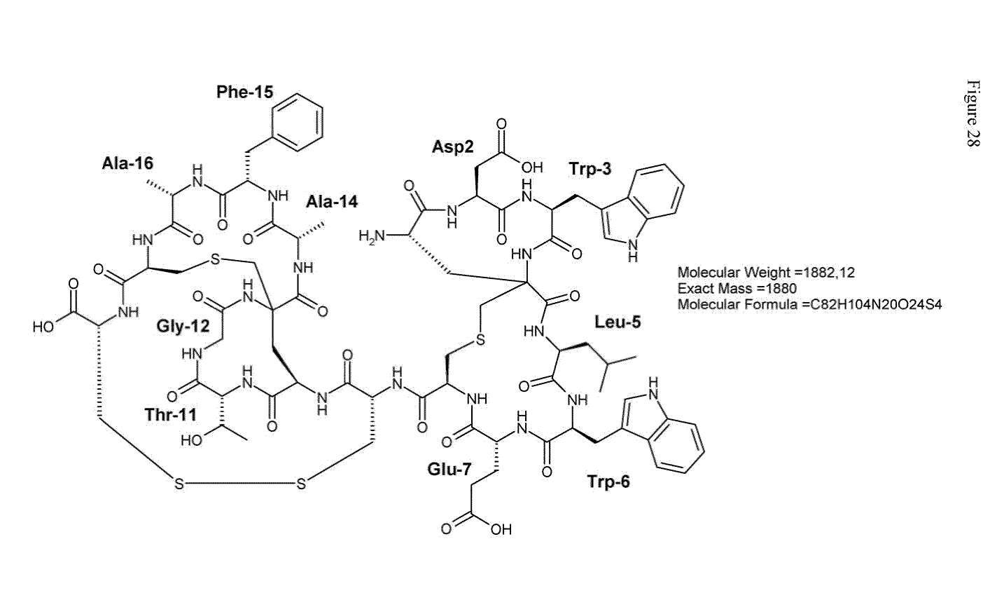

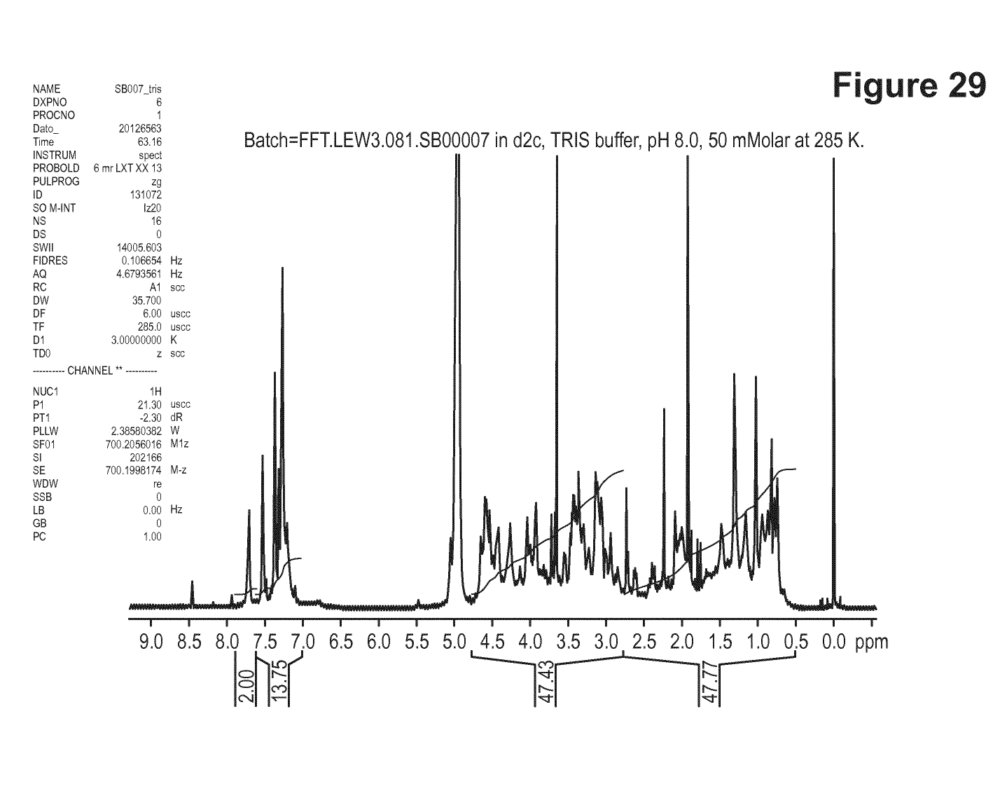

The position of substitution in the mutant peptide LabA2_L14A (Leu14 is substituted by Ala; see FIG. 28) was identified by comparison with the NMR spectra (FIG. 29) obtained for the natural Labyrinthopeptin A2. The analysis of the NMR spectra is hampered by partially extreme line broadening. A partial proton and carbon assignment is given in the following table, showing chemical shifts of LabA2_L14A and LabA2 in TRIS buffer (pH 8.0, 50 mMolar) at 285 K:

| | 1H | | 13C | |
|---|---|---|---|---|
| | LabA2 | LabA2_L14A | LabA2 | LabA2_L14A |
| Asp-2 | | | | |
| α | 5.050 | 5.044 | 47.6 | 47.6 |
| β | 2.627/2.385 | 2.616/2.374 | 38.9 | 39.0 |
| Trp-3 | | | | |
| α | 4.580 | 4.567 | 56.0 | 56.0 |
| β | 3.441/3.097 | 3.442/3.087 | 28.3 | 28.5 |
| 2 | 7.353 | 7.356 | 125.2 | 125.2 |
| 3 | | | 109.7 | |
| 3a | | | 127.4 | |
| 4 | 7.702 | 7.700 | 119.3 | 119.3 |
| 5 | 7.176 | — | 120.6 | 120.6 |
| 6 | 7.283 | 7.279 | 123.3 | 123.3 |
| 7 | 7.538 | 7.535 | 113.2 | 113.2 |
| 7a | | | 137.0 | |
| Leu-5 | | | | |
| α | 4.265 | 4.249 | 53.4 | 53.3 |
| β | 1.493/1.410 | broad | 40.6 | broad |
| γ | 1.488 | 1.480 | 24.9 | 25.0 |
| δ | 0.826 | 0.817 | 23.8 | 23.8 |
| δ′ | 0.756 | 0.758 | 20.9 | 20.9 |
| Trp-6 | | | | |
| α | 4.580 | 4.567 | 56.0 | 56.0 |
| β | 3.407/3.360 | 3.422/3.353 | 26.7 | 26.6 |
| 2 | 7.269 | 7.264 | 125.8 | 125.8 |
| 3 | | | 110.4 | |
| 3a | | | 127.4 | |
| 4 | 7.711 | 7.706 | 119.7 | 119.7 |
| 5 | 7.220 | 7.216 | 120.6 | 120.6 |
| 6 | 7.256 | 7.259 | 123.3 | 123.3 |
| 7 | 7.521 | 7.520 | 113.2 | 113.2 |
| 7a | | | 137.0 | |
| Glu-7 | | | | |
| α | 3.987 | 4.000 | 55.3 | 55.4 |
| β | 2.062/1.889 | 2.072/broad | 26.4 | 26.3 |
| | 1H | | 13C | |
| | Laby A2 | SB0007 | Laby A2 | SB0007 |
| Thr-11 | | | | |
| α | 4.511 | broad | 55.7 | broad |
| β | 4.276 | 4.266 | 67.4 | 67.3 |
| γ | 1.171 | broad | 19.7 | broad |
| Gly-12 | | | | |
| α | 3.442/3.284 | 3.403/broad | 43.2 | 42.7 |
| Leu-14/ | | | | |
| Ala-14 | | | | |
| α | 4.088 | 4.040 | 55.2 | 53.3 |
| β | 1.590/1.236 | 1.307 | 40.8 | 17.6 |
| γ | 1.405 | | 25.5 | |
| δ | 0.764 | | 21.1 | |
| δ′ | 0.859 | | 23.3 | |
| Phe-15 | | | | |
| α | 4.522 | 4.528 | 56.1 | 56.4 |
| β | 3.087/2.984 | 3.128/2.933 | 38.1 | 38.7 |
| γ | | | 137.0 | |
| δ | 7.245 | 7.252 | 130.8 | 130.8 |
| ε | 7.372 | 7.366 | 129.9 | 129.9 |
| ζ | 7.310 | 7.311 | 128.3 | 128.3 |
| Ala-16 | | | | |
| α | 3.947 | 3.918 | 50.9 | 50.9 |
| β | 1.102 | 1.027 | 15.1 | 15.2 |

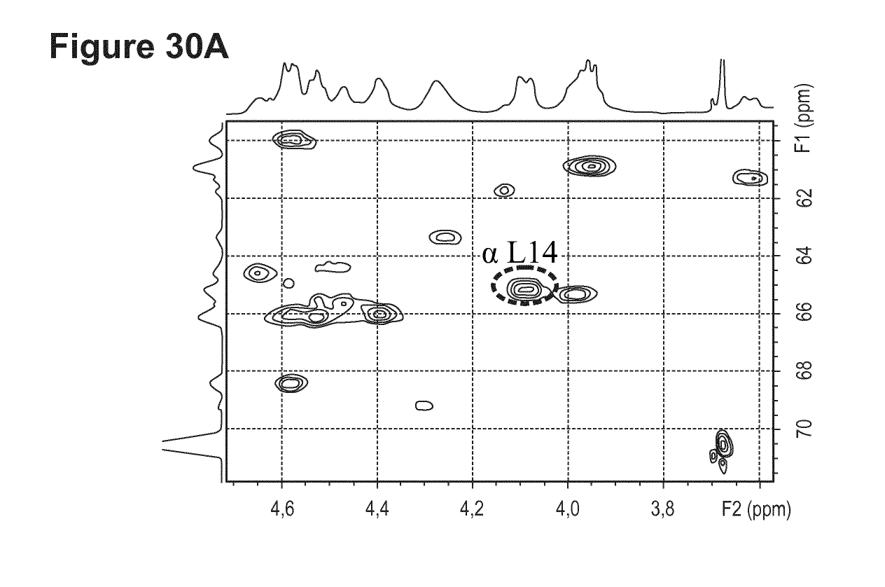

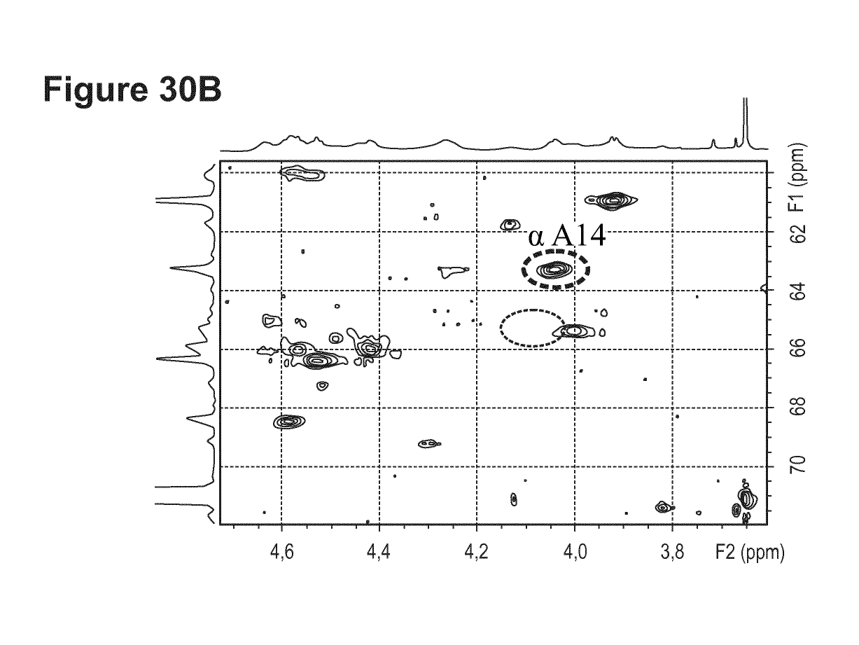

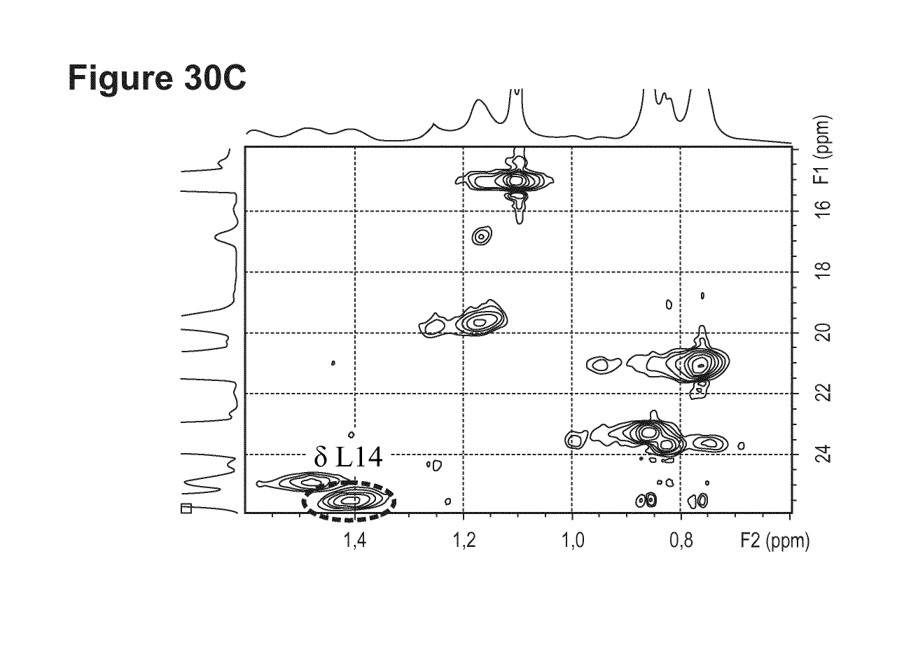

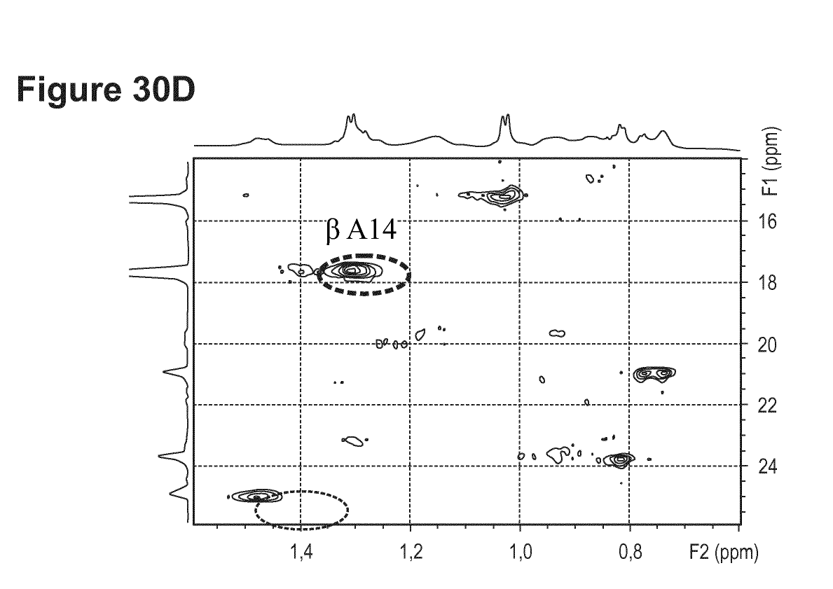

Carbon chemical shifts have been obtained from the HSQC spectrum. TSP has been used as internal standard (0.00 ppm for ¹H, −1.76 ppm for ¹³C). Quarternary carbons have not been assigned (no HMBC spectrum). Comparison of HSQC spectra for LabA2 and its mutant LabA2_L14A (FIG. 30) revealed that LabA2_L14A is substituted in the position 14 (i.e. the leucine is replaced by an alanine).

### Modulation of the Ser/Ser/Cys Motif

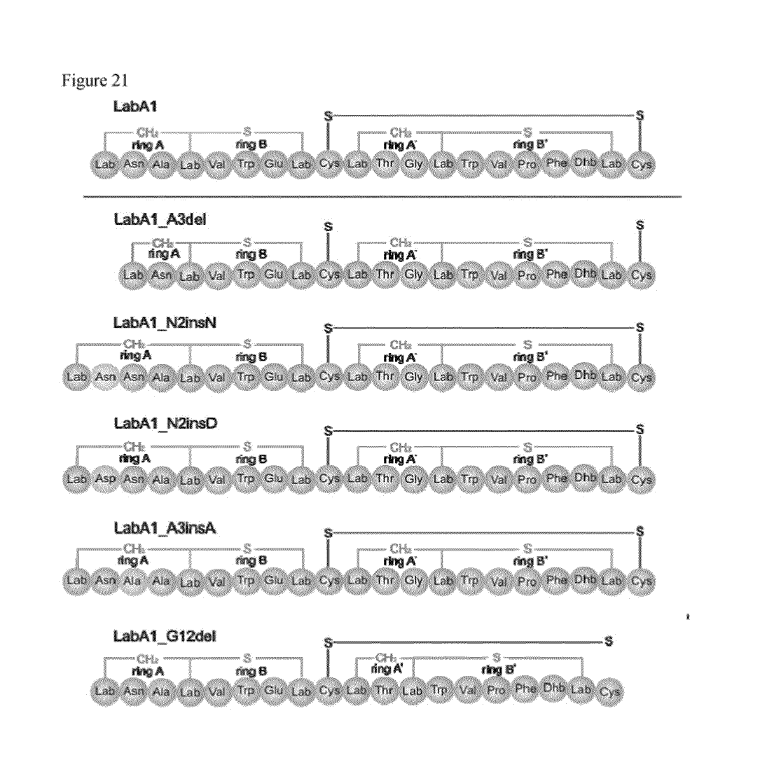

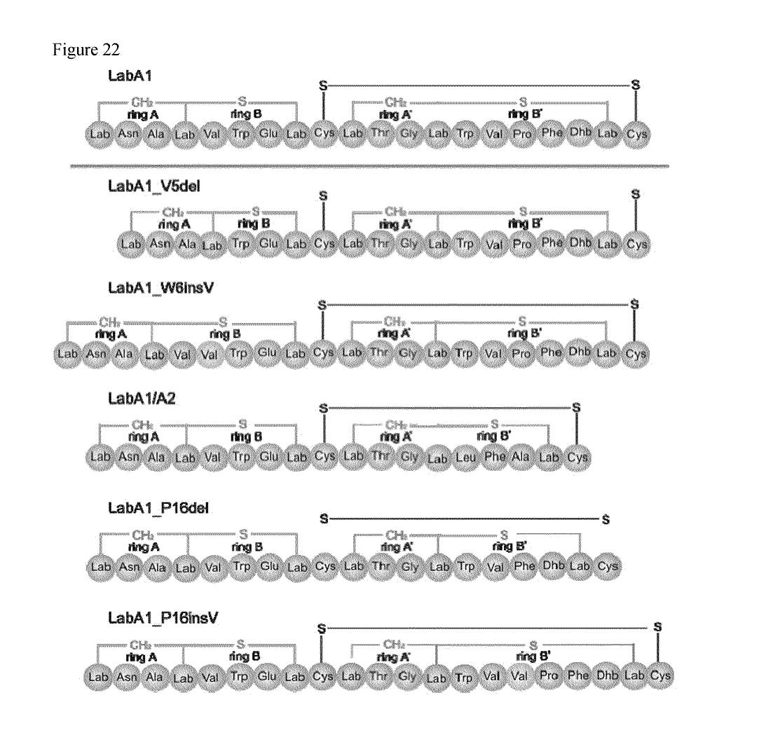

For any rational modifications of Labyrinthopeptins not only the knowledge about the importance of single residues but also the general information about the structure sensitivity to more drastic changes is significant. X-ray structure together with an alignment of structural peptides from gene clusters displaying homology to the lab gene cluster, reveals a number of conserved residues, representing a Ser-Xxx-Xxx-Ser-(Xxx)ₙ-Cys motif which appears twice in the peptide. This motif represents an essential ring forming core in the structure. It seems that Labyrinthopeptin-like peptides consist of variable B, B′ rings and conserved A, A′ rings. In order to determine to which extent rings are in fact amenable to changes, variants with extended or reduced ring sizes were generated. Only mutants with LabA1 as a model system were prepared as presented in FIGS. 21 and 22. The previously described strategy was chosen in which appropriate synthetic genes were ordered or the pMK_SG2 vector was modified by site-directed mutagenesis. In order to amplify the insert primers Infusion 1 and Infusion 2 were used. To verify if the size of A and A′ ring can be increased or decreased, six different LabA1 variants were generated. To increase ring sizes three different amino acids (Asp, Asn, Ala) were chosen for insertion at two different positions for the A ring, and one amino acid (Ala) at single position for the A′ ring. In order to decrease ring size Ala and Gly were remove in A and A′ rings respectively. None of these peptides were observed in bacterial cultures by means of HPLC-MS analysis.

### Substitution of Ser Involved in Lab Formation by Thr

It was interesting to investigate if LabKC enzyme can accept Ser→Thr substitutions. We created LabA1 mutants in which Ser residues involved in ring formation (positions 1, 4 and 13) were substituted by Thr. Analysis of fermentation samples of Thr mutants at positions 1 and 13 by LC-MS showed no evidence of lantibiotic production, suggesting a high specificity for either dehydration or ring-forming machinery. For mutant LabA1_S4T only trace amounts were detected of the expected peptide.

### Expression of Partial Structures of LabA1

In general, peptides are poor drugs mainly because of problems with degradation and bioavailability (82). With respect to large molecular weight (2073.7 Da for LabA1 and 1922.7 Da for LabA2) it would be very desirable to reduce Labyrinthopeptins size with retaining bioactivity. Herein we present results concerning size reduction. To express LabA1_west, a stop codon was introduces after Cys8 by means of site-directed mutagenesis. Experiment was performed with pMK_SG2 as a template. To express LabA1_east, a synthetic gene SG7 was ordered. pLab_SG7 allows expression of LabA1_east with a leader peptide for LabA1 where the last, C-terminal amino acid from a leader peptide was substituted with Met. Surprisingly, neither LabA1_east nor LabA1_west peptide was expressed by heterologous host S. lividans carrying appropriate vector. To overcome these expression problems a synthetic gene SG20 was ordered. It allowed expression of LabA1_M and LabA2_M with an additional Met residue. In case of LabA1_M the Met residue was inserted between rings B and C. For LabA2_M ring C was removed by substitution of Cys with Met. With these constructs

a chemical cleavage should be introduced in order to obtain a truncated variant. No expression of LabA1_M was observed. However, it is possible to express LabA2_M. This product can be subjected to cleavage by chemical reaction with CNBr (89), which cleaves C-terminally of Met.

### Ser/Ser/Ser/Cys Motif

The biosynthetic machinery of labyrinthopeptins is able to generate the labionin motif with a Ser-Xxx-Xxx-Ser-(Xxx)ₙ-Cys core. In further experiments we were interested to verify if the same machinery is able to form a “double labionin ring” from Ser-Xxx-Xxx-Ser-Xxx-Xxx-Ser-Xxx-Xxx-Xxx-Cys. To answer this question the synthetic gene SG15 was used. pLab_SG15 codes for LabA1_W6insVSA where an additional Val-Ser-Ala tripeptide is introduced into ring B, and for LabA1_P16insS where an additional Ser is introduced into ring B′. Neither expression of LabA1_W6insVSA nor of LabA1_P16insS was observed which means that this motif is not only unable to form an additional labionin ring but also disturbs production of the native like bridged form.

### Additional Substitutions

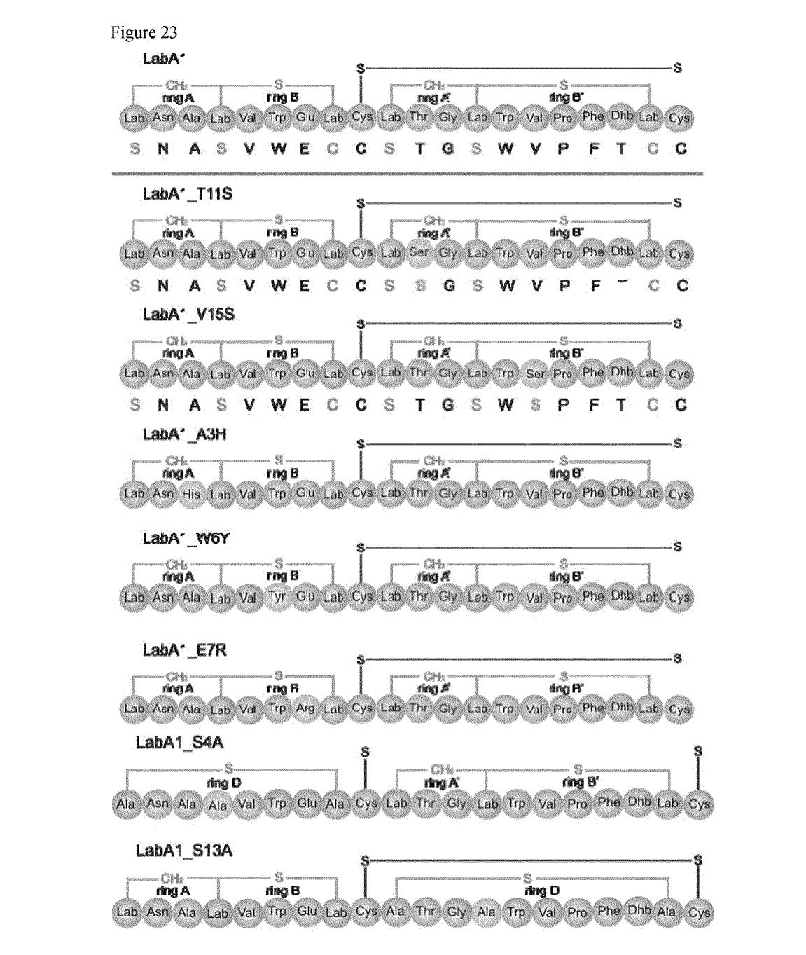

During the posttranslational modifications of Labyrinthopeptins all available Ser and Thr (except these which are located within the A′-rings) undergo dehydration. To address the question of whether the Labyrinthopeptins biosynthetic apparatus can dehydrate also additional Ser, mutants LabA1_T11S and LabA1_V15S were prepared. The main reason for conducting this study was to investigate the possibility of installation of dehydroamino acids in the structure (83). LabA1_T11S was expressed by the use of a pLab_SG16 construct. The vector coding for LabA1_V15S was prepared by means of site-directed mutagenesis with pMK_SG2 as a template and primers LabA1_V15_Sfw/rv. MS analysis revealed that additional serines in LabA1_T11S as well as in LabA1_V15S were not dehydrated. Analysis of 37 lantibiotic primary structures in the study by Rink et al. (84-85). showed that serine residues escape dehydration more often than threonines. These findings are in agreement with results presented for Labyrinthopeptins. However, it also should be taken into consideration that LabKC may distinguish between the position of serine/threonine and more mutants would have to be investigated to draw final conclusions. The effects of additional substitutions were investigated as well (see FIG. 23). Introduction of appropriate mutations to LabA1 to generate vectors LabA1_A3H/E7R/W6Y/S4A/S13A were performed by site-directed mutagenesis. As a template pMA_SG2 was used. A1a3 was substituted with His to verify if addition of ionizable amino acids can have a negative influence of Labyrinthopeptin biosynthesis. LabA1_A3H was detected which shows that such substitution is tolerated. However, the information obtained from that single experiment can not be generalized. Negatively charged Glu7 was substituted with positively charged Arg. Production of LabA1_E7R was not detected which suggest that this substitution had disturbed biosynthesis. Mutant with substitution of Trp6→Tyr (LabA1_W6Y) was produced in much higher yield than Trp6→Ala (LabA1_W6A) which indicates that it is possible to increase

Labyrinthopeptins diversity by substitutions of tryphophans but not all substitutions are accepted. This finding shows that rational bioengineering of lantibiotics is not a straightforward task because it requires the understanding of the importance of each amino acid in the molecule for the posttranslational processing and the biosynthetic machinery as a whole.

Ser at position 4 and 13 were substituted with Ala to verify if the disruption of labionin formation will result in formation of lanthionine ring between both Ser1 and Cys8, and Ser10 and Cys19. Expression of LabA1_S4A and LabA1_S13A was not observed.

### Studies on Ring C Mutations

All the mutants used to confirm the flexibility of the C-ring were generated by site-directed mutagenesis with pMK_SG2 as a template. Expression of all peptides presented in FIG. 24 was observed. A possibility to detect LabA1_C20del shows that the disulfide bridge is not necessary to obtain labyrinthopeptin which is stable in the proteolytic environment of

S. lividans. LabA1_S1insC presents a possibility to create a disulfide bridge also in the west part of a molecule. In this case occurrence of disulfide bond was deduced only on the basis of the observed molecular mass (−2 Da corresponding to two protons). Mutants LabA1_S10insA and LabA1_C20insA show that the size of ring C can be increased although the production levels of these variants were significantly lower.

### Generation of Labyrinthopeptin Hybrids

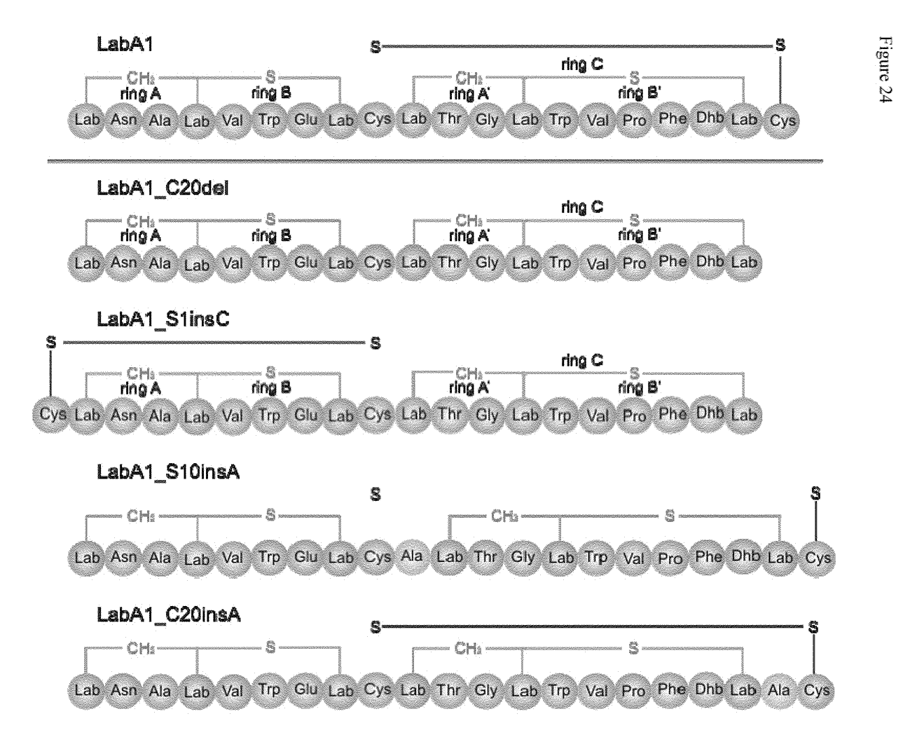

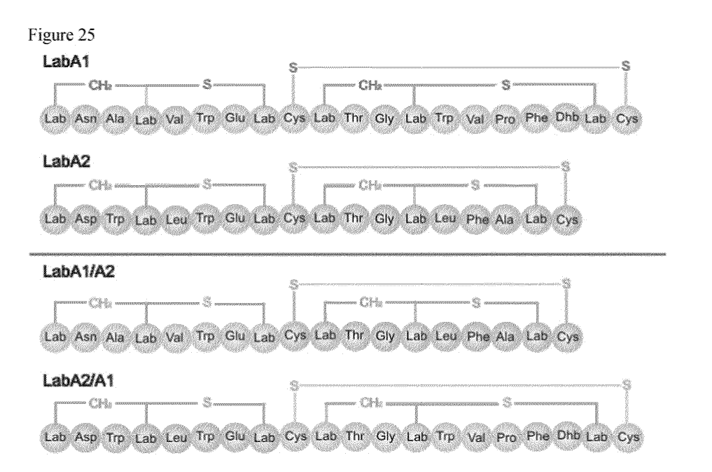

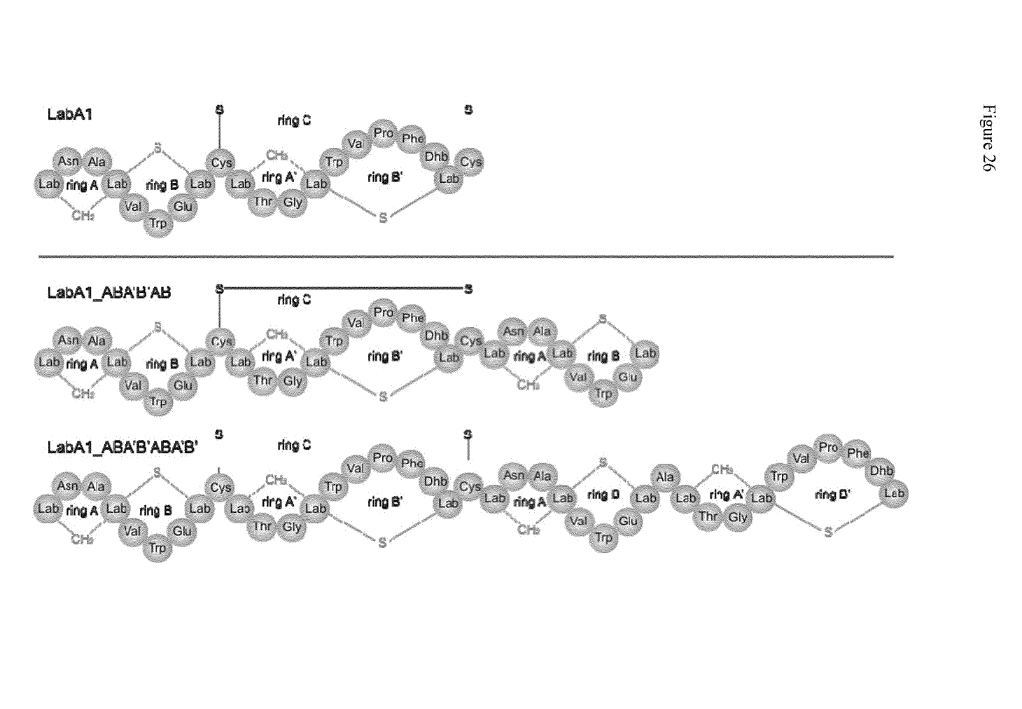

The majority of studies performed in this work concern LabA1. In this situation it was essential to evaluate if the knowledge collected for LabA1 can be also used for engineering of LabA2. In order to provide an answer to this question gene SG11 was synthesized. pLab_SG11 codes for LabA1/A2 (FIG. 25) and LabA2/A1 (FIG. 25) hybrid molecules which are expressed with unmodified LabA1 and LabA2 leader peptides, respectively. Both peptides were detected in cultures of S. lividans/pLab_SG11 which suggested that observations made for LabA1 could also be applied to LabA2. Another interesting aspect investigated in this study was to evaluate if it is possible to extended Labyrinthopeptins repeatedly. This could be possible because of the polymeric nature of the substrate. Also distributive mode of action of the biosynthesis enzyme, which was presented for LanM (69), supports this possibility. Synthetic genes SG9 and SG10 were designed to generate peptides shown in FIG. 26. Neither LabA1_ABA′B′ABA′B′ nor LabA1_ABA′B′ABA′B′ could be detected. To test if the Labyrinthopeptin biosynthetic machinery can be used for production of silent lantibiotics (those which are difficult to isolate from natural sources, for which structure and production conditions are not known) belonging to class III a synthetic gene SG8 was ordered. SG8 codes for a hypothetical lantibiotics RamS2. It was found during in silico analysis of Streptomyces scabies genomic DNA. The whole gene cluster responsible for biosynthesis of RamS2 show homology to the lab gene cluster. Analysis of a supernatant and also a cells extract from S. lividans/pLab_SG8 didn't reveal a production of a peptide which could correspond to RamS2.

In summary, alanine-scanning studies showed that most of the amino acids not involved in rings formation can be easily substituted with Ala. Only exceptions are Trp residues for which Ala substitutions inhibited production of Labyrinthopeptins or resulted in trace amounts. However, production was detected when Trp residue was substituted with Tyr, which indicates that conservation of these positions might not be absolute. Substitution of Gln to Arg suppresses production. Another investigated aspect concerned size requirements for rings formation. It was demonstrated that any change in the size of rings A and A′ is not accepted. Rings B and B′ however are more elastic, 5,6,7,8-membered rings were created. It was also shown that disulphide bond ring C is not required or that it can be moved to western part of the molecule. Attempt to form double labionin (containing two quaternary carbon atoms) failed as well as any extension of the whole molecule.

### Summary of Expected and Observed Molecular Masses of Bioengineered

### Labyrinthopeptins (ND—not Detected):

| | | Peptide masses (Da) | | |
|---|---|---|---|---|
| | | Expected | | |
| | Molecular | molecular | Expected | Observed |
| Mutant | formula | mass | [M + 2H]²⁺ | [M + 2H]²⁺ |

Alanine scanning mutagenesis

| | | | | |
|---|---|---|---|---|
| LabA1_N2A | C₉₀H₁₁₈N₂₂O₂₄S₄ | 2030.76 | 1016.38 | 1016.39 |
| LabA1_V5A | C₉₀H₁₁₅N₂₃O₂₅S₄ | 2045.73 | 1023.86 | 1023.88 |
| LabA1_W6A | C₈₄H₁₁₄N₂₂O₂₅S₄ | 1958.72 | 980.36 | 980.69 |
| LabA1_E7A | C₉₀H₁₁₇N₂₃O₂₃S₄ | 2015.76 | 1008.88 | 1008.89 |
| LabA1_T11A | C₉₁H₁₁₇N₂₃O₂₄S₄ | 2043.75 | 1022.87 | 1022.89 |
| LabA1_W14A | C₈₄H₁₁₄N₂₂O₂₅S₄ | 1958.72 | 980.36 | ND |
| LabA1_V15A | C₉₀H₁₁₅N₂₃O₂₅S₄ | 2045.73 | 1023.86 | 1023.88 |
| LabA1_P16A | C₉₀H₁₁₇N₂₃O₂₅S₄ | 2047.75 | 1024.87 | 1024.63 |
| LabA1_F17A | C₈₆H₁₁₅N₂₃O₂₅S₄ | 1997.73 | 999.86 | 999.88 |
| LabA2_D2A | C₈₄H₁₁₀N₂₀O₂₂S₄ | 1878.70 | 940.35 | 940.36 |
| LabA2_W3A | C₇₇H₁₀₅N₁₉O₂₄S₄ | 1807.65 | 904.82 | 904.84 |
| LabA2_L5A | C₈₂H₁₀₄N₂₀O₂₄S₄ | 1880.64 | 941.32 | 1881.91 |
| | | | | [M + H]⁺ |
| LabA2_W6A | C₇₇H₁₀₅N₁₉O₂₄S₄ | 1807.65 | 904.82 | ND |
| LabA2_E7A | C₈₃H₁₀₈N₂₀O₂₂S₄ | 1864.68 | 933.34 | 933.36 |
| LabA2_T11A | C₈₄H₁₀₈N₂₀O₂₃S₄ | 1892.68 | 947.34 | 1892.36 |
| | | | | [M + H]⁺ |
| LabA2_G12A | C₈₆H₁₁₂N₂₀O₂₄S₄ | 1936.70 | 969.35 | 969.36 |
| LabA2_L14A | C₈₂H₁₀₄N₂₀O₂₄S₄ | 1880.64 | 941.32 | 941.34 |
| LabA2_F15A | C₇₉H₁₀₆N₂₀O₂₄S₄ | 1846.66 | 924.33 | 924.35 |

Ser/Ser/Cys motif, Ring A and A′

| | | | | |
|---|---|---|---|---|
| LabA1_N2insD | C₉₆H₁₂₄N₂₄O₂₈S₄ | 2188.79 | 1095.39 | ND |
| LabA1_N2insN | C₉₆H₁₂₅N₂₅O₂₇S₄ | 2187.81 | 1094.90 | ND |
| LabA1_A3insA | C₉₅H₁₂₄N₂₄O₂₆S₄ | 2144.80 | 1073.40 | ND |
| LabA1_A3del | C₈₉H₁₁₄N₂₂O₂₄S₄ | 2002.73 | 1002.36 | ND |
| LabA1_G12insA | C₉₅H₁₂₄O₂₆N₂₄S₄ | 2144.80 | 1073.40 | ND |
| LabA1_G12del | C₉₀H₁₁₆N₂₂O₂₄S₄ | 2016.74 | 1009.37 | ND |

Ser/Ser/Cys motif, Ring B and B′

| | | | | |
|---|---|---|---|---|
| LabA1_V5del | C₈₇H₁₁₀N₂₂O₂₄S₄ | 1974.69 | 988.69 | 988.56 |
| LabA1_W6insV | C₉₇H₁₂₈N₂₄O₂₆S₄ | 2172.83 | 1087.41 | 1087.42 |
| LabA1_P16del | C₈₇H₁₁₂N₂₂O₂₄S₄ | 1976.71 | 989.35 | 989.36 |
| LabA1_P16insV | C₉₇H₁₂₈O₂₆N₂₄S₄ | 2172.83 | 1087.41 | 1087.50 |

Substitution of serines involved in ring formation by threonines

| | | | | |
|---|---|---|---|---|
| LabA1_S4T | C₉₃H₁₂₁N₂₃O₂₅S₄ | 2087.78 | 1044.89 | 1045.11 |
| M-LabA1_S4T | C₉₈H₁₃₀N₂₄O₂₆S₅ | 2218.98 | 1110.49 | 1110.58 |
| AM-LabA1_S4T | C₁₀₁H₁₃₅N₂₅O₂₇S₅ | 2290.06 | 1146.03 | 1146.07 |
| LabA1_S13T | C₉₃H₁₂₁N₂₃O₂₅S₄ | 2087.78 | 1044.89 | ND |
| LabA1_S1T | C₉₃H₁₂₁N₂₃O₂₅S₄ | 2087.78 | 1044.89 | ND |

Express east or west part of labyrinthopeptin

| | | | | |
|---|---|---|---|---|
| LabA1_west | C₃₇H₅₀N₁₀O₁₂S | 858.33 | 859.33 | ND |
| (LabA1_C9tga) | | | [M + H]⁺ | |
| LabA1_east | C₅₅H₇₁N₁₃O₁₄S₃ | 1233.44 | — | ND |
| (SG7) | | | | |
| LabA1_M | C₉₇H₁₂₈N₂₄O₂₆S₅ | 2204.96 | 1103.48 | ND |
| (SG20) | | | | |
| NR-LabA2_M | C₉₄H₁₂₉N₂₅O₂₆S₃ | 2120.02 | 1061.01 | 1060.92 |
| (SG20) | | | | |
| R-LabA2_M | C₉₀H₁₂₃N₂₃O₂₄S₃ | 2005.92 | 1003.96 | 1003.97 |
| (SG20) | | | | |

Ser/Ser/Ser/Cys motif

| | | | | |
|---|---|---|---|---|
| LabA1_P16insS | C₉₅H₁₂₄N₂₄O₂₇S₄ | 2160.84 | 1081.42 | ND |
| (Ser) | | | | |
| LabA1_P16insS | C₉₅H₁₂₂N₂₄O₂₆S₄ | 2142.84 | 1072.42 | ND |
| (Dha) | | | | |
| LabA1_W6insVSA | C₁₀₃H₁₃₈N₂₆O₂₉S₄ | 2331.05 | 1166.52 | ND |
| (Ser) | | | | |
| LabA1_W6insVSA | C₁₀₃H₁₃₆N₂₆O₂₈S₄ | 2313.05 | 1157.52 | ND |
| (Dha) | | | | |

Additional substitutions

| | | | | |
|---|---|---|---|---|
| LabA1_V15S | C₉₀H₁₁₅N₂₃O₂₆S₄ | 2061.73 | 1031.86 | 1031.93 |
| LabA1_T11S | C₉₁H₁₁₇N₂₃O₂₅S₄ | 2059.74 | 1030.87 | 1030.67 |
| (Ser) | | | | |
| LabA1_T11S | C₉₁H₁₁₇N₂₃O₂₅S₄ | 2041.74 | 1021.87 | ND |
| (Dha) | | | | |
| LabA1_W6Y | C₉₀H₁₁₈N₂₂O₂₆S₄ | 2050.75 | 1026.37 | 1026.38 |
| LabA1_A3H | C₉₅H₁₂₁O₂₅N₂₅S₄ | 2139.78 | 1070.89 | 1070.91 |
| LabA1_E7R | C₉₃H₁₂₄O₂₃N₂₆S4 | 2100.82 | 1051.41 | ND |
| LabA1_S4A | C₉₂H₁₂₃N₂₃O₂₆S₄ | 2093.79 | 1047.89 | ND |
| LabA1_S13A | C₉₂H₁₂₃N₂₃O₂₆S₄ | 2093.79 | 1047.89 | ND |

Ring C

| | | | | |
|---|---|---|---|---|
| LabA1_C20insA | C₉₅H₁₂₄N₂₄O₂₆S₄ | 2144.80 | 1073.40 | 1073.41 |
| LabA1_S10insA | C₉₅H₁₂₄N₂₄O₂₆S₄ | 2144.80 | 1073.40 | 1073.52 |
| LabA1_C20del | C₈₉H₁₁₆O₂₄N₂₂S₃ | 1972.77 | 987.38 | 987.49 |
| LabA1_S1insC | C₉₂H₁₁₉N₂₃O₂₅S₄ | 2073.76 | 1037.88 | 1037.98 |

Spacer

| | | | | |
|---|---|---|---|---|
| LabA1_C9insV | C₉₇H₁₂₈O₂₆N₂₄S₄ | 2172.83 | 1087.41 | ND |
| LabA1_C9insVN | C₁₀₁H₁₃₅O₂₈N₂₆S₄ | 2286.93 | 1144.46 | ND |

Hybrids

| | | | | |
|---|---|---|---|---|
| LabA1_ABA′B′ | C₁₂₉H₁₆₇O₃₆N₃₃S₅ | 2914.09 | 1458.04 | ND |
| AB | | | | |
| (SG9) | | | | |
| D-LabA1/A2 | C₈₀H₁₀₉O₂₆N₂₁S₄ | 1907.74 | 954.87 | 954.99 |
| (SG11) | | | | |
| AD-LabA1/A2 | C₈₃H₁₁₄O₂₇N₂₂S₄ | 1978.82 | 990.41 | 990.47 |
| (SG11) | | | | |
| R-LabA2/A1 | C₁₀₇H₁₃₇O₂₇N₂₇S₄ | 2359.99 | 1180.99 | 1180.90 |
| (SG11) | | | | |
| NR-LabA2/A1 | C₁₁₁H₁₄₃O₂₉N₂₉S₄ | 2474.09 | 1238.04 | 1237.85 |
| (SG11) | | | | |
| ENR-LabA2/A1 | C₁₁₆H₁₅₀O₃₂N₃₀S₄ | 2603.20 | 1302.60 | 1302.30 |
| (SG11) | | | | |

Evaluation of anti-pain activity of labyrinthopeptin A2 and its structural analogs LabA2_L14A and LabA2 F15A in an in vitro assay (IC50 determination of LabA2, LabA2_L14A and LabA2_F15A on hP2X4_HEK-FITR-cell line) The use of labyrinthopeptin A2 and its derivatives, in particular LabA2_L14A and LabA2_F15A, as analgesics was investigated for neuropathic pain based on their inhibitory effects on P2X4 receptors (Nagata K et al., 2009).

Recombinant human P2X4 receptor was expressed in HEK cells (hP2X4_HEK-FITR). hP2X4 expression was induced with doxycyclin 1 μg/ml for 24 h to 48 h. The patch clamp experiment has been performed under standard conditions using an axopatch-200B amplifier and pClamp v10 acquisition software (Axon Instruments Inc, Foster City, Calif., USA). Cells were continuously superfused with an external solution consisting of (in mM) 130 CsCl, 4 NaCl, 1 MgCl₂, 1 CaCl₂, 10 EGTA (free Ca²⁺=6.7 nM) and 10 HEPES (pH 7.4 with CsOH). This was replaced with a low divalent external solution consisting of (in mM) 145 NaCl, 4 KCl, 2 CaCl₂, 1 MgCl₂ and 10 HEPES (pH 7.4 with NaOH). 250 ms application of 10 μM ATP (Sigma) every 10 sec was performed. When current stabilization was achieved increasing concentrations of labyrinthopeptins (LabA2, LabA2_L14A and LabA2F15A) was perfused (from 0.3 to 90 μM). Currents were measured at a holding potential of −60 mV.

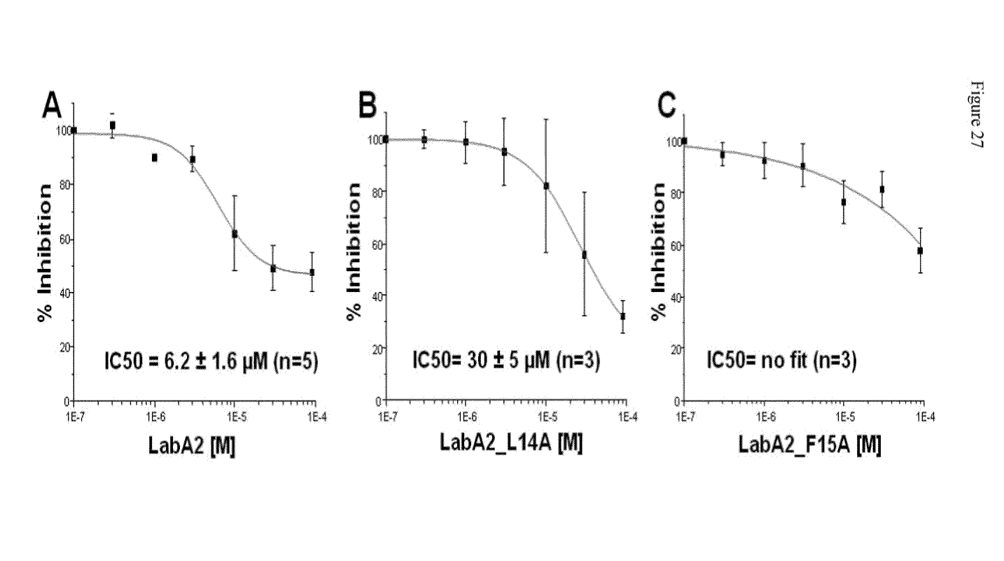

Results were calculated using Origin7.5 software (FIG. 27). It was found that LabA2 and LabA2_L14A inhibit human P2X4 receptor function with IC50 values of 6.2 μM and 30 μM respectively. IC50 value for LabA2_F15A could not be determined in used conditions. This result suggests that LabA2_F15A can not block P2X4 receptors or it can block them but with high IC50 values which discriminate it for a use as an anti-pain drug. LabA2 and LabA2_L14A were shown to be useful for the treatment of pain, in particular neuropathic pain.

### REFERENCES

1. Hanahan, D. (1983) Studies on transformation of Escherichia coli with plasmids, J Mol Biol 166, 557-580.
2. Datsenko, K. A., Wanner, B. L. (2000) One-step inactivation of chromosomal genes in Escherichia coli K-12 using PCR products, Proc Natl Acad Sci USA 97, 6640-6645.
3. Gewain, K. M., Occi, J. L., Foor, F., MacNeil, D. J. (1992) Vectors for generating nested deletions and facilitating subcloning G+C-rich DNA between Escherichia coli and Streptomyces sp., Gene 119, 149-150.
4. Kirchner, O., Tauch, A. (2003) Tools for genetic engineering in the amino acid-producing bacterium Corynebacterium glutamicum, J Biotechnol 104, 287-299.
5. Gust, B., Challis, G. L., Fowler, K., Kieser, T., Chater, K. F. (2003) PCR-targeted Streptomyces gene replacement identifies a protein domain needed for biosynthesis of the sesquiterpene soil odor geosmin, Proc Natl Acad Sci USA 100, 1541-1546.
6. Paget, M. S., Chamberlin, L., Atrih, A., Foster, S. J., Buttner, M. J. (1999) Evidence that the extracytoplasmic function sigma factor sigmaE is required for normal cell wall structure in Streptomyces coelicolor A3(2). J Bacteriol 181, 204-211.
7. Bierman, M., Logan, R., O'Brien, K., Seno, E. T., Rao, R. N., Schoner, B. E. (1992) Plasmid cloning vectors for the conjugal transfer of DNA from Escherichia coli to Streptomyces spp, Gene 116, 43-49.
8. Wehmeier, U. F. (1995) New multifunctional Escherichia coli-Streptomyces shuttle vectors allowing blue-white screening on XGal plates, Gene 165, 149-150.
9. Feitelson, J. S., Englenwood, N. J. (1991) Multifunctional plasmid vectors from Actinomadura and Escherichia coli, U.S. Pat. No. 5,002,891.
10. Kieser, T., Bibb, M. J., Buttner, M. J., Chater, K. F., Hopwood, D. A. (2000) Practical Streptomyces genetics (2nd ed.), John Innes Foundation, Norwich, UK.
11. Madoń, J., Hinter, R. (1991) Transformation system for Amycolatopsis (Nocardia) mediterranei: direct transformation of mycelium with plasmid DNA., J Bacteriol 173, 6325-6331.
12. Wang, W., Malcolm, B. A. (1999) Two-stage PCR protocol allowing introduction of multiple mutations, deletions and insertions using QuikChange site-directed mutagenesis, Biotechniques 26, 680-682.
13. Rashtchian, A. (1995) Novel methods for cloning and engineering genes using the polymerase chain reaction, Curr Opin Biotechnol 6, 30-36.
14. Aslanidis, C., de Jong, P. J. (1990) Ligation-independent cloning of PCR products (LIC-PCR). Nucleic Acids Res 18, 6069-6074.
15. Schägger, H., von Jagow, G. (1987) Tricine-sodium dodecyl sulfate-polyacrylamide gel electrophoresis for the separation of proteins in the range from 1 to 100 kDa, Anal Biochem 166, 368-379.
16. Fukase, K., Kitazawa, M., Sano, A., Shimbo, K., Horimoto, S., Fujita, H., Kubo, A., Wakamiya, T., Shiba, T. (1992) ChemInform Abstract: Synthetic Study on Peptide Antibiotic Nisin. Part 5. Total Synthesis of Nisin, ChemInform 23.
17. Dairi, T., Hamano, Y., Furumai, Tamotsu, Oki, T. (1999) Development of a self-cloning system for Actinomadura verrucosospora and identification of polyketide synthase genes essential for production of the angucyclic antibiotic pradimicin, Appl Environ Microbiol 65, 2703-2709.
18. Combes, P., Till, R., Bee, S., Smith, M. C. M. (2002) The Streptomyces genome contains multiple pseudo-attB sites for the (phi)C31-encoded site-specific recombination system, J Bacteriol 184, 5746-5752.
19. Thompson, C. J., Ward, J. M., Hopwood, D. A. (1982) Cloning of antibiotic resistance and nutritional genes in streptomycetes, J Bacteriol 151, 668-677.
20. Okanishi, M., Katagiri, K., Furumai, T., Takeda, K., Kawaguchi, K., Saitoh, M., Nabeshima, S. (1983) Basic techniques for DNA cloning and conditions required for streptomycetes as a host, J Antibiot (Tokyo) 36, 99-108.
21. Rau, A., Hogg, T., Marquardt, R., Hilgenfeld, R. (2001) A new lysozyme fold. Crystal structure of the muramidase from Streptomyces coelicolor at 1.65 A resolution, J Biol Chem 276, 31994-31999.
22. Bibb, M. J., Ward, J. M., Hopwood, D. A. (1978) Transformation of plasmid DNA into Streptomyces at high frequency, Nature 274, 398-400.
23. Baltz, R. H., Hosted, T. J. (1996) Molecular genetic methods for improving secondary-metabolite production in actinomycetes, Trends Biotechnol 14, 245-250.
24. Trieu-Cuot, P., Carlier, C., Martin, P., Courvalin, P. (1987) Plasmid transfer by conjugation from Escherichia coli to Gram-positive bacteria, FEMS Microbiol Lett 48, 289-294.
25. Mazodier, P., Petter, R., Thompson, C. (1989) Intergeneric conjugation between Escherichia coli and Streptomyces species, J Bacteriol 171, 3583-3585.
26. Smokvina, T., Mazodier, P., Boccard, F., Thompson, C. J., Guérineau, M. (1990) Construction of a series of pSAM2-based integrative vectors for use in actinomycetes, Gene 94, 53-59.
27. Stegmann, E., Pelzer, S., Wilken, K., Wohlleben, W. (2001) Development of three different gene cloning systems for genetic investigation of the new species Amycolatopsis japonicum MG417-CF17, the ethylenediaminedisuccinic acid producer, J Biotechnol 92, 195-204.
28. Heinzelmann, E., Berger, S., Puk, O., Reichenstein, B., Wohlleben, W., Schwartz, D. (2003) A glutamate mutase is involved in the biosynthesis of the lipopeptide antibiotic friulimicin in Actinoplanes friuliensis., Antimicrob Agents Chemother 47, 447-457.
29. Stinchi, S., Azimonti, S., Donadio, S., Sosio, M. (2003) A gene transfer system for the glycopeptide producer Nonomuraea sp. ATCC39727, FEMS Microbiol Lett 225, 53-57.
30. Voeykova, T., Emelyanova, L., Tabakov, V., Mkrtumyan, N. (1998) Transfer of plasmid pTO1 from Escherichia coli to various representatives of the order Actinomycetales by intergeneric conjugation, FEMS Microbiol Lett 162, 47-52.
31. Wink J., K., R. M., Seibert, G., Stackebrandt, E. (2003) Actinomadura namibiensis sp. nov., Int J Syst Evol Microbiol 53, 721-724.
32. Vrijbloed, J. W., Madon, J., Dijkhuizen, L. (1995) Transformation of the methylotrophic actinomycete Amycolatopis methanolica with plasmid DNA: stimulatory effect of a pMEA300-encoded gene, Plasmid 34, 96-104.
33. Kumar, C. V., Coque, J. J., Martin, J. F. (1994) Efficient transformation of the cephamycin C producer Nocardia lactamdurans and development of shuttle and promoter-probe cloning vectors, Appl Environ Microbiol 60, 4086-4093.
34. Yeung, M. K., Kozelsky, C. S. (1994) Transformation of Actinomyces spp. by a gram-negative broad-host-range plasmid, J Bacteriol 176, 4173-4176.
35. Pigac, J., Schrempf, H. (1995) A simple and rapid method of transformation of Streptomyces rimosus R6 and other Streptomycetes by electroporation, Appl Environ Microbiol 61, 352-356.
36. Mazy-Servais, C., Baczkowski, D., Dusart, J. (1997) Electroporation of intact cells of Streptomyces parvulus and Streptomyces vinaceus, FEMS Microbiol Lett 151, 135-138.
37. MacNeil, D. J. (1987) Introduction of plasmid DNA into Streptomyces lividans by electroporation, FEMS Microbiol Lett 42, 239-244.
38. Tyurin, M., Starodubtseva, L., Kudryavtseva, H., Voeykova, T., Livshits, V. (1995) Electrotransformation of germinating spores of Streptomyces spp., Biotechnol Tech 9, 737-740.
39. Flinspach, K., Westrich, L., Kaysser, L., Siebenberg, S., Juan Pablo Gomez-Escribano, J. P., Bibb, M., Gust, B., Heide, L. (2010) Heterologous expression of the biosynthetic gene clusters of coumermycin A1, clorobiocin and caprazamycins in genetically modified Streptomyces coelicolor strains, Biopolymers 93, 823-832.
40. Widdick, D. A., Dodd, H. M., Barraille, P., White, J., Stein, T. H., Chater, K. F., Gasson, M. J., Bibb, M. J. (2003) Cloning and engineering of the cinnamycin biosynthetic gene cluster from Streptomyces cinnamoneus cinnamoneus DSM 40005, Proc Natl Acad Sci USA 100, 4316-4321.
41. Zhang, H., Wang, Y., Pfeifer, B. A. (2008) Bacterial hosts for natural product production, Mol Pharm 5, 212-225.
42. Van Lanen, S. G., Oh, T., Wen Liu, W., Wendt-Pienkowski, E., Shen, B. (2007) Characterization of the maduropeptin biosynthetic gene cluster from Actinomadura madurae ATCC 39144 supporting a unifying paradigm for enediyne biosynthesis, J Am Chem Soc 129, 13082-13094.
43. Corvey, C., Stein, T., Duesterhus, S., Karas, M., Entian, K. D. (2003) Activation of subtilin precursor by Bacillus subtilis extracellular serine proteases subtilisin (AprE), WprA, and Vpr, Biochem Biophys Res Commun 304, 48-54.
44. Schmiederer, T. (2008) Biosynthese der Labyrinthopeptine A1, A2 and A3, einer neuen Klasse von Lantibiotika aus Actinomadura namibiensis. Dissertation, TU Berlin.
45. Kuhstoss, S., Rao, R. N. (1991) Analysis of the integration function of the streptomycete bacteriophage ΦC31, J Mol Biol 222, 897-908.
46. Combes, P., Till, R., Sally Bee, S., Smith, M. C. M. (2002) The Streptomyces genome contains multiple Pseudo-attB Sites for the phi C31-encoded site-specific recombination system, Journal of Bacteriology 184, 5746-5752.
47. Wilkinson, C. J., Hughes-Thomas, Z. A., Martin, C. J., Bohm, I., Mironenko, T., Deacon, M., Wheatcroft, M., Wirtz, G., Staunton, J., Leadlay, P. F. (2002) Increasing the efficiency of heterologous promoters in actinomycetes, J Mol Microbiol Biotechnol. 4, 417-426.
48. Keijser, B. J., van Wezel, G. P., Canters, G. W., Kieser, T., Vijgenboom, E. (2000) The ram-dependence of Streptomyces lividans differentiation is bypassed by copper, J Mol Microbiol Biotechnol 2, 565-574.
49. Boakes, S., Cortes, J., Appleyard, A. N., Rudd, B. A. M., Dawson, M. J. (2009) Organization of the genes encoding the biosynthesis of actagardine and engineering of a variant generation system, Mol Microbiol 72, 1126-1136.
50. Dodd, H. M., Horn, N., Hao, Z., Gasson, M. J. (1992) A lactococcal expression system for engineered nisins, Appl Environ Microbiol 58, 3683-3693.
51. Dodd, H. M., Horn, N., Giffard, C. J., Gasson, M. J. (1996) A gene replacement strategy for engineering nisin, Microbiology 142, 47-55.
52. Chen, P., Novak, J., Kirk, M., Barnes, S., Qi, F., Caufield, P. W. (1998) Structure-activity study of the lantibiotic mutacin II from Streptococcus mutans T8 by a gene replacement strategy, Appl Environ Microbiol 64, 2335-2340.
53. Ottenwalder, B., Kupke, T., Brecht, S., Gnau, V., Metzger, J., Jung, G., Gotz, F. (1995) Isolation and characterization of genetically engineered gallidermin and epidermin analogs, Appl Environ Microbiol 61, 3894-3903.
54. Bierbaum, G., Reis, M., Szekat, C., Sahl, H. G. (1994) Construction of an expression system for engineering of the lantibiotic Pep5, Appl Environ Microbiol 60, 4332-4338.
55. Kuipers, O. P., Bierbaum, G., Ottenwälder, B., Dodd, H. M., Horn, N., Metzger, J., Kupke, T., Gnau, V., Bongers, R., van den Bogaard, P., Kosters, H., Rollema, H. S., de Vos, W. M., Siezen, R. J., Jung, G., Götz, F., Sahl, H. G., Gasson, M. J. (1996) Protein engineering of lantibiotics, Antonie Van Leeuwenhoek 69, 161-169.
56. Lin, Y., Teng, K., Huan, L., Zhong, J. (2011) Dissection of the bridging pattern of bovicin HJ50, a lantibiotic containing a characteristic disulfide bridge, Microbiol Res 166, 146-154.
57. Altena, K., Guder, A., Cramer, C., Bierbaum, G. (2000) Biosynthesis of the lantibiotic mersacidin: organization of a type B lantibiotic gene cluster, Appl Environ Microbiol 66, 2565-2571.
58. Kuipers, O. P., Beerthuyzen, M. M., Siezen, R. J., De Vos, W. M. (1993) Characterization of the nisin gene cluster nisABTCIPR of Lactococcus lactis, Eur J Biochem 216, 281-291.
59. Pag, U., Heidrich, C., Bierbaum, G., Sahl, H. G. (1999) Molecular analysis of expression of the lantibiotic pep5 immunity phenotype, Appl Environ Microbiol 65, 591-598.
60. Kaiser, R., Metzka, L. (1999) Enhancement of cyanogen bromide cleavage yields for methionyl-serine and methionyl-threonine peptide bonds, Anal Biochem 266, 1-8.
61. Xie, L., Miller, L. M., Chatterjee, C., Averin, O., Kelleher, N. L., van der Donk, W. A. (2004) Lacticin 481: in vitro reconstitution of lantibiotic synthetase activity, Science 303, 679-681.
62. Levengood, M. R., Knerr, P. J., Oman, T. J., van der Donk, W. A. (2009) In vitro mutasynthesis of lantibiotic analogues containing nonproteinogenic amino acids, J Am Chem Soc 131, 12024-12025.
63. Fukase, K., Kitazawa, M., Sano, A., Shimbo, K., Horimoto, S., Fujita, H., Kubo, A., Wakamiya, T., Shiba, T. (1988) Total synthesis of peptide antibiotic nisin, Tetrahedron Lett 29, 795-798.
64. Ross, A. C., Liu, H., Pattabiraman, V. R., Vederas, J. C. (2009) Synthesis of the lantibiotic lactocin S using peptide cyclizations on solid phase, J Am Chem Soc 132, 462-463.
65. van Kraaij, C., de Vos, W. M., Siezena, R. J., Kuipers, O. P. (1999) Lantibiotics: biosynthesis, mode of action and applications, Nat Prod Rep 16, 575-587.
66. Cotter, P. D., Deegan, L. H., Lawton, E. M., Draper, L. A., O'Connor, P. M., Hill, C., Ross, R. P. (2006) Complete alanine scanning of the two-component lantibiotic lacticin 3147: generating a blueprint for rational drug design, Mol Microbiol 62, 735-747.
67. Appleyard, A. N., Choi, S., Read, D. M., Lightfoot, A., Boakes, S., Hoffmann, A., Chopra, I., Bierbaum, G., Rudd, B. A. M., Dawson, M. J., Cortes, J. (2009) Dissecting structural and functional diversity of the lantibiotic mersacidin, Chem Biol 16, 490-498.
68. Piper, C., Cotter, P. D., Ross, R. P., Hill, C. (2009) Discovery of medically significant lantibiotics, Curr Drug Discov Technol 6, 1-18.
69. Lee, M. V., Ihnken, L. A. F., You, Y. O., McClerren, A. L., van der Donk, W. A., Kelleher, N. L. (2009) Distributive and directional behavior of lantibiotic synthetases revealed by high-resolution tandem mass spectrometry, J Am Chem Soc 131, 12258-12264.
70. Patton, G. C., Paul, M., Cooper, L. E., Chatterjee, C., van der Donk, W. A. (2008) The importance of the leader sequence for directing lanthionine formation in lacticin 481, Biochemistry 47, 7342-7351.
71. Cheng, F., Takala, T. M., Saris, P. E. J. (2007) Nisin biosynthesis in vitro, J Mol Microbiol Biotechnol 13, 248-254.
72. McClerren, A. L., Cooper, L. E., Quan, C., Thomas, P. M., Kelleher, N. L., van der Donk, W. A. (2006) Discovery and in vitro biosynthesis of haloduracin, a two-component lantibiotic, Proc Natl Acad Sci USA. 103, 17243-17248.
73. Müller, W. M., Schmiederer, T., Ensle, P., Süssmuth, R. D. (2010) In vitro biosynthesis of the prepeptide of type-III lantibiotic labyrinthopeptin A2 including formation of a C—C bond as a post-translational modification, Angew Chem Int Ed Engl 49, 2436-2440.
74. Goto, Y., Li, B., Claesen, J., Shi, Y., Bibb, M. J., van der Donk, W. A. (2010) Discovery of unique lanthionine synthetases reveals new mechanistic and evolutionary insights PLoS Biology 8, e1000339.
75. Li, B., Sher, D., Kelly, L., Shi, Y., Huang, K., Knerr, P. J., Joewono, I., Rusch, D., Chisholm, S. W., van der Donk, W. A. (2010) Catalytic promiscuity in the biosynthesis of cyclic peptide secondary metabolites in planktonic marine cyanobacteria, Proc Natl Acad Sci USA 107, 10430-10435.
76. Osapay, G., Prokai, L., Kim, H.-S., Medzihradszky, K. F., Coy, D. H., Liapakis, G., Reisine, T., Melacini, G., Zhu, Q., Wang, S. H. H., Mattern, R.-H., Goodman, M. (1997) Lanthionine-somatostatin analogs: synthesis, characterization, biological activity, and enzymatic stability studies, J Med Chem 40, 2241-2251.
77. Rew, Y., Malkmus, S., Svensson, C., Yaksh, T. L., Chung, N. N., Schiller, P. W., Cassel, J. A., DeHaven, R. N., Taulane, J. P., Goodman, M. (2002) Synthesis and biological activities of cyclic lanthionine enkephalin analogues: delta-opioid receptor selective ligands, J Med Chem 45, 3746-3754.
78. Hegemann, P. (2002) Method for producing nucleic acid polymers. U.S. Pat. No. 6,472,184.
79. Lefèvre, F., Rémy, M.-H., Masson, J.-M. (1997) Alanine-stretch scanning mutagenesis: a simple and efficient method to probe protein structure and function, Nucleic Acids Res 25, 447-448.
80. Bierbaum, G., Szekat, C., Josten, M., Heidrich, C., Kempter, C., Jung, G., Sahl, H. G. (1996) Engineering of a novel thioether bridge and role of modified residues in the lantibiotic Pep5, Appl Environ Microbiol 62, 4332-4338.
81. Szekat, C., Jack, R. W., Skutlarek, D., Farber, H., Bierbaum, G. (2003) Construction of an expression system for site-directed mutagenesis of the lantibiotic mersacidin, Appl Environ Microbiol 69, 3777-3783
82. Groner, B., (Ed.) (2009) Peptides as drugs: discovery and development, WILEY-VCH, Weinheim.
83. Moll, G., Kuipers, A., Rink, R. (2010) Microbial engineering of dehydro-amino acids and lanthionines in non-lantibiotic peptides, Antonie Van Leeuwenhoek 97, 319-333.
84. Rink, R., Kuipers, A., de Boef, E., Leenhouts, K. J., Driessen, A. J., Moll, G. N., Kuipers, O. P. (2005) Lantibiotic structures as guidelines for the design of peptides that can be modified by lantibiotic enzymes, Biochemistry 44, 8873-8882.
85. Rink, R., Wierenga, J., Kuipers, A., Kluskens, L. D., Driessen, A. J., Kuipers, O. P., Moll, G. N. (2007) Dissection and modulation of the four distinct activities of nisin by mutagenesis of rings A and B and by C-terminal truncation, Appl Environ Microbiol 73, 5809-5816.
86. Meindl, K., Schmiederer, T., Schneider, K., Reicke, A., Butz, D., Keller, S., Gühring, H., Vértesy, L., Wink, J., Hoffmann, H., Brönstrup, M., Sheldrick, G. M., Süssmuth, R. D. (2010) Labyrinthopeptins: a new class of carbacyclic lantibiotics, Angew Chem Int Ed Engl 49, 1151.
87. Sambrook, J., Fritsch, E. F., Maniatis, T. (1989) Molecular Cloning. A Laboratory Manual (2nd ed.), Cold Spring Harbor Laboratory Press, Cold Spring Harbor, N. Y.
88. Wright, F., Bibb, M. J. (1992) Codon usage in the G+C-rich Streptomyces genome, Gene 113, 55-65.
89. Kaiser, R., Metzka, L. (1999) Enhancement of cyanogen bromide cleavage yields for methionyl-serine and methionyl-threonine peptide bonds, Anal Biochem 266, 1-8.
90. Feitelson, J. S., Englenwood, N. J. (1991) Multifunctional plasmid vectors from Actinomadura and Escherichia coli, U.S. Pat. No. 5,002,891.
91. Bierman, M., Logan, R., O'Brien, K., Seno, E. T., Nagaraja Rao, R., Schoner, B. E. (1992) Plasmid cloning vectors for the conjugal transfer of DNA from Escherichia coli to Streptomyces spp., Gene 116, 43-49.
92. Combes, P., Till, R., Bee, S., Smith, M. C. M. (2002) The Streptomyces genome contains multiple pseudo-attB sites for the (phi)C31-encoded site-specific recombination system, J Bacteriol 184, 5746-5752.

1. A nucleic acid comprising at least one Labyrinthopeptin (lab) gene cluster comprising a sequence according to SEQ ID No. 139, wherein:
(a) the nucleic acid further comprises a constitutive promoter for expression in Streptomyces cells and wherein the gene cluster is under control of said promoter, or
(b) the nucleic acid encoding LabA1 or LabA2 or a functional derivative thereof is mutated, such that the C-terminal amino acid of the leader peptide of LabA1 or LabA2 is substituted with a methionine, or
(c) the nucleic acid further comprises at least one hybrid gene sequence, wherein the hybrid gene sequence:
(i) encodes a pre-pro-peptide comprising a LabA2 leader sequence and LabA1 pre-peptide or functional derivative thereof and/or
(ii) encodes a pre-pro-peptide comprising a LabA1 leader sequence and LabA2 pre-peptide or functional derivative thereof.

2. The nucleic acid according to claim 1,
(a) wherein the Labyrinthopeptin (lab) gene cluster comprises the genes labKC, labA1, labA2, labT1 and labT2, or
(b) wherein the nucleic acid encoding LabA1 and/or LabA2 has a sequence according to SEQ ID No. 140 or SEQ ID No. 141, or has a sequence exhibiting at least 85%, sequence identity to SEQ ID No. 140 or SEQ ID No. 141.

3. A vector comprising a nucleic acid according to claim 1.

4. The vector according to claim 3, wherein the vector is:
(a) pLab, having a sequence according to SEQ ID No. 143, or pUWLab, having a sequence according to SEQ ID No. 142, or pLabAmp, having a sequence according to SEQ ID No. 144, or
(b) obtainable from pUWLoriT by inserting at least one lab gene cluster, such that the gene cluster is under control of the constitutive promotor.

5. A bacterial cell comprising a vector according to claim 3.

6. A method for producing at least one Labyrinthopeptin or functional derivative thereof, comprising the steps:
(a) culturing a Streptomyces cell comprising a vector according to claim 3 in culture medium,
(b) harvesting the culture medium, wherein the culture medium comprises at least one Labyrinthopeptin or functional derivative thereof, and
(c) optionally purifying the at least one Labyrinthopeptin or functional derivative thereof from the culture medium.

7. (canceled)

8. A mixture of more than one Labyrinthopeptin or functional derivatives thereof, obtainable by the method according to claim 6.

9. A composition comprising:
(a) a Labyrinthopeptin pre-pro-peptide Labyrinthopeptin or a functional derivative thereof, wherein the C-terminal amino acid of the leader sequence is a methionine, or
(b) a nucleic acid encoding a Labyrinthopeptin pre-pro-peptide or a functional derivative thereof, wherein the C-terminal amino acid of the leader sequence of the pre-pro-peptide is a methionine.

10. A composition comprising:
(a) a nucleic acid comprising a sequence according to any of SEQ ID No. 1 to 24, or
(b) a functional derivative of a Labyrinthopeptin pre-pro-peptide encoded by a nucleic acid of (a), and which has a sequence different from LabA1 pre-pro-peptide according to SEQ ID No. 132 or LabA2 pre-pro-peptide according to SEQ ID No. 135, or
(c) a functional derivative of a Labyrinthopeptin pre-peptide encoded by a nucleic acid of (a), and which has a sequence different from LabA1 pre-peptide according to SEQ ID No. 134 or LabA2 pre-peptide according to SEQ ID No. 137, or LabA3 pre-peptide according to SEQ ID No. 138 and wherein the derivative lacks the leader sequence or at least the N-terminal 80%, of the leader sequence, or
(d) a functional derivative of a Labyrinthopeptin peptide selected from: LabA1_N2A, LabA1_V5A, LabA1_W6A, LabA1_E7A, LabA1_T11A, LabA1_V15A, LabA1_P16A, LabA1_F17A, LabA2_D2A, LabA2_W3A, LabA2_L5A, LabA2_E7A, LabA2_T11A, LabA2_G12A, LabA2_L14A, LabA2_F15A, LabA1_V5del, LabA1_W6insV, LabA1_{—}16del, LabA1_P16insV, LabA1_S4T, M-LabA1_S4T, AM-LabA1_S4T, NR-LabA2_M (SG20), R-LabA2_M (SG20), LabA1_V15S, M-LabA1_V15S, LabA1_T11S (Ser), LabA1_W6Y, LabA1_A3H, LabA1_C20insA, LabA1_S10insA, LabA1_C20del, LabA1_S1insC, D-LabA1/A2 (SG11), AD-LabA1/A2 (SG11), R-LabA2/A1 (SG11), NR-LabA2/A1 (SG11), and ENR-LabA2/A1 (SG11).

11. Use of at least one Streptomyces lividans or Streptomyces albus cell for the recombinant expression of at least one Labyrinthopeptin or a functional derivative thereof.

12. The composition according to claim 9, for use in the treatment or prevention of a bacterial infection, HIV infection or pain.

13. The composition of claim 9, for use in the treatment or prevention of neuropathic pain.

14. The nucleic acid of claim 1, wherein the constitutive promoter is an ermE promoter.

15. The bacterial cell of claim 5, wherein the cell is a Streptomyces cell or an E. coli cell.

16. The bacterial cell of claim 5, wherein the cell is a Streptomyces albus cell or a Streptomyces lividans cell.

17. The method of claim 6, wherein the vector is:
(a) pLab, having a sequence according to SEQ ID No. 143, or pUWLab, having a sequence according to SEQ ID No. 142, or pLabAmp, having a sequence according to SEQ ID No. 144, or
(b) obtainable from pUWLoriT by inserting at least one lab gene cluster, such that the gene cluster is under control of the constitutive promotor.

18. The method of claim 6, wherein the Streptomyces cell is a Streptomyces albus cell or a Streptomyces lividans cell.
